(19) United States
(12) Patent Application Publication
(10) Pub. No.: US 2012/0149886 A1
(43) Pub. Date: Jun. 14, 2012

(54) PRODUCTION OF CAROTENOIDS IN OLEAGINOUS YEAST AND FUNGI

(75) Inventors: Richard B. Bailey, South Natick, MA (US); Kevin T. Madden, Arlington, MA (US); Joshua Trueheart, Concord, MA (US); Reed Doten, Framingham, MA (US); Maria Mayorga, Medford, MA (US); Joshua Griffin Dunn, Boston, MA (US); Dan Dueppen, Woburn, MA (US)

(73) Assignee: MICROBIA, INC., Lexington, MA (US)

(21) Appl. No.: 12/937,204

(22) PCT Filed: Apr. 10, 2009
(86) PCT No.: PCT/US2009/040214

§ 371 Date: Feb. 15, 2012

Related U.S. Application Data
(60) Provisional application No. 61/043,958, filed on Apr. 10, 2008.

(51) Int. Cl. C07H 15/10; C12P 17/02; C12P 19/02; C12P 7/26; C12P 7/22; C12P 5/00; C12P 5/02; C12N 1/15; C12N 1/19; C07D 405/06; C07D 303/12; C07C 49/653; C07C 35/00; C07C 11/02; C07C 13/28; C12P 17/04

(52) U.S. Cl. 536/4.1; 435/105; 435/123; 435/126; 435/148; 435/156; 435/166; 435/167; 435/254.11; 435/254.2; 549/320; 549/546; 568/367; 568/816; 585/16; 585/23

(57) ABSTRACT

The present disclosure provides systems for producing engineered oleaginous yeast or fungi that express carotenoids.

### CROSS REFERENCE TO RELATED APPLICATIONS

The present application is copending with, shares at least one common inventor with and claims priority to U.S. provisional patent application Ser. No. 61/043,958, filed Apr. 10, 2008, the entire contents of which are incorporated herein by reference.

### BACKGROUND OF THE DISCLOSURE

Carotenoids are organic pigments ranging in color from yellow to red that are naturally produced by certain organisms, including photosynthetic organisms (e.g., plants, algae, cyanobacteria), and some fungi. Carotenoids are responsible for the orange color of carrots, as well as the pink in flamingos and salmon, and the red in lobsters and shrimp. Animals, however, cannot produce carotenoids and must receive them through their diet.

Carotenoid pigments (e.g., β-carotene and astaxanthin) are used industrially as ingredients for food and feed stocks, both serving a nutritional function and enhancing consumer acceptability. For example, astaxanthin is widely used in salmon aquaculture to provide the orange coloration characteristic of their wild counterparts. Some carotenoids are also precursors of vitamin A. Also, carotenoids have antioxidant properties, and may have various health benefits (see, for example, Jyonouchi et al., Nutr. Cancer 16:93, 1991; Giovannucci et al., J. Natl. Cancer Inst. 87:1767, 1995; Miki, Pure Appl. Chem. 63:141, 1991; Chew et al., Anticancer Res. 19:1849, 1999; Wang et al., Antimicrob. Agents Chemother. 44:2452, 2000). Some carotenoids such as β-carotene, lycopene, and lutein are currently sold as nutritional supplements.

In general, the biological systems that produce carotenoids are industrially intractable and/or produce the compounds at such low levels that commercial scale isolation is not practicable. Thus, most carotenoids used in industry are produced by chemical synthesis. There is a need for improved biological systems that produce carotenoids. Some efforts have previously been made to genetically engineer certain bacteria or fungi to produce higher levels of carotenoids (see, for example, Misawa et al., J. Biotechnol. 59:169, 1998; Visser et al., FEMS Yeast Research 4:221, 2003). However, improved systems, allowing higher levels of production and greater ease of isolation, are needed.

### SUMMARY OF THE DISCLOSURE

The present disclosure provides improved systems for the biological production of carotenoids and/or retinolic compounds. In one aspect, the disclosure encompasses the discovery that it is desirable to produce carotenoids and/or retinolic compounds in oleaginous organisms. Without wishing to be bound by any particular theory, the present inventors propose that biological systems may be able to accumulate higher levels of carotenoids and/or retinolic compounds if the compounds are sequestered in lipid bodies. Regardless of whether absolute levels are higher, however, carotenoids and/or retinolic compounds that are accumulated within lipid bodies in oleaginous organisms are readily isolatable through isolation of the lipid bodies.

The present disclosure therefore provides oleaginous fungi (including, for example, yeast) that produce one or more carotenoids and/or retinolic compounds. The present disclosure also provides methods of constructing such yeast and fungi, methods of using such yeast and fungi to produce carotenoids and/or retinolic compounds, and methods of preparing carotenoid-containing compositions and/or retinolic compound-containing compositions, such as food or feed additives, or nutritional supplements, using carotenoids and/or retinolic compounds produced in such oleaginous yeast or fungi. In particular, the present disclosure provides systems and methods for generating yeast and fungi containing one or more oleaginic and/or carotenogenic and/or retinologenic modifications that increase the oleaginicity and/or alter their carotenoid-producing and/or retinolic compound-producing capabilities as compared with otherwise identical organisms that lack the modification(s).

The present disclosure further encompasses the general recognition that lipid-accumulating systems are useful for the production and/or isolation of lipophilic agents (such as, but not limited to isoprenoids, or isoprenoid-derived compounds such as retinolic compounds, carotenoids, ubiquinones, lanosterol, zymosterol, ergosterol, vitamins (e.g., vitamins A, E, D, K, specifically 7-dehydrocholesterol (provitamin D3), sterols (e.g., squalene), etc.). According to the present disclosure, it is desirable to engineer organisms to produce such lipophilic agents and/or to accumulate lipid.

Indeed, one aspect of the present disclosure is the recognition that host cells can be engineered to accumulate in lipid bodies any of a variety of hydrophilic and/or fat soluble compounds (e.g., retinolic compounds, carotenoids, ubiquinones, vitamins, squalene, etc.) having negligible solubility in water (whether hot or cold) and an appropriate solubility in oil. In some embodiments of the disclosure, modified host cells are engineered to produce one or more lipophilic agents characterized by negligible solubility in water and detectable solubility in one or more oils. In some embodiments, such lipophilic agents (including, but not limited to carotenoids and/or retinolic compounds) have a solubility in oil below about 0.2%. In some embodiments, such lipophilic agents have a solubility in oil within the range of about <0.001%-0.2%.

The present disclosure therefore provides engineered host cells (and methods of making and using them) that contain lipid bodies and that further contain one or more compounds accumulated in the lipid bodies, where the compounds are characterized by a negligible solubility in water and a solubility in oil within the range of about <0.001%-0.2%; 0.004%-0.15%; 0.005-0.1%; or 0.005-0.5%. For example, in some embodiments, such lipophilic agents have a solubility in oil below about 0.15%, 0.14%, 0.13%, 0.12%, 0.11%, 0.10%, 0.09, 0.08%, 0.07%, 0.06%, 0.05%, 0.04%, 0.03%, 0.02%, 0.01%, 0.05%, or less. In some embodiments, the lipophilic agents show such solubility in an oil selected from the group consisting of sesame; soybean; apricot kernel; palm; peanut; safflower; coconut; olive; cocoa butter; palm kernel; shea butter; sunflower; almond; avocado; borage; carnauba; hazel nut; castor; cotton seed; evening primrose; orange roughy; rapeseed; rice bran; walnut; wheat germ; peach kernel; babassu; mango seed; black current seed; jojoba; macademia nut; sea buckthorn; sasquana; tsubaki; mallow; meadowfoam seed; coffee; emu; mink; grape seed; thistle; tea tree; pumpkin seed; kukui nut; and mixtures thereof.

In some embodiments, the present disclosure provides a recombinant fungus. In certain embodiments, the recombinant fungus is oleaginous in that it can accumulate lipid to at least about 20% of its dry cell weight; and produces at least one carotenoid and/or retinolic compound, and can accumulate the produced carotenoid and/or retinolic compound to at least about 1% of its dry cell weight; wherein the recombinant fungus comprises at least one modification as compared with a parental fungus, which parental fungus both is not oleaginous and does not accumulate the carotenoid and/or retinolic compound to at least about 1% of its dry cell weight, the at least one modification being selected from the group consisting of retinologenic modifications, carotenogenic modifications, oleaginic modifications, and combinations thereof, and wherein the at least one modification alters oleaginicity of the recombinant fungus, confers to the recombinant fungus oleaginy, confers to the recombinant fungus the ability to produce the at least one carotenoid and/or retinolic compound to a level at least about 1% of its dry cell weight, or confers to the recombinant fungus the ability to produce at least one carotenoid and/or retinolic compound which the parental fungus does not produce.

In other embodiments, the recombinant fungus is oleaginous in that it can accumulate lipid to at least about 20% of its dry cell weight; and produces at least one carotenoid selected from the group consisting of antheraxanthin, adonirubin, adonixanthin, astaxanthin, canthaxanthin, capsorubrin, β-cryptoxanthin, α-carotene, β,ψ-carotene, δ-carotene, ε-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, a C30 carotenoid, and combinations thereof, and can accumulate the produced carotenoid to at least about 1% of its dry cell weight; wherein the recombinant fungus comprises at least one modification as compared with a parental fungus, the at least one modification being selected from the group consisting of carotenogenic modifications, oleaginic modifications, and combinations thereof, and wherein the at least one modification alters oleaginicity of the recombinant fungus, confers to the recombinant fungus oleaginy, confers to the recombinant fungus the ability to produce the at least one carotenoid to a level at least about 1% of its dry cell weight, or confers to the recombinant fungus the ability to produce at least one carotenoid which the parental fungus does not naturally produce.

In other embodiments, the recombinant fungus is oleaginous in that it can accumulate lipid to at least about 20% of its dry cell weight; and produces at least one retinolic compound selected from the group consisting of retinol, retinal, retinoic acid, and combinations thereof, and can accumulate the produced retinolic compound to at least about 1% of its dry cell weight; wherein the recombinant fungus comprises at least one modification as compared with a parental fungus, the at least one modification being selected from the group consisting of retinologenic modifications, carotenogenic modifications, oleaginic modifications, and combinations thereof, and wherein the at least one modification alters oleaginicity of the recombinant fungus, confers to the recombinant fungus oleaginy, confers to the recombinant fungus the ability to produce the at least one retinolic compound to a level at least about 1% of its dry cell weight, or confers to the recombinant fungus the ability to produce at least one retinolic compound which the parental fungus does not naturally produce.

In some embodiments, the recombinant fungus is oleaginous in that it can accumulate lipid to at least about 20% of its dry cell weight; and produces at least one carotenoid and/or retinolic compound, and can accumulate the produced carotenoid and/or retinolic compound to at least about 1% of its dry cell weight; wherein the recombinant fungus is a member of a genus selected from the group consisting of: Aspergillus, Blakeslea, Botrytis, Candida, Cercospora, Cryptococcus, Cunninghamella, Fusarium (Gibberella), Kluyveromyces, Lipomyces, Mortierella, Mucor, Neurospora, Penicillium, Phycomyces, Pichia (Hansenula), Puccinia, Pythium, Rhodosporidium, Rhodotorula, Saccharomyces, Sclerotium, Trichoderma, Trichosporon, Xanthophyllomyces (Phaffia), and Yarrowia; or is a species selected from the group consisting of: Aspergillus terreus, Aspergillus nidulans, Aspergillus niger, Blakeslea trispora, Botrytis cinerea, Candida japonica, Candida pulcherrima, Candida revkaufi, Candida tropicalis, Candida utilis, Cercospora nicotianae, Cryptococcus curvatus, Cunninghamella echinulata, Cunninghamella elegans, Fusarium fujikuroi (Gibberella zeae), Kluyveromyces lactis, Lipomyces starkeyi, Lipomyces lipoferus, Mortierella alpina, Mortierella ramanniana, Mortierella isabellina, Mortierella vinacea, Mucor circinelloides, Neurospora crassa, Phycomyces blakesleanus, Pichia pastoris, Puccinia distincta, Pythium irregulare, Rhodosporidium toruloides, Rhodotorula glutinis, Rhodotorula graminis, Rhodotorula mucilaginosa, Rhodotorula pinicola, Rhodotorula gracilis, Saccharomyces cerevisiae, Sclerotium rolfsii, Trichoderma reesei, Trichosporon cutaneum, Trichosporon pullans, Xanthophyllomyces dendrorhous (Phaffia rhodozyma), and Yarrowia lipolytica; wherein the recombinant fungus comprises at least one modification as compared with a parental fungus, the at least one modification being selected from the group consisting of retinologenic modifications, carotenogenic modifications, oleaginic modifications, and combinations thereof, and wherein the at least one modification alters oleaginicity of the recombinant fungus, confers to the recombinant fungus oleaginy, confers to the recombinant fungus the ability to produce the at least one carotenoid and/or retinolic compound to a level at least about 1% of its dry cell weight, or confers to the recombinant fungus the ability to produce at least one carotenoid and/or retinolic compound which the parental fungus does not naturally produce.

In other embodiments, the recombinant fungus is oleaginous in that it can accumulate lipid to at least about 20% of its dry cell weight; and produces at least one carotenoid selected from the group consisting of antheraxanthin, adonirubin, adonixanthin, astaxanthin, canthaxanthin, capsorubrin, β-cryptoxanthin, α-carotene, β,ψ-carotene, δ-carotene, ζ-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, a C30 carotenoid, and combinations thereof, and can accumulate the produced carotenoid to at least about 1% of its dry cell weight; wherein the recombinant fungus is a member of a genus selected from the group consisting of: Aspergillus, Blakeslea, Botrytis, Candida, Cercospora, Cryptococcus, Cunninghamella, Fusarium (Gibberella), Kluyveromyces, Lipomyces, Mortierella, Mucor, Neurospora, Penicillium, Phycomyces, Pichia (Hansenula), Puccinia, Pythium, Rhodosporidium, Rhodotorula, Saccharomyces, Sclerotium, Trichoderma, Trichosporon, Xanthophyllomyces (Phaffia), and Yarrowia, or is of a species selected from the group consisting of: Aspergillus terreus, Aspergillus nidulans, Aspergillus niger, Blakeslea trispora, Botrytis cinerea, Candida japonica, Candida pulcherrima, Candida revkaufi, Candida tropicalis, Candida utilis, Cercospora nicotianae, Cryptococcus curvatus, Cunninghamella echinulata, Cunninghamella elegans, Fusarium fujikuroi (Gibberella zeae), Kluyveromyces lactis, Lipomyces starkeyi, Lipomyces lipoferus, Mortierella alpina, Mortierella ramanniana, Mortierella isabellina, Mortierella vinacea, Mucor circinelloides, Neurospora crassa, Phycomyces blakesleanus, Pichia pastoris, Puccinia distincta, Pythium irregulare, Rhodosporidium toruloides, Rhodotorula glutinis, Rhodotorula graminis, Rhodotorula mucilaginosa, Rhodotorula pinicola, Rhodotorula gracilis, Saccharomyces cerevisiae, Sclerotium rolfsii, Trichoderma reesei, Trichosporon cutaneum, Trichosporon pullans, Xanthophyllomyces dendrorhous (Phaffia rhodozyma), and Yarrowia lipolytica, wherein the recombinant fungus comprises at least one modification as compared with a parental fungus, the at least one modification being selected from the group consisting of carotenogenic modifications, oleaginic modifications, and combinations thereof, and wherein the at least one modification alters oleaginicity of the recombinant fungus, confers to the recombinant fungus oleaginy, confers to the recombinant fungus the ability to produce the at least one carotenoid to a level at least about 1% of its dry cell weight, or confers to the recombinant fungus the ability to produce at least one carotenoid which the parental fungus does not naturally produce.

In other embodiments, the recombinant fungus is oleaginous in that it can accumulate lipid to at least about 20% of its dry cell weight; and produces at least one retinolic compound selected from the group consisting of retinol, retinal, retinoic acid, and combinations thereof, and can accumulate the produced retinolic compound to at least about 1% of its dry cell weight; wherein the recombinant fungus is a member of a genus selected from the group consisting of: Aspergillus, Blakeslea, Botrytis, Candida, Cercospora, Cryptococcus, Cunninghamella, Fusarium (Gibberella), Kluyveromyces, Lipomyces, Mortierella, Mucor, Neurospora, Penicillium, Phycomyces, Pichia (Hansenula), Puccinia, Pythium, Rhodosporidium, Rhodotorula, Saccharomyces, Sclerotium, Trichoderma, Trichosporon, Xanthophyllomyces (Phaffia), and Yarrowia, or is of a species selected from the group consisting of: Aspergillus terreus, Aspergillus nidulans, Aspergillus niger, Blakeslea trispora, Botrytis cinerea, Candida japonica, Candida pulcherrima, Candida revkaufi, Candida tropicalis, Candida utilis, Cercospora nicotianae, Cryptococcus curvatus, Cunninghamella echinulata, Cunninghamella elegans, Fusarium fujikuroi (Gibberella zeae), Kluyveromyces lactis, Lipomyces starkeyi, Lipomyces lipoferus, Mortierella alpina, Mortierella ramanniana, Mortierella isabellina, Mortierella vinacea, Mucor circinelloides, Neurospora crassa, Phycomyces blakesleanus, Pichia pastoris, Puccinia distincta, Pythium irregulare, Rhodosporidium toruloides, Rhodotorula glutinis, Rhodotorula graminis, Rhodotorula mucilaginosa, Rhodotorula pinicola, Rhodotorula gracilis, Saccharomyces cerevisiae, Sclerotium rolfsii, Trichoderma reesei, Trichosporon cutaneum, Trichosporon pullans, Xanthophyllomyces dendrorhous (Phaffia rhodozyma), and Yarrowia lipolytica, wherein the recombinant fungus comprises at least one modification as compared with a parental fungus, the at least one modification being selected from the group consisting of retinologenic modifications, carotenogenic modifications, oleaginic modifications, and combinations thereof, and wherein the at least one modification alters oleaginicity of the recombinant fungus, confers to the recombinant fungus oleaginy, confers to the recombinant fungus the ability to produce the at least one retinolic compound to a level at least about 1% of its dry cell weight, or confers to the recombinant fungus the ability to produce at least one retinolic compound which the parental fungus does not naturally produce.

In certain embodiments, the recombinant fungus is oleaginous in that it can accumulate lipid to at least about 20% of its dry cell weight; and the recombinant fungus produces at least one small molecule lipophilic agent selected from the group consisting of retinolic compounds, carotenoids, ubiquinone, vitamin K, vitamin E, squalene, lanosterol, zymosterol, ergosterol, 7-dehydrocholesterol (provitamin D3), and combinations thereof and can accumulate the produced carotenoid and/or retinolic compound to at least about 1% of its dry cell weight; wherein the recombinant fungus comprises at least one modification as compared with a parental fungus, the at least one modification being selected from the group consisting of retinologenic modifications, carotenogenic modifications, oleaginic modifications, and combinations thereof, and wherein the at least one modification alters oleaginicity of the recombinant fungus, confers to the recombinant fungus oleaginy, confers to the recombinant fungus the ability to produce the at least one carotenoid and/or retinolic compound to a level at least about 1% of its dry cell weight, or confers to the recombinant fungus the ability to produce at least one carotenoid and/or retinolic compound which the parental fungus does not naturally produce.

In some embodiments, the recombinant fungus is oleaginous in that it can accumulate lipid to at least about 20% of its dry cell weight; and the recombinant fungus produces at least one small molecule lipophilic agent characterized by a negligible solubility in water and solubility in oil within the range of about <0.001%-0.2%; 0.004%-0.15%; 0.005-0.1%; or 0.005-0.5%, and combinations thereof and can accumulate the produced small molecule lipophilic agent to at least about 1% of its dry cell weight; wherein the recombinant fungus comprises at least one modification as compared with a parental fungus, the at least one modification being selected from the group consisting of retinologenic modifications, carotenogenic modifications, oleaginic modifications, and combinations thereof, and wherein the at least one modification alters oleaginicity of the recombinant fungus, confers to the recombinant fungus oleaginy, confers to the recombinant fungus the ability to produce the at least one small molecule lipophilic agent to a level at least about 1% of its dry cell weight, or confers to the recombinant fungus the ability to produce at least one small molecule lipophilic agent which the parental fungus does not naturally produce.

In other embodiments the recombinant fungus is oleaginous in that it can accumulate lipid to at least about 20% of its dry cell weight; and the recombinant fungus produces at least one small molecule lipophilic agent selected from the group consisting of retinolic compounds, carotenoids, ubiquinone, vitamin K, vitamin E, squalene, lanosterol, zymosterol, ergosterol, 7-dehydrocholesterol (provitamin D3), and can accumulate the produced small molecule lipophilic agent to at least about 1% of its dry cell weight; wherein the recombinant fungus is a member of a genus selected from the group consisting of Candida, Cryptococcus, Cunninghamella, Lipomyces, Mortierella, Mucor, Phycomyces, Pythium, Rhodosporidium, Rhodotorula, Trichosporon, Yarrowia, Aspergillus, Botrytis, Cercospora, Fusarium (Gibberella), Kluyveromyces, Neurospora, Penicillium, Pichia (Hansenula), Puccinia, Saccharomyces, Schizosaccharomyces, Sclerotium, Trichoderms, Ustilago, and Xanthophyllomyces (Phaffia) and comprises at least one genetic modification as compared with a parental fungus, wherein the at least one modification alters oleaginicity of the recombinant fungus, confers to the recombinant fungus oleaginy, confers to the recombinant fungus the ability to produce the at least one small molecule lipophilic agent to a level at least about 1% of its dry cell weight, or confers to the recombinant fungus the ability to produce at least one small molecule lipophilic agent which the parental fungus does not naturally produce.

In some embodiments, the present disclosure provides a strain of Yarrowia lipolytica comprising one or more modifications selected from the group consisting of an oleaginic modification, a carotenogenic modification, and combinations thereof, such that the strain accumulates from 1% to 15% of its dry cell weight as at least one carotenoid. In some embodiments, the present disclosure provides a strain of Yarrowia lipolytica comprising one or more modifications selected from the group consisting of an oleaginic modification, a retinologenic modification, and combinations thereof, such that the strain accumulates from 1% to 15% of its dry cell weight as at least one retinolic compound.

In some embodiments, the present disclosure provides an engineered Y. lipolytica strain that produces β-carotene, the strain containing one or more carotenogenic modifications selected from the group consisting of: increased expression or activity of a Y. lipolytica GGPP synthase polypeptide; expression or activity of a truncated HMG CoA reductase polypeptide; expression or activity of a phytoene dehydrogenase polypeptide; expression or activity of a phytoene synthase/lycopene cyclase polypeptide; increased expression or activity of an FPP synthase polypeptide; increased expression or activity of an IPP isomerase polypeptide; increased expression or activity of an HMG synthase polypeptide; increased expression or activity of a mevalonate kinase polypeptide; increased expression or activity of a phosphomevalonate kinase polypeptide; increased expression or activity of a mevalonate pyrophosphate decarboxylate polypeptide; increased expression or activity of a malic enzyme polypeptide; increased expression or activity of a malate dehydrogenase polypeptide; increased expression or activity of an AMP deaminase polypeptide; increased expression or activity of a glucose 6 phosphate dehydrogenase polypeptide; increased expression or activity of a malate dehydrogenase homolog2 polypeptide; increased expression or activity of a GND1-6-phosphogluconate dehydrogenase polypeptide; increased expression or activity of a isocitrate dehydrogenase polypeptide; increased expression or activity of a IDH2-isocitrate dehydrogenase polypeptide; increased expression or activity of a fructose 1,6 bisphosphatase polypeptide; increased expression or activity of a Erg10-acetoacetyl CoA thiolase polypeptide; increased expression or activity of a ATP citrate lyase subunit 2 polypeptide; increased expression or activity of a ATP citrate lyase subunit 1 polypeptide; decreased expression or activity of a squalene synthase polypeptide; decreased expression or activity of a prenyldiphosphate synthase polypeptide; or decreased expression or activity of a PHB polyprenyltransferase polypeptide; and combinations thereof.

In some embodiments, the present disclosure provides an engineered Y. lipolytica strain that produces Vitamin A, the strain containing one or more retinologenic modifications selected from the group consisting of: increased expression or activity of a beta-carotene 15,15′-monooxygenase polypeptide; increased expression or activity of a retinol dehydrogenase polypeptide; and combinations thereof.

In some embodiments, the present disclosure provides an engineered Y. lipolytica strain containing a truncated HMG CoA reductase polypeptide. In some embodiments, the present disclosure provides an engineered Y. lipolytica strain having increased expression or activity of a GGPP synthase gene. In some embodiments, the present disclosure provides an engineered Y. lipolytica strain having decreased expression or activity of a squalene synthase polypeptide. In some embodiments, the present disclosure provides an engineered Y. lipolytica strain containing a heterologous phytoene dehydrogenase (carB) polypeptide and a heterologous phytoene synthase/lycopene cyclase (carRP) polypeptide.

In some embodiments, the present disclosure provides a genetically modified Y. lipolytica strain comprising an altered activity or expression of one or more enzymes when compared to an unmodified strain, wherein the altered activity or expression of one or more enzymes is selected from the group consisting of: increased activity or expression of a beta-carotene 15,15′-monooxygenase polypeptide; increased activity or expression of a retinol dehydrogenase polypeptide; increased activity or expression of acetyl-CoA thiolase, increased activity or expression of HMG-CoA synthase, increased activity or expression of HMG-CoA reductase, increased activity or expression of mevalonate kinase, increased activity or expression of phosphomevalonate kinase, increased activity or expression of mevalonate PP decarboxylase, decreased activity or expression of acetyl-CoA carboxylase, increased activity or expression of IPP isomerase, increased activity or expression of GPP synthase, increased activity or expression of FPP synthase, increased activity or expression of squalene synthase, decreased activity or expression of squalene synthase, increased activity or expression of GGPP synthase, decreased activity or expression of GGPP synthase, increased activity or expression of glucose-6-phosphate dehydrogenase, increased activity or expression of 6-phosphogluconate dehydrogenase, increased activity or expression of fructose 1, 6 bisphosphatase, increased activity or expression of NADH kinase, increased activity or expression of transhydrogenase, and combinations thereof.

In certain embodiments, the present disclosure provides a genetically modified Candida utilis strain comprising an altered activity or expression of one or more enzymes when compared to an unmodified strain, wherein the altered activity or expression of one or more enzymes is selected from the group consisting of: increased activity or expression of a beta-carotene 15,15′-monooxygenase polypeptide; increased activity or expression of a retinol dehydrogenase polypeptide; increased activity or expression of acetyl-CoA thiolase, increased activity or expression of HMG-CoA synthase, increased activity or expression of HMG-CoA reductase, increased activity or expression of mevalonate kinase, increased activity or expression of phosphomevalonate kinase, increased activity or expression of mevalonate PP decarboxylase, decreased activity or expression of acetyl-CoA carboxylase, increased activity or expression of IPP isomerase, increased activity or expression of GPP synthase, increased activity or expression of FPP synthase, increased activity or expression of squalene synthase, decreased activity or expression of squalene synthase, increased activity or expression of GGPP synthase, decreased activity or expression of GGPP synthase, increased activity or expression of glucose-6-phosphate dehydrogenase, increased activity or expression of 6-phosphogluconate dehydrogenase, increased activity or expression of fructose 1, 6 bisphosphatase, increased activity or expression of NADH kinase, increased activity or expression of transhydrogenase, and combinations thereof.

In other embodiments, the present disclosure provides a genetically modified Saccharomyces cerevisiae strain comprising an altered activity or expression of one or more enzymes when compared to an unmodified strain, wherein the altered activity or expression of one or more enzymes is selected from the group consisting of: increased activity or expression of a beta-carotene 15,15′-monooxygenase polypeptide; increased activity or expression of a retinol dehydrogenase polypeptide; increased activity or expression of acetyl-CoA thiolase, increased activity or expression of HMG-CoA synthase, increased activity or expression of HMG-CoA reductase, increased activity or expression of mevalonate kinase, increased activity or expression of phosphomevalonate kinase, increased activity or expression of mevalonate PP decarboxylase, decreased activity or expression of acetyl-CoA carboxylase, increased activity or expression of IPP isomerase, increased activity or expression of GPP synthase, increased activity or expression of FPP synthase, increased activity or expression of squalene synthase, decreased activity or expression of squalene synthase, increased activity or expression of GGPP synthase, decreased activity or expression of GGPP synthase, increased activity or expression of glucose-6-phosphate dehydrogenase, increased activity or expression of 6-phosphogluconate dehydrogenase, increased activity or expression of fructose 1, 6 bisphosphatase, increased activity or expression of NADH kinase, increased activity or expression of transhydrogenase, and combinations thereof.

In some embodiments, the present disclosure provides a genetically modified Xanthophyllomyces dendrorhous (Phaffia rhodozyma) strain comprising an altered activity or expression of one or more enzymes when compared to an unmodified strain, wherein the altered activity or expression of one or more enzymes is selected from the group consisting of: increased activity or expression of a beta-carotene 15,15′-monooxygenase polypeptide; increased activity or expression of a retinol dehydrogenase polypeptide; increased activity or expression of acetyl-CoA thiolase, increased activity or expression of HMG-CoA synthase, increased activity or expression of HMG-CoA reductase, increased activity or expression of mevalonate kinase, increased activity or expression of phosphomevalonate kinase, increased activity or expression of mevalonate PP decarboxylase, decreased activity or expression of acetyl-CoA carboxylase, increased activity or expression of IPP isomerase, increased activity or expression of GPP synthase, increased activity or expression of FPP synthase, increased activity or expression of squalene synthase, decreased activity or expression of squalene synthase, increased activity or expression of GGPP synthase, decreased activity or expression of GGPP synthase, increased activity or expression of glucose-6-phosphate dehydrogenase, increased activity or expression of 6-phosphogluconate dehydrogenase, increased activity or expression of fructose 1, 6 bisphosphatase, increased activity or expression of NADH kinase, increased activity or expression of transhydrogenase, and combinations thereof.

In other embodiments, the present disclosure provides a method of producing a carotenoid, the method comprising steps of cultivating a fungus under conditions that allow production of the carotenoid; and isolating the produced carotenoid. In some embodiments, the method includes cultivating a fungus on a carbon source comprising soybean oil. In some embodiments, the method includes cultivating a fungus serially on at least two different carbon sources; in some such embodiments, at least one of the different carbon sources comprises soybean oil. In some embodiments, the method includes cultivating a fungus under conditions that are limiting for zinc. In some embodiments, the method includes cultivating a fungus under conditions that are limiting for manganese.

In other embodiments, the present disclosure provides a method of producing a retinolic compound, the method comprising steps of cultivating a fungus under conditions that allow production of the retinolic compound; and isolating the produced retinolic compound.

In certain embodiments, the present disclosure provides an isolated carotenoid composition, prepared by a method comprising steps of cultivating the fungus under conditions that allow production of a carotenoid; and isolating the produced carotenoid. In certain embodiments, the present disclosure provides an isolated retinolic compound composition, prepared by a method comprising steps of cultivating the fungus under conditions that allow production of a retinolic compound; and isolating the produced retinolic compound.

In other embodiments, the present disclosure provides a composition comprising lipid bodies; at least one carotenoid compound; and intact fungal cells. In other embodiments, the present disclosure provides a composition comprising lipid bodies; at least one retinolic compound; and intact fungal cells.

In some embodiments, the present disclosure provides a composition comprising: an oil suspension comprising: lipid bodies; at least one carotenoid compound; intact fungal cells; and a binder or filler. In some embodiments, the present disclosure provides a composition comprising: an oil suspension comprising: lipid bodies; at least one retinolic compound; intact fungal cells; and a binder or filler.

In certain embodiments, the present disclosure provides a composition comprising: an oil suspension comprising: lipid bodies; at least one carotenoid compound; intact fungal cells; and one or more other agents selected from the group consisting of chelating agents, pigments, salts, surfactants, moisturizers, viscosity modifiers, thickeners, emollients, fragrances, preservatives, and combinations thereof. In certain embodiments, the present disclosure provides a composition comprising: an oil suspension comprising: lipid bodies; at least one retinolic compound; intact fungal cells; and one or more other agents selected from the group consisting of chelating agents, pigments, salts, surfactants, moisturizers, viscosity modifiers, thickeners, emollients, fragrances, preservatives, and combinations thereof.

In some embodiments, the present disclosure provides a feedstuff comprising a carotenoid in lipid bodies. In other embodiments, the present disclosure provides a feedstuff comprising a carotenoid in lipid bodies; wherein the carotenoid is selected from the group consisting of astaxanthin, β-carotene, canthaxanthin, zeaxanthin, lutein, lycopene, echinenone, β-cryptoxanthin and combinations thereof. In some embodiments, the present disclosure provides a feedstuff comprising a retinolic compound in lipid bodies. In other embodiments, the present disclosure provides a feedstuff comprising a retinolic compound in lipid bodies; wherein the retinolic compound is selected from the group consisting of retinol, retainal, retinoic acid, and combinations thereof.

In certain embodiments, the present disclosure provides a carotenoid composition comprising a Y. lipolytica cell containing at least 1% carotenoids by weight. In other embodiments, the present disclosure provides a carotenoid composition comprising Y. lipolytica lipid bodies; and at least one carotenoid compound, wherein the at least one carotenoid compound is present at a level that is at least 1% by weight of the lipid bodies. In certain embodiments, the present disclosure provides a retinolic compound composition comprising a Y. lipolytica cell containing at least 1% retinolic compounds by weight. In other embodiments, the present disclosure provides a retinolic compound composition comprising Y. lipolytica lipid bodies; and at least one retinolic compound, wherein the at least one retinolic compound is present at a level that is at least 1% by weight of the lipid bodies.

Additional aspects of the present disclosure will be apparent to those of ordinary skill in the art from the present description, including the appended claims.

### BRIEF DESCRIPTION OF THE DRAWING

FIGS. 1A-1D depicts certain common carotenoids.

FIG. 2 depicts how sufficient levels of acetyl-CoA and NADPH may be accumulated in the cytosol of oleaginous organisms to allow for production of significant levels of cytosolic lipids. Enzymes: 1, pyruvate decarboxylase; 2, malate dehydrogenase; 3, malic enzyme; 4, pyruvate dehydrogenase; 5, citrate synthase; 6, ATP-citrate lyase; 7, citrate/malate translocase.

FIGS. 3A and 3B depict the mevalonate isoprenoid biosynthesis pathway, which typically operates in eukaryotes, including fungi.

FIG. 4 depicts the mevalonate-independent isoprenoid biosynthesis pathway, also known as the DXP pathway, which typically operates in bacteria and in the plastids of plants.

FIG. 5 depicts intermediates in the isoprenoid biosynthesis pathway and how they feed into biosynthetic pathways of other biomolecules, including carotenoids as well as non-carotenoid compounds such as sterols, steroids, and vitamins, such as vitamin E or vitamin K.

FIGS. 6A-6D illustrate various carotenoid biosynthetic pathways. FIG. 6A highlights branches leading to various cyclic and acyclic xanthophylls; FIG. 6B shows certain X. dendrorhous pathways that generate dicyclic and monocyclic carotenoids, including astaxanthin; FIG. 6C shows interconnecting pathways for converting β-carotene into any of a variety of other carotenoids, including astaxanthin; FIG. 6D depicts possible routes of synthesis of cyclic carotenoids and common plant and algal xanthophylls from neurosporene.

FIGS. 7A-7I show an alignment of certain representative fungal HMG-CoA reductase polypeptides. As can be seen, these polypeptides show very high identity across the catalytic region, and also have complex membrane spanning domains. In some embodiments of the disclosure, these membrane-spanning domains are disrupted or are removed, so that, for example, a hyperactive version of the polypeptide may be produced.

FIGS. 8A-8P depict schematic representations of plasmids generated and described in detail in the exemplification.

FIGS. 9A-F show production characteristics of certain engineered cells according to the present disclosure. Specifically, Panel A shows β-Carotene and phytoene production by Strain MF760 when grown in glycerol, glucose or olive oil; Panel B shows dry cell weight accumulation of strain MF760 when grown in glycerol, glucose or olive oil; Panel C shows β-Carotene and dry cell weight analysis of strain MF760 when grown in the presence of a combination of olive oil and glucose; Panel D shows canthaxanthin, echinenone and β-carotene production of strain MF840; and Panel E shows canthaxanthin and echinenone production of strain MF838 in a 2 phase feeding protocol; Panel F shows β-Carotene production in cells of strain MF1212 grown in medium lacking supplemental H₃BO₃ (BC1-42), CaCl₂ (BC1-43), CuSO₄ (BC1-44), FeCl₃ (BC1-45), MnSO₄ (BC1-46), Na₂MoO₄ (BC1-47), ZnCl₂ (BC1-48), or medium supplemented with all of these compounds (BC1-41).

FIG. 10 is a Table listing certain Y. lipolytica genes representing various polypeptides (e.g., oleaginic and isoprenoid biosynthesis peptides) useful in engineering cells in accordance with the present disclosure.

FIG. 11 depicts the all-trans-retinol (Vitamin A) biosynthesis pathway, starting with beta-carotene as a substrate.

FIG. 12 depicts various characteristics of strain ML1011 (MF740 transformed with multiple integrated copies of the X. autotrophicus crtZ gene) grown under different pH conditions. FIG. 12a depicts accumulation of total carotenoid (absorbance units per unit dry cell weight) over the course of the fermentation. FIG. 12b depicts accumulation of zeaxanthin (absorbance units per dry cell weight; AU) over the course of the fermentation. FIG. 12c depicts the fraction of carotenoid as zeaxanthin (AU zeaxanthin/AU total carotenoid) over the course of the fermentation. FIG. 12d depicts carbon dioxide evolution over the course of the fermentation. FIG. 12e depicts biomass accumulation over the course of the fermentation.

### DEFINITIONS

Aromatic amino acid biosynthesis polypeptide: The term “aromatic amino acid biosynthesis polypeptide” refers to any polypeptide that is involved in the synthesis of aromatic amino acids in yeast and/or bacteria through chorismate and the shikimate pathway. For example, as discussed herein, anthranilate synthase, enzymes of the shikimate pathway, chorismate mutase, chorismate synthase, DAHP synthase, and transketolase are all aromatic amino acid biosynthesis polypeptides. Each of these polypeptides is also a ubiquinone biosynthesis polypeptide or a ubiquinone biosynthesis competitor for purposes of the present disclosure, as production of chorismate is a precursor in the synthesis of para-hydroxybenzoate for the biosynthesis of ubiquinone.

Biosynthesis polypeptide: The term “biosynthesis polypeptide” as used herein (typically in reference to a particular compound or class of compounds), refers to polypeptides involved in the production of the compound or class of compounds. In some embodiments of the disclosure, biosynthesis polypeptides are synthetic enzymes that catalyze particular steps in a synthesis pathway that ultimately produces a relevant compound. In some embodiments, the term “biosynthesis polypeptide” may also encompass polypeptides that do not themselves catalyze synthetic reactions, but that regulate expression and/or activity of other polypeptides that do so. Biosynthesis polypeptides include, for example, aromatic amino acid biosynthesis polypeptides, C₅₋₉ quinone biosynthesis polypeptides, carotenoid biosynthesis polypeptides, retinolic compound biosynthesis polypeptides, FPP biosynthesis polypeptides, isoprenoid biosynthesis polypeptides, PHB biosynthesis polypeptides, quinone biosynthesis polypeptides, sterol biosynthesis polypeptides, ubiquinone biosynthesis polypeptides, Vitamin D biosynthesis polypeptides, Vitamin E biosynthesis polypeptides, and Vitamin K biosynthesis polypeptides.

C₅₋₉ quinone biosynthesis polypeptide: The term “C₅₋₉ quinone biosynthesis polypeptide” refers to any polypeptide that is involved in the synthesis of a C₅₋₉ quinone, for example a polyprenyldiphosphate synthase polypeptide. To mention but a few, these include, for example, pentaprenyl, hexaprenyl, heptaprenyl, octaprenyl, and/or solanesyl (nonaprenyl) diphosphate synthase polypeptides (i.e., polypeptides that perform the chemical reactions performed by the pentaprenyl, hexaprenyl, heptaprenyl, octaprenyl, and solanesyl (nonaprenyl) polypeptides, respectively (see also Okada et al., Biochim. Biophys. Acta 1302:217, 1996; Okada et al., J. Bacteriol. 179:5992, 1997). As will be appreciated by those of ordinary skill in the art, in some embodiments of the disclosure, C₅₋₉ quinone biosynthesis polypeptides include polypeptides that affect the expression and/or activity of one or more other C₅₋₉ quinone biosynthesis polypeptides.

Carotenogenic modification: The term “carotenogenic modification”, as used herein, refers to a modification of a host organism that adjusts production of one or more carotenoids, as described herein. For example, a carotenogenic modification may increase the production level of one or more carotenoids, and/or may alter relative production levels of different carotenoids. In principle, an inventive carotenogenic modification may be any chemical, physiological, genetic, or other modification that appropriately alters production of one or more carotenoids in a host organism produced by that organism as compared with the level produced in an otherwise identical organism not subject to the same modification. In most embodiments, however, the carotenogenic modification will comprise a genetic modification, typically resulting in increased production of one or more selected carotenoids. In some embodiments, the carotenogenic modification comprises at least one chemical, physiological, genetic, or other modification; in other embodiments, the carotenogenic modification comprises more than one chemical, physiological, genetic, or other modification. In certain aspects where more than one modification is utilized, such modifications can comprise any combination of chemical, physiological, genetic, or other modification (e.g., one or more genetic, chemical, and/or physiological modification(s)). In some embodiments, the selected carotenoid is one or more of astaxanthin, β-carotene, canthaxanthin, lutein, lycopene, phytoene, zeaxanthin, and/or modifications of zeaxanthin or astaxanthin (e.g., glucoside, esterified zeaxanthin or astaxanthin). In some embodiments, the selected carotenoid is one or more xanthophylls, and/or a modification thereof (e.g., glucoside, esterified xanthophylls). In certain embodiments, the selected xanthophyl is selected from the group consisting of astaxanthin, lutein, zeaxanthin, lycopene, and modifications thereof. In some embodiments, the selected carotenoid is one or more of astaxanthin, β-carotene, canthaxanthin, lutein, lycopene, and zeaxanthin and/or modifications of zeaxanthin or astaxanthin. In some embodiments, the carotenoid is β-carotene. In some embodiments, the selected carotenoid is astaxanthin. In some embodiments, the selected carotenoid is other than β-carotene.

Carotenogenic polypeptide: The term “carotenogenic polypeptide”, as used herein, refers to any polypeptide that is involved in the process of producing carotenoids in a cell, and may include polypeptides that are involved in processes other than carotenoid production but whose activities affect the extent or level of production of one or more carotenoids, for example by scavenging a substrate or reactant utilized by a carotenoid polypeptide that is directly involved in carotenoid production. Carotenogenic polypeptides include isoprenoid biosynthesis polypeptides, carotenoid biosynthesis polypeptides, and isoprenoid biosynthesis competitor polypeptides, as those terms are defined herein. The term also encompasses polypeptides that may affect the extent to which carotenoids are accumulated in lipid bodies.

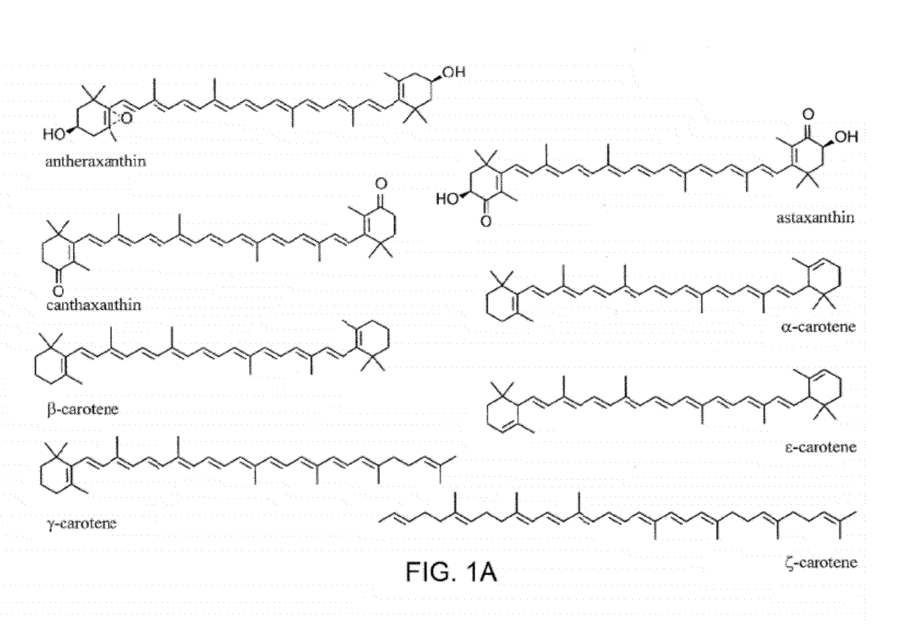

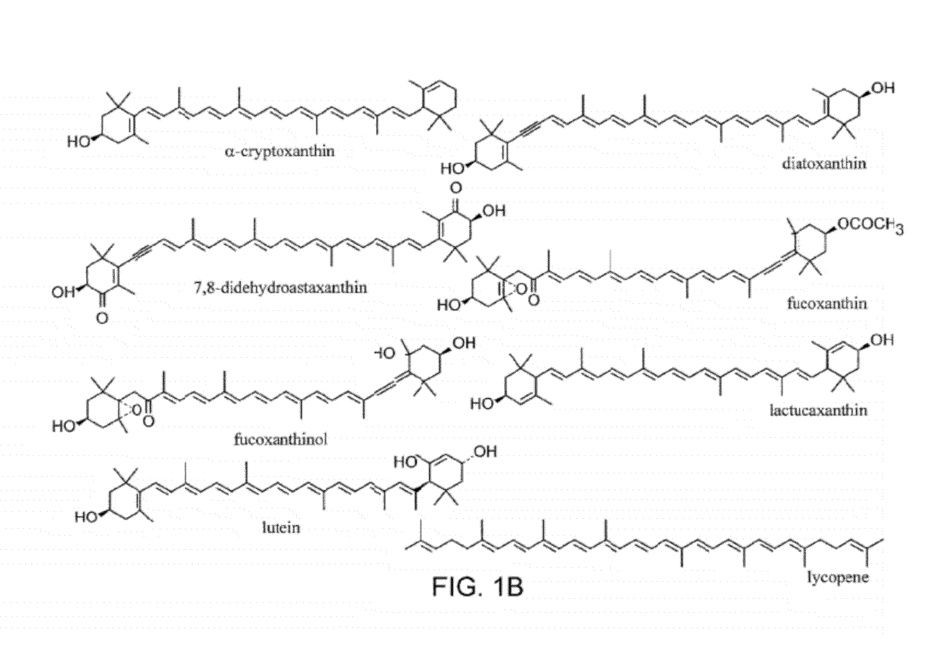

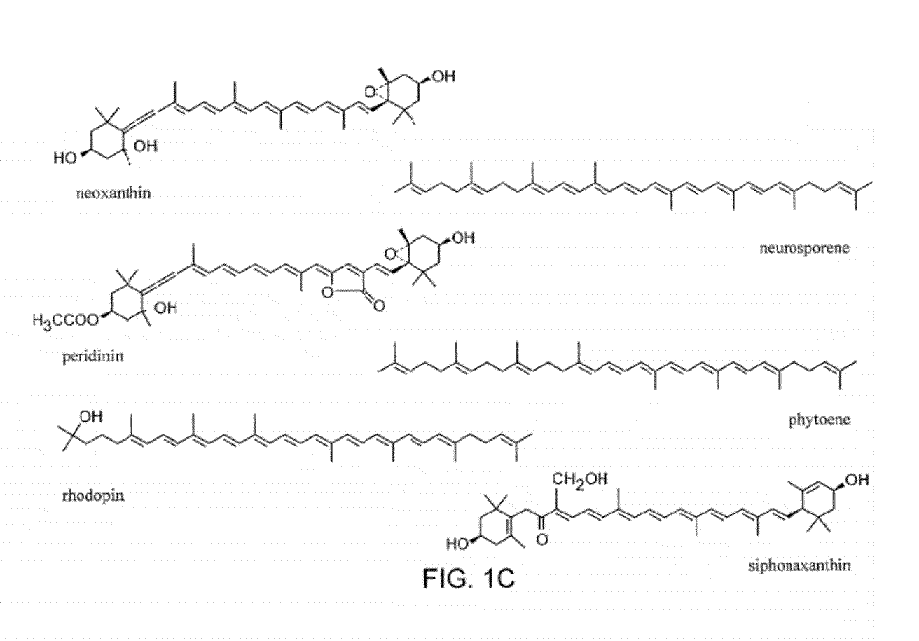

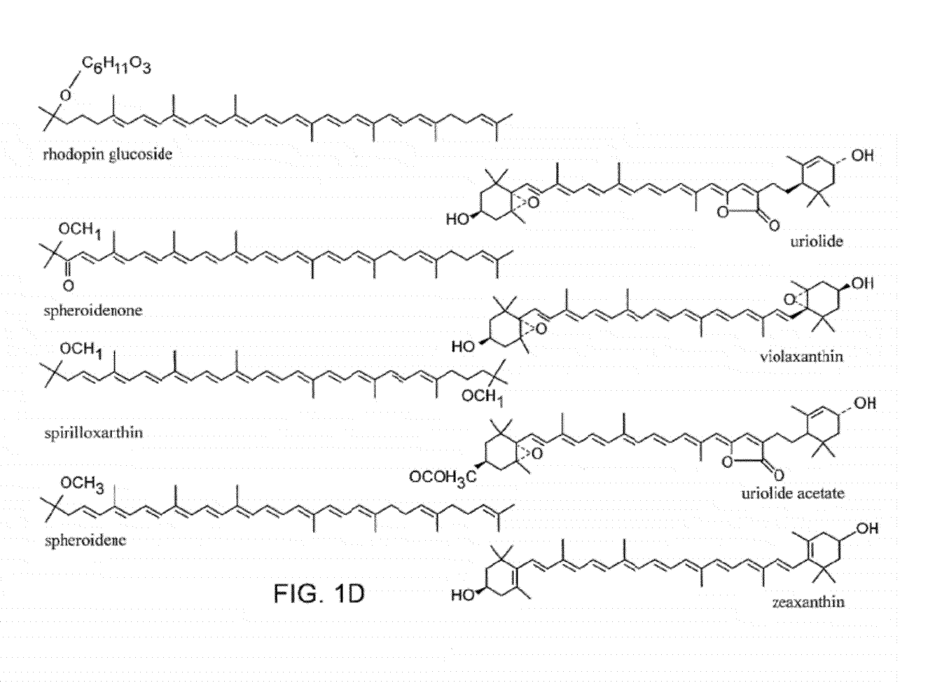

Carotenoid: The term “carotenoid” is understood in the art to refer to a structurally diverse class of pigments derived from isoprenoid pathway intermediates. The commitment step in carotenoid biosynthesis is the formation of phytoene from geranylgeranyl pyrophosphate. Carotenoids can be acyclic or cyclic, and may or may not contain oxygen, so that the term carotenoids include both carotenes and xanthophylls. In general, carotenoids are hydrocarbon compounds having a conjugated polyene carbon skeleton formally derived from the five-carbon compound IPP, including triterpenes (C₃₀ diapocarotenoids) and tetraterpenes (C₄₀ carotenoids) as well as their oxygenated derivatives and other compounds that are, for example, C₃₅, C₅₀, C₆₀, C₇₀, C₈₀ in length or other lengths. Many carotenoids have strong light absorbing properties and may range in length in excess of C₂₀₀. C₃₀ diapocarotenoids typically consist of six isoprenoid units joined in such a manner that the arrangement of isoprenoid units is reversed at the center of the molecule so that the two central methyl groups are in a 1,6-positional relationship and the remaining non-terminal methyl groups are in a 1,5-positional relationship. Such C₃₀ carotenoids may be formally derived from the acyclic C₃₀H₄₂ structure, having a long central chain of conjugated double bonds, by: (i) hydrogenation (ii) dehydrogenation, (iii) cyclization, (iv) oxidation, (v) esterification/glycosylation, or any combination of these processes. C₄₀ carotenoids typically consist of eight isoprenoid units joined in such a manner that the arrangement of isoprenoid units is reversed at the center of the molecule so that the two central methyl groups are in a 1,6-positional relationship and the remaining non-terminal methyl groups are in a 1,5-positional relationship. Such C₄₀ carotenoids may be formally derived from the acyclic C₄₀H₅₆ structure, having a long central chain of conjugated double bonds, by (i) hydrogenation, (ii) dehydrogenation, (iii) cyclization, (iv) oxidation, (v) esterification/glycosylation, or any combination of these processes. The class of C₄₀ carotenoids also includes certain compounds that arise from rearrangements of the carbon skeleton, or by the (formal) removal of part of this structure. More than 600 different carotenoids have been identified in nature; certain common carotenoids are depicted in FIG. 1. Carotenoids include but are not limited to: antheraxanthin, adonirubin, adonixanthin, astaxanthin, canthaxanthin, capsorubrin, β-cryptoxanthin, α-carotene, β-carotene, β,ψ-carotene, δ-carotene, s-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, and C30 carotenoids. Additionally, carotenoid compounds include derivatives of these molecules, which may include hydroxy-, methoxy-, oxo-, epoxy-, carboxy-, or aldehydic functional groups. Further, included carotenoid compounds include ester (e.g., glycoside ester, fatty acid ester) and sulfate derivatives (e.g., esterified xanthophylls).

Carotenoid biosynthesis polypeptide: The term “carotenoid biosynthesis polypeptide” refers to any polypeptide that is involved in the synthesis of one or more carotenoids. To mention but a few, these carotenoid biosynthesis polypeptides include, for example, polypeptides of phytoene synthase, phytoene dehydrogenase (or desaturase), lycopene cyclase, carotenoid ketolase, carotenoid hydroxylase, astaxanthin synthase, carotenoid epsilon hydroxylase, lycopene cyclase (beta and epsilon subunits), carotenoid glucosyltransferase, and acyl CoA:diacyglycerol acyltransferase. In some instances, a single gene may encode a protein with multiple carotenoid biosynthesis polypeptide activities. Representative examples of carotenoid biosynthesis polypeptide sequences are presented in Tables 17a-25. As will be appreciated by those of ordinary skill in the art, in some embodiments of the disclosure, carotenoid biosynthesis polypeptides include polypeptides that affect the expression and/or activity of one or more other carotenoid biosynthesis polypeptides.

FPP biosynthesis polypeptides: The term “FPP biosynthesis polypeptide” refers to any polypeptide that is involved in the synthesis of farnesyl pyrophosphate. As discussed herein, farnesyl pyrophosphate represents the branchpoint between the sterol biosynthesis pathway and the carotenoid and other biosynthesis pathways. One specific example of an FPP biosynthesis polypeptide is FPP synthase. Representative examples of FPP synthase polypeptide sequences are presented in Table 14. As will be appreciated by those of ordinary skill in the art, in some embodiments of the disclosure, FPP biosynthesis polypeptides include polypeptides that affect the expression and/or activity of one or more other FPP biosynthesis polypeptides.

Gene: The term “gene”, as used herein, generally refers to a nucleic acid encoding a polypeptide, optionally including certain regulatory elements that may affect expression of one or more gene products (i.e., RNA or protein).

Heterologous: The term “heterologous”, as used herein to refer to genes or polypeptides, refers to a gene or polypeptide that does not naturally occur in the organism in which it is being expressed. It will be understood that, in general, when a heterologous gene or polypeptide is selected for introduction into and/or expression by a host cell, the particular source organism from which the heterologous gene or polypeptide may be selected is not essential to the practice of the present disclosure. Relevant considerations may include, for example, how closely related the potential source and host organisms are in evolution, or how related the source organism is with other source organisms from which sequences of other relevant polypeptides have been selected. Where a plurality of different heterologous polypeptides are to be introduced into and/or expressed by a host cell, different polypeptides may be from different source organisms, or from the same source organism. To give but one example, in some cases, individual polypeptides may represent individual subunits of a complex protein activity and/or may be required to work in concert with other polypeptides in order to achieve the goals of the present disclosure. In some embodiments, it will often be desirable for such polypeptides to be from the same source organism, and/or to be sufficiently related to function appropriately when expressed together in a host cell. In some embodiments, such polypeptides may be from different, even unrelated source organisms. It will further be understood that, where a heterologous polypeptide is to be expressed in a host cell, it will often be desirable to utilize nucleic acid sequences encoding the polypeptide that have been adjusted to accommodate codon preferences of the host cell and/or to link the encoding sequences with regulatory elements active in the host cell. For example, when the host cell is a Yarrowia strain (e.g., Yarrowia lipolytica), it will often be desirable to alter the gene sequence encoding a given polypeptide such that it conforms more closely with the codon preferences of such a Yarrowia strain. In certain embodiments, a gene sequence encoding a given polypeptide is altered to conform more closely with the codon preference of a species related to the host cell. For example, when the host cell is a Yarrowia strain (e.g., Yarrowia lipolytica), it will often be desirable to alter the gene sequence encoding a given polypeptide such that it conforms more closely with the codon preferences of a related fungal strain. Such embodiments are advantageous when the gene sequence encoding a given polypeptide is difficult to optimize to conform to the codon preference of the host cell due to experimental (e.g., cloning) and/or other reasons. In certain embodiments, the gene sequence encoding a given polypeptide is optimized even when such a gene sequence is derived from the host cell itself (and thus is not heterologous). For example, a gene sequence encoding a polypeptide of interest may not be codon optimized for expression in a given host cell even though such a gene sequence is isolated from the host cell strain. In such embodiments, the gene sequence may be further optimized to account for codon preferences of the host cell. Those of ordinary skill in the art will be aware of host cell codon preferences and will be able to employ inventive methods and compositions disclosed herein to optimize expression of a given polypeptide in the host cell.

Host cell: As used herein, the “host cell” is a fungal cell or yeast cell that is manipulated according to the present disclosure to accumulate lipid and/or to express one or more carotenoids as described herein. A “modified host cell”, as used herein, is any host cell which has been modified, engineered, or manipulated in accordance with the present disclosure as compared with a parental cell. In some embodiments, the modified host cell has at least one carotenogenic and/or at least one oleaginic modification. In some embodiments, the modified host cell containing at least one oleaginic modification and/or one carotenogenic modification further has at least one sterologenic modification and/or at least one quinonogenic modification. In some embodiments, the parental cell is a naturally occurring parental cell.

Isolated: The term “isolated”, as used herein, means that the isolated entity has been separated from at least one component with which it was previously associated. When most other components have been removed, the isolated entity is “purified” or “concentrated”. Isolation and/or purification and/or concentration may be performed using any techniques known in the art including, for example, fractionation, extraction, precipitation, or other separation.

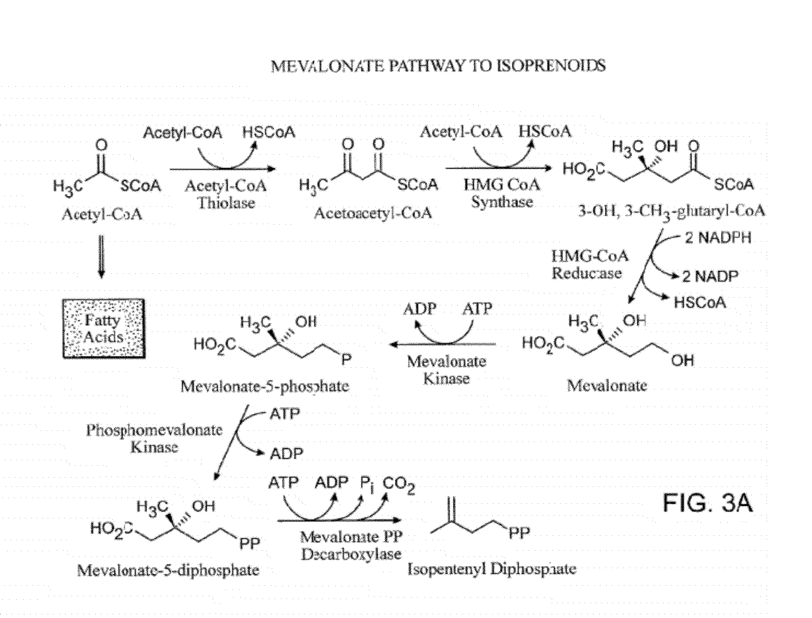

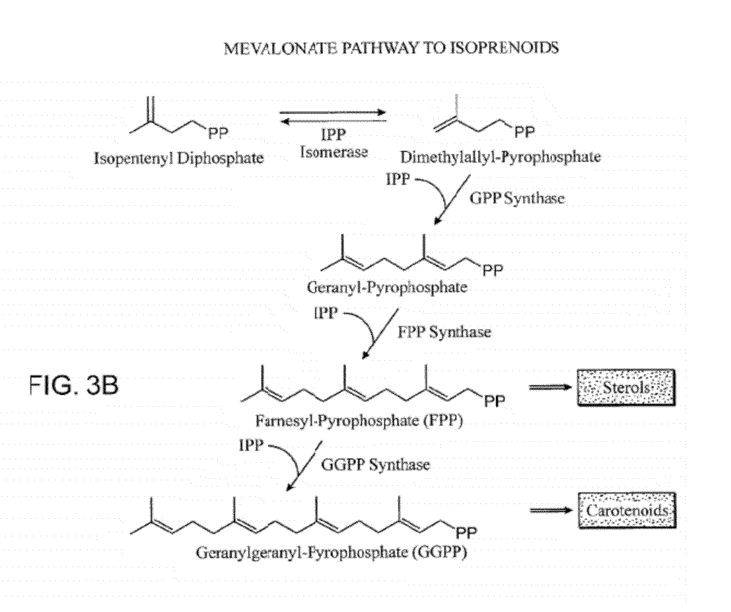

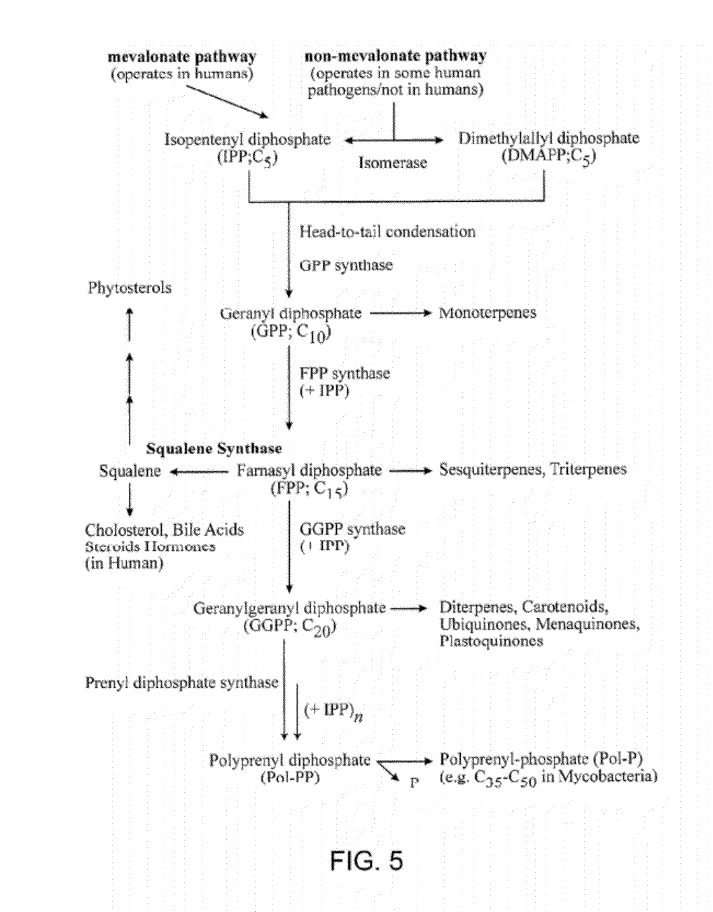

Isoprenoid biosynthesis competitor: The term “isoprenoid biosynthesis competitor”, as used herein, refers to an agent whose presence or activity in a cell reduces the level of geranylgeranyl diphosphate (GGPP) available to enter the carotenoid biosynthesis pathway. The term “isoprenoid biosynthesis competitor” encompasses both polypeptide and non-polypeptide (e.g., small molecule) inhibitor agents. Those of ordinary skill in the art will appreciate that certain competitor agents that do not act as inhibitors of isoprenoid biosynthesis generally can nonetheless act as inhibitors of biosynthesis of a particular isoprenoid compound. Particular examples of isoprenoid biosynthesis competitor agents act on isoprenoid intermediates prior to GGPP, such that less GGPP is generated (see, for example, FIG. 5). Squalene synthase is but one isoprenoid biosynthesis competitor polypeptide according to the present disclosure; representative squalene synthase sequences are presented in Table 16. Prenyldiphosphate synthase enzymes and para-hydroxybenzoate (PHB) polyprenyltransferase are yet additional isoprenoid biosynthesis competitor polypeptides according to the present disclosure; representative prenyldiphosphate synthase enzymes and PHB polyprenyltransferase polypeptides are presented in Tables 29 and 30, respectively. In certain embodiments, one or more polypeptide components of the SAGA complex are isoprenoid biosynthesis competitors according to the present disclosure. Genes encoding SAGA complex polypeptides are presented in Table 69. In certain embodiments, a polypeptide encoded by these and other SAGA complex genes is an isoprenoid biosynthesis competitor polypeptide according to the present disclosure. Those of ordinary skill in the art, considering the known metabolic pathways relating to isoprenoid production and/or metabolism (see, for example, FIGS. 3-6 and other Figures and references herein) will readily appreciate a variety of other particular isoprenoid biosynthesis competitors, including isoprenoid biosynthesis polypeptides.

Isoprenoid biosynthesis polypeptide: The term “isoprenoid biosynthesis polypeptide” refers to any polypeptide that is involved in the synthesis of isoprenoids. For example, as discussed herein, acetoacetyl-CoA thiolase, HMG-CoA synthase, HMG-CoA reductase, mevalonate kinase, phosphomevalonate kinase, mevalonate pyrophosphate decarboxylase, IPP isomerase, FPP synthase, and GGPP synthase, are all involved in the mevalonate pathway for isoprenoid biosynthesis. Each of these proteins is also an isoprenoid biosynthesis polypeptide for purposes of the present disclosure, and sequences of representative examples of these enzymes are provided in Tables 7-15. As will be appreciated by those of ordinary skill in the art, in some embodiments of the disclosure, isoprenoid biosynthesis polypeptides include polypeptides that affect the expression and/or activity of one or more other isoprenoid biosynthesis polypeptides (e.g., of one or more enzymes that participates in isoprenoid synthesis). Thus, for instance, transcription factors that regulate expression of isoprenoid biosynthesis enzymes can be isoprenoid biosynthesis polypeptides for purposes of the present disclosure. To give but a couple of examples, the S. cerevisae Upc2 and YLR228c genes, and the Y. lipolytica YALI0B00660g gene encode transcription factors that are isoprenoid biosynthesis polypeptides according to certain embodiments of the present disclosure. For instance, the semidominant upc2-1 point mutant (G888D) exhibits increases sterol levels (Crowley et al. J. Bacteriol. 180: 4177-4183, 1998). Corresponding YLR228c mutants have been made and tested (Shianna et al. J Bacteriology 183:830-834, 2001); such mutants may be useful in accordance with the present disclosure, as may be YALI0B00660g derivatives with corresponding upc2-1 mutation(s).

Isoprenoid pathway: The term “isoprenoid pathway” is understood in the art to refer to a metabolic pathway that either produces or utilizes the five-carbon metabolite isopentyl pyrophosphate (IPP). As discussed herein, two different pathways can produce the common isoprenoid precursor IPP—the “mevalonate pathway” and the “non-mevalonate pathway”. The term “isoprenoid pathway” is sufficiently general to encompass both of these types of pathway. Biosynthesis of isoprenoids from IPP occurs by polymerization of several five-carbon isoprene subunits. Isoprenoid metabolites derived from IPP are of varying size and chemical structure, including both cyclic and acyclic molecules. Isoprenoid metabolites include, but are not limited to, monoterpenes, sesquiterpenes, diterpenes, sterols, and polyprenols such as carotenoids.

Oleaginic modification: The term “oleaginic modification”, as used herein, refers to a modification of a host organism that adjusts the desirable oleaginy of that host organism, as described herein. In some cases, the host organism will already be oleaginous in that it will have the ability to accumulate lipid to at least about 20% of its dry cell weight. It may nonetheless be desirable to apply an oleaginic modification to such an organism, in accordance with the present disclosure, for example to increase (or, in some cases, possibly to decrease) its total lipid accumulation, or to adjust the types or amounts of one or more particular lipids it accumulates (e.g., to increase relative accumulation of triacylglycerol). In other cases, the host organism may be non-oleaginous (though may contain some enzymatic and regulatory components used in other organisms to accumulate lipid), and may require oleaginic modification in order to become oleaginous in accordance with the present disclosure. The present disclosure also contemplates application of oleaginic modification to non-oleaginous host strains such that their oleaginicity is increased even though, even after being modified, they may not be oleaginous as defined herein. In principle, the oleaginic modification may be any chemical, physiological, genetic, or other modification that appropriately alters oleaginy of a host organism as compared with an otherwise identical organism not subjected to the oleaginic modification. In most embodiments, however, the oleaginic modification will comprise a genetic modification, typically resulting in increased production and/or activity of one or more oleaginic polypeptides. In some embodiments, the oleaginic modification comprises at least one chemical, physiological, genetic, or other modification; in other embodiments, the oleaginic modification comprises more than one chemical, physiological, genetic, or other modification. In certain aspects where more than one modification is utilized, such modifications can comprise any combination of chemical, physiological, genetic, or other modification (e.g., one or more genetic, chemical and/or physiological modification(s)).

Oleaginic polypeptide: The term “oleaginic polypeptide”, as used herein, refers to any polypeptide that is involved in the process of lipid accumulation in a cell and may include polypeptides that are involved in processes other than lipid biosynthesis but whose activities affect the extent or level of accumulation of one or more lipids, for example by scavenging a substrate or reactant utilized by an oleaginic polypeptide that is directly involved in lipid accumulation. For example, as discussed herein, acetyl-CoA carboxylase, pyruvate decarboxylase, isocitrate dehydrogenase, ATP-citrate lyase, malic enzyme, malate dehydrogenase, and AMP deaminase, among other proteins, are all involved in lipid accumulation in cells. In general, reducing the activity of pyruvate decarboxylase or isocitrate dehydrogenase, and/or increasing the activity of acetyl CoA carboxylase, ATP-citrate lyase, malic enzyme, malate dehydrogenase, and/or AMP deaminase is expected to promote oleaginy. Each of these proteins is an oleaginic peptide for the purposes of the present disclosure, and sequences of representative examples of these enzymes are provided in Tables 1-6, and 30. Other peptides that can be involved in regenerating NADPH may include, for example, 6-phosphogluconate dehydrogenase (gnd); Fructose 1,6 bisphosphatase (fbp); Glucose 6 phosphate dehydrogenase (g6pd); NADH kinase (EC 2.7.1.86); and/or transhydrogenase (EC 1.6.1.1 and 1.6.1.2). Alternative or additional strategies to promote oleaginy may include one or more of the following: (1) increased or heterologous expression of one or more of acyl-CoA:diacylglycerol acyltransferase (e.g., DGA1; YALI0E32769g); phospholipid: diacylglycerol acyltransferase (e.g., LRO1; YALI0E16797g); and acyl-CoA:cholesterol acyltransferase (e.g., ARE genes such as ARE1, ARE2, YALI0F06578g), which are involved in triglyceride synthesis (Kalscheuer et al. Appl Environ Microbiol p. 7119-7125, 2004; Oelkers et al. J Biol Chem 277:8877-8881, 2002; and Sorger et al. J Biol Chem 279:31190-31196, 2004), (2) decreased expression of triglyceride lipases (e.g., TGL3 and/or TGL4; YALI0D17534g and/or YALI0F10010g (Kurat et al. J Biol Chem 281:491-500, 2006); and (3) decreased expression of one or more acyl-coenzyme A oxidase activities, for example encoded by POX genes (e.g., POX1, POX2, POX3, POX4, POX5; YALI0C23859g, YALI0D24750g, YALI0E06567g, YALI0E27654g, YALI0E32835g, YALI0F10857g; see, for example, Mlickova et al. Appl Environ Microbiol 70: 3918-3924, 2004; Binns et al. J Cell Biol 173:719, 2006). Each of these proteins is an oleaginic peptide for the purposes of the present disclosure, and sequences of representative examples of these enzymes are provided in Tables 31-43 and 45-47.

Oleaginous: The term “oleaginous”, refers to the ability of an organism to accumulate lipid to at least about 20% of its dry cell weight. In certain embodiments of the disclosure, oleaginous yeast or fungi accumulate lipid to at least about 25% of their dry cell weight. In other embodiments, inventive oleaginous yeast or fungi accumulate lipid within the range of about 20-45% (e.g., about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, or more) of their dry cell weight. In some embodiments, oleaginous organisms may accumulate lipid to as much as about 70% of their dry cell weight. In some embodiments of the disclosure, oleaginous organisms may accumulate a large fraction of total lipid accumulation in the form of triacylglycerol. In certain embodiments, the majority of the accumulated lipid is in the form of triacylglycerol. Alternatively or additionally, the lipid may accumulate in the form of intracellular lipid bodies, or oil bodies. In certain embodiments, the present disclosure utilizes yeast or fungi that are naturally oleaginous. In some aspects, naturally oleaginous organisms are manipulated (e.g., genetically, chemically, or otherwise) so as to father increase the level of accumulated lipid in the organism. In other embodiments, yeast or fungi that are not naturally oleaginous are manipulated (e.g., genetically, chemically, or otherwise) to accumulate lipid as described herein. For example, for the purposes of the present disclosure, Saccharomyces cerevisiae, Xanthophyllomyces dendrorhous (Phaffia rhodozyma), and Candida utilis are not naturally oleaginous fungi.

PHB polypeptide or PHB biosynthesis polypeptide: The terms “PHB polypeptide” or “PHB biosynthesis polypeptide” as used herein refers to a polypeptide that is involved in the synthesis of para-hydroxybenzoate from chorismate. In prokaryotes and lower eukaryotes, synthesis of para-hydroxybenzoate occurs by the action of chorismate pyruvate lyase. Biosynthesis of para-hydroxybenzoate from tyrosine or phenylalanine occurs through a five-step process in mammalian cells. Lower eukaryotes such as yeast can utilize either method for production of para-hydroxybenzoate. For example, enzymes of the shikimate pathway, chorismate synthase, DAHP synthase, and transketolase are all PHB biosynthesis polypeptides. Each of these polypeptides is also a ubiquinone biosynthesis polypeptide or a ubiquinone biosynthesis competitor polypeptide for purposes of the present disclosure.

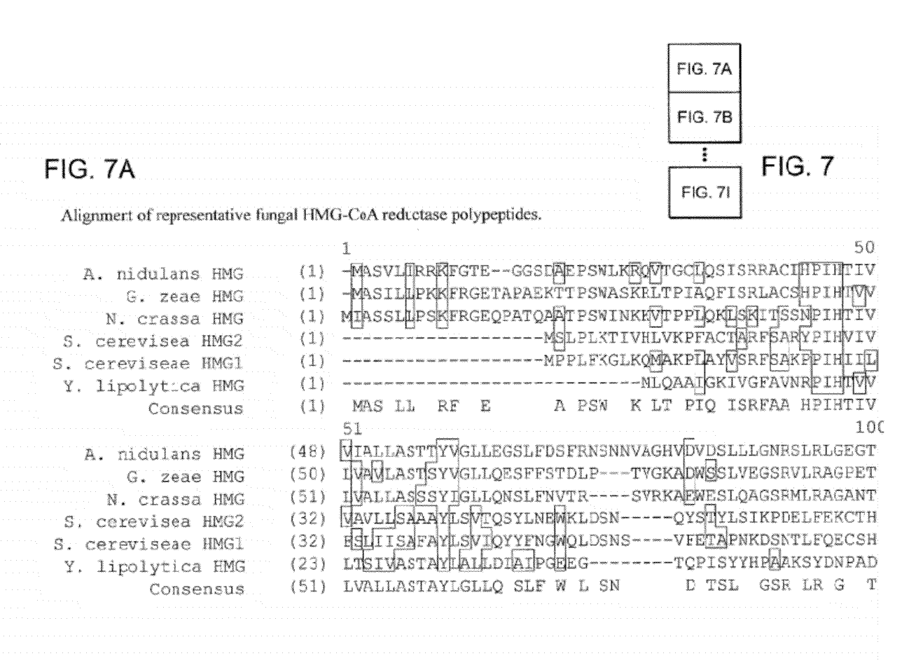

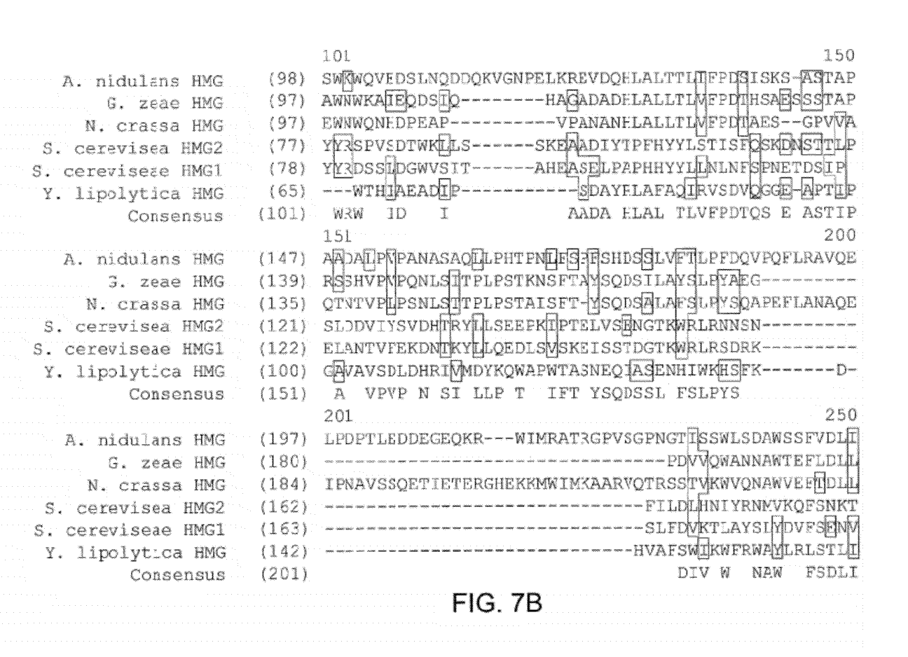

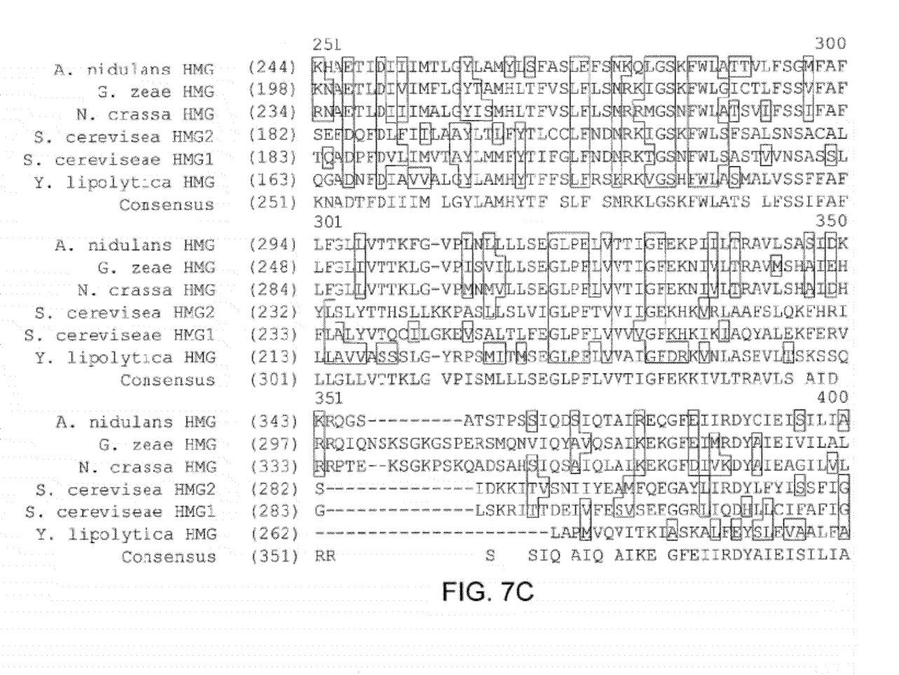

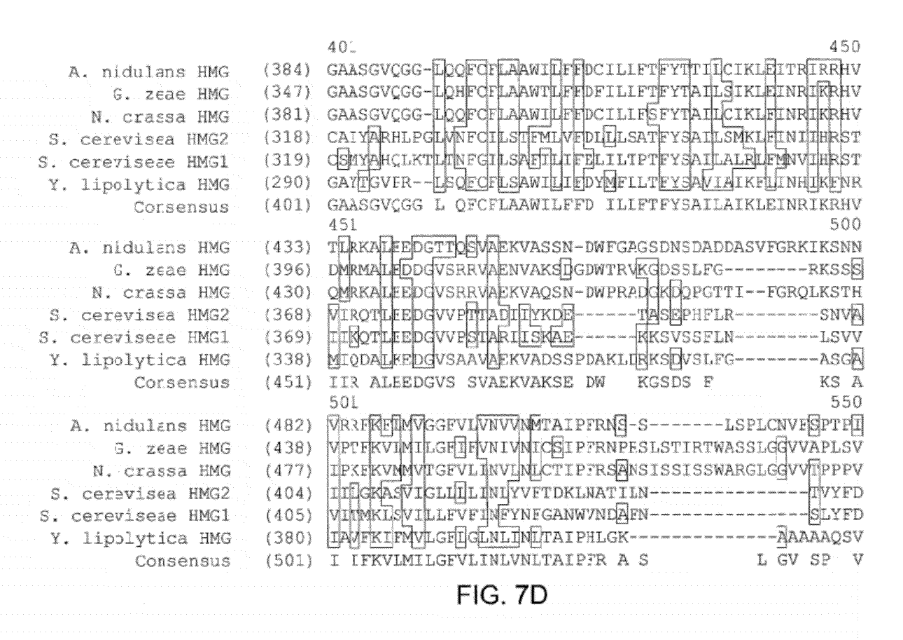

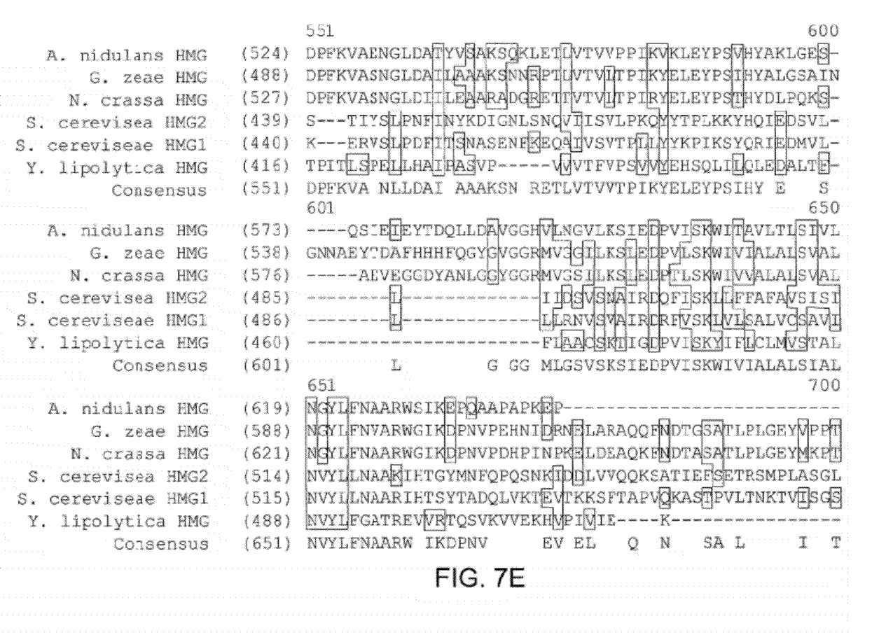

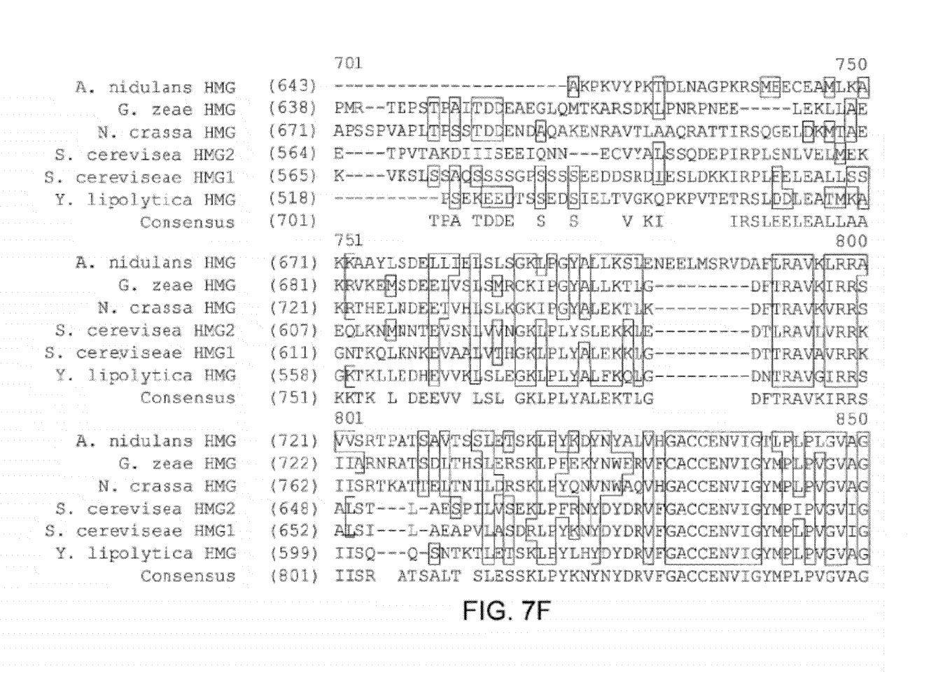

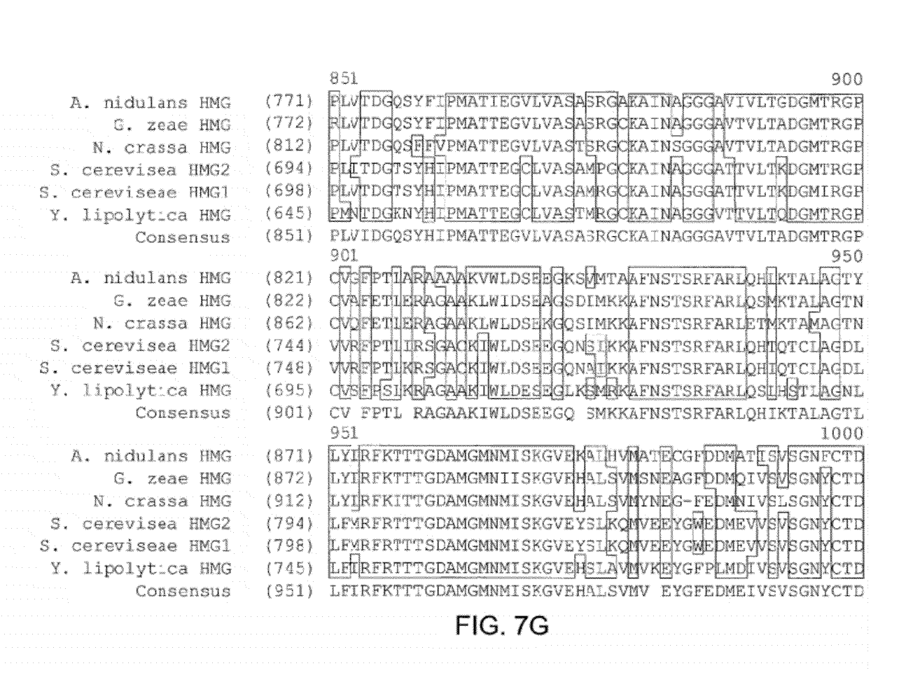

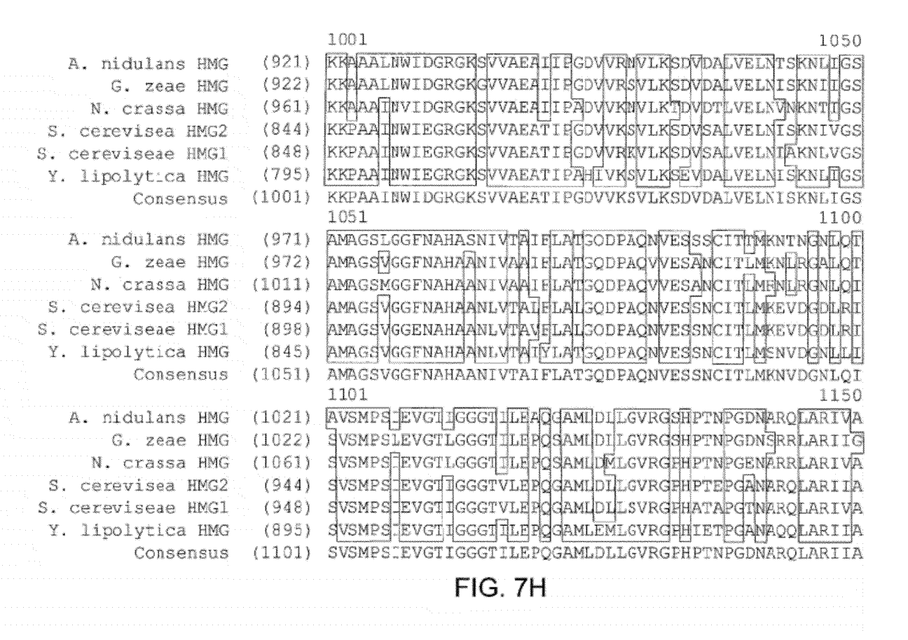

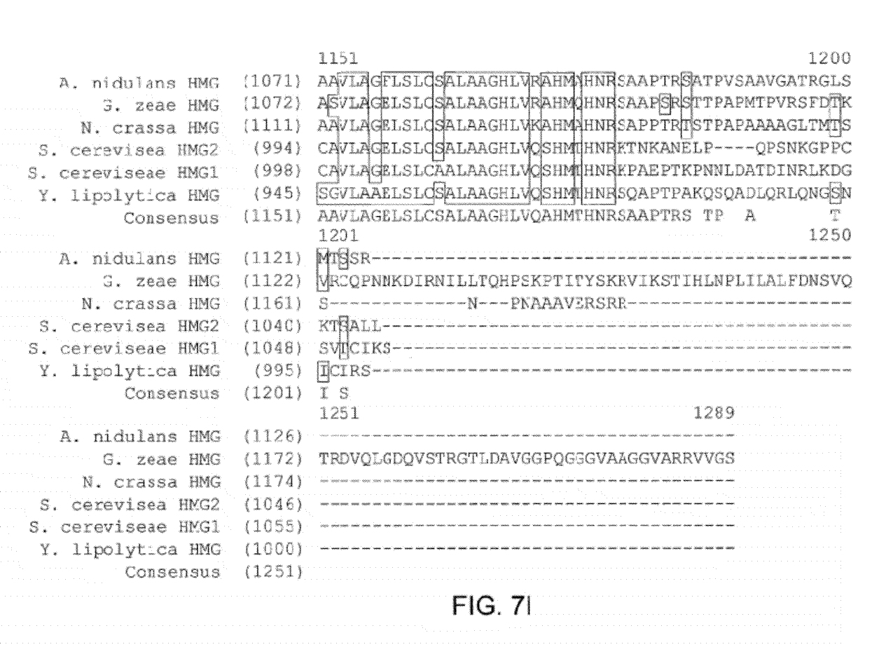

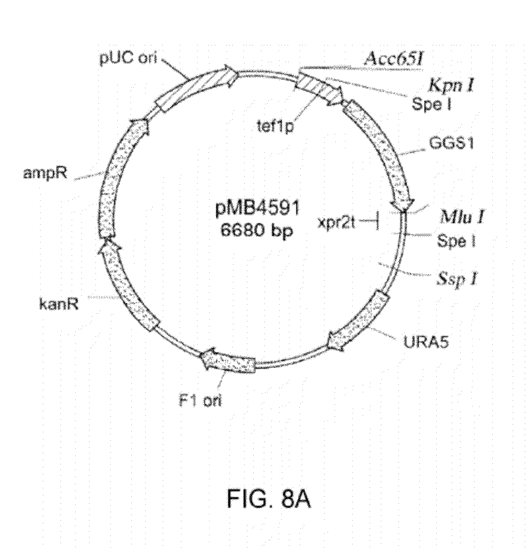

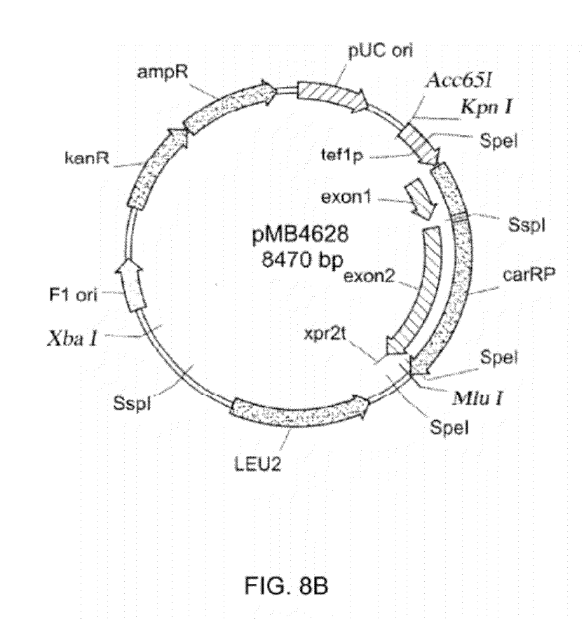

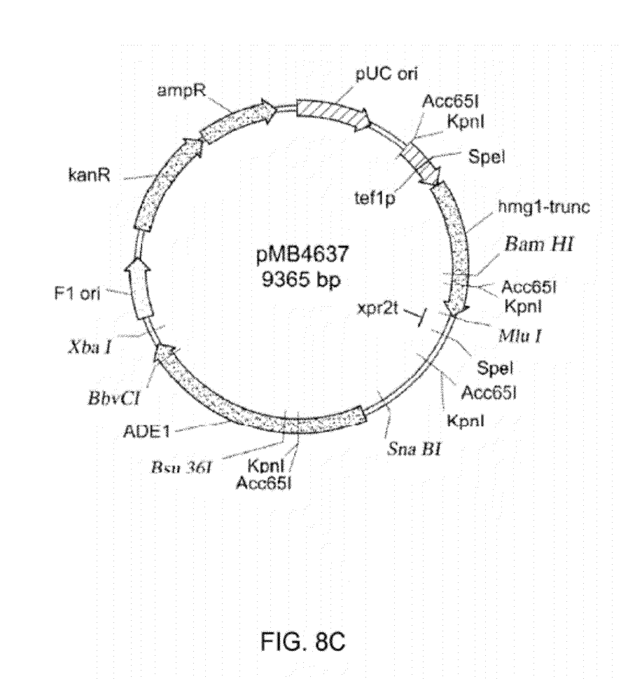

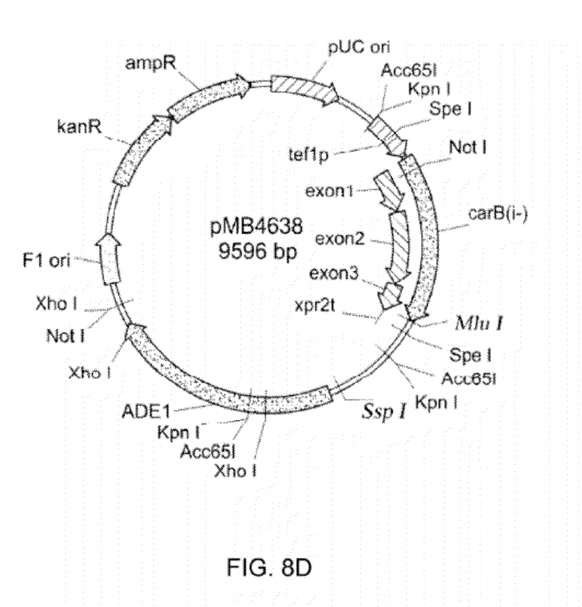

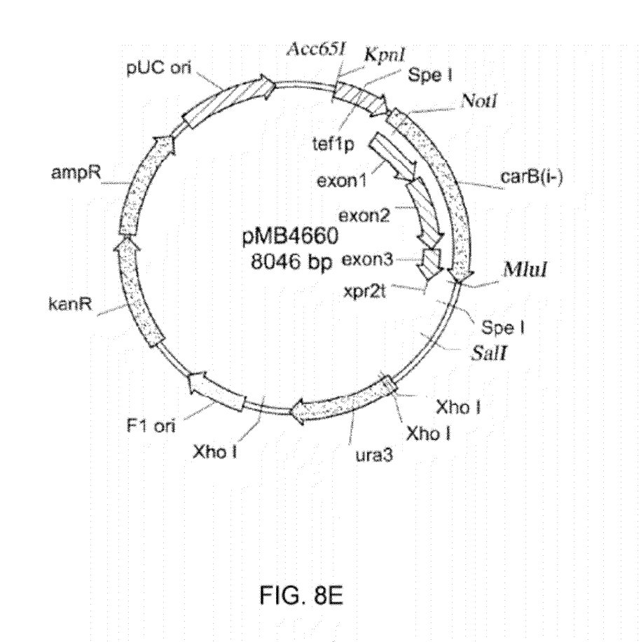

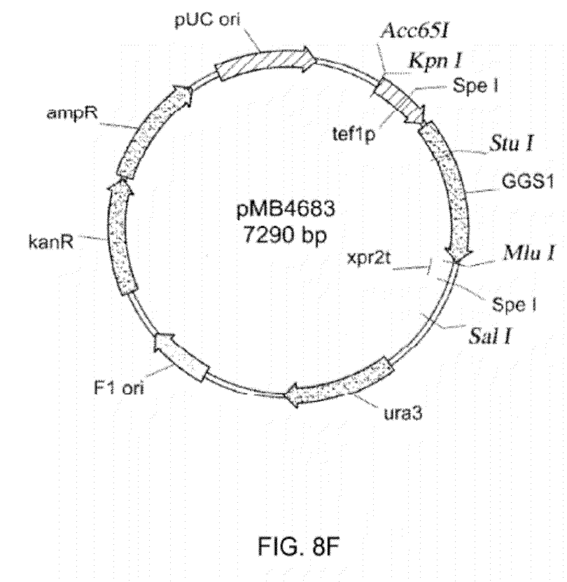

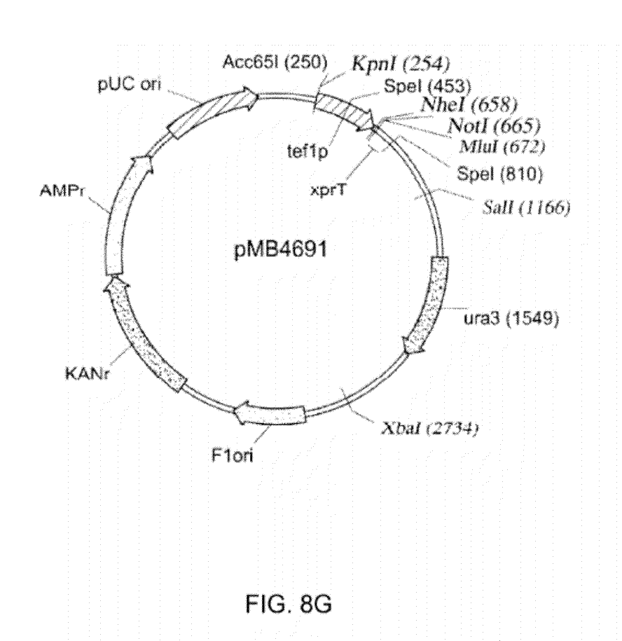

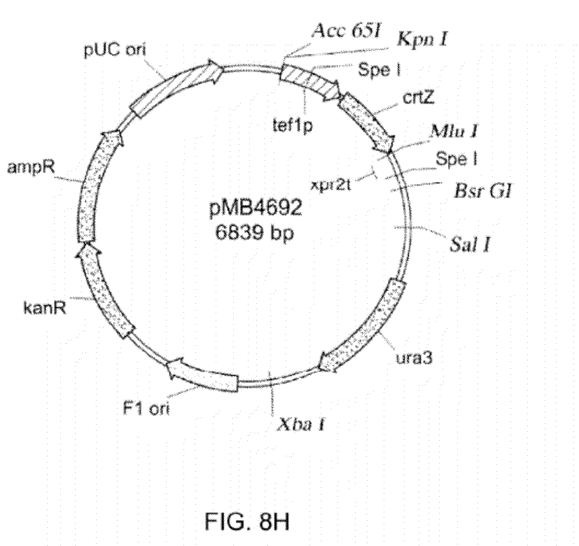

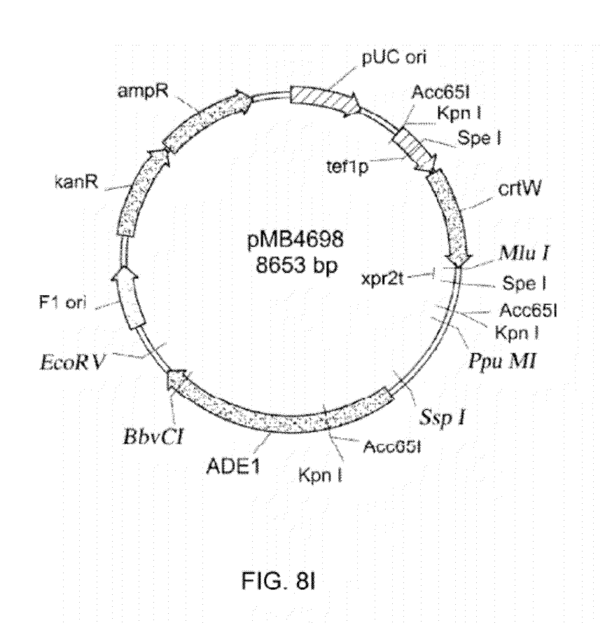

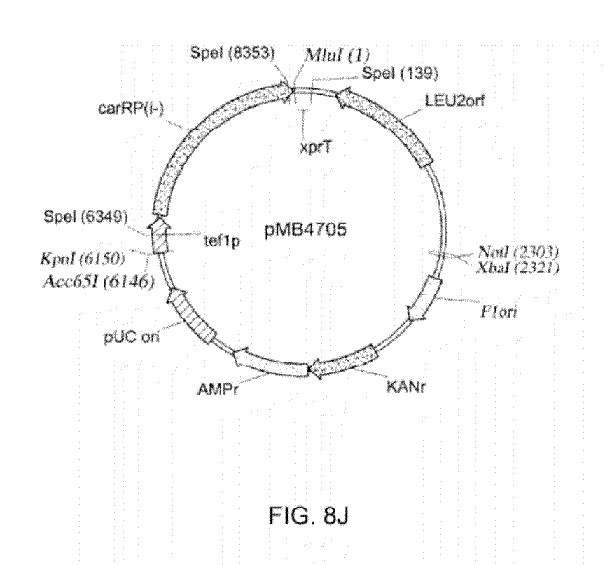

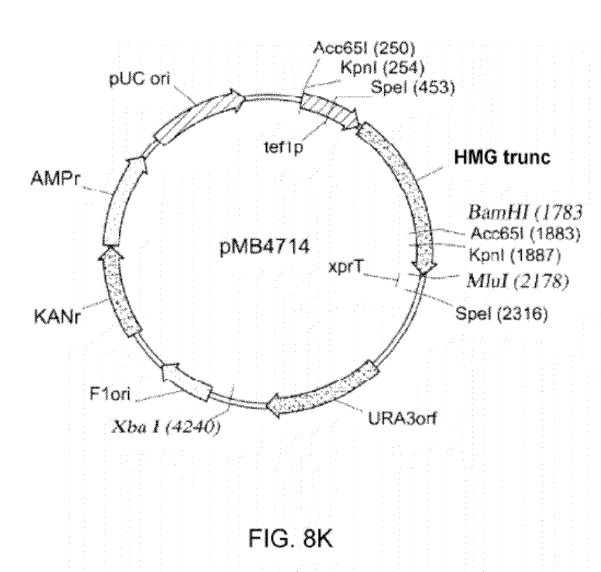

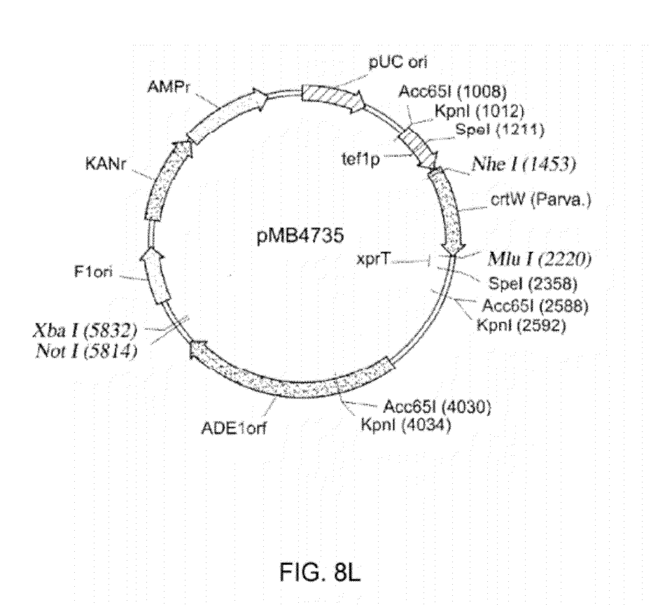

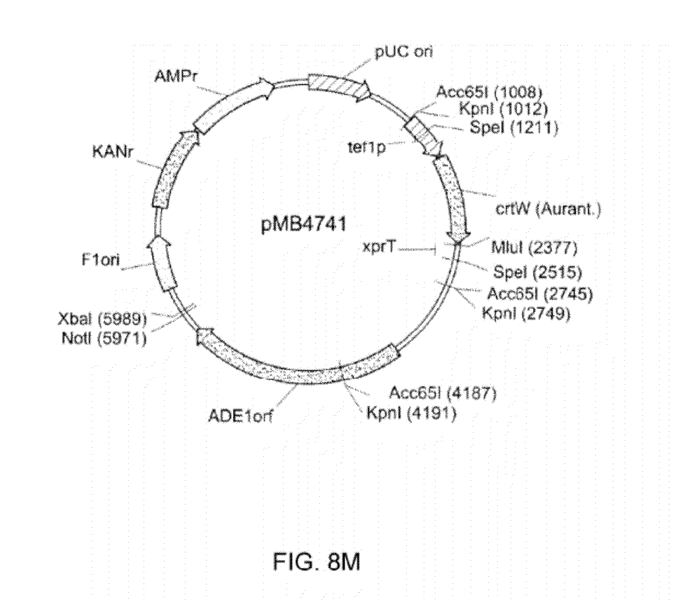

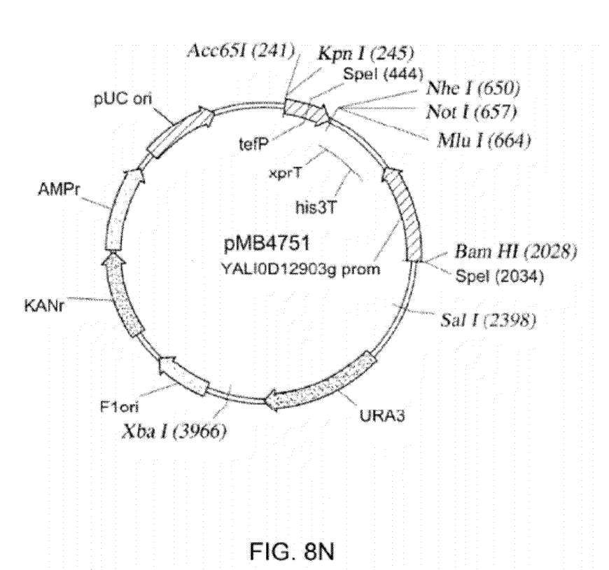

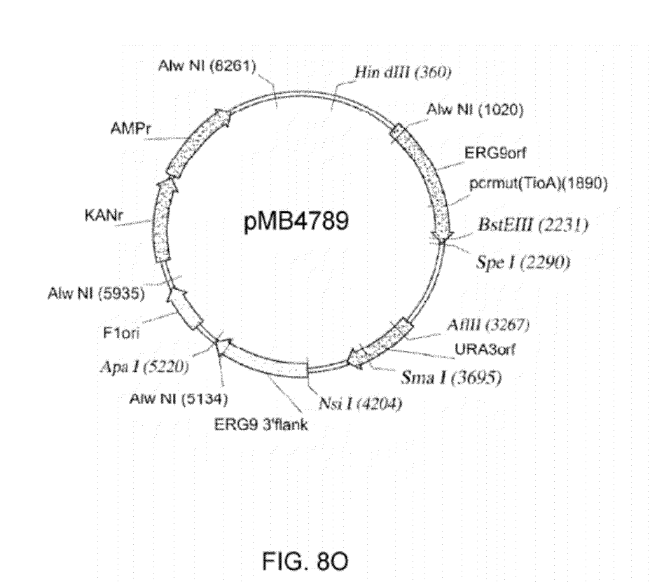

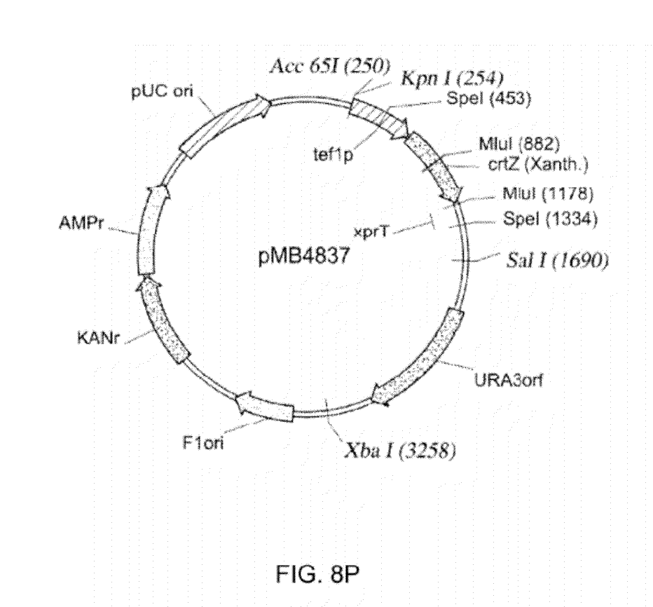

Polypeptide: The term “polypeptide”, as used herein, generally has its art-recognized meaning of a polymer of at least three amino acids. However, the term is also used to refer to specific functional classes of polypeptides, such as, for example, oleaginic polypeptides, carotenogenic polypeptides, isoprenoid biosynthesis polypeptides, carotenoid biosynthesis polypeptides, etc. For each such class, the present specification provides several examples of known sequences of such polypeptides. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions (e.g., isocitrate dehydrogenase polypeptides often share a conserved AMP-binding motif; HMG-CoA reductase polypeptides typically include a highly conserved catalytic domain (see, for example, FIG. 7); acetyl coA carboxylase typically has a carboxyl transferase domain; see, for example, Downing et al., Chem. Abs. 93:484, 1980; Gil et al., Cell 41:249, 1985; Jitrapakdee et al. Curr Protein Pept Sci. 4:217, 2003; U.S. Pat. No. 5,349,126, each of which is incorporated herein by reference in its entirety), usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented in the Tables herein.

Quinone biosynthesis polypeptide: A “quinone biosynthesis polypeptide”, as that term is used herein, refers to any polypeptide involved in the synthesis of one or more quinone derived compound, as described herein. In particular, quinone biosynthesis polypeptides include ubiquinone biosynthesis polypeptides, C₅₋₉ quinone biosynthesis polypeptides, vitamin K biosynthesis polypeptides, and vitamin E biosynthesis polypeptides.

Quinonogenic modification: The term “quinonogenic modifiaction, as used herein, refers to a modification of a host organism that adjusts production of one or more quinone derived compounds (e.g., ubiquinone, vitamin K compounds, vitamin E compounds, etc.), as described herein. For example, a quinonogenic modification may increase the production level of a particular quinone derived compound, or of a variety of different quinone derived compounds. In some embodiments of the disclosure, production of a particular quinone derived compound may be increased while production of other quinone derived compounds is decreased. In some embodiments of the disclosure, production of a plurality of different quinone derived compounds is increased. In principle, an inventive quinonogenic modification may be any chemical, physiological, genetic, or other modification that appropriately alters production of one or more quinone derived compounds in a host organism produced by that organism as compared with the level produced in an otherwise identical organism not subject to the same modification. In most embodiments, however, the quinonogenic modification will comprise a genetic modification, typically resulting in increased production of one or more quinone derived compounds (e.g., ubiquinone, vitamin K compounds, vitamin E compounds). In some embodiments, the quinonogenic modification comprises at least one chemical, physiological, genetic, or other modification; in other embodiments, the quinonogenic modification comprises more than one chemical, physiological, genetic, or other modification. In certain aspects where more than one modification is utilized, such modifications can comprise any combination of chemical, physiological, genetic, or other modification (e.g., one or more genetic, chemical and/or physiological modification(s)).

Retinologenic modification: The term “retinologenic modification”, as used herein, refers to a modification of a host organism that adjusts production of one or more retinolic compounds, as described herein. For example, a retinologenic modification may increase the production level of one or more retinolic compounds, and/or may alter relative production levels of different retinolic compounds. In principle, an inventive retinologenic modification may be any chemical, physiological, genetic, or other modification that appropriately alters production of one or more retinolic compounds in a host organism produced by that organism as compared with the level produced in an otherwise identical organism not subject to the same modification. In most embodiments, however, the retinologenic modification will comprise a genetic modification, typically resulting in increased production of one or more selected retinolic compounds. In some embodiments, the retinologenic modification comprises at least one chemical, physiological, genetic, or other modification; in other embodiments, the retinologenic modification comprises more than one chemical, physiological, genetic, or other modification. In certain aspects where more than one modification is utilized, such modifications can comprise any combination of chemical, physiological, genetic, or other modification (e.g., one or more genetic, chemical, and/or physiological modification(s)). In some embodiments, the selected retinolic compound is one or more of retinol, retinal, and retinoic acid. In some embodiments, the selected retinolic compound is retinol or esters of retinol, including but not limited to retinyl palmitate or retinyl acetate. In some embodiments, the selected retinolic compound is retinoic acid. In some embodiments, the selected retinolic compound is other than retinol.

Retinologenic polypeptide: The term “retinologenic polypeptide”, as used herein, refers to any polypeptide that is involved in the process of producing retinolic compounds in a cell, and may include polypeptides that are involved in processes other than retinolic compound production but whose activities affect the extent or level of production of one or more retinolic compounds, for example by scavenging a substrate or reactant utilized by a retinologenic polypeptide that is directly involved in retinolic compound production. Retinologenic polypeptides include retinolic compound biosynthesis polypeptides, isoprenoid biosynthesis polypeptides, carotenoid biosynthesis polypeptides, and isoprenoid biosynthesis competitor polypeptides, as those terms are defined herein. The term also encompasses polypeptides that may affect the extent to which retinolic compounds are accumulated in lipid bodies.

Retinolic compounds: The term “retinolic compound” is understood in the art to refer to a structurally similar class of compounds derived from certain carotenoids, collectively referred to as Vitamin A. All forms of Vitamin A have a beta-ionone ring to which an isoprenoid chain is attached. Retinolic compounds include, for example, retinol (the alcohol form), retinal (the aldehyde form), and retinoic acid (the acid form). Many different geometric isomers of retinol, retinal and retinoic acid are possible as a result of either a trans or cis configuration of four of the five double bonds found in the polyene chain. The cis isomers are less stable and can readily convert to the all-trans configuration. Nevertheless, some cis isomers are found naturally and carry out essential functions. For example, the 11-cis-retinal isomer is the chromophore of rhodopsin, the vertebrate photoreceptor molecule. The term retinolic compound also includes esters of retinol such as retinyl palmitate or retinyl acetate. Hydrolysis of retinyl esters results in retinol. Retinal, also known as retinaldehyde, can be reversibly reduced to produce retinol or it can be irreversibly oxidized to produce retinoic acid. The best described active retinoid metabolites are 11-cis-retinal and the all-trans and 9-cis-isomers of retinoic acid.

Retinolic compound biosynthesis polypeptides: The term “retinolic compound biosynthesis polypeptide” refers to any polypeptide that is involved in the synthesis of one or more retinolic compounds. To mention but a few, these retinolic compound biosynthesis polypeptides include, for example, polypeptides of beta-carotene 15,15′-monooxygenase (also known as beta-carotene dioxygenase) and/or beta-carotene retinol dehydrogenase. In some instances, a single gene may encode a protein with multiple retinolic compound biosynthesis polypeptide activities. Representative examples of retinolic compound biosynthesis polypeptide sequences are presented in Tables 67 and 68. As will be appreciated by those of ordinary skill in the art, in some embodiments of the disclosure, retinolic compound biosynthesis polypeptides include polypeptides that affect the expression and/or activity of one or more other retinolic compound biosynthesis polypeptides.

Small Molecule: In general, a small molecule is understood in the art to be an organic molecule that is less than about 5 kilodaltons (Kd) in size. In some embodiments, the small molecule is less than about 3 Kd, 2 Kd, or 1 Kd. In some embodiments, the small molecule is less than about 800 daltons (D), 600 D, 500 D, 400 D, 300 D, 200 D, or 100 D. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Source organism: The term “source organism”, as used herein, refers to the organism in which a particular polypeptide sequence can be found in nature. Thus, for example, if one or more heterologous polypeptides is/are being expressed in a host organism, the organism in which the polypeptides are expressed in nature (and/or from which their genes were originally cloned) is referred to as the “source organism”. Where multiple heterologous polypeptides are being expressed in a host organism, one or more source organism(s) may be utilized for independent selection of each of the heterologous polypeptide(s). It will be appreciated that any and all organisms that naturally contain relevant polypeptide sequences may be used as source organisms in accordance with the present disclosure. Representative source organisms include, for example, animal, mammalian, insect, plant, fungal, yeast, algal, bacterial, cyanobacterial, archaebacterial and protozoal source organisms.

Sterol biosynthesis polypeptide: The term “sterol biosynthesis polypeptide”, as used herein, refers to any polypeptide that is involved in the synthesis of one or more sterol compounds. Thus, sterol biosynthesis polypeptides can include isoprenoid biosynthesis polypeptides to the extent that they are involved in production of isopentyl pyrophosphate. Moreover, the term refers to any polypeptide that acts downstream of farnesyl pyrophosphate and in involved in the production of one or more sterol compounds. For example, sterol biosynthesis polypeptides include squalene synthase, which catalyses conversion of farnesyl pyrophosphate to presqualene pyrophosphate, and further catalyzes conversion of presqualene pyrophosphate to squalene, e.g., the enzyme with EC number 2.5.1.21. In some embodiments of the disclosure, sterol biosynthesis polypeptides further include one or more polypeptides involved in metabolizing squalene into a vitamin D compound. Thus, sterol biosynthesis polypeptides can include one or more of the polypeptides designated by EC number 1.14.99.7, 5.4.99.7, 5.4.99.8, 5.3.3.5, 1.14.21.6, 1.14.15.-, and/or 1.14.13.13, as well as other enzyme polypeptides involved in the sterol biosynthesis pathways. Furthermore, sterol biosynthesis polypeptides can include one or more enzyme polypeptides including, for example, C-14 demethylase (ERG9), squalene monooxygenase (ERG1), 2,3-oxidosqualene-lanosterol synthase (ERG7), C-1 demethylase (ERG11), C-14 reductase (ERG24), C-4 methyloxidase (ERG25), C-4 decarboxylase (ERG26), 3-ketoreductase (ERG27), C-24 methyltransferase (ERG6), 48-7 isomerase (ERG2), C-5 desaturase (ERG3), C-22 desaturase (ERG5) and/or C-24 reductase (ERG4) polypeptides, and/or other polypeptides involved in producing one or more vitamin D compounds (e.g., vitamin D2, vitamin D3, or a precursor thereof). As will be appreciated by those of ordinary skill in the art, in some embodiments of the disclosure, sterol biosynthesis polypeptides include polypeptides that affect the expression and/or activity of one or more other sterol biosynthesis polypeptides. Thus, for instance, transcription factors that regulate expression of sterol biosynthesis enzymes can be sterol biosynthesis polypeptides for purposes of the present disclosure. To give but a couple of examples, the S. cerevisiae Upc2 and YLR228c genes, and the Y. lipolytica YALI0B00660g gene encode transcription factors that are sterol biosynthesis polypeptides according to certain embodimentsof the present disclosure. For instance, the semidominant upc2-1 point mutation (G888D) exhibits increased sterol levels (Crowley et al., J. Bacteriol 180:4177-4183, 1998). Corresponding YLR228c mutants have been made and tested (Shianna et al., J Bacteriol 183:830, 2001); such mutants may be useful in accordance with the present disclosure, as may be YALI0B00660g derivatives with corresponding upc2-1 mutation(s). Representative examples of sterol biosynthesis polypeptide sequences are presented in Tables 53-66. As will be appreciated by those of ordinary skill in the art, in some embodiments of the disclosure, sterol biosynthesis polypeptides include polypeptides that affect the expression and/or activity of one or more other sterol biosynthesis polypeptides.

Sterologenic modification: The term “sterologenic modification”, as used herein, refers to a modification of a host organism that adjusts production of one or more sterol compounds (e.g., squalene, lanosterol, zymosterol, ergosterol, 7-dehydrocholesterol (provitamin D3), vitamin D compound(s), etc.), as described herein. For example, a sterologenic modification may increase the production level of a particular sterol compound, or of a variety of different sterol compounds. In some embodiments of the disclosure, production of a particular sterol compound may be increased while production of other sterol compounds is decreased. In some embodiments of the disclosure, production of a plurality of different sterol compounds is increased. In principle, an inventive sterologenic modification may be any chemical, physiological, genetic, or other modification that appropriately alters production of one or more sterol compounds in a host organism produced by that organism as compared with the level produced in an otherwise identical organism not subject to the same modification. In most embodiments, however, the sterologenic modification will comprise a genetic modification, typically resulting in increased production of one or more sterol compounds (e.g., squalene, lanosterol, zymosterol, ergosterol, 7-dehydrocholesterol (provitamin D3) or vitamin D compound(s)). In certain aspects where more than one modification is utilized, such modifications can comprise any combination of chemical, physiological, genetic, or other modification (e.g., one or more genetic modification and chemical or physiological modification).

Ubiquinone biosynthesis polypeptide: The term “ubiquinone biosynthesis polypeptide” refers to any polypeptide that is involved in the synthesis of ubiquinone. To mention but a few, these ubiquinone biosynthesis polypeptides include, for example, polypeptides of prenyldiphosphate synthase, PHB-polyprenyltransferase, and O-methyltransferase, as well as C₅₋₉ quinone biosynthesis polypeptides. As will be appreciated by those of ordinary skill in the art, in some embodiments of the disclosure, ubiquinone biosynthesis polypeptides include polypeptides that affect the expression and/or activity of one or more other ubiquinone biosynthesis polypeptides.

Ubiquinogenic modification: The term “ubiquinogenic modification”, as used herein, refers to a modification of a host organism that adjusts production of ubiquinone (e.g., CoQ10), as described herein. For example, a ubiquinogenic modification may increase the production level of ubiquinone (e.g., CoQ10), and/or may alter relative levels of ubiquinone and/or ubiquinol. In principle, an inventive ubiquinogenic modification may be any chemical, physiological, genetic, or other modification that appropriately alters production of ubiquinone (e.g., CoQ10) in a host organism produced by that organism as compared with the level produced in an otherwise identical organism not subject to the same modification. In most embodiments, however, the ubiquinogenic modification will comprise a genetic modification, typically resulting in increased production of ubiquinone(CoQ10).

Vitamin D biosynthesis polypeptide: The term “vitamin D biosynthesis polypeptide” refers to any polypeptide that is involved in the synthesis of one or more vitamin D compounds. To mention but a few, these include, for example, polypeptides enzymes with EC numbers the 1.14.99.7, 5.4.99.7, 5.4.99.8, 5.3.3.5, and/or 1.14.21.6. They further can include the hydroxylases that convert vitamin D₃ to calcitriol (e.g., polypeptides enzymes with EC numbers 1.14.15.- and 1.14.13.13). As will be appreciated by those of ordinary skill in the art, in some embodiments of the disclosure, vitamin D biosynthesis polypeptides include polypeptides that affect the expression and/or activity of one or more other vitamin D biosynthesis polypeptides.

Vitamin E biosynthesis polypeptide: The term “vitamin E biosynthesis polypeptide” refers to any polypeptide that is involved in the synthesis of vitamin E. To mention but a few, these include, for example, tyrA, pds1(hppd), VTE1, HPT1(VTE2), VTE3, VTE4, and/or GGH polypeptides (i.e., polypeptides that perform the chemical reactions performed by tyrA, pds1(hppd), VTE1, HPT1(VTE2), VTE3, VTE4, and/or GGH, respectively). As will be appreciated by those of ordinary skill in the art, in some embodiments of the disclosure, vitamin E biosynthesis polypeptides include polypeptides that affect the expression and/or activity of one or more other vitamin E biosynthesis polypeptides.

Vitamin K biosynthesis polypeptide: The term “vitamin K biosynthesis polypeptide” refers to any polypeptide that is involved in the synthesis of vitamin K. To mention but a few, these include, for example, MenF, MenD, MenC, MenE, MenB, MenA, UbiE, and/or MenG polypeptides (i.e., polypeptides that perform the chemical reactions performed by MenF, MenD, MenC, MenE, MenB, MenA, UbiE, and/or MenG, respectively). As will be appreciated by those of ordinary skill in the art, in some embodiments of the disclosure, vitamin K biosynthesis polypeptides include polypeptides that affect the expression and/or activity of one or more other carotenoid biosynthesis polypeptides.

### DETAILED DESCRIPTION OF CERTAIN PREFERRED EMBODIMENTS OF THE DISCLOSURE

As noted above, the present disclosure encompasses the discovery that carotenoids and/or retinolic compounds can desirably be produced in oleaginous yeast and fungi. According to the present disclosure, strains that both (i) accumulate lipid, often in the form of cytoplasmic oil bodies and typically to at least about 20% of their dry cell weight; and (ii) produce carotenoid(s) and/or retinolic compound(s) at a level at least about 1%, and in some embodiments at least about 3-20%, of their dry cell weight, are generated through manipulation of host cells (i.e., strains, including, e.g., naturally-occurring strains, strains which have been previously modified, etc.). These manipulated host cells are then used to produce carotenoids and/or retinolic compounds, so that carotenoids and/or retinolic compounds that partition into the lipid bodies can readily be isolated.

In general, it will be desirable to balance oleaginy and carotenoid production in inventive cells such that, as soon as a minimum desirable level of oleaginy is achieved, substantially all further carbon which is capable of being utilized and diverted into biosynthesis of products is diverted into a carotenoid and/or retinolic compounds production pathway. In some embodiments of the disclosure, this strategy involves engineering cells to be oleaginous; in other embodiments, it involves engineering cells to accumulate a higher level of lipid, particularly cytoplasmic lipid, than they would accumulate in the absence of such engineering even though the engineered cells may not become “oleaginous” as defined herein. In other embodiments, the extent to which an oleaginous host cell accumulates lipid is actually reduced so that remaining carbon can be utilized in carotenoid and/or retinolic compound production. According to the present disclosure, the extent of lipid accumulation in a host cell can be adjusted by modifying the level and/or activity of one or more polypeptides involved in lipid accumulation. Such modification can take the form of genetic engineering and/or exposure to particular growth conditions that induce or inhibit lipid accumulation.

To give but one example of adjustments that could be made to achieve a desired balance between oleaginy and carotenoid and/or retinolic compound production, we note that, while increasing acetyl CoA carboxylase expression (and/or activity) promotes oleaginy, decreasing its expression and/or activity can promote carotenoid and/or retinolic compound production. Those of ordinary skill in the art will appreciate that the expression and/or activity of acetyl CoA carboxylase, or of other polypeptides may be adjusted up or down as desired according to the characteristics of a particular host cell of interest.

We note that engineered cells and processes of using them as described herein may provide one or more advantages as compared with unmodified cells. Such advantages may include, but are not limited to: increased yield (e.g., carotenoid and/or retinolic compound content expressed as either % dry cell weight (mg/mg) or parts per million), titer (g carotenoid/L and/or g retinolic compound/L), specific productivity (mg carotenoid g⁻¹ biomass hour⁻¹ and/or mg retinolic compound g⁻¹ biomass hour⁻¹), and/or volumetric productivity (g carotenoid liter⁻¹ hour⁻¹ and/or g retinolic compound liter⁻¹ hour⁻¹)) of the desired carotenoid and/or retinolic compound (and/or intermediates thereof), and/or decreased formation of undesirable side products (for example, undesirable intermediates).

Thus, for example, the specific productivity for one or more desired carotenoids (e.g., β-carotene, astaxanthin), retinolic compound (e.g., retinol, retinal, retinoic acid), total carotenoids and/or total retinolic compounds may be at or about 0.1, at or about 0.11, at or about 0.12, at or about 0.13, at or about 0.14, at or about 0.15, at or about 0.16, at or about 0.17, at or about 0.18, at or about 0.19, at or about 0.2, at or about 0.21, at or about 0.22, at or about 0.23, at or about 0.24, at or about 0.25, at or about 0.26, at or about 0.27, at or about 0.28, at or about 0.29, at or about 0.3, at or about 0.31, at or about 0.32, at or about 0.33, at or about 0.34, at or about 0.35, at or about 0.36, at or about 0.37, at or about 0.38, at or about 0.39, at or about 0.4, at or about 0.41, at or about 0.42, at or about 0.43, at or about 0.44, at or about 0.45, at or about 0.46, at or about 0.47, at or about 0.48, at or about 0.49, at or about 0.5, at or about 0.51, at or about 0.52, at or about 0.53, at or about 0.54, at or about 0.55, at or about 0.56, at or about 0.57, at or about 0.58, at or about 0.59, at or about 0.6, at or about 0.61, at or about 0.62, at or about 0.63, at or about 0.64, at or about 0.65, at or about 0.66, at or about 0.67, at or about 0.68, at or about 0.69, at or about 0.7, at or about 0.71, at or about 0.72, at or about 0.73, at or about 0.74, at or about 0.75, at or about 0.76, at or about 0.77, at or about 0.78, at or about 0.79, at or about 0.8, at or about 0.81, at or about 0.82, at or about 0.83, at or about 0.84, at or about 0.85, at or about 0.86, at or about 0.87, at or about 0.88, at or about 0.89, at or about 0.9, at or about 0.91, at or about 0.92, at or about 0.93, at or about 0.94, at or about 0.95, at or about 0.96, at or about 0.97, at or about 0.98, at or about 0.99, at or about 1, 1.05, at or about 1.1, at or about 1.15, at or about 1.2, at or about 1.25, at or about 1.3, at or about 1.35, at or about 1.4, at or about 1.45, at or about 1.5, at or about 1.55, at or about 1.6, at or about 1.65, at or about 1.7, at or about 1.75, at or about 1.8, at or about 1.85, at or about 1.9, at or about 1.95, at or about 2 mg g⁻¹ hour⁻¹ or more.

Thus, for example, the volumetric productivity for one or more desired carotenoids (e.g., β-carotenoid, astaxanthin), retinolic compound (e.g., retinol, retinal, retinoic acid), total carotenoids and/or total retinolic compounds may be at or about 0.01, at or about 0.011, at or about 0.012, at or about 0.013, at or about 0.014, at or about 0.015, at or about 0.016, at or about 0.017, at or about 0.018, at or about 0.019, at or about 0.02, at or about 0.021, at or about 0.022, at or about 0.023, at or about 0.024, at or about 0.025, at or about 0.026, at or about 0.027, at or about 0.028, at or about 0.029, at or about 0.03, at or about 0.031, at or about 0.032, at or about 0.033, at or about 0.034, at or about 0.035, at or about 0.036, at or about 0.037, at or about 0.038, at or about 0.039, at or about 0.04, at or about 0.041, at or about 0.042, at or about 0.043, at or about 0.044, at or about 0.045, at or about 0.046, at or about 0.047, at or about 0.048, at or about 0.049, at or about 0.05, at or about 0.051, at or about 0.052, at or about 0.053, at or about 0.054, at or about 0.055, at or about 0.056, at or about 0.057, at or about 0.058, at or about 0.059, at or about 0.06, at or about 0.061, at or about 0.062, at or about 0.063, at or about 0.064, at or about 0.065, at or about 0.066, at or about 0.067, at or about 0.068, at or about 0.069, at or about 0.07, at or about 0.071, at or about 0.072, at or about 0.073, at or about 0.074, at or about 0.075, at or about 0.076, at or about 0.077, at or about 0.078, at or about 0.079, at or about 0.08, at or about 0.081, at or about 0.082, at or about 0.083, at or about 0.084, at or about 0.085, at or about 0.086, at or about 0.087, at or about 0.088, at or about 0.089, at or about 0.09, at or about 0.091, at or about 0.092, at or about 0.093, at or about 0.094, at or about 0.095, at or about 0.096, at or about 0.097, at or about 0.098, at or about 0.099, at or about 0.1, 0.105, at or about 0.110, at or about 0.115, at or about 0.120, at or about 0.125, at or about 0.130, at or about 0.135, at or about 0.14, at or about 0.145, at or about 0.15, at or about 0.155, at or about 0.16, at or about 0.165, at or about 0.17, at or about 0.175, at or about 0.18, at or about 0.185, at or about 0.19, at or about 0.195, at or about 0.20 grams liter⁻¹ hour⁻¹ or more.

### Host Cells

Those of ordinary skill in the art will readily appreciate that a variety of yeast and fungal strains exist that are naturally oleaginous or that naturally produce carotenoids. Yeast and fungal strains do not naturally produce retinolic compounds. Any of such strains may be utilized as host strains according to the present disclosure, and may be engineered or otherwise manipulated to generate inventive oleaginous, carotenoid-producing strains and/or oleaginous, retinolic acid compound-producing strains. Alternatively, strains that naturally are neither oleaginous nor: i) carotenoid-producing and/or ii) retinolic compound-producing may be employed. Furthermore, even when a particular strain has a natural capacity for oleaginy or for carotenoid production, its natural capabilities may be adjusted as described herein, so as to change the production level of lipid, carotenoid and/or retinolic compound. In certain embodiments engineering or manipulation of a strain results in modification of a type of lipid, carotenoid and/or retinolic compound which is produced. For example, a strain may be naturally oleaginous and/or carotenogenic, however engineering or modification of the strain may be employed so as to change the type of lipid which is accumulated and or to change the type of carotenoid which is produced. Additionally or alternatively, naturally oleaginous strain may be engineered to permit retinolic compound prouction. Moreover, further engineering or modification of the strain may be employed so as to change the type of lipid which is accumulated and/or to change the type of retinolic compound which is produced.

When selecting a particular yeast or fungal strain for use in accordance with the present disclosure, it will generally be desirable to select one whose cultivation characteristics are amenable to commercial scale production. For example, it will generally (though not necessarily always) be desirable to avoid filamentous organisms, or organisms with particularly unusual or stringent requirements for growth conditions. However, where conditions for commercial scale production can be applied which allow for utilization of filamentous organisms, these may be selected as host cells. In some embodiments of the disclosure, it will be desirable to utilize edible organisms as host cells, as they may optionally be formulated directly into food or feed additives, or into nutritional supplements, as desired. For ease of production, some embodiments of the disclosure utilize host cells that are genetically tractable, amenable to molecular genetics (e.g., can be efficiently transformed, especially with established or available vectors; optionally can incorporate and/or integrate multiple genes, for example sequentially; and/or have known genetic sequence; etc), devoid of complex growth requirements (e.g., a necessity for light), mesophilic (e.g., prefer growth temperatures with in the range of about 20-32° C.) (e.g., 20, 21, 22, 23, 24, 25, 26, 27, 28, 29, 30, 31, 32° C.), able to assimilate a variety of carbon and nitrogen sources and/or capable of growing to high cell density. Alternatively or additionally, various embodiments of the disclosure utilize host cells that grow as single cells rather than multicellular organisms (e.g., as mycelia).

In general, when it is desirable to utilize a naturally oleaginous organism in accordance with the present disclosure, any modifiable and cultivatable oleaginous organism may be employed. In certain embodiments of the disclosure, yeast or fungi of genera including, but not limited to, Blakeslea, Candida, Cryptococcus, Cunninghamella, Lipomyces, Mortierella, Mucor, Phycomyces, Pythium, Rhodosporidium, Rhodotorula, Trichosporon, and Yarrowia are employed. In certain particular embodiments, organisms of species that include, but are not limited to, Blakeslea trispora, Candida pulcherrima, C. revkaufi, C. tropicalis, Cryptococcus curvatus, Cunninghamella echinulata, C. elegans, C. japonica, Lipomyces starkeyi, L. lipoferus, Mortierella alpina, M. isabellina, M ramanniana, M vinacea, Mucor circinelloides, Phycomyces blakesleanus, Pythium irregulare, Rhodosporidium toruloides, Rhodotorula glutinis, R. gracilis, R. graminis, R. mucilaginosa, R. pinicola, Trichosporon pullans, T. cutaneum, and Yarrowia lipolytica are used.

Of these naturally oleaginous strains, some also naturally produce carotenoids and some do not; these strains do not naturally produced retinolic compounds. In most cases, only low levels (less than about 0.05% dry cell weight) of carotenoids are produced by naturally-occurring carotenogenic, oleaginous yeast or fungi. Higher levels of β-carotene are sometimes produced, but high levels of other carotenoids are generally not observed.

In general, any organism that is naturally oleaginous and non-carotenoid-producing (e.g., produce less than about 0.05% dry cell weight, do not produce the carotenoid of interest) may be utilized as a host cell in accordance with the present disclosure. Additionally or alternatively, any organism that is naturally oleaginous and non-retinolic compound-producing (e.g., produce less than about 0.05% dry cell weight, do not produce the retinolic compound of interest) may be utilized as a host cell in accordance with the present disclosure. For example, introduction of one or more retinologenic modifications (e.g., increased expression of one or more endogenous or heterologous retinologenic polypeptides), in accordance with the present disclosure, can achieve the goals for retinolic compound production. In some embodiments, the organism is a yeast or fungus from a genus such as, but not limited to, Candida, Cryptococcus, Cunninghamella, Lipomyces, Mortierella, Pythium, Trichosporon, and Yarrowia; in some embodiments, the organism is of a species including, but not limited to, Mortierella alpina and Yarrowia lipolytica.

Comparably, the present disclosure may utilize any naturally oleaginous, carotenoid-producing organism as a host cell. In general, the present disclosure may be utilized to increase carbon flow into the isoprenoid pathway in naturally carotenoid-producing organisms (particularly for organisms other than Blakeslea and Phycomyces), and/or to shift production from one carotenoid (e.g., β-carotene) to another (e.g., astaxanthin). Introduction of one or more carotenogenic modifications (e.g., increased expression of one or more endogenous or heterologous carotenogenic polypeptides), in accordance with the present disclosure, can achieve these goals. Additionally or alternatively, the present disclosure may be utilized to introduce the ability to produce one or more retinolic compounds in such naturally carotenoid-producing host cells.

In certain embodiments of the disclosure, the utilized oleaginous, carotenoid-producing organism is a yeast or fungus, for example of a genus such as, but not limited to, Blakeslea, Mucor, Phycomyces, Rhodosporidium, and Rhodotorula; in some embodiments, the organism is of a species such as, Mucor circinelloides and Rhodotorula glutinis.

When it is desirable to utilize strains that are naturally non-oleaginous as host cells in accordance with the present disclosure, genera of non-oleaginous yeast or fungi include, but are not limited to, Aspergillus, Botrytis, Cercospora, Fusarium (Gibberella), Kluyveromyces, Neurospora, Penicillium, Pichia (Hansenula), Puccinia, Saccharomyces, Sclerotium, Trichoderma, and Xanthophyllomyces (Phaffia); in some embodiments, the organism is of a species including, but not limited to, Candida utilis, Aspergillus nidulans, A. niger, A. terreus, Botrytis cinerea, Cercospora nicotianae, Fusarium fujikuroi (Gibberella zeae), Kluyveromyces lactis, K. lactis, Neurospora crassa, Pichia pastoris, Puccinia distincta, Saccharomyces cerevisiae, Sclerotium rolfsii, Trichoderma reesei, and Xanthophyllomyces dendrorhous (Phaffia rhodozyma).

It will be appreciated that the term “non-oleaginous”, as used herein, encompasses both strains that naturally have some ability to accumulate lipid, especially cytoplasmically, but do not do so to a level sufficient to qualify as “oleaginous” as defined herein, as well as strains that do not naturally have any ability to accumulate extra lipid, e.g., extra-membranous lipid. It will further be appreciated that, in some embodiments of the disclosure, it will be sufficient to increase the natural level of oleaginy of a particular host cell, even if the modified cell does not qualify as oleaginous as defined herein. In some embodiments, the cell will be modified to accumulate at least about 19%, 18%, 17%, 16%, 15%, 14%, 13%, 12%, 11%, 10%, 9%, 8%, 7%, 6%, or 5% in dry cell weight as lipid, so long as the accumulation level is more than that observed in the unmodified parental cell.

As with the naturally oleaginous organisms, some of the naturally non-oleaginous fungi naturally produce carotenoids, whereas others do not; these strains do not naturally produced retinolic compounds. Genera of naturally non-oleaginous fungi that do not naturally produce carotenoids (e.g., produce less than about 0.05% dry cell weight, do not produce a carotenoid or retinolic compound of interest) may desirably be used as host cells in accordance with the present disclosure include, but are not limited to, Aspergillus, Kluyveromyces, Penicillium, Saccharomyces, and Pichia; species include, but are not limited to, Candida utilis, Aspergillus niger and Saccharomyces cerevisiae. Genera of naturally non-oleaginous fungi that do naturally produce carotenoids or retinolic compounds and that may desirably be used as host cells in accordance with the present disclosure include, but are not limited to, Botrytis, Cercospora, Fusarium (Gibberella), Neurospora, Puccinia, Sclerotium, Trichoderma, and Xanthophyllomyces (Phaffia); species include, but are not limited to, Xanthophyllomyces dendrorhous (Phaffia rhodozyma).

As discussed above, any of a variety of organisms may be employed as host cells in accordance with the present disclosure. In certain embodiments of the disclosure, host cells will be Yarrowia lipolytica cells. Advantages of Y. lipolytica include, for example, tractable genetics and molecular biology, availability of genomic sequence (see, for example. Sherman et al. Nucleic Acids Res. 32(Database issue):D315-8, 2004), suitability to various cost-effective growth conditions, and ability to grow to high cell density. In addition, Y. lipolytica is naturally oleaginous, such that fewer manipulations may be required to generate an oleaginous, carotenoid-producing and/or retinolic compound-producing Y. lipolytica strain than might be required for other organisms. Furthermore, there is already extensive commercial experience with Y. lipolytica.

Saccharomyces cerevisiae is also a useful host cell in accordance with the present disclosure, particularly due to its experimental tractability and the extensive experience that researchers have accumulated with the organism. Although cultivation of Saccharomyces under high carbon conditions may result in increased ethanol production, this can generally be managed by process and/or genetic alterations.

Additional useful hosts include Xanthophyllomyces dendrorhous (Phaffia rhodozyma), which is experimentally tractable and naturally carotenogenic. Xanthophyllomyces dendrorhous (Phaffia rhodozyma) strains can produce several carotenoids, including astaxanthin.

Aspergillus niger and Mortierella alpina accumulate large amounts of citric acid and fatty acid, respectively; Mortierella alpina is also oleaginous.

Neurospora or Gibberella are also useful. They are not naturally oleaginous and tend to produce very low levels of carotenoids, thus extensive modification may be required in accordance with the present disclosure. Neurospora and Gibberella are considered relatively tractable from an experimental standpoint. Both are filamentous fungi, such that production at commercial scales can be a challenge necessary to overcome in utilization of such strains.

Mucor circinelloides is another available useful species. While its molecular genetics are generally less accessible than are those of some other organisms, it naturally produces β-carotene, thus may require less modification than other species available.

Candida utilis is a further useful species. Although it is not naturally oleaginous and produces little or no carotenoids, it is amenable to genetic manipulation (for example, see Iwakiri et al. (2006) Yeast 23:23-34, Iwakiri et al. (2005) Yeast 2005 22:1079-87, Iwakiri et al. (2005) Yeast 22:1049-60, Rodriquez et al. (1998) Yeast 14:1399-406, Rodriquez et al. (1998) FEMS Microbiol Lett. 165:335-40, and Kondo et al. (1995) J. Bacteriol. 177:7171-7) and furthermore is edible.

Molecular genetics can be performed in Blakeslea, though significant effort may be required. Furthermore, cost-effective fermentation conditions can be challenging, as, for example, it may be required that the two mating types are mixed. Fungi of the genus Phycomyces are also possible sources which have the potential to pose fermentation process challenges, and these fungi may be less amenable to manipulate than several other potential host organisms.

Additional useful hosts include strains such as Schizosaccharomyces pombe, Saitoella complicata, and Sporidiobolus ruineniae.

Those of ordinary skill in the art will appreciate that the selection of a particular host cell for use in accordance with the present disclosure will also affect, for example, the selection of expression sequences utilized with any heterologous polypeptide to be introduced into the cell, codon bias that can optionally be engineered into any nucleic acid to be expressed in the cell, and will also influence various aspects of culture conditions, etc. Much is known about the different gene regulatory requirements, protein targeting sequence requirements, and cultivation requirements, of different host cells to be utilized in accordance with the present disclosure (see, for example, with respect to Yarrowia, Barth et al. FEMS Microbiol Rev. 19:219, 1997; Madzak et al. J Biotechnol. 109:63, 2004; see, for example, with respect to Xanthophyllomyces, Verdoes et al. Appl Environ Microbiol 69: 3728-38, 2003; Visser et al. FEMS Yeast Res 4: 221-31, 2003; Martinez et al. Antonie Van Leeuwenhoek. 73(2):147-53, 1998; Kim et al. Appl Environ Microbiol. 64(5):1947-9, 1998; Wery et al. Gene. 184(1):89-97, 1997; see, for example, with respect to Saccharomyces, Guthrie and Fink Methods in Enzymology 194:1-933, 1991). In certain aspects, for example, targeting sequences of the host cell (or closely related analogs) may be useful to include for directing heterologous proteins to subcellular localization. Thus, such useful targeting sequences can be added to heterologous sequence for proper intracellular localization of activity. In other aspects (e.g., addition of mitochondrial targeting sequences), heterologous targeting sequences may be eliminated or altered in the selected heterologous sequence (e.g., alteration or removal of source organism plant chloroplast targeting sequences).

To give but a few specific examples, of promoters and/or regulatory sequences that may be employed in expression of polypeptides according to the present disclosure, useful promoters include, but are not limited to, the Leu2 promoter and variants thereof (see, for example, see U.S. Pat. No. 5,786,212); the EF1alpha protein and ribosomal protein S7 gene promoters (see, for example, PCT Application WO 97/44470); the Gpm (see US20050014270), Xpr2 (see U.S. Pat. No. 4,937,189), Tef1, Gpd1 (see, for example, US Application 2005-0014270A1), Cam1 (YALI0C24420g), YALI0D16467g, Tef4 (YALI0B12562g), Yef3 (YALI0E13277g), Pox2, Yat1 (see, for example US Application 2005-0130280; PCT Application WO 06/052754), Fbal (see, for example WO05049805), and/or Gpat (see WO06031937) promoters; the sequences represented by SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12, subsequences thereof, and hybrid and tandem derivatives thereof (e.g., as disclosed in US Application 2004-0146975); the sequences represented by SEQ ID NO: 1, 2, or 3 including fragments (e.g., by 462-1016 and by 197-1016 of SEQ ID NO: 1; by 5-523 of SEQ ID NO:3) and complements thereof (e.g., as disclosed in U.S. Pat. No. 5,952,195); CYP52A2A (see, for example, US Application 2002-0034788); promoter sequences from fungal (e.g., C. tropicalis) catalase, citrate synthase, 3-ketoacyl-CoA thiolase A, citrate synthase, O-acetylhornserine sulphydrylase, protease, carnitine O-acetyltransferase, hydratase-dehydrogenase, epimerase genes; promoter sequences from Pox4 genes (see, for example, US application 2004-0265980); and/or promoter sequences from Met2, Met3, Met6, Met25 and YALI0D12903g genes. Any such promoters can be used in conjunction with endogenous genes and/or heterologous genes for modification of expression patterns of endogenous polypeptides and/or heterologous polypeptides in accordance with the present disclosure.

Alternatively or additionally, regulatory sequences useful in accordance with the present disclosure may include one or more Xpr2 promoter fragments, for example as described in U.S. Pat. No. 6,083,717 (e.g., SEQ ID NOS: 1-4 also including sequences with 80% or more identity to these SEQ ID NOs) (e.g., see Example 11) in one or more copies either in single or in tandem. Similarly, exemplary terminator sequences include, but are not limited to, Y. lipolytica Xpr2 (see U.S. Pat. No. 4,937,189) and Pox2 (YALI0F10857g) terminator sequences.

In some embodiments of this disclosure, it may be desirable to fused sequences encoding specific targeting signals to bacterial source genes. For example, in certain embodiments mitochondrial signal sequences are useful in conjunction with, e.g., bacterial polypeptides for effective targeting of polypeptides for proper functioning. Mitochondrial signal sequences are known in the art, and include, but are not limited to example, mitochondrial signal sequences provided in Table 52 below. In other embodiments, it may be desirable to utilize genes from other source organisms such as animals, plants, alga, or microalgae, fungi, yeast, insect, protozoa, and mammals.

TABLE 52
Examples of mitochondrial targeting sequences.

| | | | |
|---|---|---|---|
| | Protein | | |
| Species | (residues) | GI | Sequence |

| | | | |
|---|---|---|---|
| Yarrowia | NUAM | 6689648 | MLSRNLSKFARAGLIRPATTSTHTRLFSVSARR |
| lipoylitica | (AA 1-34) | | L (SEQ ID NO: 1) |
| Yarrowia | NUHM | 50549567 | MLRLIRPRLAALARPTTRAPQALNARTHIVSV |
| lipoylitica | (AA 1-32) | | (SEQ ID NO: 2) |
| Saccharomyces | Coq1 | 536190 | MFQRSGAAHHIKLISSRRCRFKSSFAVALNAA |
| cerevisiae | (AA 1-53) | | SKLVTPKILWNNPISLVSKEM (SEQ ID NO: 3) |
| Yarrowia | Coq1 | 60389562 | MLRVGRIGTKTLASSSLRFVAGARPKSTLTEA |
| lipoylitica | (AA 1-77) | | VLETTGLLKTTPQNPEWSGAVKQASRLVETD |
| | | | TPIRDPFSIVSQEM (SEQ ID NO: 4) |

### Engineering Oleaginy

All living organisms synthesize lipids for use in their membranes and various other structures. However, most organisms do not accumulate in excess of about 10% of their dry cell weight as total lipid, and most of this lipid generally resides within cellular membranes.

Significant biochemical work has been done to define the metabolic enzymes necessary to confer oleaginy on microorganisms (primarily for the purpose of engineering single cell oils as commercial sources of arachidonic acid and docosahexaenoic acid; see for example Ratledge Biochimie 86:807, 2004, the entire contents of which are incorporated herein by reference). Although this biochemical work is compelling, there only have been a limited number of reports of de novo oleaginy being established through genetic engineering with the genes encoding the key metabolic enzymes. It should be noted that oleaginous organisms typically accumulate lipid only when grown under conditions of carbon excess and nitrogen limitation. The present disclosure further establishes that the limitation of other nutrients (e.g., phosphate and/or magnesium) can also induce lipid accumulation. The present disclosure establishes, for example, that limitation of nutrients such as phosphate and/or magnesium can induce lipid accumulation, much as is observed under conditions of nitrogen limitation. Under these conditions, the organism readily depletes the limiting nutrient but continues to assimilate the carbon source. The “excess” carbon is channeled into lipid biosynthesis so that lipids (usually triacylglycerols) accumulate in the cytosol, typically in the form of bodies. It should be noted that oleaginous organisms typically only accumulate lipid when grown under conditions of carbon excess and nitrogen or other nutrient limitation (e.g., phosphate or magnesium). Under these conditions, the organism readily depletes the limiting nutrient but continues to assimilate the carbon source. The “excess” carbon is channeled into lipid biosynthesis so that lipids (usually triacylglycerols) accumulate in the cytosol, typically in the form of bodies.

In general, it is thought that, in order to be oleaginous, an organism must produce both acetyl-CoA and NADPH in the cytosol, which can then be utilized by the fatty acid synthase machinery to generate lipids. In at least some oleaginous organisms, acetyl-CoA is generated in the cytosol through the action of ATP-citrate lyase, which catalyzes the reaction:

citrate+CoA+ATP→acetyl-CoA+oxaloacetate+ADP+Pᵢ. (1)

Of course, in order for ATP-citrate lyase to generate appropriate levels of acetyl-CoA in the cytosol, it must first have an available pool of its substrate citric acid. Citric acid is generated in the mitochondria of all eukaryotic cells through the tricarboxylic acid (TCA) cycle, and can be moved into the cytosol (in exchange for malate) by citrate/malate translocase.

In most oleaginous organisms, and in some non-oleaginous organisms, the enzyme isocitrate dehydrogenase, which operates as part of the TCA cycle in the mitochondria, is strongly AMP-dependent. Thus, when AMP is depleted from the mitochondria, this enzyme is inactivated. When isocitrate dehydrogenase is inactive, isocitrate accumulates in the mitochondria. This accumulated isocitrate is then equilibrated with citric acid, presumably through the action of aconitase. Therefore, under conditions of low AMP, citrate accumulates in the mitochondria. As noted above, mitochondrial citrate is readily transported into the cytosol.

AMP depletion, which in oleaginous organisms is believed to initiate the cascade leading to accumulation of citrate (and therefore acetyl-CoA) in the cytoplasm, occurs as a result of the nutrient depletion mentioned above. When oleaginous cells are grown in the presence of excess carbon source but under conditions limiting for nitrogen or some other nutrient(s) (e.g., phosphate or magnesium), the activity of AMP deaminase, which catalyzes the reaction:

AMP→inosine 5′-monophosphate+NH₃ (2)

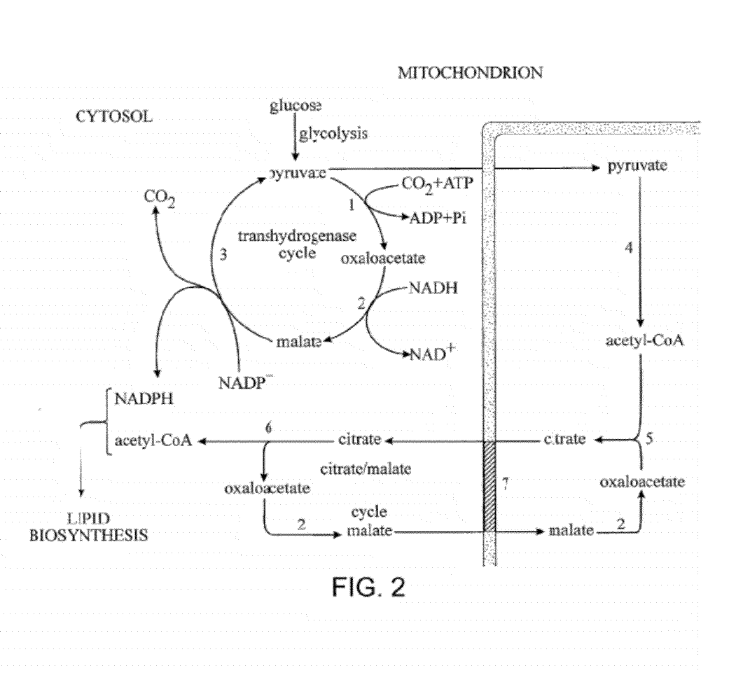

is strongly induced. The increased activity of this enzyme depletes cellular AMP in both the cytosol and the mitochondria. Depletion of AMP from the mitochondria is thought to inactivate the AMP-dependent isocitrate dehydrogenase, resulting in accumulation of citrate in the mitochondria and, therefore, the cytosol. This series of events is depicted diagrammatically in FIG. 2.

As noted above, oleaginy requires both cytosolic acetyl-CoA and cytosolic NADPH. It is believed that, in many oleaginous organisms, appropriate levels of cytosolic NADPH are provided through the action of malic enzyme (Enzyme 3 in FIG. 2). Some oleaginous organisms (e.g., Lipomyces and some Candida) do not appear to have malic enzymes, however, so apparently other enzymes can provide comparable activity, although it is expected that a dedicated source of NADPH is probably required for fatty acid synthesis (see, for example, Wynn et al., Microbiol 145:1911, 1999; Ratledge Adv. Appl. Microbiol. 51:1, 2002, each of which is incorporated herein by reference in its entirety).

Other activities which can be involved in regenerating NADPH include, for example, 6-phosphogluconate dehydrogenase (gnd); Fructose 1,6 bisphosphatase (fbp); Glucose 6 phosphate dehydrogenase (g6pd); NADH kinase (EC 2.7.1.86); and/or transhydrogenase (EC 1.6.1.1 and 1.6.1.2).

Gnd is part of the pentose phosphate pathway and catalyses the reaction:

6-phospho-D-gluconate+NADP+→D-ribulose 5-phosphate+CO₂+NADPH

Fbp is a hydrolase that catalyses the gluconeogenic reaction:

D-fructose 1,6-bisphosphate+H₂O→D-fructose 6-phosphate+phosphate

Fbp redirects carbon flow from glycolysis towards the pentose phosphate pathway. The oxidative portion of the pentose phosphate pathway, which includes glucose 6 phosphate dehydrogenase and 6-phosphogluconate dehydrogenase, enables the regeneration of NADPH. G6pd is part of the pentose phosphate pathway and catalyses the reaction:

D-glucose 6-phosphate+NADP⁺→D-glucono-1,5-lactone 6-phosphate+NADPH+H⁺ NADH

kinase catalyzes the reaction:

ATP+NADH→ADP+NADPH

Transhydrogenase catalyzes the reaction:

NADPH+NAD⁺ [symbol] NADP⁺+NADH

Thus, enhancing the expression and/or activity of any of these enzymes can increase NADPH levels and promote anabolic pathways requiring NADPH.

Alternative or additional strategies to promote oleaginy may include one or more of the following: (1) increased or heterologous expression of one or more of acyl-CoA:diacylglycerol acyltransferase (e.g., DGA1; YALI0E32769g); phospholipid: diacylglycerol acyltransferase (e.g., LRO1; YALI0E16797g); and acyl-CoA:cholesterol acyltransferase (e.g., ARE genes such as ARE1, ARE2, YALI0F06578g), which are involved in triglyceride synthesis (Kalscheuer et al. Appl Environ Microbiol p. 7119-7125, 2004; Oelkers et al. J Biol Chem 277:8877-8881, 2002; and Sorger et al. J Biol Chem 279:31190-31196, 2004), (2) decreased expression of triglyceride lipases (e.g., TGL3 and/or TGL4; YALI0D17534g and/or YALI0F10010g (Kurat et al. J Biol Chem 281:491-500, 2006); and (3) decreased expression of one or more acyl-coenzyme A oxidase activities, for example encoded by POX genes (e.g., POX1, POX2, POX3, POX4, POX5; YALI0C23859g, YALI0D24750g, YALI0E06567g, YALI0E27654g, YALI0E32835g, YALI0F10857g; see for example Mlickova et al. Appl Environ Microbiol 70: 3918-3924, 2004; Binns et al. J Cell Biol 173:719, 2006).

Thus, according to the present disclosure, the oleaginy of a host organism may be enhanced by modifying the expression or activity of one or more polypeptides involved in generating cytosolic acetyl-CoA and/or NADPH and/or altering lipid levels through other mechanisms. For example, modification of the expression or activity of one or more of acetyl-CoA carboxylase, pyruvate decarboxylase, isocitrate dehydrogenase, ATP-citrate lyase, malic enzyme, AMP-deaminase, glucose-6-phosphate dehydrogenase, 6-phosphogluconate dehydrogenase, fructose 1, 6 bisphosphatase, NADH kinase, transhydrogenase, acyl-CoA: diacylglycerol acyltransferase, phospholipid:diacylglycerol acyltransferase, acyl-CoA:cholesterol acyltransferase, triglyceride lipase, acyl-coenzyme A oxidase can enhance oleaginy in accordance with the present disclosure. Exemplary polypeptides which can be utilized or derived so as to enhance oleaginy in accordance with the present disclosure include, but are not limited to those acetyl-CoA carboxylase, pyruvate decarboxylase, isocitrate dehydrogenase, ATP-citrate lyase, malic enzyme, AMP-deaminase, glucose-6-phosphate dehydrogenase, 6-phosphogluconate dehydrogenase, fructose 1, 6 bisphosphatase, NADH kinase, transhydrogenase, acyl-CoA:diacylglycerol acyltransferase, phospholipid:diacylglycerol acyltransferase, acyl-CoA:cholesterol acyltransferase, triglyceride lipase, acyl-coenzyme A oxidase polypeptides provided in Tables 1-6, and 31-47, respectively.

In some embodiments of the disclosure, where an oleaginous host cell is employed, enzymes and regulatory components relevant to oleaginy are already in place but could be modified, if desired, by for example altering expression or activity of one or more oleaginic polypeptides and/or by introducing one or more heterologous oleaginic polypeptides. In those embodiments of the disclosure where a non-oleaginous host cell is employed, it is generally expected that at least one or more heterologous oleaginic polypeptides will be introduced.

The present disclosure contemplates not only introduction of heterologous oleaginous polypeptides, but also adjustment of expression or activity levels of heterologous or endogenous oleaginic polypeptides, including, for example, alteration of constitutive or inducible expression patterns. In some embodiments of the disclosure, expression patterns are adjusted such that growth in nutrient-limiting conditions is not required to induce oleaginy. For example, genetic modifications comprising alteration and/or addition of regulatory sequences (e.g., promoter elements, terminator elements) and/or regulatory factors (e.g., polypeptides that modulate transcription, splicing, translation, modification, etc.) may be utilized to confer particular regulation of expression patterns. Such genetic modifications may be utilized in conjunction with endogenous genes (e.g., for regulation of endogenous oleaginic polypeptide(s)); alternatively, such genetic modifications may be included so as to confer regulation of expression of at least one heterologous polypeptide (e.g., oleaginic polypeptide(s)).

In some embodiments, at least one oleaginic polypeptide is introduced into a host cell. In some embodiments of the disclosure, a plurality (e.g., two or more) of different oleaginic polypeptides is introduced into the same host cell. In some embodiments, the plurality of oleaginic polypeptides contains polypeptides from the same source organism; in other embodiments, the plurality includes polypeptides independently selected from different source organisms.

Representative examples of a variety of oleaginic polypeptides that may be introduced into or modified within host cells according to the present disclosure, include, but are not limited to, those provided in Tables 1-6, and Tables 31-47. As noted above, it is expected that at least some of these polypeptides (e.g., malic enzyme and ATP-citrate lyase) should desirably act in concert, and possibly together with one or more components of fatty acid synthase, such that, in some embodiments of the disclosure, it will be desirable to utilize two or more oleaginic polypeptides from the same source organism.

In certain embodiments, the oleaginy of a host organism is enhanced by growing the organism on a carbon source comprising one or more oils. For example, an organism may be grown on a carbon source comprising one or more oils selected from the group consisting of, for example, olive, canola, corn, sunflower, soybean, cottonseed, rapeseed, etc., and combinations thereof. In some embodiments, an organism is grown on a carbon source comprising soybean oil. In certain embodiments, the oleaginy of a host organism is enhanced by growing the organism on a carbon source comprising one or more oils in combination with modifying the expression or activity of one or more polypeptides such as any of those described above (e.g., oleaginic polypeptides such as polypeptides involved in generating cytosolic acetyl-CoA and/or NADPH) and/or altering lipid levels through other mechanisms.

In general, source organisms for oleaginic polypeptides to be used in accordance with the present disclosure include, but are not limited to, Blakeslea, Candida, Cryptococcus, Cunninghamella, Lipomyces, Mortierella, Mucor, Phycomyces, Pythium, Rhodosporidium, Rhodotorula, Trichosporon, Yarrowia, Aspergillus, Botrytis, Cercospora, Fusarium (Gibberella), Kluyveromyces, Neurospora, Penicillium, Pichia (Hansenula), Puccinia, Saccharomyces, Sclerotium, Trichoderma, and Xanthophyllomyces (Phaffia). In some embodiments, the source species for acetyl CoA carboxylase, ATP-citrate lyase, malice enzyme and/or AMP deaminase polypeptides include, but are not limited to, Aspergillus nidulans, Cryptococcus neoformans, Fusarium fujikuroi, Kluyveromyces lactis, Neurospora crassa, Saccharomyces cerevisiae, Schizosaccharomyces pombe, Ustilago maydis, and Yarrowia lipolytica; in some embodiments, source species for pyruvate decarboxylase or isocitrate dehydrogenase polypeptides include, but are not limited to Neurospora crassa, Xanthophyllomyces dendrorhous (Phaffia rhodozyma), Aspergillus niger, Saccharomyces cerevisiae, Mucor circinelloides, Rhodotorula glutinis, Candida utilis, Mortierella alpina, and Yarrowia lipolytica.

Aspergillus niger accumulates large amounts of citric acid, whereas Mortierella alpina and Thraustochytrium sp. accumulate large amounts of fatty acid, respectively; Mortierella alpina and Thraustochytrium are also oleaginous.

To give but one particular example of a host cell engineered to be oleaginous (or at least to accumulate increased levels of lipid) in accordance with the present disclosure, S. cerevisiae can be engineered to express one or more oleaginic polypeptides, e.g., from heterologous source organisms. In some embodiments, a plurality of different oleaginic polypeptides are expressed, optionally from different source organisms. For instance, in some embodiments, S. cerevisiae cells are engineered to express (and/or to increase expression of) ATP-citrate lyase (e.g., from N. crassa), AMP deaminase (e.g., from S. cerevisiae), and/or malic enzyme (e.g., from M. circinelloides). In other embodiments, Candida utilis and Phaffia rhodozyma can be similarly modified. Modified S. cerevisiae, C. utilis, and P. rhodozyma strains can be further modified as described herein to increase production of one or more carotenoids.

In certain embodiments, host cells are engineered to be olegaginous by introducing one or more oleaginic polypeptides. In general, any oleaginic polypeptide can be introduced into any host cell of the present disclosure. In certain embodiments, such oleaginic polypeptides are codon-optimized to accommodate the codon preferences of the host cell. In certain embodiments, an oleaginic polypeptide introduced into a host cell is from the same organsim as the host cell and/or a related organism. For example, without limitation, the present disclosure encompasses the recognition that it may be desirable to introduce a fungal oleaginic polypeptide into a fungal host cell (e.g., from the same and/or a related fungal species). In certain embodiments, the host cell is a Y. lipolytica host cell. In certain aspects of such embodiments, a Y. lipolytica olegainic polypeptide is introduced into the Y. lipolytica host cell. In certain aspects, a S. cerevisiae olegainic polypeptide is introduced into the Y. lipolytica host cell. In certain aspects, any of a variety of fungal olegainic polypeptides is introduced into the Y. lipolytica host cell.

### Engineering Carotenoid Production

Carotenoids are synthesized from isoprenoid precursors, some of which are also involved in the production of steroids and sterols. The most common isoprenoid biosynthesis pathway, sometimes referred to as the “mevalonate pathway”, is generally depicted in FIG. 3. As shown, acetyl-CoA is converted, via hydroxymethylglutaryl-CoA (HMG-CoA), into mevalonate. Mevalonate is then phosphorylated and converted into the five-carbon compound isopentenyl pyrophosphate (IPP). Following isomerization of IPP into dimethylallyl pyrophosphate (DMAPP), three sequential condensation reactions with additional molecules of IPP generate the ten-carbon molecule geranyl pyrophosphate (GPP), followed by the fifteen-carbon molecule farnesyl pyrophosphate (FPP), and finally the twenty-carbon compound geranylgeranyl pyrophosphate (GGPP).

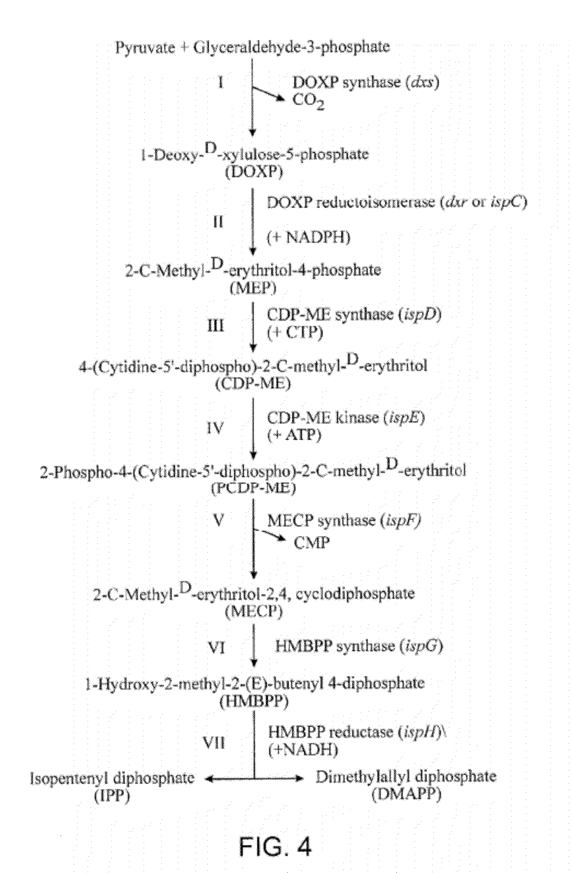

An alternative isoprenoid biosynthesis pathway, that is utilized by some organisms (particularly bacteria) and is sometimes called the “mevalonate-independent pathway”, is depicted in FIG. 4. This pathway is initiated by the synthesis of 1-deoxy-D-xyloglucose-5-phosphate (DOXP) from pyruvate and glyceraldehyde-3-phosphate. DOXP is then converted, via a series of reactions shown in FIG. 4, into IPP, which isomerizes into DMAPP and is then converted, via GPP and FPP, into GGPP as shown in FIG. 3 and discussed above.

Various proteins involved in isoprenoid biosynthesis have been identified and characterized in a number of organisms. Moreover, various aspects of the isoprenoid biosynthesis pathway are conserved throughout the fungal, bacterial, plant and animal kingdoms. For example, polypeptides corresponding to the acetoacetyl-CoA thiolase, HMG-CoA synthase, HMG-CoA reductase, mevalonate kinase, phosphomevalonate kinase, mevalonate pyrophosphate decarboxylase, IPP isomerase, FPP synthase, and GGPP synthase shown in FIG. 3 have been identified in and isolated from a wide variety of organisms and cells. Representative examples of a wide variety of such polypeptides are provided in Tables 7-15. One or more of the polypeptides selected from those provided in any one of Tables 7-15 may be utilized or derived for use in the methods and compositions in accordance with the present disclosure.

Alternatively or additionally, modified mevalonate kinase polypeptides that exhibit decreased feedback inhibition properties (e.g., to farnesyl pyrophosphate (FPP)) may be utilized in accordance with the present disclosure. Such modified mevalonate kinase polypeptides may be of eukaryotic or prokaryotic origin. For example, modified versions of mevalonate kinase polypeptides from animals (including humans), plants, algae, fungi (including yeast), and/or bacteria may be employed; for instance, modified versions of mevalonate kinase polypeptides disclosed in Table 10 herein may be utilized.

Particular examples of modified mevalonate kinase polypeptides include “feedback-resistant mevalonate kinases” disclosed in PCT Application WO 2006/063752. Thus, for example, a modified mevalonate kinase polypeptide may include one or more mutation (s) at one or more amino acid position (s) selected from the group consisting of amino acid positions corresponding to positions 17, 47, 93, 94, 132, 167, 169, 204, and 266 of the amino acid sequence of Paracoccus zeaxanthinifaciens mevalonate kinase as shown in SEQ ID NO: 1 of PCT Application WO 2004/111214. For example, the modified mevalonate kinase polypeptide may contain one or more substitutions at positions corresponding to one or more of I17T, G47D, K93E, V94I, R204H and C266S.

To give but a few specific examples, when a modified mevalonate kinase polypeptide comprises 2 amino acid changes as compared with a parent mevalonate kinase polypeptide, it may comprise changes at positions corresponding to the following positions 132/375, 167/169, 17/47 and/or 17/93 of SEQ ID NO: 1 of WO/2004/111214 (e.g., P132A/P375R, R167W/K169Q, I17T/G47D or I17T/K93E); when a modified mevalonate kinase polypeptide comprises 3 amino acid changes as compared with a parent mevalonate kinase, it may comprise changes at positions corresponding to the following positions 17/167/169, 17/132/375, 93/132/375, and/or 17/47/93 of SEQ ID NO: 1 of WO/2004/111214 (e.g., I17T/R167W/K169Q, I17T/P132A/P375R, K93E/P132A/P375R, I17T/R167W/K169H, I17T/R167T/K169M, I17T/R167T/K169Y, I17T/R167F/K169Q, I17T/R167I/K169N, I17T/R167H/K169Y, I17T/G47D/K93E or I17T/G47D/K93Q).

Thus, for example, a modified mevalonate kinase polypeptide may include one or more mutation(s) (particularly substitutions), as compared with a parent mevalonate kinase polypeptide, at one or more amino acid position (s) selected from the group consisting of amino acid positions corresponding to positions 55, 59, 66, 83, 106, 111, 117, 142, 152, 158, 218, 231, 249, 367 and 375 of the amino acid sequence of Saccharomyces cerevisiae mevalonate kinase as shown in SEQ ID NO: 1 of PCT application WO 2006/063752. For example, such corresponding substitutions may comprise one or more of P55L, F59S, N66K, C117S, or I152M. A modified mevalonate kinase may comprise a substitution corresponding to F59S substitution. A modified mevalonate kinase polypeptide comprising 2 amino acid changes as compared with its parent mevalonate kinase polypeptide may, for example, comprise changes at positions corresponding to the following positions 55/117, 66/152, 83/249, 111/375 or 106/218 of to SEQ ID NO: 1 of WO2006/063752 (e.g., P55L/C117S, N66K/I152M, K83E/S249P, H111N/K375N or L106P/S218P). A modified mevalonate kinase may comprise a substitution corresponding to N66K/1152M. A modified mevalonate kinase polypeptide comprising 4 amino acid changes as compared with its parent mevalonate kinase polypeptide may have changes at positions corresponding to one or more of the following positions 42/158/231/367 of SEQ ID NO:1 of WO2006/063752 (e.g., I142N/L158S/L231I/T367S).

According to the present disclosure, carotenoid production in a host organism may be adjusted by modifying the expression or activity of one or more proteins involved in isoprenoid biosynthesis. In some embodiments, such modification involves introduction of one or more heterologous isoprenoid biosynthesis polypeptides into the host cell; alternatively or additionally, modifications may be made to the expression or activity of one or more endogenous or heterologous isoprenoid biosynthesis polypeptides. Given the considerable conservation of components of the isoprenoid biosynthesis polypeptides, it is expected that heterologous isoprenoid biosynthesis polypeptides will often function even in significantly divergent organisms. Furthermore, should it be desirable to introduce more than one heterologous isoprenoid biosynthesis polypeptide (e.g., more than one version of the same polypeptide and/or more that one different polypeptides), in many cases polypeptides from different source organisms will function together. In some embodiments of the disclosure, a plurality of different heterologous isoprenoid biosynthesis polypeptides is introduced into the same host cell. In some embodiments, this plurality contains only polypeptides from the same source organism (e.g., two or more sequences of, or sequences derived from, the same source organism); in other embodiments the plurality includes polypeptides independently selected from from different source organisms (e.g., two or more sequences of, or sequences derived from, at least two independent source organisms).

In some embodiments of the present disclosure that utilize heterologous isoprenoid biosynthesis polypeptides, the source organisms include, but are not limited to, fungi of the genera Blakeslea, Candida, Cryptococcus, Cunninghamella, Lipomyces, Mortierella, Mucor, Phycomyces, Pythium, Rhodosporidium, Rhodotorula, Trichosporon, Yarrowia, Aspergillus, Botrytis, Cercospora, Fusarium (Gibberella), Kluyveromyces, Neurospora, Penicillium, Pichia (Hansenula), Puccinia, Saccharomyces, Schizosaccharomyces, Sclerotium, Trichoderms, Ustilago, and Xanthophyllomyces (Phaffia). In certain embodiments, the source organisms are of a species including, but not limited to, Cryptococcus neoformans, Fusarium fujikuroi, Kluyverimyces lactis, Neurospora crassa, Saccharomyces cerevisiae, Schizosaccharomyces pombe, Ustilago maydis, and Yarrowia lipolytica.

As noted above, the isoprenoid biosynthesis pathway is also involved in the production of non-carotenoid compounds, such as sterols, steroids, and vitamins, such as vitamin E or vitamin K. Proteins that act on isoprenoid biosynthesis pathway intermediates, and divert them into biosynthesis of non-carotenoid compounds are therefore indirect inhibitors of carotenoid biosynthesis (see, for example, FIG. 5, which illustrates points at which isoprenoid intermediates are channeled into other biosynthesis pathways). Such proteins are therefore considered isoprenoid biosynthesis competitor polypeptides. Reductions of the level or activity of such isoprenoid biosynthesis competitor polypeptides are expected to increase carotenoid production in host cells according to the present disclosure. Additionally or alternatively, since disruption of the SAGA complex component SPT-8 increases carotenoid production (see e.g., Example 16), increased expression or activity of one or more components of the SAGA complex such as, without limitation, the SPT8 gene, may decrease production of carotenoids and/or retinolic compounds. Thus, polypeptides that comprise the SAGA complex can be considered competitor polypeptides in the situation where they decrease production of carotenoids and/or retinolic compounds. Without wishing to be bound by theory, the present disclosure encompasses the recognition that increased expression or activity of one or more components of the SAGA complex may act as isoprenoid biosynthesis competitors, thus reducing the amount of carotenoid produced. For example, one or more components of the SAGA complex may act on isoprenoid intermediates prior to GGPP, such that less GGPP is generated and available for the carotenoid generation pathway. In such embodiments, it will be understood that the SAGA polypeptide(s) components whose activity or expression is increased functions as isoprenoid biosynthesis competitor polypeptide(s). Thus, for example, one or more of the polypeptides encoded by the genes listed in Table 69 may function as isoprenoid biosynthesis competitor polypeptides. Such SAGA polypeptides can be expressed individually or in combination with one another. In certain embodiments, SAGA isoprenoid biosynthesis competitor polypeptides are expressed (and/or their activity increased) in combination with an increase in expression and/or activity of one or more additional isoprenoid biosynthesis competitor polypeptides, such as, without limitation, those isoprenoid biosynthesis competitor polypeptides listed in Tables 7-15.

In some embodiments of the present disclosure, production or activity of endogenous isoprenoid biosynthesis competitor polypeptides may be reduced or eliminated in host cells. In some embodiments, this reduction or elimination of the activity of an isoprenoid biosynthesis competitor polypeptide can be achieved by treatment of the host organism with small molecule inhibitors of enzymes of the ergosterol biosynthetic pathway. Enzymes of the ergosterol biosynthetic pathway include, for example, squalene synthase (Erg9), squalene epoxidase (Erg1), 2,3-oxidosqualene-lanosterol cyclase (Erg7), cytochrome P450 lanosterol 14α-demethylase (Erg11), C-14 sterol reductase (Erg24), C-4 sterol methyl oxidase (Erg25), SAM:C-24 sterol methyltransferase (Erg6), C-8 sterol isomerase (Erg2), C-5 sterol desaturase (Erg3), C-22 sterol desaturase (Erg5), and C-24 sterol reductase (Erg4) polypeptides. Each of these enzymes is considered an isoprenoid biosynthesis competitor polypeptide. Regulators of these enzymes may also be considered isoprenoid biosynthesis competitor polypeptides (e.g., the yeast proteins Sut1 (Genbank Accession JC4374 GI:2133159) and Mot3 (Genbank Accession NP_{—}013786 GI:6323715), which may or may not have homologs in other organisms.

In other embodiments, reduction or elimination of the activity of an isoprenoid biosynthesis competitor polypeptide can be achieved by decreasing activity of the ubiquinone biosynthetic pathway. The commitment step in ubiquinone biosynthesis is the formation of para-hydroxybenzoate (PHB) from tyrosine or phenylalanine in mammals or chorismate in bacteria, followed by condensation of PHB and isoprene precursor, resulting in addition of the prenyl group. This reaction is catalyzed by PHB-polyprenyltransferase. The isoprenoid side chain of ubiquinone, which can be of varying length in different organisms, is determined by the prenyldiphosphate synthase enzyme. In organisms that produce the coenzyme Q10 form of ubiquinone, the 3-decaprenyl-4-hydroxybenzoic acid resulting from the condensation of PHB and decaprenyldiphosphate reaction undergoes further modifications, which include hydroxylation, methylation and decarboxylation, in order to form ubiquinone (CoQ10). Thus, reducing the activity of prenyldiphosphate synthase leading from farnesyldiphosphate to extended isoprenoids, or reducing the activity of PHB polyprenyltransferase may be useful in increasing the amount of isoprenoid available for carotenoid biosynthesis. (Examples of prenyldiphosphate synthase and PHB-polyprenyltransferase enzymes are depicted in Tables 29 and 30, respectively).

Known small molecule inhibitors of isoprenoid biosynthesis competitor enzymes include, but are not limited to, zaragosic acid (including analogs thereof such as TAN1607A (Biochem Biophys Res Commun 1996 Feb. 15; 219(2):515-520)), RPR 107393 (3-hydroxy-3-[4-(quinolin-6-yl)phenyl]-1-azabicyclo[2-2-2]octane dihydrochloride; J Pharmacol Exp Ther. 1997 May; 281(2):746-52), ER-28448 (5-{N-[2-butenyl-3-(2-methoxyphenyl)]-N-methylamino}-1,1-penthylidenebis(phosphonic acid) trisodium salt; Journal of Lipid Research, Vol. 41, 1136-1144, July 2000), BMS-188494 (The Journal of Clinical Pharmacology, 1998; 38:1116-1121), TAK-475 (1-[2-[(3R,5S)-1-(3-acetoxy-2,2-dimethylpropyl)-7-chloro-1,2,3,5-tetrahydro-2-oxo-5-(2,3-dimethoxyphenyl)-4,1-benzoxazepine-3-yl]acetyl]piperidin-4-acetic acid; Eur J. Pharmacol. 2003 Apr. 11; 466(1-2):155-61), YM-53601 ((E)-2-[2-fluoro-2-(quinuclidin-3-ylidene)ethoxy]-9H-carbazole monohydrochloride; Br J. Pharmacol. 2000 September; 131(1):63-70), or squalestatin I that inhibit squalene synthase; terbinafine (e.g., LAMISIL®), naftifine (NAFTIN®), S-allylcysteine, garlic, resveratrol, NB-598 (e.g., from Banyu Pharmaceutical Co), and/or green tea phenols that inhibit squalene epoxidase (see, for example, J. Biol Chem 265:18075, 1990; Biochem. Biophys. Res. Commun. 268:767, 2000); various azoles that inhibit cytochrome P450 lanosterol 14α-demethylase; and fenpropimorph that inhibits the C-14 sterol reductase and the C-8 sterol isomerase. In other embodiments, heterologous isoprenoid biosynthesis competitor polypeptides may be utilized (whether functional or non-functional; in some embodiments, dominant negative mutants are employed).

One particular isoprenoid biosynthesis competitor polypeptide useful according to the present disclosure is squalene synthase which has been identified and characterized from a variety of organisms; representative examples of squalene synthase polypeptide sequences are included in Table 16. In some embodiments of the disclosure that utilize squalene synthase (or modifications of squalene synthase) source organisms include, but are not limited to, Neurospora crassa, Xanthophyllomyces dendrorhous (Phaffia rhodozyma), Aspergillus niger, Saccharomyces cerevisiae, Mucor circinelloides, Rhotorula glutinis, Candida utilis, Mortierella alpina, and Yarrowia lipolytica.

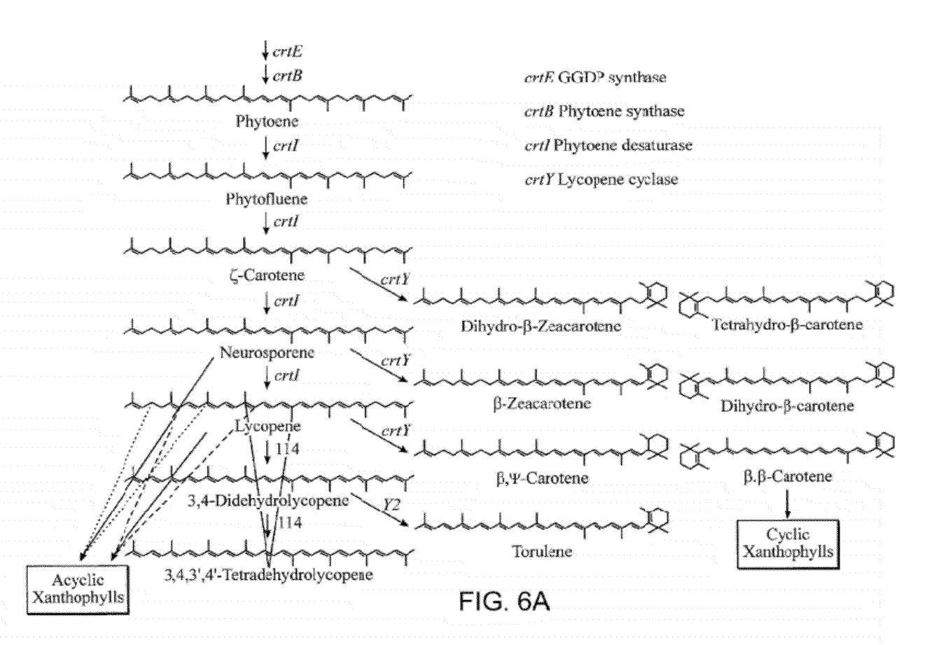

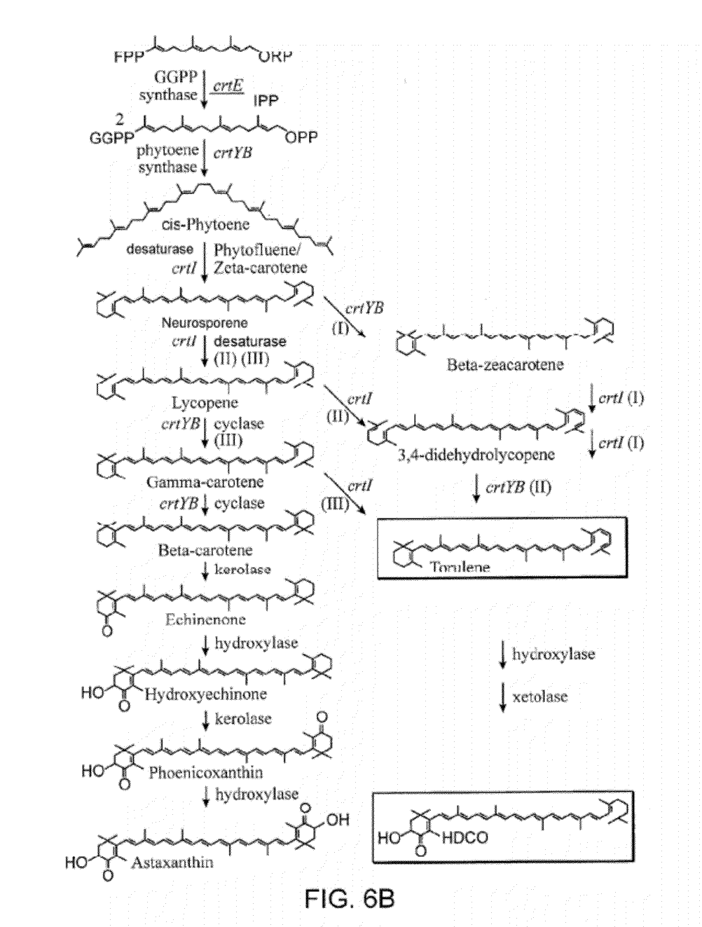

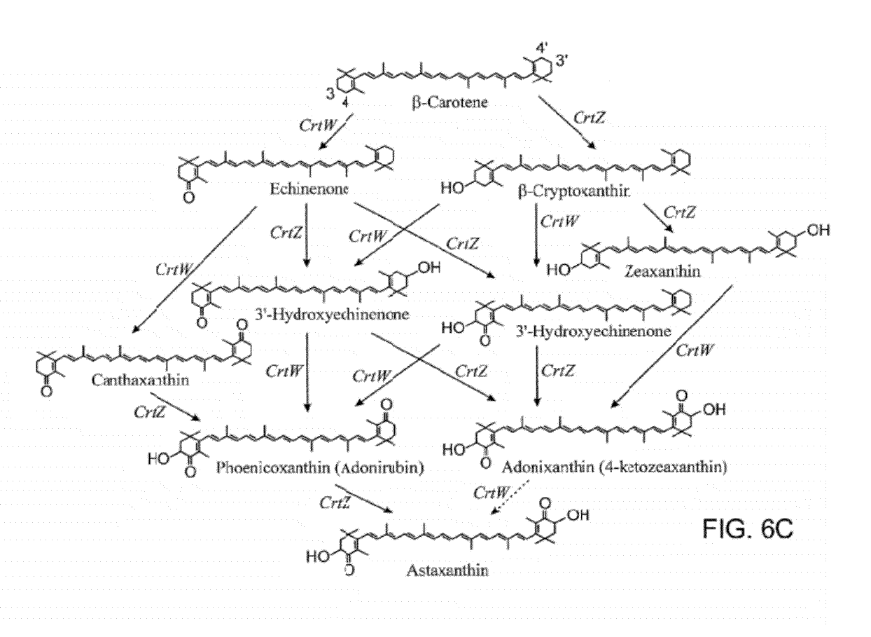

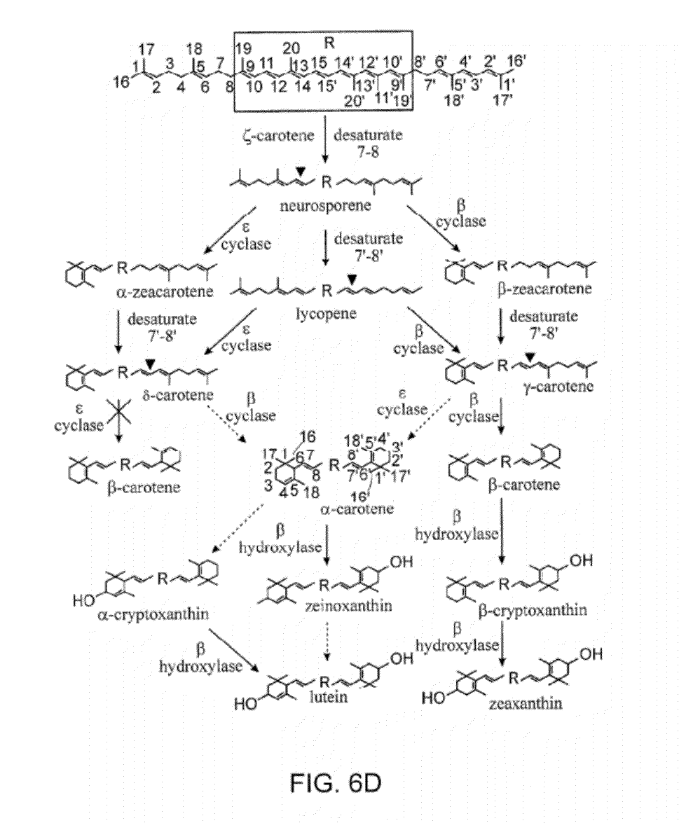

The carotenoid biosynthesis pathway branches off from the isoprenoid biosynthesis pathway at the point where GGPP is formed. The commitment step in carotenoid biosynthesis is the formation of phytoene by the head-to-head condensation of two molecules of GGPP, catalyzed by phytoene synthase (often called crtB; see FIG. 6). A series of dehydrogenation reactions, each of which increases the number of conjugated double bonds by two, converts phytoene into lycopene via neurosporene. The pathway branches at various points, both before and after lycopene production, so that a wide range of carotenoids can be generated. For example, action of a cyclase enzyme on lycopene generates γ-carotene; action of a desaturase instead produces 3,4-didehydrolycopene. γ-carotene is converted to β-carotene through the action of a cyclase. β-carotene can be processed into any of a number of products (see, for example, FIG. 6C), including astaxanthin (via echinenone, hydroxyechinenone, and phoenicoxanthin).

According to the present disclosure, carotenoid production in a host organism may be adjusted by modifying the expression or activity of one or more proteins involved in carotenoid biosynthesis. As indicated, in some embodiments, it will be desirable to utilize as host cells organisms that naturally produce one or more carotenoids. In some such cases, the focus will be on increasing production of a naturally-produced carotenoid, for example by increasing the level and/or activity of one or more proteins involved in the synthesis of that carotenoid and/or by decreasing the level or activity of one or more proteins involved in a competing biosynthetic pathway. Alternatively or additionally, in some embodiments it will be desirable to generate production of one or more carotenoids not naturally produced by the host cell.

According to some embodiments of the disclosure, it will be desirable to introduce one or more heterologous carotenogenic polypeptides into a host cell. As will be apparent to those of ordinary skill in the art, any of a variety of heterologous polypeptides may be employed; selection will consider, for instance, the particular carotenoid whose production is to be enhanced. The present disclosure contemplates not only introduction of heterologous carotenogenic polypeptides, but also adjustment of expression or activity levels of heterologous or endogenous carotenogenic polypeptides, including, for example, alteration of constitutive or inducible expression patterns. In some embodiments of the disclosure, expression patterns are adjusted such that growth in nutrient-limiting conditions is not required to induce oleaginy. For example, genetic modifications comprising alteration and/or addition of regulatory sequences (e.g., promoter elements, terminator elements) may be utilized to confer particular regulation of expression patterns. Such genetic modifications may be utilized in conjunction with endogenous genes (e.g., for regulation of endogenous carotenogenic); alternatively, such genetic modifications may be included so as to confer regulation of expression of at least one heterologous polypeptide (e.g., carotenogenic polypeptide(s)). For example, promoters including, but not limited to those described herein, such as Tef1, Gpd1 promoters can be used in conjunction with endogenous genes and/or heterolous genes for modification of expression patterns of endogenous carotenogenic polypeptide(s) and/or heterologous carotenogenic polypeptide(s). Similarly, exemplary terminator sequences include, but are not limited to, use of Y. lipolytica XPR2 terminator sequences.

As indicated in FIG. 6 and in the literature, proteins involved in carotenoid biosynthesis include, but are not limited to, phytoene synthase, phytoene dehydrogenase, lycopene cyclase, carotenoid ketolase, carotenoid hydroxylase, astaxanthin synthase (a single multifunctional enzyme found in some source organisms that typically has both ketolase and hydroxylase activities), carotenoid epsilon hydroxylase, lycopene cyclase (beta and epsilon subunits), carotenoid glucosyltransferase, and acyl CoA:diacyglycerol acyltransferase. Representative example sequences for these carotenoid biosynthesis polypeptides are provided in Tables 17a-25.

Alternatively or additionally, modified carotenoid ketolase polypeptides that exhibit improved carotenoid production activity may be utilized in accordance with the present disclosure. For example, carotenoid ketolase polypeptides comprising one more mutations to corresponding to those identified Sphingomonas sp. DC18 which exhibited improved astaxanthin production (Tao et al. 2006 Metab Eng. 2006 Jun. 27) and Paracoccus sp. strain N81106 which exhibited altered carotenoid production (Ye et al. 2006 Appl Environ Microbiol 72:5829-37).

Xanthophylls can be distinguished from other carotenoids by the presence of oxygen containing functional groups on their cyclic end groups. For instance, lutein and zeaxanthin contain a single hydroxyl group on each of their terminal ring structures, while astaxanthin contains both a keto group and a hydroxyl on each terminal ring. This property makes xanthophylls more polar than carotenes such as beta-carotene and lycopene, and thus dramatically reduces their solubility in fats and lipids. Naturally occurring xanthophylls are often found as esters of the terminal hydroxyl groups, both mono- and diesters of fatty acids. They also occur as glucosides in certain species of bacteria. The solubility and dispersibility of xanthophylls can be greatly modified by the addition of ester moieties, and it is known that esterification can also affect the absorbability and/or bioavailability of a given carotenoid. It is an objective of this disclosure to maximize the amount of a particular xanthophyll accumulating within the intracellular triacylglyceride fraction of oleaginous yeasts, and one mechanism for achieving this goal is to increase the hydrophobic nature of the xanthophyll product that accumulates. One way of achieving this is to engineer the production of fatty-acyl mono- and/or diesters of the target xanthophyll compound.

A variety of enzymes can function to esterify carotenoids. For example, carotenoid glucosyltransferases have been identified in several bacterial species (see, e.g., Table 24). In addition, acyl CoA:diacyglycerol acyltransferase (DGAT) and acyl CoA:monoacylglycerol acyltransferases (MGAT), which function in the final steps of triacylglycerol biosynthesis, are likely to serve an additional role in the esterification of xanthophylls. Representative DGAT polypetides are shown in Table 25. Furthermore, other enzymes may specifically modify carotenoids and molecules of similar structure (e.g., sterols) and be available for modification and ester production.

In some embodiments of the disclosure, potential source organisms for carotenoid biosynthesis polypeptides include, but are not limited to, genera of naturally oleaginous or non-oleaginous fungi that naturally produce carotenoids. These include, but are not limited to, Botrytis, Cercospora, Fusarium (Gibberella), Mucor, Neurospora, Phycomyces, Puccina, Rhodotorula, Sclerotium, Trichoderma, and Xanthophyllomyces. Exemplary species include, but are not limited to, Neurospora crassa, Xanthophyllomyces dendrorhous (Phaffia rhodozyma), Mucor circinelloides, and Rhodotorula glutinis. Of course, carotenoids are produced by a wide range of diverse organisms such as plants, algae, yeast, fungi, bacteria, cyanobacteria, etc. Any such organisms may be source organisms for carotenoid biosynthesis polypeptides according to the present disclosure.

In certain embodiments of the disclosure, carotenoid production in a host organism may be adjusted by modifying the activity of one or more endogenous genes that affect carotenoid biosynthesis. For example, as shown in Example 16, disruption of the endogenous SPT8 gene (YALI0E23804g) in Yarrowia lipolytica results in increased carotenoid production. SPT8 functions as part of the SAGA histone acetyltransferase complex, which is required for normal expression of some fungal genes and is thought to function as a coactivator complex in a multistep pathway leading to gene activation. Thus, without wishing to be bound by theory, the present disclosure encompasses the recognition that alteration of the expression and/or activity of one or more components of the SAGA histone acetyltransferase complex result in increased carotenoid production. Additionally, it will be appreciated by those of ordinary skill in the art that by increasing production of carotenoid(s) in a host organism by altering the expression and/or activity of one or more components of the SAGA histone acetyltransferase complex, production of a retinolic compound(s) in a host organism able to utilize such a carotenoid(s) as a substrate may also be increased since more of the cartenoid substrate will be available.

In Saccharomyces cerevisiae, the SAGA complex is a 1.8-MDa complex comprising a variety of components including distinct classes of transcription factors, such as Ada proteins (Ada1p, Ada2p, Ngg1p/Ada3p, and Ada4p/Gcn5p), TATA-binding protein (TBP)-related SPT proteins (Spt3p, Spt7p, Spt8p, and Spt20p/Ada5p), and TBP-associated factors or (TAFIIs) (TAFII90, TAFII68/61, TAFII60, TAFII25/23, and TAFII17). The SAGA complex also comprises the DNA-dependent protein kinase related molecule Tra1p, acetyltransferase and ubiquitin protease activities. The SAGA complex core comprises Ada and Spt subunits, a subset of Tafs, acetyltransferase and ubiquitin protease activities, the essential factor Tra1p, and two factors related to TBP function, Spt3 and Spt8. Several components of the Saccharomyces cerevisiae SAGA complex and their corresponding Yarrowia lipolytica homologs, are listed in Table 69. Each of these SAGA complex components is encompassed by the recombinant fungal strains, methods and compositions of the present disclosure. Those of ordinary skill in the art will be aware of these and other SAGA components, and will be able to modify such components in accordance with the present disclosure.

Certain SAGA components are essential. For example, in Saccharomyces cerevisiae, the TRA1 gene is essential. Thus, in certain embodiments, production of a carotenoid is increased by altering expression and/or activity of the TRA1 such that the host organism remains viable. For example, the expression and/or activity of the TRA1 gene or gene product may be decreased to a level below the expression and/or activity of wild type TRA1, but not to such an extent as to result in lethality. Those of ordinary skill in the art will be aware of tra1 mutations that result in decreased expression and/or activity but that do not result in lethality. Furthermore, it will be within the capability of one of ordinary skill in the art to identify such mutations without undue experimentation, for example by employing standard mutatgenesis/screening techniques.

In certain embodiments of the present disclosure, production of one or more carotenoids is increased by alteration of the expression and/or activity of one or more components of the SAGA histone acetyltransferase complex in one or more of the following host organisms: Aspergillus, Blakeslea, Botrytis, Candida, Cercospora, Cryptococcus, Cunninghamella, Fusarium (Gibberella), Kluyveromyces, Lipomyces, Mortierella, Mucor, Neurospora, Penicillium, Phycomyces, Pichia (Hansenula), Puccinia, Pythium, Rhodosporidium, Rhodotorula, Saccharomyces, Sclerotium, Trichoderma, Trichosporon, Xanthophyllomyces (Phaffia), and Yarrowia; or is a species selected from the group consisting of: Aspergillus terreus, Aspergillus nidulans, Aspergillus niger, Blakeslea trispora, Botrytis cinerea, Candida japonica, Candida pulcherrima, Candida revkaufi, Candida tropicalis, Candida utilis, Cercospora nicotianae, Cryptococcus curvatus, Cunninghamella echinulata, Cunninghamella elegans, Fusarium fujikuroi (Gibberella zeae), Kluyveromyces lactis, Lipomyces starkeyi, Lipomyces lipoferus, Mortierella alpina, Mortierella ramanniana, Mortierella isabellina, Mortierella vinacea, Mucor circinelloides, Neurospora crassa, Phycomyces blakesleanus, Pichia pastoris, Puccinia distincta, Pythium irregulare, Rhodosporidium toruloides, Rhodotorula glutinis, Rhodotorula graminis, Rhodotorula mucilaginosa, Rhodotorula pinicola, Rhodotorula gracilis, Saccharomyces cerevisiae, Sclerotium rolfsii, Trichoderma reesei, Trichosporon cutaneum, Trichosporon pullans, Xanthophyllomyces dendrorhous (Phaffia rhodozyma), and/or Yarrowia lipolytica.

In certain embodiments, production of one or more carotenoids is increased by altering expression and/or activity of one or more components of the SAGA histone acetyltransferase complex in a host organism, in combination with one or more additional carotenogenic modifications as described herein. For example, such one or more additional carotenogenic modifications may comprise heterologous expression of one or more carotenogenic polypeptides, isoprenoid biosynthesis polypeptides, carotenoid biosynthesis polypeptides, etc.

In certain embodiments, production of one or more carotenoids is increased by altering expression and/or activity of one or more components of the SAGA histone acetyltransferase complex in a host organism, in combination with one or more oleaginic modifications, as described herein. In certain embodiments, production of one or more carotenoids is increased by altering expression and/or activity of one or more components of the SAGA histone acetyltransferase complex in a host organism that is not naturally oleaginous. In certain embodiments, production of one or more carotenoids is increased by altering expression and/or activity of one or more components of the SAGA histone acetyltransferase complex in a host organism that is naturally oleaginous.

It will be appreciated that the particular carotenogenic modification to be applied to a host cell in accordance with the present disclosure will be influenced by which carotenoid(s) is desired to be produced. For example, isoprenoid biosynthesis polypeptides are relevant to the production of most carotenoids. Carotenoid biosynthesis polypeptides are also broadly relevant. Carotenoid ketolase activity is particularly relevant for production of canthaxanthin, as carotenoid hydroxylase activity is for production of lutein and zeaxanthin, among others. Both carotenoid hydroxylase and ketolase activities (and astaxanthin synthase) are particularly useful for production of astaxanthin.

In certain embodiments, host cells are engineered to produce carotenoids by introducing one or more carotenoid biosynthesis polypeptides. In general, any carotenoid biosynthesis polypeptide can be introduced into any host cell of the present disclosure. In certain embodiments, such carotenoid biosynthesis polypeptides are codon-optimized to accommodate the codon preferences of the host cell. In certain embodiments, a carotenoid biosynthesis polypeptide introduced into a host cell is from the same organism as the host cell and/or a related organism. For example, without limitation, the present disclosure encompasses the recognition that it may be desirable to introduce a fungal carotenoid biosynthesis polypeptide into a fungal host cell (e.g., from the same and/or a related fungal species). In certain embodiments, the host cell is a Y. lipolytica host cell. In certain aspects of such embodiments, a Y. lipolytica carotenoid biosynthesis polypeptide is introduced into the Y. lipolytica host cell. In certain aspects, a S. cerevisiae carotenoid biosynthesis polypeptide is introduced into the Y. lipolytica host cell. In certain aspects, any of a variety of fungal carotenoid biosynthesis polypeptides is introduced into the Y. lipolytica host cell.

### Engineering Retinolic Compound Production

Retinolic compounds are synthesized from certain carotenoid precursors, which are themselves synthesized from isoprenoid precursors, some of which are also involved in the production of steroids and sterols (see description under section entitled “Engineering Carotenoid Production”). Thus, any carotenogenic modification that results in the increased production of a carotenoid from which a retinolic compound can be produced may similarly result in an increased production of a retinolic compound. Retinolic compounds comprise retinol, retinal, and retinoic acid, which together are collectively referred to as “Vitamin A”. In certain embodiments, the retinolic compound retinol is synthesized from the carotenoid precursor beta-carotene. Other carotenoid compounds that contain at least one beta-ionone ring structure, such as alpha-carotene and beta-cryptoxanthin, can be precursor compounds for synthesis of retinolic compounds.

According to the present disclosure, retinolic compound production in a host organism may be adjusted by modifying the expression or activity of one or more proteins involved in retinolic compound biosynthesis. As indicated, in some embodiments, it will be desirable to utilize as host cells organisms that naturally produce one or more retinolic compounds. In some such cases, the focus will be on increasing production of a naturally-produced retinolic compound, for example by increasing the level and/or activity of one or more proteins involved in the synthesis of that retinolic compound and/or by decreasing the level or activity of one or more proteins involved in a competing biosynthetic pathway. Alternatively or additionally, in some embodiments it will be desirable to generate production of one or more retinolic compounds not naturally produced by the host cell.

According to some embodiments of the disclosure, it will be desirable to introduce one or more heterologous retinologenic polypeptides into a host cell. As will be apparent to those of ordinary skill in the art, any of a variety of heterologous polypeptides may be employed; selection will consider, for instance, the particular retinolic compound whose production is to be enhanced. The present disclosure contemplates not only introduction of heterologous retinologenic polypeptides, but also adjustment of expression or activity levels of heterologous retinologenic polypeptides, including, for example, alteration of constitutive or inducible expression patterns. In some embodiments of the disclosure, expression patterns are adjusted such that growth in nutrient-limiting conditions is not required to induce oleaginy. For example, genetic modifications comprising alteration and/or addition of regulatory sequences (e.g., promoter elements, terminator elements) may be utilized to confer particular regulation of expression patterns. Such genetic modifications may be utilized in conjunction with endogenous genes (e.g., for regulation of endogenous carotenogenic); alternatively, such genetic modifications may be included so as to confer regulation of expression of at least one heterologous polypeptide (e.g., retinologenic polypeptide(s)). For example, promoters including, but not limited to those described herein, such as Tef1, Gpd1 promoters can be used in conjunction with endogenous genes and/or heterolous genes for modification of expression patterns of endogenous retinologenic polypeptide(s) and/or heterologous retinologenic polypeptide(s). Similarly, exemplary terminator sequences include, but are not limited to, use of Y. lipolytica XPR2 terminator sequences.

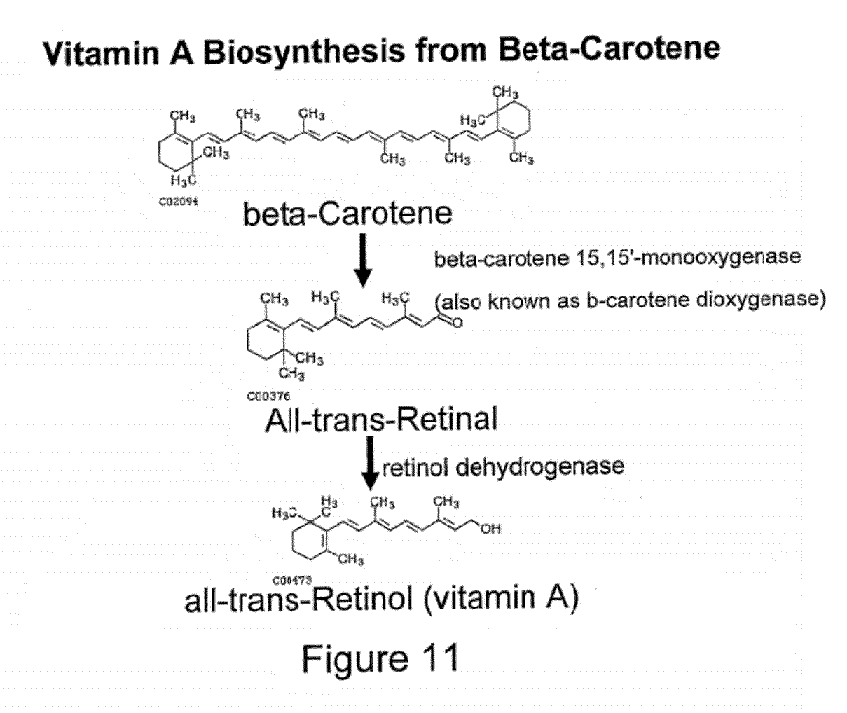

As indicated in FIG. 11 and in the literature, proteins involved in retinologenic biosynthesis include, but are not limited to, beta-carotene 15, 15′-monooxygenase (also known as beta-carotene dioxygenase) and beta carotene retinol dehydrogenase. Representative example sequences for these retinolic compound biosynthesis polypeptides are provided in Tables 67-68.

In some embodiments of the disclosure, potential source organisms for retinolic compound biosynthesis polypeptides include, but are not limited to, genera of naturally oleaginous or non-oleaginous fungi that naturally produce carotenoids. These include, but are not limited to, Botrytis, Cercospora, Fusarium (Gibberella), Mucor, Neurospora, Phycomyces, Puccina, Rhodotorula, Sclerotium, Trichoderma, and Xanthophyllomyces. Exemplary species include, but are not limited to, Neurospora crassa, Xanthophyllomyces dendrorhous (Phaffia rhodozyma), Mucor circinelloides, and Rhodotorula glutinis. Of course, retinolic compounds are produced by a wide range of diverse organisms such as mammals, bacteria, cyanobacteria, etc. Any such organisms may be source organisms for retinolic compound biosynthesis polypeptides according to the present disclosure.

In certain embodiments of the disclosure, retinolic compound production in a host organism that is able to produce retinolic compounds from carotenoid substrates is adjusted by modifying the activity of one or more endogenous genes that affect carotenoid biosynthesis. For example, as shown in Example 16, disruption of the endogenous SPT8 gene (YALI0E23804g) in Yarrowia lipolytica results in increased carotenoid production. As will be appreciated by those of ordinary skill in the art, increasing production of a carotenoid(s) in a host organism by altering the expression and/or activity of one or more components of the SAGA histone acetyltransferase complex will result in a greater abundance of such a carotenoid(s); hence, production of a retinolic compound(s) in a host organism able to utilize such a carotenoid(s) as a substrate may similarly be increased.

Without wishing to be bound by theory, the present disclosure contemplates that alteration of the expression and/or activity of one or more components of the SAGA histone acetyltransferase complex may result in increased retinolic compound production. In certain embodiments, retinolic compound production is increased in a host organism by altering the expression and/or activity of one or more of: Ada proteins (Ada1p, Ada2p, Ngg1p/Ada3p, and Ada4p/Gcn5p), TATA-binding protein (TBP)-related SPT proteins (Spt3p, Spt7p, Spt8p, and Spt20p/Ada5p), TBP-associated factors or (TAFIIs) (TAFII90, TAFII68/61, TAFII60, TAFII25/23, and TAFII17), Tra1p, and/or proteins encoding the acetyltransferase and/or ubiquitin protease activities. In certain embodiments, retinolic compound production is increased in a host organism by altering the expression and/or activity of one or more polypeptides listed in Table 69. Those of ordinary skill in the art will be aware of these and other SAGA components, and will be able to modify such components in accordance with the present disclosure.

In certain embodiments, host cells are engineered to produce retinolic compounds by introducing one or more carotenoid biosynthesis polypeptides. In general, any retinolic compound biosynthesis polypeptide can be introduced into any host cell of the present disclosure. In certain embodiments, such retinolic compound biosynthesis polypeptides are codon-optimized to accommodate the codon preferences of the host cell. In certain embodiments, a retinolic compound biosynthesis polypeptide introduced into a host cell is from the same organsim as the host cell and/or a related organism. For example, without limitation, the present disclosure encompasses the recognition that it may be desirable to introduce a fungal retinolic compound biosynthesis polypeptide into a fungal host cell (e.g., from the same and/or a related fungal species). In certain embodiments, the host cell is a Y. lipolytica host cell. In certain aspects of such embodiments, a Y. lipolytica retinolic compound biosynthesis polypeptide is introduced into the Y. lipolytica host cell. In certain aspects, a S. cerevisiae retinolic compound biosynthesis polypeptide is introduced into the Y. lipolytica host cell. In certain aspects, any of a variety of fungal retinolic compound biosynthesis polypeptides is introduced into the Y. lipolytica host cell.

Production and Isolation of Carotenoids and/or Retinolic Compounds

As discussed above, accumulation of lipid bodies in oleaginous organisms is generally induced by growing the relevant organism in the presence of excess carbon source and limiting nitrogen and/or other nutrients (eg. phosphate and magnesium). Specific conditions for inducing such accumulation have previously been established for a number of different oleaginous organisms (see, for example, Wolf (ed.) Nonconventional yeasts in biotechnology Vol. 1, Springer-Verlag, Berlin, Germany, pp. 313-338; Lipids 18(9):623, 1983; Indian J. Exp. Biol. 35(3):313, 1997; J. Ind. Microbiol. Biotechnol. 30(1):75, 2003; Bioresour Technol. 95(3):287, 2004, each of which is incorporated herein by reference in its entirety).

In general, it will be desirable to cultivate inventive modified host cells under conditions that allow accumulation of at least about 20% of their dry cell weight as lipid. In other embodiments, the inventive modified host cells are grown under conditions that permit accumulation of at least about 15%, 16%, 17%, 18%, 19%, 20%, 21%, 22%, 23%, 24%, 25%, 30%, 35%, 40%, 45%, 50%, 55%, 60%, 65%, 70%, 75%, or even 80% or more of their dry cell weight as lipid. In certain embodiments, the host cells utilized are cells which are naturally oleaginous, and induced to produce lipid to the desired levels. In other embodiments, the host cells are cells which naturally produce lipid, but have been engineered to increase production of lipid such that desired levels of lipid production and accumulation are achieved.

In certain embodiments, the host cells of the disclosure are not naturally oleaginous, but have been engineered to produce lipid such that desired levels of lipid production are obtained. Those of ordinary skill in the art will appreciate that, in general, growth conditions that are effective for inducing lipid accumulation in a source organism, may well also be useful for inducing lipid accumulation in a host cell into which the source organism's oleaginic polypeptides have been introduced. Of course, modifications may be required in light of characteristics of the host cell, which modifications are within the skill of those of ordinary skill in the art.

It will also be appreciated by those of ordinary skill in the art that it will often be desirable to ensure that production of the desired carotenoid and/or retinolic compound by the inventive modified host cell occurs at an appropriate time in relation to the induction of oleaginy such that the carotenoid(s) and/or retinolic compound(s) accumulate(s) in the lipid bodies. In some embodiments, it will be desirable to induce production of the carotenoid(s) and/or retinolic compound(s) in a host cell which does not naturally produce the carotenoid(s) and/or retinolic compound(s), such that detectable levels of the carotenoid(s) and/or retinolic compound(s) is/are produced. In certain aspects the host cells which do not naturally produce a certain carotenoid(s) and/or retinolic compound(s) are capable of production of other carotenoid(s) (e.g., certain host cells may, for example, naturally produce β-carotene but may not naturally produce astaxanthin) and/or retinolic compound(s), (e.g., certain host cells may, for example, naturally produce retinal but may not naturally produce retinol); in other aspects the host cells do not naturally produce any carotenoid(s) and/or retinolic compound(s). In other embodiments, it will be desirable to increase production levels of carotenoid(s) and/or retinolic compound(s) in a host cell which does naturally produce low levels of the carotenoid(s) and/or retinolic compound(s), such that increased detectable levels of the carotenoid(s) and/or retinolic compound(s) are produced. In certain aspects, the host cells which do naturally produce the carotenoid(s) (e.g., β-carotene) also produce additional carotenoid(s) (e.g., astaxanthin, etc.) and/or retinolic compound(s) (e.g., retinal); in still other aspects, the cells which naturally produce the carotenoid(s) (e.g., β-carotene) do not produce additional carotenoid(s) and/or retinolic compound(s).

In certain embodiments of the disclosure, it will be desirable to accumulate carotenoids and/or retinolic compounds to levels (i.e., considering the total amount of all produced carotenoids and/or retinolic compounds together or considering a particular carotenoid and/or retinolic compound) that are greater than at least about 1% of the dry weight of the cells. In some embodiments, the total carotenoid and/or retinolic compound accumulation will be to a level at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, at least about 10%, at least about 10.5%, at least about 11%, at least about 11.5%, at least about 12%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20% or more of the total dry weight of the cells.

In some embodiments, accumulation of a particular carotenoid and/or retinolic compound (e.g., a carotenoid selected from antheraxanthin, adonirubin, adonixanthin, astaxanthin, canthaxanthin, capsorubrin, β-cryptoxanthin, α-carotene, β-carotene, β,ψ-carotene, δ-carotene, ε-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, a C30 carotenoid, and combinations thereof) will be to a level at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, at least about 10%, at least about 10.5%, at least about 11%, at least about 11.5%, at least about 12%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20% or more of the total dry weight of the cells.

In some embodiments of the disclosure, a particular carotenoid and/or retinolic compound may comprise a high percentage of total carotenoids and/or retinolic compounds produced by cells. In some embodiments, a particular carotenoid and/or retinolic compound may comprise at least 50%, 60%, 70%, 80%, 85%, 86%, 87%, 88%, 89%, 90%, 91%, 92%, 93%, 94%, 95%, 96%, 97%, 98%, or 99%, of the total carotenoid and/or retinolic compounds produced by cells. For example, in some embodiments, at least 50%, 60%, 70%, 80%, 85%, 86%, 87%, 88%, 89%, 90%, 91%, 92%, 93%, 94%, 95%, 96%, 97%, 98%, or 99%, of the total carotenoids produced by cells is β-carotene. In another example, at least 50%, 60%, 70%, 80%, 85%, 86%, 87%, 88%, 89%, 90%, 91%, 92%, 93%, 94%, 95%, 96%, 97%, 98%, or 99%, of the total carotenoids produced by cells is astaxanthin. In other examples, a high percentage of total carotenoids produced by cells is a carotenoid selected from antheraxanthin, adonirubin, adonixanthin, astaxanthin, canthaxanthin, capsorubrin, β-cryptoxanthin, α-carotene, β-carotene, β,ψ-carotene, δ-carotene, ε-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, and a C30 carotenoid.

In some embodiments of the disclosure, a particular carotenoid and/or retinolic compound may not accumulate to a level as high as 1% of the total dry weight of the cells; appropriately engineered cells according to the present disclosure, and any lipid bodies, carotenoids and/or retinolic compounds they produce, remain within the scope of the present disclosure. Thus, in some embodiments, the cells accumulate a given carotenoid and/or retinolic compound to a level below about 1% of the dry weight of the cells. In some embodiments, the carotenoid and/or retinolic compound accumulates to a level below about 0.9%, 0.8%, 0.7%, 0.6%, 0.5%, 0.4%, 0.3%, 0.2%, 0.1%, 0.05%, or lower, of the dry cell weight of the cells.

In some embodiments of the disclosure, carotenoids and/or retinolic compounds accumulate both within lipid bodies and elsewhere in the cells. In some embodiments, carotenoids and/or retinolic compounds accumulate primarily within lipid bodies. In some embodiments, carotenoids and/or retinolic compounds accumulate substantially exclusively within lipid bodies. In some embodiments, at least about 10%, 15%, 20%, 25%, 30%, 35%, 40%, 45%, 50%, 55%, 60%, 65%, 70%, 75%, 80%, 85%, 90%, 95% or more of a desired produced carotenoid and/or retinolic compound accumulates in lipid bodies.

In some embodiments of the disclosure, modified host cells are engineered to produce one or more carotenoid compound(s) and/or retinolic compound(s) characterized by negligible solubility in water (whether hot or cold) and detectable solubility in one or more oils. In some embodiments, such compounds have a solubility in oil below about 0.2%. In some embodiments, such compounds have a solubility in oil within the range of about <0.001%-0.2%.

The present disclosure therefore provides engineered host cells (and methods of making and using them) that contain lipid bodies and that further contain one or more carotenoid compounds and/or retinolic compounds accumulated in the lipid bodies, where the compounds are characterized by a negligible solubility in water and a solubility in oil within the range of about <0.001%-0.2%; 0.004%-0.15%; 0.005-0.1%; or 0.005-0.5%. For example, in some embodiments, such compounds have a solubility in oil below about 0.15%, 0.14%, 0.13%, 0.12%, 0.11%, 0.10%. 0.09, 0.08%, 0.07%, 0.06%, 0.05%, 0.04%, 0.03%, 0.02%, 0.01%, 0.05%, or less. In some embodiments, the compounds show such solubility in an oil selected from the group consisting of sesame; soybean; apricot kernel; palm; peanut; safflower; coconut; olive; cocoa butter; palm kernel; shea butter; sunflower; almond; avocado; borage; carnauba; hazel nut; castor; cotton seed; evening primrose; orange roughy; rapeseed; rice bran; walnut; wheat germ; peach kernel; babassu; mango seed; black current seed; jojoba; macademia nut; sea buckthorn; sasquana; tsubaki; mallow; meadowfoam seed; coffee; emu; mink; grape seed; thistle; tea tree; pumpkin seed; kukui nut; and mixtures thereof.

Bacterial carotenogenic genes have already been demonstrated to be transferrable to other organisms, and are therefore particularly useful in accordance with the present disclosure (see, for example, Miura et al., Appl. Environ. Microbiol. 64:1226, 1998). In other embodiments, it may be desirable to utilize genes from other source organisms such as plant, alga, or microalgae; these organisms provide a variety of potential sources for ketolase and hydroxylase polypeptides. Still additional useful source organisms include fungal, yeast, insect, protozoal, and mammalian sources of polypeptides.

In some embodiments of the present disclosure, isoprenoid production is increased in host cells (e.g., in Y. lipolytica cells) through expression of a truncated variant of a hydroxymethylglutaryl-CoA (HMG CoA) reductase polypeptide. In some embodiments, the truncated variant is a truncated variant of a Y. lipolytica HMG CoA reductase polypeptide. According to the present disclosure, expression of such a truncated HMG CoA reductase polypeptide can result in increased isoprenoid and/or carotenoid production in host cells (e.g., Y. lipolytica cells).

Alternatively or additionally, in some embodiments of the present disclosure, isoprenoid production is increased in host cells (e.g., in Y. lipolytica cells) through application of one or more carotenogenic modification(s) that increase(s) level and/or activity of a polypeptide selected from the group consisting of farnesyl pyrophosphate synthase polypeptides, geranylgeranylpyrophosphate synthase polypeptides, and combinations thereof. In some embodiments, the source organism for the selected polypeptide is Y. lipolytica.

Alternatively or additionally, in some embodiments of the present disclosure, isoprenoid production is increased in host cells (e.g., in Y. lipolytica cells) through application of one or more carotenogenic modification(s) that decrease(s) expression or activity of an isoprenoid biosynthesis competitor polypeptide (e.g., of a squalene synthase polypeptide), for example thereby reducing diversion of one or more intermediates away from the isoprenoid and/or carotenoid biosynthesis pathways. In some embodiments, the polypeptide whose expression or activity is reduced is endogenous to the host cell.

In some embodiments of the present disclosure, more than one carotenogenic modification is applied to the same host cell. For example, isoprenoid production may be increased in host cells (e.g., Y. lipolytica cells) through application of at least two or more carotenogenic modifications selected from the group consisting of: expression of a truncated HMG CoA reductase polypeptide, increase in expression and/or activity of farnesyl pyrophosphate synthase polypeptide, increase in expression and/or activity of a geranylgeranylpyrophosphate synthase polypeptide, decrease in expression and/or activity of a squalene synthase polypeptide, and combinations thereof.

Furthermore, in some embodiments of the disclosure, carotenoid production (e.g., production of β-carotene) is increased in host cells (e.g., in Y. lipolytica cells) through application of one or more carotenogenic modification(s) that increase(s) expression and/or activity of a polypeptide selected from the group consisting of phytoene synthase, lycopene cyclase, phytoene dehydrogenase, and combinations thereof. In some embodiments, such increase in expression comprises introduction of one or more genes encoding heterologous polypeptides. In some embodiments, phytoene synthase and lycopene cyclase activities are provided in a single polypeptide or complex (e.g., by the Mucor circinelloides or Neurospora crassa multifunctional phytoene synthase/lycopene cyclase). In some embodiments, phytoene dehydrogenase from Mucor circinelloides or Neurospora crassa is utilized.

In some embodiments, production of one or more carotenoids downstream of β-carotene (e.g., of one or more hydroxylated xanthophylls) is increased in host cells that produce β-carotene (including host cells that have been engineered to produce β-carotene, e.g., through application of one or more carotenogenic modifications as described herein) through application of one or more carotenogenic modifications that increase(s) level and/or activity of one or more carotenoid ketolase polypeptides (e.g., from Parvularcula bermudensis and/or Aurantimonase sp. SI85-9A1) to produce one or more ketone-containing carotenoids (e.g., canthaxanthin, echinenone, astaxanthin, and combinations thereof).

In some embodiments, production of one or more hydroxylated carotenoids is increased in host cells that produce (including having been engineered to produce) β-carotene and/or one or more ketone-containing carotenoids though application of one or more carotenogenic modifications that increase(s) the level and/or activity of one or more carotenoid hydroxylase polyeptides (e.g., from Xanthobacter autotrophicus and/or Erythrobacter litoralis) to increase production of one or more hydroxylated carotenoids (e.g., zeaxanthin, lutein, β-cryptoxanthin, astaxanthin, and combinations thereof).

Similar approaches to enhance carotenoid production may be employed in other oleaginous or non-oleaginous host organisms (e.g., S. cerevisiae, C. utilis, P. rhodozyma) can be undertaken, using the same, homologous, or functionally similar carotogenic polypeptides.

In some embodiments, the present disclosure provides modified Y. lipolytica strains that have been engineered to express one or more carotenoid biosynthesis polypeptides and/or isoprenoid biosynthesis polypeptides. For example, in some embodiments, a modified Y. lipolytica strain is engineered to increase expression and/or activity of one or more of phytoene synthase, phytoene dehydrogenase, lycopene cyclase, and GGPP synthase, and/or to decrease expression and/or activity of squalene synthase. In some embodiments, a modified Y. lipolytica strain is engineered to express all of these polypeptides. Such a modified Y. lipolytica strain produces β-carotene (see, for example, Example 2).

In some embodiments, inventive modified Y. lipolytica strains that have been engineered to produce β-carotene are further engineered to express a truncated HMG CoA reductase; in some such embodiments, the strains are engineered so that expression of the truncated HMG CoA reductase increases β-carotene several fold (for example, 3-4 fold or more).

In some embodiments, inventive modified Y. lipolytica strains that have been engineered to produce β-carotene are further engineered to express a beta-carotene 15, 15′-monooxygenase and/or a retinol dehydrogenase to increase retinolic compound production.

In some embodiments, inventive modified Y. lipolytica strains that have been engineered to produce β-carotene are further engineered to express carotenoid hydroxylase (to achieve production of zeaxanthin and/or β-cryptoxanthin), carotenoid ketolase (to achieve production of canthaxanthin and/or echinenone), or both (to achieve production of astaxanthin).

In some embodiments, inventive modified Y. lipolytica strains that have been engineered to produce, for example, β-carotene, zeaxanthin, canthaxanthin, echinenone, and/or astaxanthin are also engineered to have increased expression of, for example, malic enzyme, mevalonate kinase, etc.

It will be appreciated that, in some embodiments of the disclosure, it may be desirable to engineer a particular host cell by expressing more than one version of a given polypeptide (e.g., isoprenoid biosynthesis polypeptide, carotenoid biosynthesis polypeptide, oleaginic polypeptide, isoprenoid biosynthesis competitor polypeptides, retinolic compound biosynthesis polypeptide, etc.). For example, a given host cell may be engineered to express versions of a given polypeptide from two or more different sources. Where a particular enzyme may be comprised of more than one polypeptide chains, it will often be desirable to utilize all chains from a single source, although this is not required so long as activity is achieved. Also, whenever a host cell is engineered to express a polypeptide from a different source, it may be desirable to alter the gene sequence encoding the polypeptide to account for codon preferences of the host cell.

To give but a few specific examples, the present disclosure provides modified Y. lipolytica strains that have been engineered to express the phytoene synthase/lycopene cyclase bifunctional (carB) polypeptide from M. circinelloides (see, for example, Example 1B), and also to express the phytoene dehydrogenase (carRP) polypeptide from M. circinelloides (see, for example, Example 1A). In some embodiments, the present disclosure provides such carB+carRP-expressing Y. lipolytica strains that have been engineered to modify expression and/or activity of a truncated HMG-CoA reductase polypeptide from Y. lipolytica and/or one or more Y. lipolytica polypeptides selected from the group consisting of GGPP synthase, FPP synthase (Erg20), IPP isomerase (IDI), HMG synthase (Erg13), mevalonate kinase (Erg12), squalene synthase (Erg9), phosphimevalonate kinase (Erg8), mevalonate pyrophosphate decarboxylase (MVD1), malic enzyme, malate dehydrogenase, glucose 6 phosphate dehydrogenase, malate dehydrogenase homolog 2,6-phosphogluconate dehydrogenase (GND1), isocitrate dehydrogenase, fructose 1,6 bisphosphatase, acetoacetyl CoA thiolase (Erg10), ATP citrate lyase subunit 1, ATP citrate lyase subunit 2, and combinations thereof. The present disclosure therefore specifically provides Y. lipolytica strains that have been engineered to produce β-carotene.

The present disclosure also specifically provides modified Y. lipolytica strains that have been engineered to express at least one carotenoid ketolase (e.g., crtO/crtW) polypeptide, and in some embodiments more than one, for example from a source selected from the group consisting of Parvularcula bermudensis (see, for example, Example 1H), Aurantimonas (see, for example, Example 1G), and/or an environmental isolate identified from the Sargasso Sea (see, for example, Example 1F). The present disclosure therefore specifically provides Y. lipolytica strains that have been engineered to produce canthaxanthin, astaxanthin, and/or echinenone.

The present disclosure further specifically provides modified Y. lipolytica strains that have been engineered to express at least one carotenoid hydroxylase (e.g., crtZ) polypeptide, and in some embodiments more than one, from Erythrobacter litoralis (see, for example, Examples 1J and 1L), Novosphingobium aromaticivarans (see, for example, Example 1E), Parvularcula bermudensis (see, for example, Example 1I), Xanthobacter autotrophicus (see, for example, Example 1O), Sphingopyxis alaskensis (see, for example, Example 1M), Chlamydomonas rheinhardtii, Erythrobacter longus, Robiginitalea biformata (see, or example, Example 1N) and/or Pseudomonas putida (see, for example, Example 1P). The present disclosure therefore specifically provides Y. lipolytica strains that have been engineered to produce zeaxanthin, lutein, β-cryptoxanthin, and/or astaxanthin.

The present disclosure further specifically provides modified Y. lipolytica strains that have been engineered to express at least one carotenoid ketolase (e.g., crtO/crtW) polypeptide in combination with at least one carotenoid hydroxylase (e.g., crtZ) polypeptide. In certain embodiments, the at least one carotenoid ketolatse polypeptide and at least one carotenoid hydroxylase polypeptide are encoded by nucleic acid sequences present in separate nucleic acid molecules. In certain embodiments, the at least one carotenoid ketolatse polypeptide and at least one carotenoid hydroxylase polypeptide are encoded by nucleic acid sequences present in the same nucleic acid molecule. For example, a host organism may be transformed or transfected with a single expression vector, which expression vector comprises both a carotenoid ketolatse polypeptide and a carotenoid hydroxylase polypeptide, each of which comprises sequences sufficient to direct their expression in the host organism.

In certain embodiments, the at least one carotenoid ketolase (e.g., crtO/crtW) polypeptide and the at least one carotenoid hydroxylase (e.g., crtZ) polypeptide are expressed as a fusion protein. A representative example of such embodiments is presented in Example 17. In certain embodiments, such a fusion polypeptide is designed such that the carotenoid ketolatse polypeptide is positioned N-terminal to the carotenoid hydroxylase polypeptide. In certain embodiments, such a fusion polypeptide is designed such that the carotenoid ketolatse polypeptide is positioned C-terminal to the carotenoid hydroxylase polypeptide.

In embodiments in which the carotenoid ketolatse polypeptide and the carotenoid hydroxylase polypeptide are expressed concurrently (whether from separate nucleic acid molecules or from the same nucleic acid molecule), the polypeptides may be selected from any of a variety of source organisms. As non-limiting examples, the carotenoid hydroxylase polypeptide may be selected from an organism such as Erythrobacter litoralis (see, for example, Examples 1J and 1L), Novosphingobium aromaticivarans (see, for example, Example 1E), Parvularcula bermudensis (see, for example, Example 1I), Xanthobacter autotrophicus (see, for example, Example 1O), Sphingopyxis alaskensis (see, for example, Example 1M), Chlamydomonas rheinhardtii, Erythrobacter longus, Robiginitalea biformata (see, or example, Example 1N) and/or Pseudomonas putida (see, for example, Example 1P). As further non-limiting examples, the carotenoid ketolase polypeptide may be selected from an organism such as Parvularcula bermudensis (see, for example, Example 1H), Aurantimonas (see, for example, Example 1G), and/or an environmental isolate identified from the Sargasso Sea (see, for example, Example 1F).

It should be noted that, for inventive organisms that produce more than one carotenoid, it will sometimes be possible to adjust the relative amounts of individual carotenoids produced by adjusting growth conditions. For example, it has been reported that controlling the concentration of dissolved oxygen in a culture during cultivation can regulate relative production levels of certain carotenoids such as β-carotene, echinenone, β-cryptoxanthin, 3-hydroxyechinenone, asteroidenone, canthaxanthin, zeaxanthin, adonirubin, adonixanthin and astaxanthin (see, for example, U.S. Pat. No. 6,825,002 to Tsubokura et al., the entire contents of which are incorporated herein by reference). Additionally or alternatively, the present disclosure encompasses the recognition that controlling the pH in a culture during cultivation can regulate relative production levels of these and/or other carotenoids (see e.g., Example 18).

Particularly for embodiments of the present disclosure directed toward production of astaxanthin, it will often be desirable to utilize one or more genes from a natural astaxanthin-producing organism. Where multiple heterologous polypeptides are to be expressed, it may be desirable to utilize the same source organism for all, or to utilize closely related source organisms.

Inventive modified cells, that have been engineered to produce carotenoids and/or to accumulate lipid (including to be oleaginous), can be cultured under conditions that achieve carotenoid production and/or oleaginy. In some embodiments, it will be desirable to control growth conditions so in order to maximize production of a particular carotenoid or set of carotenoids (including all carotenoids) and/or to optimize accumulation of the particular carotenoid(s) in lipid bodies. In some embodiments it will be desirable to control growth conditions to adjust the relative amounts of different carotenoid products produced.

Inventive modified cells, that have been engineered to produce retinolic compounds and/or to accumulate lipid (including to be oleaginous), can be cultured under conditions that achieve retinolic compound production and/or oleaginy. In some embodiments, it will be desirable to control growth conditions so in order to maximize production of a particular retinolic compound or set of retinolic compounds (including all retinolic compounds) and/or to optimize accumulation of the particular retinolic compound (s) in lipid bodies. In some embodiments it will be desirable to control growth conditions to adjust the relative amounts of different retinolic compound products produced.

In some embodiments, it will be desirable to limit accumulation of a particular intermediate, for example ensuring that substantially all of a particular intermediate compound is converted so that accumulation is limited. For example, particularly in situations where a downstream enzyme may be less efficient than an upstream enzyme and it is desirable to limit accumulation of the product of the upstream enzyme (e.g., to avoid its being metabolized via a competitive pathway and/or converted into an undesirable product), it may be desirable to grow cells under conditions that control (e.g., slow) activity of the upstream enzyme so that the downstream enzyme can keep pace.

Those of ordinary skill in the art will appreciate that any of a variety of growth parameters, including for example amount of a particular nutrient, pH, temperature, pressure, oxygen concentration, timing of feeds, content of feeds, etc can be adjusted as is known in the art to control growth conditions as desired.

To give but a few examples, in some embodiments, growth and/or metabolism is/are limited by limiting the amount of biomass accumulation. For example, growth and/or metabolism can be limited by growing cells under conditions that are limiting for a selected nutrient. The selected limiting nutrient can then be added in a regulated fashion, as desired. In some embodiments, the limiting nutrient is carbon, nitrogen (e.g., via limiting ammonium or protein), phosphate, magnesium, one or more trace elements or metals (e.g., one or more of zinc or manganese), or combinations thereof. In some embodiments, the limiting nutrient is carbon. In some embodiments, the limiting nutrient is one or more trace metals (e.g., zinc, manganese, or iron). In particular embodiments, the limiting nutrient is zinc. Growth conditions are “limiting” for a trace element or metal when the growth medium has low levels of the trace element or metal and is not supplemented with a compound containing the trace element or metal. In some embodiments, medium containing less than about 2000 ug/L boric acid (e.g., less than about 1500 or 1100 ug/L) is limiting for boron. In some embodiments, medium containing less than about 200 ug/L copper sulfate (e.g., less than about 150 or 100 ug/L) is limiting for copper. In some embodiments, medium containing less than about 500 ug/L potassium iodide (e.g., less than about 300 or 250 ug/L) is limiting for iodine. In some embodiments, medium containing less than about 1000 ug/L ferric chloride (e.g., less than about 750 or 500 ug/L) is limiting for iron. In some embodiments, medium containing less than about 1000 ug/L sodium molybdate (e.g., less than about 750 or 500 ug/L) is limiting for molybdenum. In some embodiments, medium containing less than about 2000 ug/L zinc sulfate (e.g., less than about 1500 or 1000 ug/L) is limiting for zinc. In one example, Yeast Nitrogen Base that is not supplemented with one of the following compounds is limiting for the trace metal or element contained in the compound: boric acid, copper sulfate, potassium iodide, ferric chloride, manganese sulfate, sodium molybdate, or zinc sulfate. The approximate concentration of each of these compounds in Yeast Nitrogen Base, used in medium at 4 g/L, is as follows: boric acid, 1176 ug/L; copper sulfate, 94 ug/L; potassium iodide, 235 ug/L; ferric chloride, 470 ug/L; manganese sulfate, 941 ug/L; sodium molybdate, 470 ug/L; zinc sulfate, 940 ug/L.

In some embodiments, use of a limiting nutrient can by utilized to control metabolism of a particular intermediate and/or to adjust relative production of particular carotenoid compounds and/or retinolic compounds. In some embodiments, this result can be achieved by controlling metabolism of a particular intermediate as discussed above; in some embodiments, it can be achieved, for example, by limiting progress through the carotenoid and/or retinolic compound biosynthesis pathway so that a desired carotenoid product (e.g., β-carotene, canthaxanthin, astaxanthin, etc.) or retinolic compound (e.g., retinal) is not converted to a downstream compound. To give but one example, phosphate limitation can slow the overall rate of clux through the carotenoid biosynthesis pathway and can be utilized to change the ratio of canthaxanthin to echinenone produced.

In some embodiments, cells are grown in the presence of excess carbon source and limiting nitrogen, phosphate, and/or magnesium to induce oleaginy. In some embodiments cells are grown in the presence of excess carbon source and limiting nitrogen. In some embodiments, the carbon:nitrogen ratio is within the range of about 200:1, 150:1, 125:1, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, or less. Those of ordinary skill in the art are aware of a wide variety of carbon sources, including, for example, glycerol, glucose, galactose, dextrose, any of a variety of oils (e.g., olive, canola, corn, sunflower, soybean, cottonseed, rapeseed, etc., and combinations thereof) that may be utilized in accordance with the present disclosure. Combinations of such may also be utilized. For example, common carbon source compositions contain oil:glucose in a ratio within the range of about 5:95 to 50:50 (e.g., about 5:95, about 10:90, about 15:85, about 20:80, about 25:75, about 30:70, about 35:65, about 40:60, about 45:55, about 50:50).

Those of ordinary skill in the art are also aware of a variety of different nitrogen sources (e.g., ammonium sulfate, proline, sodium glutamate, soy acid hydrolysate, yeast extract-peptone, yeast nitrogen base, corn steep liquor, etc, and combinations thereof) that can be utilized in accordance with the present disclosure.

In some embodiments, cultures are grown at a selected oxygen concentration (e.g., within a selected range of oxygen concentrations). In some embodiments, oxygen concentration may be varied during culture. In some embodiments, oxygen concentration may be controlled during some periods of culture and not controlled, or controlled at a different point, during others. In some embodiments, oxygen concentration is not controlled. In some embodiments, cultures are grown at an oxygen concentration within the range of about 5-30%, 5-20%, 10-25%, 10-30%, 15-25%, 15-30%, including at about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or more. In some embodiments, oxygen concentration is maintained above about 20%, at least for some period of the culture.

In some embodiments, cells are grown via fed-batch fermentation. In some embodiments, feed is continued until feed exhaustion and/or the feed is controlled to initiate or increase once a certain level of dissolved oxygen is detected in the culture medium (e.g., about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or more dissolved oxygen). The feed rate can be modulated to maintain the dissolved oxygen at a specific level (e.g., about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or more dissolved oxygen).

In some embodiments, inventive modified cells are grown in a two-phase feeding protocol in which the first phase is designed to maintain conditions of excess carbon and limiting oxygen, and the second phase results in conditions of excess oxygen and limiting carbon. The carbon sources in each phase can be the same (e.g., both glucose, both oil, such as soybean oil) or different (e.g., glucose then glucose-oil mixture, oil then glucose, or glucose-oil mixture then glucose). The present disclosure demonstrates that such conditions can achieve high levels of carotenoid production (see, for example, Examples 5D, 5E, and 5F). For example, high levels of carotenoid production can be achieved under conditions in which an oil (e.g., soybean oil) is the main carbon source in the first phase, and glucose is the main carbon source in the second phase. Carotenoid production can also be enhanced when cells are grown under conditions that are limiting for zinc (e.g., when cells are grown in medium that contains low levels of trace metals, and that is not supplemented with a zinc-containing compound such as ZnCl₂). Additionally or alternatively, such conditions also result in high levels of retinolic compound production. For example, high levels of retinolic compound(s) production may be achieved by increasing the levels of a particular carotenoid that is used as a substrate for the production of such a retinolic compound(s).

In some embodiments, inventive modified cells are cultivated at constant temperature (e.g., between about 20-40, or 20-30 degrees, including for example at about 20, 20.5, 21, 21.5, 22, 22.5, 23, 23.5, 24, 24.5, 25, 25.5, 26, 26.5, 27, 27.5, 28, 28.5, 29, 29.5, 30° C. or above) and/or pH (e.g., within a range of about 4-7.5, or 4-6.5, 3.5-7, 3.5-4, 4-4.5, 4.5-5, 5-5.5, 5.5-6, 6-6.5, 6.5-7, 7-7.5, 7-8, etc., including at about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5 or above); in other embodiments, temperature and/or pH may be varied during the culture period, either gradually or in a stepwise fashion.

For example, in some embodiments, the pH is 7.0 at inoculation and is increased to pH 8.0 during the course of the fermentation. The pH may be increased either continuously or in discrete steps. For example, in Example 19, the pH of the culture in increased continuously. In certain embodiments, the pH in increased continuously by increasing the pH at a rate of 0.001, 0.002, 0.003, 0.004, 0.005, 0.006, 0.007, 0.008, 0.009, 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050 or more units/hour.

In certain embodiments, the pH in increased in discrete steps by increasing the pH by 0.001, 0.002, 0.003, 0.004, 0.005, 0.006, 0.007, 0.008, 0.009, 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050 or more at each step.

In certain embodiments, the pH is increased employing a combination of continuous increase and discrete steps.

In certain embodiments, increasing the pH during the course of fermentation results in one or more beneficial effects such as, without limitation, an increase in total biomass accumulation, an increase in the percentage of biomass representing carotenoid accumulation, and, in the case of zeaxanthin production, an increase in the hydroxylation of beta-carotene to zeaxanthin. Those of ordinary skill in the art will be able to select without undue experimentation an appropriate rate of increase, an appropriate type of increase (e.g., continuous, discrete steps or a combination of the two), and/or an optimum pH within the selected range to maximize these and/or other beneficial effects.

In some embodiments, the temperature at which inventive cells are cultivated is selected so that production of one or more particular carotenoid compound(s) and/or retinolic compound(s) is adjusted (e.g., so that production of one or more particular compound(s) is increased and/or production of one or more other compound(s) is decreased). In some embodiments, the temperature at which inventive cells are cultivated is selected so that the ratio of one carotenoid compound and/or retinolic compound to another, is adjusted. To give but one example, in some embodiments, a temperature is selected to be sufficiently low that β-carotene levels are reduced and the level of at least one other carotenoid compound(s) (e.g., zeaxanthin) is increased.

In some embodiments, cultures are grown at about pH 5.5, at about pH 7.0, and or at a temperature between about 28-30° C. In some embodiments, it may be desirable to grow inventive modified cells under low pH conditions, in order to minimize growth of other cells. In some embodiments, it will be desirable to grow inventive modified cells under relatively higher temperature conditions in order to slow growth rate and/or increase the ultimate dry cell weight output of carotenoids and/or retinolic compounds. In some embodiments, it will be desirable to grow inventive modified cells under conditions in which the pH in increased (e.g., continuously, in discrete steps, or both) during the course of fermentation (e.g., increased from pH 7.0 to pH 8.0). In some embodiments, it will be desirable to grow inventive modified cells under two or more of these conditions. For example, inventive modified cells can be grown under relatively higher temperature conditions while simultaneously increasing the pH over the course of the fermentation. Those of ordinary skill in the art will be able to select appropriate growth conditions to achieve their experimental, production and/or other cell culture goals.

One advantage provided by the present disclosure is that, in addition to allowing the production of high levels of carotenoids and/or retinolic compounds, certain embodiments of the present disclosure allow produced compounds to be readily isolated because they accumulate in the lipid bodies within oleaginous organisms. Methods and systems for isolating lipid bodies have been established for a wide variety of oleaginous organisms (see, for example, U.S. Pat. Nos. 5,164,308; 5,374,657; 5,422,247; 5,550,156; 5,583,019; 6,166,231; 6,541,049; 6,727,373; 6,750,048; and 6,812,001, each of which is incorporated herein by reference in its entirety). In brief, cells are typically recovered from culture, often by spray drying, filtering or centrifugation.

Of course, it is not essential that lipid bodies be specifically isolated in order to collect carotenoid compounds and/or retinolic compounds produced according to the present disclosure. Any of a variety of approaches can be utilized to isolate and/or purify carotenoids and/or retinolic compounds. Many useful extraction and/or purification procedures for particular carotenoid compounds, and/or for carotenoids generally, are known in the art (see, for example, EP670306, EP719866, U.S. Pat. No. 4,439,629, U.S. Pat. No. 4,680,314, U.S. Pat. No. 5,310,554, U.S. Pat. No. 5,328,845, U.S. Pat. No. 5,356,810, U.S. Pat. No. 5,422,247, U.S. Pat. No. 5,591,343, U.S. Pat. No. 6,166,231, U.S. Pat. No. 6,750,048, U.S. Pat. No. 6,812,001, U.S. Pat. No. 6,818,239, U.S. Pat. No. 7,015,014, US2003/0054070, US2005/0266132, each of which is incorporated herein by reference).

In many typical isolation procedures, cells are disrupted (e.g., mechanically (for example using a bead mill, mashing), enzymatically (e.g., using zymolyase or a β-1,3 glucanase such as Glucanex 200G (Novozyme), chemically (e.g., by exposure to a mild caustic agent such as a detergent or 0.1 N NaOH, for example at room temperature or at elevated temperature), using a reducing agent (e.g., dithiothreitol, β-mercaptoethanol), using high pressure homogenization/shearing, by changing pH, etc. and combinations thereof) to allow access of intracellular carotenoid and/or retinolic compound(s) to an extraction solvent, and are then extracted one or more times. In certain embodiments, cells are disrupted mechanically using a bead mill/mashing at high pressure (e.g., at 25K, 10K-30K, 15K-25K, or 20-25K, pound-force per square inch (psi)). Cells may optionally be concentrated (e.g., to at least about 100 g/L or more, including to at least about 120 g/l, 150 g/l, 175 g/L, 200 g/L or more) and/or dried (e.g., with a spray dryer, double drum dryer (e.g., Blaw Knox double drum dryer), single drum vacuum dryer, etc.), prior to exposure to extraction solvent (and/or prior to disruption or homogenization). Disruption can, of course, be performed prior to and/or during exposure to extraction solvent. After extraction, solvent is typically removed (e.g., by evaporation, for example by application of vacuum, change of temperature, etc.).

In some instances, cells are disrupted and then subjected to supercritical liquid extraction or solvent extraction. Typical liquids or solvents utilized in such extractions include, for example, organic or non-organic liquids or solvents. To give but a few specific examples, such liquids or solvents may include acetone, supercritical fluids (e.g., carbon dioxide, propane, xenon, ethane, propylene, methane, ethylene, ethanol), carbon dioxide, chloroform, ethanol, ethyl acetate, heptane, hexane, isopropanol, methanol, methylene chloride, octane, tetrahydrofuran (THF), cyclohexane, isobutyl acetate, methyl ketone, ethyl ketone, toluene, cyclohexanone, benzene, propylene glycol, vegetable oils (e.g., soybeen soybean oil, rapeseed oil, corn oil, cottonseed oil, canola oil, etc.) and combinations thereof (e.g., hexane:ethyl acetate, combination of a polar and non-polar solvent, combination of an alcohol with either hexane or ethyl acetate). Particular solvents may be selected, for example, based on their ability to solubilize particular carotenoid compounds and/or retinolic compounds, or sets of carotenoid compounds (e.g., all carotenoids) and/or retinolic compounds (e.g., all retinolic compounds), and/or based on regulatory or other considerations (e.g., toxicity, cost, ease of handling, ease of removal, ease of disposal, etc.). For example, more polar carotenoids (e.g., xanthophylls) are known to be extracted more efficiently into extraction solvents with increased polarity. Craft (1992) J. Agric. Food Chem 40, 431-434 which is herein incorporated by reference discusses the relative solubility of two carotenoids, lutein and β-carotene in different solvents.

In some embodiments, combinations of solvents may be utilized. In some embodiments, combinations of a relatively polar solvent (e.g., alcohols, acetone, chloroform, methylene chloride, ethyl acetate, etc.) and a relatively non-polar solvent (e.g., hexane, cyclohexane, oils, etc.) are utilized for extraction. Those of ordinary skill in the art will readily appreciate that different ratios of polar to non-polar solvent may be employed as appropriate in a particular situation. Just to give a few examples, common ratios include 1:1, 2:1, 3:1, 3:2, 95:5, 90:10, 85:15, 80:20, 75:25, 70:30, 65:45, 60:40, 55:45, and 50:50. It will be appreciated that solvents or solvent mixtures of different polarities may be more effective at extracting particular carotenoids (e.g., based on their polarities and/or as a function of other attributes of the host cell material from which they are being extracted). Those of ordinary skill in the art are well able to adjust the overall polarity of the extracting solvent, for instance by adjusting the relative amounts of polar and non-polar solvents in a solvent blend, in order to achieve more efficient extraction.

Extraction may be performed under any of a variety of environmental conditions, including any of a variety of temperatures. For example, extraction may be performed on ice (for example at 4° C., 0° C., less than 0° C.), at room temperature, or at any of a variety of other temperatures. For example, a solvent may be maintained at a selected temperature (e.g., about less than 0, 0, 4, 25, 28, 30, 37, 68, 70, 75, 80, 85, 90, 95, or 100° C.) in order to improve or adjust extraction of a particular desired carotenoid.

Extraction typically yields a crude oil suspension. In some embodiments, the crude oil suspension contains some intact host cells but is at least about 95% free of intact host cells. In some embodiments, the crude oil suspension is at least about 96%, 97%, 98%, or 99% or more free of intact host cells. In some embodiments, the suspension is substantially free of water-soluble cell components (e.g., nucleic acids, cell wall or storage carbohydrates, etc.). In some embodiments, the suspension contains less than about 5%, 4%, 3%, 2%, or 1% or less water-soluble cell components.

Extraction conditions that yield a crude oil suspension will enrich for lipophilic components that accumulate in the lipid bodies within oleaginous organisms. In general, the major components of the lipid bodies consist of triacylglycerols, ergosteryl esters, other steryl esters, free ergosterol, phospholipids, and some proteins, which often function in the synthesis or regulation of the levels of other lipid body components. C16 and C18 (e.g., C16:0, C16:1, C18:0, C18:1, and C18:2) are generally the major fatty acids present in lipid bodies, mainly as components of triacylglycerol and steryl esters.

In some embodiments of the disclosure, the crude oil suspension contains at least about 2.5% by weight carotenoid compound(s) and/or retinolic compound(s); in some embodiments, the crude oil suspension contains at least about 5% by weight carotenoid compound(s) and/or retinolic compound(s), at least about 10% by weight carotenoid compound(s) and/or retinolic compound(s), at least about 20% by weight carotenoid compound(s) and/or retinolic compound(s), at least about 30% by weight carotenoid compound(s) and/or retinolic compound(s), at least about 40% by weight carotenoid compound(s) and/or retinolic compound(s), or at least about 50% by weight carotenoid compound(s) and/or retinolic compound(s).

The crude oil suspension may optionally be refined as known in the art. Refined oils may be used directly as feed or food additives. Alternatively or additionally, carotenoids and/or retinolic compound can be isolated from the oil using conventional techniques.

Given the sensitivity of carotenoids and retinolic compounds generally to oxidation, many embodiments of the disclosure employ oxidative stabilizers (e.g., ascorbyl palmitate, tocopherols, vitamin C (e.g., sodium ascorbate), ethoxyquin, vitamin E, BHT, BHA, TBHQ, etc., or combinations thereof) during and/or after carotenoid isolation. Alternatively or additionally, nitrogen or an inert gas can be utilized to purge oxygen from the process lines of any tanks or equipment. Alternatively or additionally, microencapsulation, (for example with a microencapsulation ingredients such as proteins, carbohydrates (e.g., maltodextrin, gum acacia, xanthan gum, starches/sugars like sucrose), or gelatins, or any other substance which creates a physical barrier to air and/or light) may be employed to add a physical barrier to oxidation and/or to improve handling (see, for example, U.S. Patent Applications 2004/0191365 and 2005/0169999). For example, carotenoids and/or retinolic compounds produced according to the present disclosure may be microencapsulated after isolation during the formulation of commercial products (e.g., pharmaceuticals, food supplements, electro-optic applications, animal feed additives, cosmetics, etc.) to minimize or eliminate oxidation during production, storage, transport, etc.

Extracted carotenoids and/or retinolic compounds may be further isolated and/or purified, for example, by crystallization, washing, recrystallization, and/or other purification strategies. In some embodiments, carotenoid and/or retinolic compound crystals are collected by filtration and/or centrifugation. Isolated or purified carotenoids and/or retinolic compound may be dried and/or formulated for storage, transport, sale, and/or ultimate use. To give but a few specific examples, carotenoids and/or retinolic compounds may be prepared as a water (e.g., cold water) dispersible powder (e.g., 1%-20% carotenoid: microencapsulation ingredient), as a suspension of crystals in oil (e.g., vegetable oil, e.g., about 1%-30%, 5%-30%, 10%-30% w/w), etc.

### Uses

Carotenoids and/or retinolic compounds produced according to the present disclosure can be utilized in any of a variety of applications, for example exploiting their biological or nutritional properties (e.g., anti-oxidant, anti-proliferative, etc.) and/or their pigment properties. For example, according to the present disclosure, carotenoids may be used in pharmaceuticals (see, for example, Bertram, Nutr. Rev. 57:182, 1999; Singh et al., Oncology 12:1643, 1998; Rock, Pharmacol. Ther. 75:185, 1997; Edge et al, J. Photochem Photobiol 41:189, 1997; U.S. Patent Application 2004/0116514; U.S. Patent Application 2004/0259959), food supplements (see, for example, Koyama et al, J. Photochem Photobiol 9:265, 1991; Bauernfeind, Carotenoids as colorants and vitamin A precursors, Academic Press, NY, 1981; U.S. Patent Application 2004/0115309; U.S. Patent Application 2004/0234579), electro-optic applications, animal feed additives (see, for example, Krinski, Pure Appl. Chem. 66:1003, 1994; Polazza et al., Meth. Enzymol. 213:403, 1992), cosmetics (as anti-oxidants and/or as cosmetics, including fragrances; see for example U.S. Patent Application 2004/0127554), etc. Carotenoids produced in accordance with the present disclosure may also be used as intermediates in the production of other compounds (e.g., steroids, etc.).

For example, astaxanthin and/or esters thereof may be useful in a variety of pharmaceutical applications and health foods including treatment of inflammatory diseases, asthma, atopic dermatitis, allergies, multiple myeloma, arteriosclerosis, cardiovascular disease, liver disease, cerebrovascular disease, thrombosis, neoangiogenesis-related diseases, including cancer, rheumatism, diabetic retinopathy; macular degeneration and brain disorder, hyperlipidemia, kidney ischemia, diabetes, hypertension, tumor proliferation and metastasis; and metabolic disorders. Additionally, carotenoids and astaxanthin may be useful in the prevention and treatment of fatigue, for improving kidney function in nephropathy from inflammatory diseases, as well as prevention and treatment of other life habit-related diseases. Still further, astaxanthin has been found to play a role as inhibitors of various biological processes, including interleukin inhibitors, phosphodiesterase inhibitors inhibitors, phospholipase A2 inhibitors, cyclooxygenase-2 inhibitors, matrix metalloproteinase inhibitors, capillary endothelium cell proliferation inhibitors, lipoxygenase inhibitors. See, e.g., Japanese Publication No. 2006022121, published 20060126(JP Appl No. 2005-301156 filed 20051017); Japanese Publication No. 2006016408, published 20060119(JP Appl No. 2005-301155 filed 20051017); Japanese Publication No. 2006016409, published 20060119(JP Appl No. 2005-301157 filed 20051017); Japanese Publication No. 2006016407, published 20060119(JP Appl No. 2005-301153 filed 20051017); Japanese Publication No. 2006008717, published 20060112(JP Appl No. 2005-301151 filed 20051017); Japanese Publication No. 2006008716, published 20060112(JP Appl No. 2005-301150 filed 20051017); Japanese Publication No. 2006008720, published 20060112(JP Appl No. 2005-301158 filed 20051017); Japanese Publication No. 2006008719, published 20060112(JP Appl No. 2005-301154 filed 20051017); Japanese Publication No. 2006008718, published 20060112(JP Appl No. 2005-301152 filed 20051017); Japanese Publication No. 2006008713, published 20060112(JP Appl No. 2005-301147 filed 20051017); Japanese Publication No. 2006008715, published 20060112(JP Appl No. 2005-301149 filed 20051017); Japanese Publication No. 2006008714, published 20060112(JP Appl No. 2005-301148 filed 20051017); and Japanese Publication No. 2006008712, published 20060112 (JP Appl No. 2005-301146 filed 20051017).

As other non-limiting examples, retinolic compounds produced according to the present disclosure may be used in pharmaceuticals, foodstuff, dietary supplements, electro-optic applications, animal feed additives, cosmetics, etc.

It will be appreciated that, in some embodiments of the disclosure, carotenoids and/or retinolic compounds produced by manipulated host cells as described herein are incorporated into a final product (e.g., food or feed supplement, pharmaceutical, cosmetic, dye-containing item, etc.) in the context of the host cell. For example, host cells may be lyophilized, freeze dried, frozen or otherwise inactivated, and then whole cells may be incorporated into or used as the final product. The host cell may also be processed prior to incorporation in the product to increase bioavailability (e.g., via lysis). Alternatively or additionally, a final product may incorporate only a portion of the host cell (e.g., fractionated by size, solubility), separated from the whole. For example, in some embodiments of the disclosure, lipid droplets are isolated from the host cells and are incorporated into or used as the final product. For instance, inventive carotenoid-containing and/or retinolic compound-containing lipid bodies (e.g., from engineered cells, and particularly from engineered fungal cells) may be substituted for the plant oil bodies described in U.S. Pat. No. 6,599,513 (the entire contents of which are hereby incorporated by reference) and incorporated into emulsion or emulsion formulations, as described therein. In other embodiments, the carotenoids and/or retinolic compounds themselves, or individual carotenoid and/or retinolic compounds are isolated and reformulated into a final product.

As stated above, fatty acid and glucoside esters are the predominant carotenoid esters found in nature, whereas additional esters (e.g., with organic acids or inorganic phosphate) can be synthesized to generate useful product forms. For delivery, carotenoid esters can also be formulated as salts of the ester form. See, e.g., US Publication No. 20050096477.

The amount of carotenoid and/or retinolic compound incorporated into a given product may vary dramatically depending on the product, and the particular carotenoid(s) and/or retinolic compound(s) involved. Amounts may range, for example, from less than 0.01% by weight of the product, to more than 1%, 10%, 20%, 30% or more; in some cases the carotenoid and/or retinolic compound may comprise 100% of the product. Thus, amount of carotenoid and/or retinolic compound incorporated into a given product may be, for example, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%.

In some embodiments of the disclosure, one or more produced carotenoids and/or retinolic compounds is incorporated into a component of food or feed (e.g., a food supplement). Types of food products into which carotenoids and/or retinolic compounds can be incorporated according to the present disclosure are not particularly limited, and include beverages such as milk, water, sports drinks, energy drinks, teas, juices, and liquors; confections such as jellies and biscuits; fat-containing foods and beverages such as dairy products; processed food products such as rice and soft rice (or porridge); infant formulas; breakfast cereals; or the like. In some embodiments, one or more produced carotenoids and/or retinolic compounds is incorporated into a dietary supplements, such as for example a multivitamin. In certain embodiments, beta-carotene produced according to the present disclosure is included in a dietary supplement. In certain embodiments, lutein produced according to the present disclosure is included in a dietary supplement. In certain embodiments, retinol, retinal, retinyl palmitate, retinyl acetate, and/or retinoic acid produced according to the present disclosure is included in a dietary supplement. In some embodiments of this aspect of the disclosure, it may be useful to incorporate the carotenoids and/or retinolic compounds within bodies of edible lipids as it may facilitate incorporation into certain fat-containing food products. Thus, for example, when the edible fungus, Candida utilis is used as a host, its' carotenoid and/or retinolic compound containing lipids may be directly incorporated into a component of food or feed (e.g., a food supplement).

Examples of feedstuffs into which carotenoids and/or retinolic compounds produced in accordance with the present disclosure may be incorporated include, for instance, pet foods such as cat foods, dog foods and the like, feeds for aquarium fish, cultured fish or crustaceans, etc., feed for farm-raised animals (including livestock and further including fish or crustaceans raised in aquaculture). Food or feed material into which the carotenoid(s) and/or retinolic compound(s) produced in accordance with the present disclosure is incorporated is preferably palatable to the organism which is the intended recipient. This food or feed material may have any physical properties currently known for a food material (e.g., solid, liquid, soft).

In some embodiments, feedstuffs containing carotenoids and/or retinolic compounds produced in accordance with the present disclosure are substantially free of intact host cells. For example, feedstuffs of the present disclosure may be at least about 95% free of intact host cells. In some embodiments, feedstuffs of the present disclosure are at least about 96%, 97%, 98%, or 99% or more free of intact host cells. Such embodiments are typical when the carotenoids and/or retinolic compounds are highly purified away from the host cell in which they were produced (see section entitled “Production and Isolation of Carotenoids and/or Retinolic Compounds”).

In some embodiments, feedstuffs containing carotenoids and/or retinolic compounds produced in accordance with the present disclosure are not substantially free of intact host cells. For example, feedstuffs of the present disclosure may comprise greater than about 95% intact host cells. In certain embodiments, feedstuffs of the present disclosure comprise greater than about 70%, 75%, 85%, or 90% intact host cells. In certain embodiments, feedstuffs of the present disclosure comprise nearly intact host cells. For example, feedstuffs of the present disclosure may comprise greater than about 70%, 75%, 85%, 90%, or 95% nearly intact host cells. As will be appreciated by those of ordinary skill in the art, carotenoid and/or retinolic compound-containing feedstuffs of the present disclosure that contain intact cells and/or nearly intact cells will have great utility in providing the carotenoids and/or retinolic compounds of interest present in such host cells to an animal Such embodiments are advantageous when host cells that produce the carotenoids and/or retinolic compounds of interest contain additional vitamins, nutrients, etc. that benefit the animal.

In some embodiments of the disclosure, one or more produced carotenoids and/or retinolic compounds is incorporated into a cosmetic product. Examples of such cosmetics include, for instance, skin cosmetics (e.g., lotions, emulsions, creams and the like), lipsticks, anti-sunburn cosmetics, makeup cosmetics, fragrances, products for daily use (e.g., toothpastes, mouthwashes, bad breath preventive agents, solid soaps, liquid soaps, shampoos, conditioners), etc.

In some embodiments, one or more produced carotenoids and/or retinolic compounds is incorporated into a pharmaceutical. Examples of such pharmaceuticals include, for instance, various types of tablets, capsules, drinkable agents, troches, gargles, etc. In some embodiments, the pharmaceutical is suitable for topical application. Dosage forms are not particularly limited, and include capsules, oils, granula, granula subtilae, pulveres, tabellae, pilulae, trochisci, or the like. Oils and oil-filled capsules may provide additional advantages both because of their lack of ingredient decomposition during manufacturing, and because inventive carotenoid-containing and/or retinolic compound-containing lipid droplets may be readily incorporated into oil-based formulations.

Pharmaceuticals according to the present disclosure may be prepared according to techniques established in the art including, for example, the common procedure as described in the United States Pharmacopoeia, for example.

Carotenoids and/or retinolic compounds produced according to the present disclosure may be incorporated into any pigment-containing product including, for example, fabric, paint, etc. They may also be incorporated into a product which is an environmental indicator, or an instrument such as a biosensor for use as a detection agent.

Carotenoids and/or retinolic compounds produced according to the present disclosure (whether isolated or in the context of lipid droplets or of cells, e.g., fungal cells) may be incorporated into products as described herein by combinations with any of a variety of agents. For instance, such carotenoids and/or retinolic compounds may be combined with one or more binders or fillers. In some embodiments, inventive products will include one or more chelating agents, pigments, salts, surfactants, moisturizers, viscosity modifiers, thickeners, emollients, fragrances, preservatives, etc., and combinations thereof.

Useful surfactants include, for example, anionic surfactants such as branched and unbranched alkyl and acyl hydrocarbon compounds, sodium dodecyl sulfate (SDS); sodium lauryl sulfate (SLS); sodium lauryl ether sulfate (SLES); sarconisate; fatty alcohol sulfates, including sodium, potassium, ammonium or triethanolamine salts of C₁₀ to C₁₈ saturated or unsaturated forms thereof; ethoxylated fatty alcohol sulfates, including alkyl ether sulfates; alkyl glyceryl ether sulfonate, alpha sulpho fatty acids and esters; fatty acid esters of isethionic acid, including Igepon A; acyl (fatty) N-methyltaurides, including Igepon T; dialkylsulfo succinate esters, including C₈, C₁₀ and C₁₂ forms thereof; Miranot BT also referred to as lauroamphocarboxyglycinate and sodium tridecath sulfate; N-acylated amino acids, such as sodium N-lauroyl sarconisate or gluconate; sodium coconut monoglyceride sulfonate; and fatty acid soaps, including sodium, potassium, DEA or TEA soaps.

Among the cationic surfactants that are useful are monoalkyl trimethyl quartenary salts; dialkyl dimethyl quartenary salts; ethoxylated or propoxylated alkyl quaternary amonium salts, also referred to in the art as ethoquats and propoquats; cetyl benzylmethylalkyl ammonium chloride; quaternized imidazolines, which are generally prepared by reacting a fat or fatty acid with diethylenetriamine followed by quaternization, and non-fat derived cationic polymers such as the cellulosic polymer, Polymer JR (Union Carbide).

Further useful cationic surfactants include lauryl trimethyl ammonium chloride; cetyl pyridinium chloride; and alkyltrimethylammonium bromide. Cationic surfactants are particularly useful in the formulation of hair care products, such as shampoos, rinses and conditioners.

Useful nonionic surfactants include polyethoxylated compounds and polypropoxylated products. Examples of ethoxylated and propoxylated non-ionic surfactants include ethoxylated anhydrohexitol fatty esters, for example Tween 20; mono- and diethanolamides; Steareth-20, also known as Volpo20; polyethylene glycol fatty esters (PEGs), such as PEG-8-stearate, PEG-8 distearate; block co-polymers, which are essentially combinations of hydrophylic polyethoxy chains and lipophilic polypropoxy chains and generically known as Poloaxamers.

Still other useful non-ionic surfactants include fatty esters of polyglycols or polyhydric alcohols, such as mono and diglyceride esters; mono- and di-ethylene glycol esters; diethylene glycol esters; sorbitol esters also referred to as Spans; sucrose esters; glucose esters; sorbitan monooleate, also referred to as Span80; glyceryl monostearate; and sorbitan monolaurate, Span20 or Arlacel 20.

Yet other useful nonionic surfactants include polyethylene oxide condensates of alkyl phenols and polyhydroxy fatty acid amide surfactants which may be prepared as for example disclosed in U.S. Pat. No. 2,965,576.

Examples of amphoteric surfactants which can be used in accordance with the present disclosure include betaines, which can be prepared by reacting an alkyldimethyl tertiary amine, for example lauryl dimethylamine with chloroacetic acid. Betaines and betaine derivatives include higher alkyl betaine derivatives including coco dimethyl carboxymethyl betaine; sulfopropyl betaine; alkyl amido betaines; and cocoamido propyl betaine. Sulfosultaines which may be used include for example, cocoamidopropyl hydroxy sultaine. Still other amphoteric surfactants include imidazoline derivatives and include the products sold under the trade name “Miranol” described in U.S. Pat. No. 2,528,378 which is incorporated herein by reference in its entirety. Still other amphoterics include phosphates for example, cocamidopropyl PG-dimonium chloride phosphate and alkyldimethyl amine oxides.

Suitable moisturizers include, for example, polyhydroxy alcohols, including butylene glycol, hexylene glycol, propylene glycol, sorbitol and the like; lactic acid and lactate salts, such as sodium or ammonium salts; C₃ and C₆ diols and triols including hexylene glycol, 1,4 dihydroxyhexane, 1,2,6-hexane triol; aloe vera in any of its forms, for example aloe vera gel; sugars and starches; sugar and starch derivatives, for example alkoxylated glucose; hyaluronic acid; lactamide monoethanolamine; acetamide monoethanolamine; glycolic acid; alpha and beta hydroxy acids (e.g., lactic, glycolic salicylic acid); glycerine; pantheol; urea; vaseline; natural oils; oils and waxes (see: the emollients section herein) and mixtures thereof.)

Viscosity modifiers that may be used in accordance with the present disclosure include, for example, cetyl alcohol; glycerol, polyethylene glycol (PEG); PEG-stearate; and/or Keltrol.

Appropriate thickeners for use in inventive products include, for example, gelling agents such as cellulose and derivatives; Carbopol and derivatives; carob; carregeenans and derivatives; xanthane gum; sclerane gum; long chain alkanolamides; bentone and derivatives; Kaolin USP; Veegum Ultra; Green Clay; Bentonite NFBC; etc.

Suitable emollients include, for example, natural oils, esters, silicone oils, polyunsaturated fatty acids (PUFAs), lanoline and its derivatives and petrochemicals.

Natural oils which may be used in accordance with the present disclosure may be obtained from sesame; soybean; apricot kernel; palm; peanut; safflower; coconut; olive; cocoa butter; palm kernel; shea butter; sunflower; almond; avocado; borage; carnauba; hazel nut; castor; cotton seed; evening primrose; orange roughy; rapeseed; rice bran; walnut; wheat germ; peach kernel; babassu; mango seed; black current seed; jojoba; macademia nut; sea buckthorn; sasquana; tsubaki; mallow; meadowfoam seed; coffee; emu; mink; grape seed; thistle; tea tree; pumpkin seed; kukui nut; and mixtures thereof.

Esters which may be used include, for example, C₈-C₃₀ alkyl esters of C₉-C₃₀ carboxylic acids; C₁-C₆ diol monoesters and diesters of C₉-C₃₀ carboxylic acids; C₁₀-C₂₀ alcohol monosorbitan esters, C₁₀-C₂₀ alcohol sorbitan di- and tri-esters; C₁₀-C₂₀ alcohol sucrose mono-, di-, and tri-esters and C₁₀-C₂₀ fatty alcohol esters of C₂-C₆ 2-hydroxy acids and mixtures thereof. Examples of these materials include isopropyl palmitate; isopropyl myristate; isopropyl isononate; C₁₂/C₁₄ benzoate ester (also known as Finesolve); sorbitan palmitate, sorbitan oleate; sucrose palmitate; sucrose oleate; isostearyl lactate; sorbitan laurate; lauryl pyrrolidone carboxylic acid; panthenyl triacetate; and mixtures thereof.

Further useful emollients include silicone oils, including non-volatile and volatile silicones. Examples of silicone oils that may be used in the compositions of the present disclosure are dimethicone; cyclomethycone; dimethycone-copolyol; aminofunctional silicones; phenyl modified silicones; alkyl modified silicones; dimethyl and diethyl polysiloxane; mixed C₁-C₃₀ alkyl polysiloxane; and mixtures thereof. Additionally useful silicones are described in U.S. Pat. No. 5,011,681 to Ciotti et al., incorporated by reference herein.

A yet further useful group of emollients includes lanoline and lanoline derivatives, for example lanoline esters.

Petrochemicals which may be used as emollients in the compositions of the present disclosure include mineral oil; petrolatum; isohexdecane; permethyl 101; isododecanol; C₁₁-C₁₂ Isoparrafin, also known as Isopar H.

Among the waxes which may be included in inventive products are animal waxes such as beeswax; plant waxes such as carnauba wax, candelilla wax, ouricurry wax, Japan wax or waxes from cork fibres or sugar cane. Mineral waxes, for example paraffin wax, lignite wax, microcrystalline waxes or ozokerites and synthetic waxes may also be included.

Exemplary fragrances for use in inventive products include, for instance, linear and cyclic alkenes (i.e. terpenes); primary, secondary and tertiary alcohols; ethers; esters; ketones; nitrites; and saturated and unsaturated aldehydes; etc.

Examples of synthetic fragrances that may be used in accordance with the present disclosure include without limitation acetanisole; acetophenone; acetyl cedrene; methyl nonyl acetaldehyde; musk anbrette; heliotropin; citronellol; sandella; methoxycitranellal; hydroxycitranellal; phenyl ethyl acetate; phenylethylisobutarate; gamma methyl ionone; geraniol; anethole; benzaldehyde; benzyl acetate; benzyl salicate; linalool; cinnamic alcohol; phenyl acetaldehyde; amyl cinnamic aldehyde; caphore; p-tertiairy butyl cyclohexyl acetate; citral; cinnamyl acetate; citral diethyl acetal; coumarin; ethylene brasslate; eugenol; l-menthol; vanillin; etc.

Examples of natural fragrances of use herein include without limitation lavandin; heliotropin; sandlewood oil; oak moss; pathouly; ambergris tincture; ambrette seed absolute; angelic root oil; bergamont oil; benzoin Siam resin; buchu leaf oil; cassia oil; cedarwood oil; cassia oil; castoreum; civet absolute; chamomile oil; geranium oil; lemon oil; lavender oil; Ylang Ylang oil; etc.

A list of generally used fragrance materials can be found in various reference sources, for example, “Perfume and Flavor Chemicals”, Vols. I and II; Steffen Arctander Allured Pub. Co. (1994) and “Perfumes: Art, Science and Technology”; Muller, P. M. and Lamparsky, D., Blackie Academic and Professional (1994) both incorporated herein by reference.

Suitable preservatives include, among others, (e.g., sodium metabisulfite; Glydant Plus; Phenonip; methylparaben; Germall 115; Germaben II; phytic acid; sodium lauryl sulfate (SLS); sodium lauryl ether sulfate (SLES); Neolone; Kathon; Euxyl and combinations thereof), anti-oxidants (e.g., butylated hydroxytoluene (BHT); butylated hydroxyanisol (BHA); ascorbic acid (vitamin C); tocopherol; tocopherol acetate; phytic acid; citric acid; pro-vitamin A.

In some embodiments, inventive products will comprise an emulsion (e.g., containing inventive lipid bodies), and may include one or more emulsifying agents (e.g., Arlacel, such as Alacel 165; Glucamate; and combinations thereof) and/or emulsion stabilizing agents.

In some embodiments, inventive products will include one or more biologically active agents other than the carotenoid(s). To give but a few examples, inventive cosmetic or pharmaceutical products may include one or more biologically active agents such as, for example, sunscreen actives, anti-wrinkle actives, anti-aging actives, whitening actives, bleaching actives, sunless tanning actives, anti-microbial actives, anti-acne actives, anti-psoriasis actices, anti-eczema actives, antioxidants, anesthetics, vitamins, protein actives, etc.

### EXEMPLIFICATION

Table 26 below describes certain Yarrowia lipolytica strains used in the following exemplification:

TABLE 26
Yarrowia lipolytica strains.

| | | |
|---|---|---|
| NRRL Y-1095 | Wild type diploid | |
| ATCC 76861 | MATB ura2-21 lyc1-5 LYS1-5B | |
| ATCC 76982 | MATB ade1 leu2-35 lyc1-5 xpr2 | |
| ATCC 201249 | MATA ura3-302 leu2-270 | |
| | lys8-11 PEX17-HA | |
| MF346 | MATA ura2-21 | ATCC76861 × |
| | | ATCC201249 |
| MF350 | MATB ura2-21 leu2-35 ade1 | ATCC76982 × MF346 |

(The genotypes at LYC1, LYS1, XPR2, and PEX17 were not determined in crosses nor verified for ATCC strains.)

All basic molecular biology and DNA manipulation procedures described herein are generally performed according to Sambrook et al. or Ausubel et al. (Sambrook J, Fritsch E F, Maniatis T (eds). 1989. Molecular Cloning: A Laboratory Manual. Cold Spring Harbor Laboratory Press: New York; Ausubel F M, Brent R, Kingston R E, Moore D D, Seidman J G, Smith J A, Struhl K (eds). 1998. Current Protocols in Molecular Biology. Wiley: New York).

### Example 1

### Production of Plasmids for Carotenoid Strain Construction

Plasmids were generated for construction of carotenoid producing strains. The following subparts describe production of plasmids encoding carotenogenic polypeptides. Plasmids used in these studies and details of their construction are described in Table 27. Additional plasmid construction details and descriptions of their use are found in the text of the relevant subsection. All PCR amplifications used NRRL Y-1095 genomic DNA as template unless otherwise specified. The URA5 gene described below is allelic with the ura2-21 auxotrophy above. The GPD1 and TEF1 promoters are from Y. lipolytica as is the XPR2 terminator.

GGSJ is the gene encoding the Y. lipolytica gene encoding geranylgeranylpyrophosphate synthase. The nucleic acid coding sequence and encoded Ggsl protein of pMB4591 and pMB4683 are as follows:

(SEQ ID NO: 5)

atggattataacagcgcggatttcaaggagatatggggcaaggccgccgacaccgcgctgctgggaccgtacaactacc
tcgccaacaaccggggccacaacatcagagaacacttgatcgcagcgttcggagcggttatcaaggtggacaagagcgatctcgagaccatttcg
cacatcaccaagattttgcataactcgtcgctgcttgttgatgacgtggaagacaactcgatgctccgacgaggcctgccggcagcccattgtctgttt
ggagtcccccaaaccatcaactccgccaactacatgtactttgtggctctgcaggaggtgctcaagctcaagtcttatgatgccgtctccattttcacc
gaggaaatgatcaacttgcatagaggtcagggtatggatctctactggagagaaacactcacttgcccctcggaagacgagtatctggagatggtg
gtgcacaagaccggtggactgtttcggctggctctgagacttatgctgtcggtggcatcgaaacaggaggaccatgaaaagatcaactttgatctca
cacaccttaccgacacactgggagtcatttaccagattctggatgattacctcaacctgcagtccacggaattgaccgagaacaagggattctgcga
agatatcagcgaaggaaagttttcgtttccgctgattcacagcatacgcaccaacccggataaccacgagattctcaacattctcaaacagcgaaca
agcgacgcttcactcaaaaagtacgccgtggactacatgagaacagaaaccaagagtttcgactactgcctcaagaggatacaggccatgtcactc
aaggcaagttcgtacattgatgatctagcagcagctggccacgatgtctccaagctacgagccattttgcattattttgtgtccacctctgactgtgagg
agagaaagtactttgaggatgcgcagtga

(SEQ ID NO: 6)

mdynsadfkeiwgkaadtallgpynylannrghnirehliaafgavikvdksdletishitkilhnssllvddvednsm
lrrglpaahclfgvpqtinsanymyfvalqevlklksydavsifteeminlhrgqgmdlywretltcpsedeylemvvhktgglfrlalrlmlsv
askqedhekinfdlthltdtlgviyqilddylnlqsteltenkgfcedisegkfsfplihsirtnpdnheilnilkqrtsdaslkkyavdymrtetksf
dyclkriqamslkassyiddlaaaghdvsklrailhyfvstsdceerkyfedaq

TABLE 27
Plasmids

| Plasmid | Backbone | Insert | Oligos or source |
|---|---|---|---|
| pMB4529 | PCR2.1 | 3.4 kb ADE1 PCR | MO4475 & |
| | | product | MO4476 |
| pMB4534 | PCR2.1 | 2.1 kb LEU2 PCR | MO4477 & |
| | | product | MO4478 |
| pMB4535 | PCR2.1 | 1.2 kb URA5 PCR | MO4471 & |
| | | product | MO4472 |
| pMB4589 | pMB4535 (KpnI + SpeI) | 1.2 kb GPD1 promoter | MO4568 & |
| | | (KpnI + NotI); 0.14 kb | MO4591; |
| | | XPR2 terminator (NotI + | MO4566 & |
| | | SpeI) | MO4593 |
| pMB4590 | pMB4535 (KpnI + SpeI) | 0.4 kb TEF1 promoter | MO4571 & |
| | | (KpnI + NotI); 0.14 kb | MO4592; |
| | | XPR2 terminator (NotI + | MO4566 & |
| | | SpeI) | MO4593 |
| pMB4591 | pMB4590 (NheI + MluI) | 1.0 kb GGS1 ORF (XbaI+ MluI) | MO4534 & |
| | | | MO4544 |
| pMB4597 | pMB4534 (Acc65I + | GPD1 promoter & XPR2 | From pMB4589 |
| | SpeI) | terminator (Acc65I + | |
| | | SpeI) | |
| pMB4603 | pMB4597 (RsrII + MluI) | Residual backbone | From pMB4590 |
| | | & TEF1 promoter (RsrII + MluI) | |
| pMB4616 | pMB4529 (RsrII + SpeI) | Residual backbone | From pMB4589 |
| | | & GPD1 promoter & | |
| | | XPR2 terminator (RsrII + | |
| | | SpeI) | |
| pMB4629 | pMB4616 (RsrII + MluI) | Residual backbone | From pMB4590 |
| | | & TEF1 promoter (RsrII+ MluI) | |
| pMB4631 | pMB4603 (KpnI + NheI) | 1.2 kb GPD1 promoter | MO4568 & |
| | | (KpnI + NheI); | MO4659 |
| pMB4628 | pMB4603 | carRP | See 1A |
| pMB4637 | pMB4629 (NheI + MluI} | 1.5 kb hmg1^{trunc} ORF | See 1D |
| | | (XbaI + MluI) | |
| pMB4714 | pMB4691 (NheI + MluI) | l.5 kb hmg1^{trunc} ORF | See 1D |
| | | (XbaI + MluI) | |
| pMB4638 | pMB4629 | carB(i⁻) | See 1B |
| pMB4660 | pMB4638 (+URA3) | carB(i⁻) | See 1C |
| pMB4662 | pMB4631 (SpeI + XhoI) | 1.8 kb URA3 fragment | See 1C |
| | | (SpeI + BsaI) | |
| pMB4683 | pMB4662 (Acc65I + | 1.4 kb tef1p-GGS1 | From pMB4591 |
| | MluI) | fragment (Acc65I + | |
| | | MluI) | |
| pMB4692 | pMB4662 (Acc65I + | 0.4 kb TEF1 promoter | See 1E |
| | MluI) | (Acc65I + NheI); 0.55 kb | |
| | | crtZ ORF (XbaI + MluI) | |
| pMB4698 | pMB4629 (NheI + MluI) | 0.9 kb crtW ORF (XbaI + MluI) | See 1F |
| pMB4599 | pBluescriptSKII- | 1.9 kb carRP gene | See 1A |
| | EcoRV) | | |
| pMB4606 | pBluescriptSKII- | 1.9 kb carB gene | See 1B |
| | (EcoRV) | | |
| pMB4613 | pMB4599 (Acc65I + | carRP(i⁻) | See 1A |
| | PpuMI) | | |
| pMB4619 | pBluescriptSKII- | carB(i⁻) | See 1A |
| | (BamHI + Acc65I)) | | |
| pMB4705 | pMB4603 (NheI + MluI) | carRP(i⁻) | See 1A |
| pMB4691 | pMB4662 (Acc65I + | 0.4 kb TEF1 promoter | From pMB4629 |
| | MluI) | (Acc65I + MluI) | |
| pMB4751 | pMB4691 | 0.75 kb YALI0D12903g | Inserted between |
| | | promoter + 0.45 Y. | TEF1p/XPRt and |
| | | lipolytica HIS3 | URA3 |
| | | terminator | |
| pMB4719 | pMB4691 (NheI + MluI) | crtZ (E. litoralis) | See 1J |
| pMB4778 | pMB4751 (NheI + MluI) | crtZ (P. bermudensis) | See 1I |
| pMB4741 | pMB4629 (NheI + MluI) | crtW (Aurantimonas) | See 1G |
| pMB4735 | pMB4629 (NheI + MluI) | crtW (P. bermudensis) | See 1H |
| pMB4812 | pMB4603 (NheI + MluI) | al-2 (N. crassa) | See 1K |
| pMB4846 | pMB4691 (NheI + MluI) | crtZ (Erythrobacter sp. | See 1L |
| | | NAP1) | |
| pMB4835 | pMB4691 (NheI + MluI) | crtZ (S. alaskensis) | See 1M |
| pMB4845 | pMB4691 (NheI + MluI) | crtZ (R. biformata) | See 1N |
| pMB4837 | pMB4691 (NheI + MluI) | crtZ (X. autrophicus) | See 1O |
| pMB4850 | pMB4691 (NheI + MluI) | crtZ (P. putida) | See 1P |

Certain oligonucleotides referred to in Table 27 above are as follows:

MO4471

(SEQ ID NO: 7)

5′-CTGGGTGACCTGGAAGCCTT
MO4472

(SEQ ID NO: 8)

5′-AAGATCAATCCGTAGAAGTTCAG
MO4475

(SEQ ID NO: 9)

5′-AAGCGATTACAATCTTCCTTTGG
MO4476

(SEQ ID NO: 10)

5′-CCAGTCCATCAACTCAGTCTCA
MO4477

(SEQ ID NO: 11)

5′-GCATTGCTTATTACGAAGACTAC
MO4478

(SEQ ID NO: 12)

5′-CCACTGTCCTCCACTACAAACAC
MO4534

(SEQ ID NO: 13)

5′-CACAAACGCGTTCACTGCGCATCCTCAAAGT
MO4544

(SEQ ID NO: 14)

5′-CACAATCTAGACACAAATGGATTATAACAGCGCGGAT
MO4566

(SEQ ID NO: 15)

5′-CACAAACTAGTTTGCCACCTACAAGCCAGAT
MO4568

(SEQ ID NO: 16)

5′-CACAAGGTACCAATGTGAAAGTGCGCGTGAT
MO4571

(SEQ ID NO: 17)

5′-CACAAGGTACCAGAGACCGGGTTGGCGG
MO4591

(SEQ ID NO: 18)

5′-CACAAGCGGCCGCGCTAGCATGGGGATCGATCTCTTATAT
MO4592

(SEQ ID NO: 19)

5′-CACAAGCGGCCGCGCTAGCGAATGATTCTTATACTCAGAAG
MO4593

(SEQ ID NO: 20)

5′-CACAAGCGGCCGCACGCGTGCAATTAACAGATAGTTTGCC
MO4659

(SEQ ID NO: 21)

5′-CACAAGCTAGCTGGGGATGCGATCTCTTATATC

### 1A: Production of pMB4628 (tef1p-carRP LEU2) and pMB4705 (tef1p-carRP[i⁻] LEU2) Encoding Phytoene Synthase/Lycopene Cyclase

Intron-containing carRP was amplified from M. circinelloides (ATCC 90680) genomic DNA using MO4525 and MO4541:

MO4525

(SEQ ID NO: 22)

5′-CACAAACGCGTTTAAATGGTATTTAGATTTCTCATT
MO4541

(SEQ ID NO: 23)

5′-CACAATCTAGACACAAATGCTGCTCACCTACATGGA

and the resulting 1.9 kb fragment was phosphorylated with T4 polynucleotide kinase. The resulting fragment was blunt-end ligated into pBluescriptSKII—cleaved with EcoRV, yielding pMB4599. The 1.9 kb XbaI-MluI fragment from pMB4599 was inserted into NheI- and MluI-cleaved pMB4603, yielding pMB4628. The intron containing nucleic acid coding sequence and encoded CarRP protein (assuming correctly predicted splicing) of pMB4628 are as follows:

(SEQ ID NO: 24)

atgctgctcacctacatggaagtccacctctactacacgctgcctgtgctgggcgtcctgtcctggctgtcgcggccgtact
acacagccaccgatgcgctcaaattcaaatttctgacactggttgccttcacgaccgcctccgcctgggacaactacattgtctaccacaaggcgtg
gtcctactgccccacctgcgtcaccgctgtcattggctacgtgcccttggaggagtacatgttcttcatcatcatgactctgttgaccgtggcattcacc
aatctggtgatgcgctggcacctgcacagcttctttatcaggcctgaaacgcccgtcatgcagtccgtcctggtccgtcttgtccccataacagcctta
ttaatcactgcatacaaggcttgggtaagcaaacaaacaaatgatgtgccgcatcgcattttaatattaaccattgcatacacagcatttggcggtccct
ggaaagccactgttctacggatcatgcattttgtggtacgcctgtccggttttggccttattgtggtttggtgctggcgagtacatgatgcgtcgtccgct
ggcggtgctcgtctccattgcgctgcccacgctgtttctctgctgggtcgatgtcgtcgctattggcgccggcacatgggacatttcgctggccacaa
gcaccggcaagttcgtcgtgccccacctgcccgtggaggaattcatgttctttgcgctaattaataccgttttggtatttggtacgtgtgcgatcgatcg
cacgatggcgatcctccacctgttcaaaaacaagagtccttatcagcgcccataccagcacagcaagtcgttcctccaccagatcctcgagatgacc
tgggccttctgtttacccgaccaagtgctgcattcagacacattccacgacctgtccgtcagctgggacatcctgcgcaaggcctccaagtccttttac
acggcctctgctgtctttcccggcgacgtgcgccaagagctcggtgtgctatacgccttttgcagagccacggacgatctctgcgacaacgagcag
gtccctgtgcagacgcgaaaggagcagctgatactgacacatcagttcgtcagcgatctgtttggccaaaagacaagcgcgccgactgccattga
ctgggacttttacaacgaccaactgcctgcctcgtgcatctctgccttcaagtcgttcacccgtttgcgccatgtgctggaagctggagccatcaagg
aactgctcgacgggtacaagtgggatttggagcgtcgctccatcagggatcaggaggatctcagatattactcagcttgtgtcgccagcagtgttgg
tgaaatgtgcactcgcatcatactggcccacgccgacaagcccgcctcccgccagcaaacacagtggatcattcagcgtgcgcgtgaaatgggtc
tggtactccaatatacaaacattgcaagagacattgtcaccgacagcgaggaactgggcagatgctacctgcctcaggattggcttaccgagaagg
aggtggcgctgattcaaggcggccttgcccgagaaattggcgaggagcgattgctctcactgtcgcatcgcctcatctaccaggcagacgagctc
atggtggttgccaacaagggcatcgacaagctgcccagccattgtcaaggcggcgtgcgtgcggcctgcaacgtctatgcttccattggcaccaa
gctcaagtcttacaagcaccactatcccagcagagcacatgtcggcaattcgaaacgagtggaaattgctcttcttagcgtatacaacctttacaccg
cgccaattgcgactagtagtaccacacattgcagacagggaaaaatgagaaatctaaataccatttaa

(SEQ ID NO: 25)

mlltymevhlyytlpvlgvlswlsrpyytatdalkfkfltlvafttasawdnyivyhkawsycptcvtavigyvpleey
mffiimtlltvaftnlvmrwhlhsffirpetpvmqsvlvrlvpitallitaykawhlavpgkplfygscilwyacpvlallwfgageymmrrpla
vlvsialptlflcwvdvvaigagtwdislatstgkfvvphlpveefmffalintvlvfgtcaidrtmailhlfknkspyqrpyqhsksflhqilemt
wafclpdqvlhsdtfhdlsvswdilrkasksfytasavfpgdvrqelgvlyafcratddlcdneqvpvqtrkeqlilthqfvsdlfgqktsaptaid
wdfyndqlpascisafksftrlrhvleagaikelldgykwdlerrsirdqedlryysacvassvgemctriilahadkpasrqqtqwiiqrarem
glvlqytniardivtdseelgrcylpqdwltekevaliqgglareigeerllslshrliyqadelmvvankgidklpshcqggvraacnvyasigt
klksykhhypsrahvgnskrveiallsvynlytapiatsstthcrqgkmrnlnti

Alternatively, pMB4599 was also used as a template for PCR amplification using MO4318, MO4643, MO4644, and MO4639:

MO4318

(SEQ ID NO: 26)

5′-GTAAAACGACGGCCAGT
MO4643

(SEQ ID NO: 27)

5′-CACACGGTCTCATGCCAAGCCTTGTATGCAGTGATTAA
MO4639

(SEQ ID NO: 28)

5′-CCACTGTGTTTGCTGGCGG
MO4644

(SEQ ID NO: 29)

5′-CACACGGTCTCTGGCATTTGGCGGTCCCTGGAAA

producing fragments of 0.5 and 0.95 kb, that were subsequently cleaved with Acc65I and BsaI; and BsaI and PpuMI, respectively. These fragments were ligated to pMB4599 that had been digested with Acc65I and PpuMI, yielding pMB4613, harboring intronless carRP. The 1.85 kb XbaI-MluI fragment from pMB4613 was inserted into NheI- and MluI-cleaved pMB4603 to yield pMB4705.

The intronless nucleic acid coding sequence of pMB4705 is as follows, and encodes the same CarRP protein as above:

(SEQ ID NO: 30)

atgctgctcacctacatggaagtccacctctactacacgctgcctgtgctgggcgtcctgtcctggctgtcgcggccgtact
acacagccaccgatgcgctcaaattcaaatttctgacactggttgccttcacgaccgcctccgcctgggacaactacattgtctaccacaaggcgtg
gtcctactgccccacctgcgtcaccgctgtcattggctacgtgcccttggaggagtacatgttcttcatcatcatgactctgttgaccgtggcattcacc
aatctggtgatgcgctggcacctgcacagcttctttatcaggcctgaaacgcccgtcatgcagtccgtcctggtccgtcttgtccccataacagcctta
ttaatcactgcatacaaggcttggcatttggcggtccctggaaagccactgttctacggatcatgcattttgtggtacgcctgtccggttttggccttatt
gtggtttggtgctggcgagtacatgatgcgtcgtccgctggcggtgctcgtctccattgcgctgcccacgctgtttctctgctgggtcgatgtcgtcgc
tattggcgccggcacatgggacatttcgctggccacaagcaccggcaagttcgtcgtgccccacctgcccgtggaggaattcatgttctttgcgcta
attaataccgttttggtatttggtacgtgtgcgatcgatcgcacgatggcgatcctccacctgttcaaaaacaagagtccttatcagcgcccataccag
cacagcaagtcgttcctccaccagatcctcgagatgacctgggccttctgtttacccgaccaagtgctgcattcagacacattccacgacctgtccgt
cagctgggacatcctgcgcaaggcctccaagtccttttacacggcctctgctgtctttcccggcgacgtgcgccaagagctcggtgtgctatacgcc
ttttgcagagccacggacgatctctgcgacaacgagcaggtccctgtgcagacgcgaaaggagcagctgatactgacacatcagttcgtcagcga
tctgtttggccaaaagacaagcgcgccgactgccattgactgggacttttacaacgaccaactgcctgcctcgtgcatctctgccttcaagtcgttcac
ccgtttgcgccatgtgctggaagctggagccatcaaggaactgctcgacgggtacaagtgggatttggagcgtcgctccatcagggatcaggagg
atctcagatattactcagcttgtgtcgccagcagtgttggtgaaatgtgcactcgcatcatactggcccacgccgacaagcccgcctcccgccagca
aacacagtggatcattcagcgtgcgcgtgaaatgggtctggtactccaatatacaaacattgcaagagacattgtcaccgacagcgaggaactggg
cagatgctacctgcctcaggattggcttaccgagaaggaggtggcgctgattcaaggcggccttgcccgagaaattggcgaggagcgattgctct
cactgtcgcatcgcctcatctaccaggcagacgagctcatggtggttgccaacaagggcatcgacaagctgcccagccattgtcaaggcggcgtg
cgtgcggcctgcaacgtctatgcttccattggcaccaagctcaagtcttacaagcaccactatcccagcagagcacatgtcggcaattcgaaacga
gtggaaattgctcttcttagcgtatacaacctttacaccgcgccaattgcgactagtagtaccacacattgcagacagggaaaaatgagaaatctaaat
accatttaa

### 1B: Production of pMB4638 (tef1p-carB ADE1), Encoding Phytoene Dehydrogenase

Intron-containing carB was amplified from M. circinelloides (ATCC 90680) genomic DNA using MO4530 and MO4542:

MO4530

(SEQ ID NO: 31)

5′-CACAAACGCGTTTAAATGACATTAGAGTTATGAAC
MO4542

(SEQ ID NO: 32)

5′-CACAATCTAGACACAAATGTCCAAGAAACACATTGTC

and the resulting 1.9 kb fragment was phosphorylated with T4 polynucleotide kinase and blunt-end ligated into pBS-SKII-cleaved with EcoRV, yielding pMB4606. pMB4606 was then used as a template for PCR amplification using MO4318 and MO4648; MO4646 and MO4647; and MO4343 and MO4645:

MO4318

(SEQ ID NO: 33)

5′-GTAAAACGACGGCCAGT
MO4648

(SEQ ID NO: 34)

5′-CACAAGGTCTCAAGCACGCATCCCGGAACTG
MO4646

(SEQ ID NO: 35)

5′-CACACGGTCTCAGGCATGTCGCCCTACGATGC
MO4647

(SEQ ID NO: 36)

5′-CACACGGTCTCATGCTTGCACCCACAAAGAATAGG
MO4343

(SEQ ID NO: 37)

5′-CAGGAAACAGCTATGAC
MO4645

(SEQ ID NO: 38)

5′-CACACGGTCTCTTGCCCATATACATGGTCTGAAACG

producing fragments of 0.4 and 0.85 and 0.7 kb, that were subsequently cleaved with Acc65I and BsaI; BsaI; and BsaI and BamHI, respectively. These fragments were ligated to pBS-SKII—that had been cut with Acc65I and BamHI, yielding pMB4619, harboring intronless carB. The 1.75 kb XbaI-MluI fragment from pMB4619 was inserted into NheI- and MluI-cleaved pMB4629, yielding pMB4638. The resulting nucleic acid coding sequence and encoded CarB protein of pMB4638 are as follows:

(SEQ ID NO: 39)

atgtccaagaaacacattgtcattatcggtgctggcgtgggtggcacggctacagctgctcgtttggcccgcgaaggcttca
aggtcactgtggtggagaaaaacgactttggtggcggccgctgctccttgatccatcaccagggccatcgctttgatcagggcccgtcgctctacct
gatgcccaagtactttgaggacgcctttgccgatctggacgagcgcattcaagaccacctggagctgctgcgatgcgacaacaactacaaggtgc
actttgacgacggtgagtcgatccagctgtcgtctgacttgacacgcatgaaggctgaattggaccgcgtggagggcccccttggttttggccgatt
cctggatttcatgaaagagacacacatccactacgaaagcggcaccctgattgcgctcaagaagaatttcgaatccatctgggacctgattcgcatc
aagtacgctccagagatctttcgcttgcacctgtttggcaagatctacgaccgcgcttccaagtacttcaagaccaagaagatgcgcatggcattcac
gtttcagaccatgtatatgggcatgtcgccctacgatgcgcctgctgtctacagcctgttgcagtacaccgagttcgctgaaggcatctggtatcccc
gtggcggcttcaacatggtggttcagaagctagaggcgattgcaaagcaaaagtacgatgccgagtttatctacaatgcgcctgttgccaagattaa
caccgatgatgccaccaaacaagtgacaggtgtaaccttggaaaatggccacatcatcgatgccgatgcggttgtgtgtaacgcagatctggtctat
gcttatcacaatctgttgcctccctgccgatggacgcaaaacacactggcttccaagaaattgacgtcttcttccatttccttctactggtccatgtccac
caaggtgcctcaattggacgtgcacaacattctttttggccgaggcttatcaggagagctttgacgaaatcttcaaggactttggcctgccttctgaagc
ctccttctacgtcaatgtgccctctcgcatcgatccttctgctgctcccgacggcaaggactctgtcattgtcttggtgcctattggtcatatgaagagca
agacgggcgatgcttccaccgagaactacccggccatggtggacaaggcacgcaagatggtgctggctgtgattgagcgtcgtctgggcatgtc
gaatttcgccgacttgattgagcatgagcaagtcaatgatcccgctgtatggcagagcaagttcaatctgtggagaggctcaattctgggtttgtctca
tgatgtgcttcaggtgctgtggttccgtcccagcacaaaggattctaccggtcgttatgataacctattctttgtgggtgcaagcacgcatcccggaact
ggtgttcccattgtccttgcaggaagcaagctcacctctgaccaagttgtcaagagctttggaaagacgcccaagccaagaaagatcgagatggag
aacacgcaagcacctttggaggagcctgatgctgaatcgacattccctgtgtggttctggttgcgcgctgccttttgggtcatgtttatgttcttttacttct
tccctcaatccaatggccaaacgcccgcatcttttatcaataatttgttacctgaagtattccgcgttcataactctaatgtcatttaa

(SEQ ID NO: 40)

mskkhiviigagvggtataarlaregfkvtvvekndfgggrcslihhqghrfdqgpslylmpkyfedafadlderiqdh
lellrcdnnykvhfddgesiqlssdltrmkaeldrvegplgfgrfldfmkethihyesgtlialkknfesiwdlirikyapeifrlhlfgkiydrask
yfktkkmrmaftfqtmymgmspydapavysllqytefaegiwyprggfnmvvqkleaiakqkydaefiynapvakintddatkqvtgvtl
enghiidadavvcnadlvyayhnllppcrwtqntlaskkltsssisfywsmstkvpqldvhniflaeayqesfdeifkdfglpseasfyvnvps
ridpsaapdgkdsvivlvpighmksktgdastenypamvdkarkmvlavierrlgmsnfadlieheqvndpavwqskfnlwrgsilglshd
vlqvlwfrpstkdstgrydnlffvgasthpgtgvpivlagskltsdqvvksfgktpkprkiementqapleepdaestfpvwfwlraafwvmf
mffyffpqsngqtpasfinnllpevfrvhnsnvi

### 1C. Production of pMB4660 (tef1p-carB URA3) Encoding Phytoene Dehydrogenase

The 4.3 kb XhoI-NotI fragment and the 1.8 kb NotI-SpeI fragment from pMB4638 were ligated to the 1.9 kb BsaI- and SpeI-cleaved URA3 gene generated by PCR amplification of Y. lipolytica genomic DNA using MO4684 and MO4685 to create pMB4660:

MO4684

(SEQ ID NO: 41)

5′-CATTCACTAGTGGTGTGTTCTGTGGAGCATTC
MO4685

(SEQ ID NO: 42)

5′-CACACGGTCTCATCGAGGTGTAGTGGTAGTGCAGTG

The resulting nucleic acid coding sequence and encoded CarB(i) protein of pMB4660 are as follows:

(SEQ ID NO: 43)

atgtccaagaaacacattgtcattatcggtgctggcgtgggtggcacggctacagctgctcgtttggcccgcgaaggcttca
aggtcactgtggtggagaaaaacgactttggtggcggccgctgctccttgatccatcaccagggccatcgctttgatcagggcccgtcgctctacct
gatgcccaagtactttgaggacgcctttgccgatctggacgagcgcattcaagaccacctggagctgctgcgatgcgacaacaactacaaggtgc
actttgacgacggtgagtcgatccagctgtcgtctgacttgacacgcatgaaggctgaattggaccgcgtggagggcccccttggttttggccgatt
cctggatttcatgaaagagacacacatccactacgaaagcggcaccctgattgcgctcaagaagaatttcgaatccatctgggacctgattcgcatc
aagtacgctccagagatctttcgcttgcacctgtttggcaagatctacgaccgcgcttccaagtacttcaagaccaagaagatgcgcatggcattcac
gtttcagaccatgtatatgggcatgtcgccctacgatgcgcctgctgtctacagcctgttgcagtacaccgagttcgctgaaggcatctggtatcccc
gtggcggcttcaacatggtggttcagaagctagaggcgattgcaaagcaaaagtacgatgccgagtttatctacaatgcgcctgttgccaagattaa
caccgatgatgccaccaaacaagtgacaggtgtaaccttggaaaatggccacatcatcgatgccgatgcggttgtgtgtaacgcagatctggtctat
gcttatcacaatctgttgcctccctgccgatggacgcaaaacacactggcttccaagaaattgacgtcttcttccatttccttctactggtccatgtccac
caaggtgcctcaattggacgtgcacaacatctttttggccgaggcttatcaggagagctttgacgaaatcttcaaggactttggcctgccttctgaagc
ctccttctacgtcaatgtgccctctcgcatcgatccttctgctgctcccgacggcaaggactctgtcattgtcttggtgcctattggtcatatgaagagca
agacgggcgatgcttccaccgagaactacccggccatggtggacaaggcacgcaagatggtgctggctgtgattgagcgtcgtctgggcatgtc
gaatttcgccgacttgattgagcatgagcaagtcaatgatcccgctgtatggcagagcaagttcaatctgtggagaggctcaattctgggtttgtctca
tgatgtgcttcaggtgctgtggttccgtcccagcacaaaggattctaccggtcgttatgataacctattctttgtgggtgcaagcacgcatcccggaact
ggtgttcccattgtccttgcaggaagcaagctcacctctgaccaagttgtcaagagctttggaaagacgcccaagccaagaaagatcgagatggag
aacacgcaagcacctttggaggagcctgatgctgaatcgacattccctgtgtggttctggttgcgcgctgccttttgggtcatgtttatgttcttttacttct
tccctcaatccaatggccaaacgcccgcatcttttatcaataatttgttacctgaagtattccgcgttcataactctaatgtcatttaa

(SEQ ID NO: 44)

mskkhiviigagvggtataarlaregfkvtvvekndfgggrcslihhqghrfdqgpslylmpkyfedafadlderiqdh
lellrcdnnykvhfddgesiqlssdltrmkaeldrvegplgfgrfldfmkethihyesgtlialkknfesiwdlirikyapeifrlhlfgkiydrask
yfktkkmrmaftfqtmymgmspydapavysllqytefaegiwyprggfnmvvqkleaiakqkydaefiynapvakintddatkqvtgvtl
enghiidadavvcnadlvyayhnllppcrwtqntlaskkltsssisfywsmstkvpqldvhniflaeayqesfdeifkdfglpseasfyvnvps
ridpsaapdgkdsvivlvpighmksktgdastenypamvdkarkmvlavierrlgmsnfadlieheqvndpavwqskfnlwrgsilglshd
vlqvlwfrpstkdstgrydnlffvgasthpgtgvpivlagskltsdqvvksfgktpkprkiementqapleepdaestfpvwfwlraafwvmf
mffyffpqsngqtpasfinnllpevfrvhnsnvi

### 1D. Production of pMB4637, pMB4714 and pTef-HMG encoding a truncated HMG1

For production of a truncated variant of the HMG-CoA reductase gene, which also encodes a 77 amino acid leader sequence derived from S. cerevisiae, the following oligonucleotides are synthesized:

PRIMER O

(SEQ ID NO: 45)

5′-TTCTAGACACAAAAATGGCTGCAGACCAATTGGTGA
PRIMER P

(SEQ ID NO: 46)

5′-CATTAATTCTTCTAAAGGACGTATTTTCTTATC
PRIMER Q

(SEQ ID NO: 47)

5′-GTTCTCTGGACGACCTAGAGG
MO4658

(SEQ ID NO: 48)

5′-CACACACGCGTACACCTATGACCGTATGCAAAT

Primers O and P are used to amplify a 0.23 kb fragment encoding Met-Ala followed by residues 530 to 604 of the Hmg1 protein of S. cerevisiae, using genomic DNA as template. Primers Q and MO4658 are used to amplify a 1.4 kb fragment encoding the C-terminal 448 residues of the Hmg1 protein of Y. lipolytica, using genomic DNA as template. These fragments are ligated to the appropriate cloning vector, and the resultant plasmids, designated pOP and pQMO4658, are verified by sequencing. The OP fragment is liberated with XbaI and AseI, and the QMO4658 fragment is liberated with MaeI and MluI. These fragments are then ligated to the ADE1 TEF1p expression vector pMB4629 cut with XbaI and MluI to produce pTefHMG.

Alternatively, the native HMG1 gene from Y. lipolytica was amplified without S. cerevisiae sequences using primers MO4658 (described above) and MO4657 (5′-CACACTCTAGACACAAAAATGACCCAGTCTGTGAAGGTGG) (SEQ ID NO:49). The 1.5 kb product was phosphorylated and ligated to pBluescriptSK⁻ that had been cleaved with EcoRV to create pMB4623. The XbaI-Mlul fragment containing hmg1^{trunc} was ligated both to NheI-MluI-cleaved MB4629 and to NheI-MluI-cleaved pMB4691 to create pMB4637 and pMB4714, respectively.

The resulting nucleic acid coding sequence and encoded Hmg1^{trunc} protein of pMB4637 and pMB4714 are as follows:

(SEQ ID NO: 50)

atgacccagtctgtgaaggtggttgagaagcacgttcctatcgtcattgagaagcccagcgagaaggaggaggacacctc
ttctgaagactccattgagctgactgtcggaaagcagcccaagcccgtgaccgagacccgttctctggacgacctagaggctatcatgaaggcag
gtaagaccaagcttctggaggaccacgaggttgtcaagctctctctcgagggcaagcttcctttgtatgctcttgagaagcagcttggtgacaacacc
cgagctgttggcatccgacgatctatcatctcccagcagtctaataccaagactttagagacctcaaagcttccttacctgcactacgactacgaccgt
gtttttggagcctgttgcgagaacgttattggttacatgcctctccccgttggtgttgctggccccatgaacattgatggcaagaactaccacattcctat
ggccaccactgagggttgtcttgttgcctcaaccatgcgaggttgcaaggccatcaacgccggtggcggtgttaccactgtgcttactcaggacggt
atgacacgaggtccttgtgtttccttcccctctctcaagcgggctggagccgctaagatctggcttgattccgaggagggtctcaagtccatgcgaaa
ggccttcaactccacctctcgatttgctcgtctccagtctcttcactctacccttgctggtaacctgctgtttattcgattccgaaccaccactggtgatgc
catgggcatgaacatgatctccaagggcgtcgaacactctctggccgtcatggtcaaggagtacggcttccctgatatggacattgtgtctgtctcgg
gtaactactgcactgacaagaagcccgcagcgatcaactggatcgaaggccgaggcaagagtgttgttgccgaagccaccatccctgctcacatt
gtcaagtctgttctcaaaagtgaggttgacgctcttgttgagctcaacatcagcaagaatctgatcggtagtgccatggctggctctgtgggaggtttc
aatgcacacgccgcaaacctggtgaccgccatctaccttgccactggccaggatcctgctcagaatgtcgagtcttccaactgcatcacgctgatga
gcaacgtcgacggtaacctgctcatctccgtttccatgccttctatcgaggtcggtaccattggtggaggtactattttggagccccagggggctatgc
tggagatgcttggcgtgcgaggtcctcacatcgagacccccggtgccaacgcccaacagcttgctcgcatcattgcttctggagttcttgcagcgga
gctttcgctgtgttctgctcttgctgccggccatcttgtgcaaagtcatatgacccacaaccggtcccaggctcctactccggccaagcagtctcagg
ccgatctgcagcgtctacaaaacggttcgaatatttgcatacggtcatag

(SEQ ID NO: 51)

mtqsvkvvekhvpiviekpsekeedtssedsieltvgkqpkpvtetrslddleaimkagktklledhevvklslegklp
lyalekqlgdntravgirrsiisqqsntktletsklpylhydydrvfgaccenvigymplpvgvagpmnidgknyhipmattegclvastmrg
ckainagggvttvltqdgmtrgpcvsfpslkragaakiwldseeglksmrkafnstsrfarlqslhstlagnllfirfrtttgdamgmnmiskgve
hslavmvkeygfpdmdivsvsgnyctdkkpaainwiegrgksvvaeatipahivksvlksevdalvelnisknligsamagsvggfnahaa
nlvtaiylatgqdpaqnvessncitlmsnvdgnllisvsmpsievgtigggtilepqgamlemlgvrgphietpganaqqlariiasgvlaaelsl
csalaaghlvqshmthnrsqaptpakqsqadlqrlqngsnicirs

### 1E. Production of pMB4692 (URA3 tef1p-crtZ) encoding N. aromaticovans Carotene Hydroxylase

The following carotene hydroxylase (CrtZ) ORF sequence was synthesized de novo based on protein sequence of Novosphingobium aromaticivorans, using Y. lipolytica codon bias:

(SEQ ID NO: 52)
5′-
ttctagacacaaaaatgggtggagccatgcagaccctcgctgctatcctgatcgtcctcggtacagtgctcgctatggagtttgtc
gcttggtcttctcataagtatatcatgcatggcttcggatggggatggcatagagaccatcacgagccccatgagggatttcttg
agaagaatgacttatacgccatcgttggcgctgccctctcgatactcatgtttgccctcggctctcccatgatcatgggcgctga
cgcctggtggcccggaacctggatcggactcggtgtcctcttctatggtgtcatctataccctcgtgcacgacggtctggtgcac
caacgatggtttagatgggtgcctaaacgaggttacgccaaacgactcgtgcaggcccataagctgcaccacgccaccattg
gcaaggaaggaggcgtctcattcggtttcgtgttcgcccgagatcccgccgttctgaagcaggagcttcgagctcaacgagaa
gcaggtatcgccgtgctgcgagaggctgtggacggctagacgcgt

This sequence was cleaved using XbaI and MluI and ligated, along with an Acc65I-NheI TEF1 promoter fragment from pMB4629, to pMB4662 cut with Acc65I and MluI to produce pMB4692. The nucleic acid coding sequence is depicted in bold underline above. The resulting encoded CrtZ protein of pMB4692 is as follows:

(SEQ ID NO: 53)

mggamqtlaailivlgtvlamefvawsshkyimhgfgwgwhrdhhepheg
flekndlyaivgaalsilmfalgspmimgadawwpgtwiglgvlfygviy
tlvhdglvhqrwfrwvpkrgyakrlvqahklhhatigkeggvsfgfvfar
dpavlkqelraqreagiavlreavdg

### 1F. Production of pMB4698 (ADEJ tef1p-crtW), Encoding Carotene Ketolase Derived from an Environmental Sample

The following carotene ketolase (CrtW) ORF sequence was synthesized de novo, based on protein sequence of an environmental sequence isolated from the Sargasso Sea (Genbank accession AACY01034193.1):

(SEQ ID NO: 54)
5′-
ttctagacacaaaaatgactcgatctatttcctggccttccacctactggcacctccagccctcctgttcttcttgggtcgcaaacga
attctctcctcaagcccgaaaaggtctcgtcctcgctggtctcattggttccgcttggctgcttactctcggacttggcttttccctt
cccctccatcaaacgagctggcttctcatcggttgtctcgttctccttagatctttcctgcacaccggactttttatcgttgcccatg
acgctatgcacgcttctcttgttcctgaccaccctggccttaaccgttggattggacgtgtctgtcttctcatgtatgctggactctc
ctacaaaagatgctgccgaaatcaccgtcgacaccaccaagcccctgaaacagttgaagaccctgactaccaacgatgcact
aacaacaatatcctcgactggtacgttcactttatgggaaattacctcggatggcaacaattgcttaatctctcttgcgtttggct
cgctctcaccttccgtgtttctgactactctgctcaattcttccacctgctccttttctctgtccttcctctcatcgtctcctcctgtcaa
ctcttcctcgtgggaacctggctgccacaccgacgaggcgctactactcgacccggcgttaccactcgatccctgaacttccac
cctgctctttccttcgctgcttgctaccacttcggttaccaccgtgaacaccatgaatctccctctactccttggttccaacttccta
aactccgagaaggttctctcatctaaacgcgt

This sequence was cleaved using XbaI and MluI and ligated to pMB4629 cut with NheI and MluI to produce pMB4698. The nucleic acid coding sequence is depicted in bold underline above. The resulting encoded CrtW protein of pMB4698 is as follows:

(SEQ ID NO: 55)

mtrsiswpstywhlqpscsswvanefspqarkglvlagligsawlltlgl
gfslplhqtswlligclvllrsflhtglfivahdamhaslvpdhpglnrw
igrvcllmyaglsykrccrnhrrhhqapetvedpdyqrctnnnildwyvh
fmgnylgwqqllnlscvwlaltfrvsdysaqffhlllfsvlplivsscql
flvgtwlphrrgattrpgvttrslnfhpalsfaacyhfgyhrehhespst
pwfqlpklregsli

Mutant alleles of this protein (e.g., L200M, F238L/I/V, including combinations thereof) can also be constructed and tested.

### 1G. Production of pMB4741 (ADE1 tef-crtW), Encoding Aurantimonas carotene Ketolase

The following carotene ketolase (CrtW) ORF sequence was synthesized de novo based on protein sequence of Aurantimonas sp. SI85-9A1, using Y. lipolytica codon bias:

(SEQ ID NO: 56)

5′-
ctctagacacaaaaatgtcttcctttgcccctatgaatgatgttgctattcctgccggtcaagctcctttctctgcctgtactagaaaacctgtcct
gagaccttttcaagctgccatcggtcttacactcgccggatgtgttatctctgcttggattgcaatccacgttggagctgtctttttcctcgatgt
cggttggcgaacccttcctgttgttcctgtcctcattgccgttcagtgctggctcacggtcggtctttttattgtcgcacacgatgctatgcacg
gctccctcgctcctggttggccacgacttaacgctcgaattggtgccttcatcctcaccatctacgctggattcgcttggagacgtgtccgag
gagctcacatggcccatcacgacgcccctggtactgccgatgaccctgacttctttgttgatgaacctgaccgattttggccttggtttcgagc
tttcttccttagatattttggacgtcgatctattctctttgtttgcacagttgtcaccgtttacattctggtccttggagcccctgttcttaatgttgt
tctcttttacggtcttccttcccttctgtcttctcttcaactcttttactttggaacttttcgtcctcaccgtcatgaagaagatgatttcgttgacgc
ccataatgcccgatctaatgaatttggttacatcgcctccctcctttcttgctttcactttggataccatcacgaacatcatgccgagccgtggg
tcccttggtggggtcttccttctcaatggcgccagagacaagcctcttcttcccgacaggtcccgggcggccgagacgctgctgacgccgct
ggagcatctcgacaacctgccggacgataccgatctgtttcttctcgaggtcgaaatcaggcccgttctcccgcttctggtcgaaacgaacaaatgagataa
acgcgt

This sequence was cleaved using XbaI and MluI and ligated to pMB4629 cut with NheI and MluI to produce pMB4741. The nucleic acid coding sequence is depicted in bold underline above. The resulting encoded CrtW protein of pMB4741 is as follows:

(SEQ ID NO: 57)

mssfapmndvaipagqapfsactrkpvlrpfqaaigltlagcvisawiai
hvgavffldvgwrtlpvvpvliavqcwltvglfivahdamhgslapgwpr
lnarigafiltiyagfawrrvrgahmahhdapgtaddpdffvdepdrfwp
wfrafflryfgrrsilfvctvvtvyilvlgapvlnvvlfyglpsllsslq
lfyfgtfrphrheeddfvdahnarsnefgyiasllscfhfgyhhehhaep
wvpwwglpsqwrqrqasssrqvpggrdaadaagasrqpagryrsvssrgr
nqarspasgrneqmr

Mutant alleles of this protein (e.g., L201M, A232V/I/L, F240L/I/V, including combinations thereof) can also be constructed and tested.

### 1H. Production of pMB4735 (ADE1 tef-crtW), Encoding P. bermudensis Carotene Ketolase

The following carotene ketolase (CrtW) ORF sequence was synthesized de novo based on protein sequence of Parvularcula bermudensis, using Y. lipolytica codon bias:

(SEQ ID NO: 58)

5′-
ctctagacacaaaaatggaccctaccggagacgttactgctagccctcgacctcaaaccaccattcctgtccgacaagcactctggggactt
agccttgctggagccatcatcgccgcatgggtttttatgcacattggtttcgttttttttgccccccttgatcctatcgttctcgccctcgccccag
ttattattcttcttcaatcctggctttctgttggtctttttattatttctcacgacgcaattcacggttccctcgcccctggacgacccgcctttaat
agagccatgggacgactctgcatgacactttacgccggtttcgactttgaccgtatggccgctgcacatcaccgacatcacagatcccctgg
aaccgccgctgaccccgatttttctgttgactcccctgatcgacctctcccttggtttggagctttcttccgacgttactttggctggagaccttt
tcttaccgttaacgctgtcgtctttacctactggcttgttcttggagctaaccctgttaatattgttctcttttatggcgttcctgcactcctttccg
ccggacagctattttactttggtacatttctccctcaccgacacgaacgacaaggctttgctgatcaccaccgagcacgatccgtccgatccc
cttacatgctttctcttgttacttctaccactttggaggctatcatcacgaacatcatctctttccacacgaaccctggtggcgcctgcctcaacgaggagg
ttgggaacgtgacagacgaaagagaaccggcccttaacgcgt

This sequence was cleaved using XbaI and MluI and ligated to pMB4629 cut with NheI and MluI to produce pMB4735. The nucleic acid coding sequence is depicted in bold underline above. The resulting encoded CrtW protein of pMB4735 is as follows:

(SEQ ID NO: 59)

mdptgdvtasprpqttipvrqalwglslagaiiaawvfmhigfvffapld
pivlalapviillqswlsvglfiishdaihgslapgrpafnramgrlcmt
lyagfdfdrmaaahhrhhrspgtaadpdfsvdspdrplpwfgaffrryfg
wrpfltvnavvftywlvlganpvnivlfygvpallsagqlfyfgtflphr
herqgfadhhrarsvrspymlslvtcyhfggyhhehhlfphepwwrlpqr
ggwerdrrkrtgp

Mutant alleles of this protein (e.g., L190M, M110I/V/L, F229L/I/V, including combinations thereof) can also be constructed and tested.

### 1I. Production of pMB4778 (URA3 tef-crtZ), Encoding P. bermudensis Carotene Hydroxylase

The following carotene hydroxylase (CrtZ) ORF sequence was synthesized de novo based on protein sequence of Parvularcula bermudensis, using Y. lipolytica codon bias:

(SEQ ID NO: 60)

5′-
ctctagacacaaaaatgactctcgctctctggcaaagatcaccctcgtccttggttccgctgctctgatggaaggatttgcttggtgggccca
tagatatattatgcacggttggggatgggcttggcatagagatcatcatgaacctcacgacaaagtttttgaaaaaaatgacctgtttgctgt
ggtttttggctcgttcgcatttggtttgttcatcgtcggttacctttattggccacctgtttggtacgttgctgctggcatcactctttacggacttc
tttacgcatttgttcatgacggtttggttcatcaacgttggccctggcatttcatgcctaaacgaggatacctccgaagactggttcaagctca
caaacttcatcatgctgttacaacacaaggcggaaatgtttcgtttggattcgtccttgcccctgaccctagacatcttagagaaaaacttag
acaatttcgtgctgaaagacatcgtgcccttgccgccgaaggtgcttcctcctctgaccctcgtgttcccccttttcgaaaagttcaagacgtttaaacgcgt

This sequence was cleaved using XbaI and MluI and ligated to pMB4751 cut with NheI and MluI to produce pMB4778. The nucleic acid coding sequence is depicted in bold underline above. The resulting encoded CrtZ protein of pMB4778 is as follows:

(SEQ ID NO: 61)

mtlalwqkitlvlgsaalmegfawwahryimhgwgwawhrdhhephdkvf
ekndlfavvfgsfafglfivgylywppywyvaagitlygllyafvhdglv
hqrwpwhfmpkrgylrrlvqahklhhavttqggnvsfgfvlapdprhlre
klrqfraerhralaaegasssdprvppfrkvqdv

### 1J. Production of pMB4719 (URA3 tef-crtZ), Encoding E. litoralis Carotene Hydroxylase

The following carotene hydroxylase (CrtZ) ORF sequence was synthesized de novo based on protein sequence of Erythrobacter litoralis, using Y. lipolytica codon bias:

(SEQ ID NO: 62)

5′-
ctctagacacaaaaatgagctggtgggctatcgctcttattgtctttggtgctgtcgttggaatggaattttttgcttggttcgctcataagtacat
tatgcatggttggggatggagctggcaccgagatcatcacgaacctcacgataatactcttgaaaaaaacgaccttttcgccgttgtctttg
gctcggttgccgcacttctgtttgttattggagctctctggtctgatcctctctggtgggcagcagttggtattacattgtatggcgtcatttaca
ctctggttcacgacggacttgttcatcaacgttactggcgttggacccctaagcgaggttatgctaagagacttgtccaggcccatcgacttc
atcacgctactgttggaaaggaaggaggtgtttcttttggttttgtgttcgcccgagatcctgctaagttgaaagccgaattgaaacaacaaagagaacagg
gacttgccgtcgttcgagattctatgggagcataaacgcgt

This sequence was cleaved using XbaI and MluI and ligated to pMB4691 cut with NheI and MluI to produce pMB4719. The nucleic acid coding sequence is depicted in bold underline above. The resulting encoded CrtZ protein of pMB4719 is as follows:

(SEQ ID NO: 63)

mswwaialivfgavvgmeffawfahkyimhgwgwswhrdhhephdntlek
ndlfavvfgsvaallfvigalwsdplwwaavgitlygviytlvhdglvhq
rywrwtpkrgyakrlvqahrlhhatvgkeggvsfgfvfardpaklkaelk
qqreqglavvrdsmga

### 1K. Production of pMB4812, Encoding N. crassa Phytoene Synthase/Lycopene Cyclase, a1-2

Exon 1 of a1-2 was synthesized by annealing the following oligonucleotides:

(SEQ ID NO: 64)

MO5017: 5′-CTAGACACAAAAATGTACGACTACGCCTTCGT;

(SEQ ID NO: 65)

MO5018: 5′-GCACCTGAAGTTCACCGTGCCCGCGGTTCCAA;

(SEQ ID NO: 66)

MO5019: 5′-GTGCACGAAGGCGTAGTCGTACATTTTTGTGT;

(SEQ ID NO: 67)

MO5020: 5′-CGCGTTGGAACCGCGGGCACGGTGAACTTCAG,

and ligating them to pMB4603 that had been cleaved with NheI and MluI, to create pMB4811. Exon2 was amplified from N. crassa (Fungal Genetic Stock Center #3200) genomic DNA, using MO5016 (5′-CCCGCGGCGGTACTTCT) (SEQ ID NO:68) and MO5013 (5′-CCGTCTCTACAGCAGGATCAGGTCAATGC) (SEQ ID NO:69), and inserted into pCR-TOPO (Invitrogen), to create pMB4809. Exon 3 was similarly amplified with MO5014 (5′-CCGTCTCACTGTACTCCTTCTGTCGCCTG) (SEQ ID NO:70) and MO5015 (5′-CACGCGTCTACTGCTCATACAACGCCCT) (SEQ ID NO:71), and cloned into the same vector to create pMB4810. The 0.9 kb SacII-BsmBI fragment from pMB4809 was ligated together with the 0.9 kb BsmBI-MluI fragment from pMB4810 into SacII-MluI-cleaved pMB4811, to create pMB4812, which expresses a1-2 from the TEFL promoter. The resulting nucleic acid coding sequence and encoded a1-2 protein of pMB4812 are as follows:

(SEQ ID NO: 72)

atgtacgactacgccttcgtgcacctgaagttcaccgtgcccgcggcggtacttctcaccgctatcgcctaccccattctcaa
caggatacatctcatccaaacaggcttcctcgtcgtcgtcgcctttaccgccgctctgccatgggatgcctacttgattaagcacaaagtatggtcttac
ccaccagaagccattgttgggccgcgtttgcttggaattccctttgaagagctgttcttctttgtgatacagacttacatcacggcgctcgtatacatcct
cttcaacaagccggtgctgcacgcgttgcacctcaacaatcaacaaaacccgccagcatggatgagggttgtcaaggttaccggccaggtagtcct
cgtagccttgtcggtatggggatggaatgccgctcaggttcatcaggaaacaagctatctcggcttgatccttgtttgggcttgtccgttcttactggct
atctggaccctcgctgggcgcttcattctcagcctaccctggtacgcgacggtgctcccgatgttcctacccaccttctatctttgggcggtagacgag
tttgccttgcacaggggtacttggtccatcggatcggggacgaagctcgatttttgtctgtttggcaagttggacattgaagaagccacgttcttcctgg
tgaccaacatgctcatcgttggcggtatggccgcgttcgatcaatatctggccgtcatttacgctttcccaactctgttccccaaggtcaaccggtatcc
gacaactcatatgcttcttcaaagccgtcttatcaacacttccaggtacgatcttgagcgcattgagggcctgagagaagcggtcgagagactgcgc
ctgaagagcaggagtttttacctggccaattcgctcttttctggtcgactccgcattgacctgatcctgctgtactccttctgtcgcctggctgatgatcta
gtcgacgacgccaaatctcgccgtgaggtcttgtcctggaccgcgaagctgaaccacttccttgatctgcactacaaggacgcggacgccaccga
ggaccccaagaaaaaggcggagcgaatcgacgcctacatcaagacagcgttccctccctgtgcctaccaagccctccacctcctgcccactcaca
ttcttcctcccaagcctctttacgatctcatcaagggtttcgagatggactctcaattcaccttccacggtacttccgactctacggatctccaatacccc
atcgccgacgacaaggaccttgagaactacgctatctatgtcgccggtaccgtcggcgagctctgcatcgccctcatcatctaccactgcctgccag
acatgtcggacactcagaagcgcgagctcgagaccgccgcgtgccggatgggcatcgcgctgcagtacgtcaacatcgctcgtgacatcgtcgt
cgacgcacgtatcgggcgcgtttacttgcctaccacctggctcaagaaggaagggttgacgcacaagatggtcttggagaaccccgagggtcccg
aggtcattgagcggatgagaagacggcttttggaaaatgcgtttgagctgtatgggggcgcgaggcctgagatgcaacggataccgagcgaggc
taggggcccgatgattggtgccgttgaaaattacatggcgattggaagggtgttgagggagaggaaggaggggacggtgtttgtgaggatggag
gggagggctacggtcccgaagcgaaggaggttgagcacgctgttgagggcgttgtatgagcagtag;

(SEQ ID NO: 73)

mydyafvhlkftvpaavlltaiaypilnrihliqtgflvvvaftaalpwdaylikhkvwsyppeaivgprllgipfeelfff
viqtyitalvyilfnkpvlhalhlnnqqnppawmrvvkvtgqvvlvalsvwgwnaaqvhqetsylglilvwacpfllaiwtlagrfilslpwya
tvlpmflptfylwavdefalhrgtwsigsgtkldfclfgkldieeatfflvtnmlivggmaafdqylaviyafptlfpkvnryptthmllqsrlints
rydlerieglreaverlrlksrsfylanslfsgrlridlillysfcrladdlvddaksrrevlswtaklnhfldlhykdadatedpkkkaeridayiktaf
ppcayqalhllpthilppkplydlikgfemdsqftfhgtsdstdlqypiaddkdlenyaiyvagtvgelcialiiyhclpdmsdtqkreletaacr
mgialqyvniardivvdarigrvylpttwlkkeglthkmvlenpegpeviermrrrllenafelyggarpemqripseargpmigavenyma
igrvlrerkegtvfvrmegratvpkrrrlstllralyeq

### 1L. Production of pMB4846 (URA3 tef-crtZ), Encoding an Erythrobacter carotene Hydroxylase

The following carotene hydroxylase (CrtZ) ORF sequence was synthesized de novo based on protein sequence of Erythrobacter sp. NAP 1, using Y. lipolytica codon bias:

(SEQ ID NO: 74)

5′-
ctctagacacaaaaatgtcttggcctgccgctattgcagttacacttggtgcccttatttttatggaattctttgcttggtacgctcacaaatacat
tatgcatggatggggatggggttggcacagagaccatcacgaacctcacgacaacaaactggaaaaaaatgacctgttcgctgtggtttt
cggaacaattaacgctggtatgtatatttttggtgctctttattgggatgctttgtggtgggctgcacttggagttaatctttacggagtgattta
cgcccttgttcatgacggactggttcatcaaagatttggaagatacgtccctaaaaacgcatacgctaaacgacttgttcaagcacacagat
tgcatcacgctactatcggtaaagaaggaggagtgtccttcggattcgttcttgctcgagaccctgctaaacttaaagccgaacttaaacgacaatctcaat
ccggagaagctattgttcgagaatccgccggagcctaaacgcgt

This sequence was cleaved using XbaI and MluI and ligated to pMB4691 cut with NheI and MluI to produce pMB4846. The nucleic acid coding sequence is depicted in bold underline above. The resulting encoded CrtZ protein of pMB4846 is as follows:

(SEQ ID NO: 75)

mswpaaiavtlgalifmeffawyahkyimhgwgwgwhrdhhephdnklek
ndlfavvfgtinagmyifgalywdalwwaalgvnlygviyalvhdglvhq
rfgryvpknayakrlvqahrlhhatigkeggvsfgfvlardpaklkaelk
rqsqsgeaivresaga

### 1M. Production of pMB4835 (URA3 tef-crtZ), Encoding an S. alaskensis Carotene Hydroxylase

The following carotene hydroxylase (CrtZ) ORF sequence was synthesized de novo based on protein sequence of Sphingopyxis alaskensis, using Y. lipolytica codon bias:

(SEQ ID NO: 76)
5′-
ctctagacacaaaaatgagccaccgaagagatccaggacttagaagagacgacgcacgatctatggcctcctgtctcagacgagcttaca
acccccacatgtccctgcctgcaattttgtttttggttcttgctactgtcattgcaatggaaggagtcgcctgggcatcccacaaatacatcat
gcacggatttggatgggcctggcacagagaccaccatgaaccccacgacaatcgactcgagaaaaacgacctgtttgccctgttcggagc
cgctatgtctatttctgccttcgctattggttctcctatgattatgggtgcagctgcctggaagcctggaacttggattggacttggtattctt
ctttacggtattatctacacactcgttcacgacggccttgtgcaccaaagatactttcgatgggtcccacgacgaggttacgcaaaacgacttg
ttcaagcacacaaacttcatcacgctacaatcggaaaagagggaggagtttctttcggatttgtttttgctcgtgaccctgctaaacttaaagc
cgaactgaaagcacaacgagaagctggtattgcagtcgtcagagaagcccttgctgactaaacgcgt

This sequence was cleaved using XbaI and MluI and ligated to pMB4691 cut with NheI and MluI to produce pMB4835. The nucleic acid coding sequence is depicted in bold underline above. The resulting encoded CrtZ protein of pMB4835 is as follows:

(SEQ ID NO: 77)

mshrrdpglrrddarsmasclrraynphmslpailflvlatviamegvaw
ashkyimhgfgwawhrdhhephdnrlekndlfalfgaamsisafaigspm
imgaaawkpgtwiglgillygiiytlvhdglvhqryfrwvprrgyakrlv
qahklhhatigkeggvsfgfvfardpaklkaelkaqreagiavvrealad

### 1N. Production of pMB4845 (URA3 tef-crtZ), Encoding an R. biformata Carotene Hydroxylase

The following carotene hydroxylase (CrtZ) ORF sequence was amplified from genomic DNA extracted from Robiginitalea biformata:

(SEQ ID NO: 78)
5′-
cacaatctagacacaaaaatgacagtcttgatttggatcgcaattttcctggccaccttctgcttcatggaattcatggcctggtttacgcataa
atatatcatgcacggtttcctctggagccttcataaggaccaccataaaaaggaccacgacagttggtttgagcgaaacgacgccttctttc
tattttatgcgatagtctccatgtcctttatcggggccgccgtgaacacgggattctggcaggggtggcccatcggcctgggcatcctcgctt
acgggattgcctactttatcgtacacgatatctttatccatcagcggttcaagctctttcgcaatgcgaataactggtacgcgcggggtatcc
gcagggcccataaaatccaccacaagcacctgggaaaagaggaaggggaatgcttcgggatgctgtttgtcccatttaagtacttccgga
agacctgaacgcgtttgtg

This sequence was phosphorylated and ligated to pBluescriptSK⁻ that had been cleaved with EcoRV and dephosphorylated, to create pMB4824. The XbaI-Mlul fragment from pMB4824 that contains crtZ was ligated to pMB4691 cut with NheI and MluI to produce pMB4845. The nucleic acid coding sequence is depicted in bold underline above. The resulting encoded CrtZ protein of pMB4845 is as follows:

(SEQ ID NO: 79)

mtvliwiaiflatfcfmefmawfthkyimhgflwslhkdhhkkdhdswfe
rndafflfyaivsmsfigaavntgfwqgwpiglgilaygiayfivhdifi
hqrfklfrnannwyargirrahkihhkhlgkeegecfgmlfvpfkyfrkt

### 1O. Production of pMB4837 (URA3 tef-crtZ), Encoding an X. autotrophicus Carotene Hydroxylase

The following carotene hydroxylase (CrtZ) ORF sequence was amplified from genomic DNA extracted from Xanthobacter autotrophicus:

(SEQ ID NO: 80)
5′-
cacaatctagacacaaaaatgtccaccagcctcgccttcctcgtcaacgcgctcatcgtgatcgccacggtcgccgccatggaaggggtggc
ctgggccgcgcacaaatatgtcatgcacggcttcggctggggctggcacaagtcccaccacgagccgcgcgagggcgtgttcgagcgca
acgacctttatgcgctgctgttcgcaggcatcgccatcgccctcatctacgcgttccgcaatggcggcgcgctgctgtgggtgggcgtgggg
atgacggtctacggcttcctttatttcttcgtgcacgacggcatcacccaccagcgctggccgttccgctacgtgccgcgcaacggctatctc
aagcgcctggtgcaggcccaccggctgcaccatgcggtggatggcaaggagggctgcgtctccttcggcttcatctatgccccgccgcctg
ccgacctgaaggccaagctgaagaagctgcacggcggcagcctgaacagaacgaggcggcggaatagacgcgtttgtg

This sequence was phosphorylated and ligated to pBluescriptSK⁻ that had been cleaved with EcoRV and dephosphorylated, to create pMB4823. The XbaI-HindIII (filled in with Klenow) fragment from pMB4823 that contains crtZ was ligated to pMB4691 cut with NheI and MluI (filled in with Klenow) to produce pMB4837. The nucleic acid coding sequence is depicted in bold underline above. The resulting encoded CrtZ protein of pMB4837 is as follows:

(SEQ ID NO: 81)

mstslaflvnaliviatvaamegvawaahkyvmhgfgwgwhkshhepreg
vferndlyallfagiaialiyafrnggallwvgvgmtvygflyffvhdgi
thqrwpfryvprngylkrlvqahrlhhavdgkegcvsfgfiyapppadlk
aklkklhggslkqneaae

### 1P. Production of pMB4850 (URA3 tef-crtZ), Encoding a P. putida Carotene Hydroxylase

The following carotene hydroxylase (CrtZ) ORF sequence was amplified from genomic DNA extracted from Pseudomonas putida (this sequence encodes a valine rather than a leucine at the second position, due to N-end rule considerations):

(SEQ ID NO: 82)
tctctctagacacaaaaatggtgttcaatctcgccatattgttcggcaccctggtggccatggagggcgttggtacgct
ggctcacaagtacatcatgcatggctggggctggtggctgcaccgatcgcaccatgagccacacctgggcatgctcgaaaccaacgacct
gtacctggtggccctggggctgatcgccacggcgctggtggcgctgggcaaaagtggttatgcgcctttgcagtgggtgggcggtggtgtg
gcaggctatggagcactgtatgtactggcccacgacggtttctttcaccggcactggccgcgcaagccgcggccggtcaaccgctacctga
aacgcttgcaccgcgcgcaccgcttgcaccatgcggtgaaggggcgcacggggagcgtgtcgttcgggttcttctatgcgccgccgctgaa
ggtgttgaagcagcaattgcgcagcaggcgcagccaatcgtgaacgcgtgagacgttgtg

This sequence was phosphorylated and ligated to pBluescriptSK⁻ that had been cleaved with EcoRV and dephosphorylated, to create pMB4847. The XbaI-MluI fragment from pMB4847 that contains crtZ was ligated to pMB4691 cut with NheI and MluI to produce pMB4850. The nucleic acid coding sequence is depicted in bold underline above. The resulting encoded CrtZ protein of pMB4850 is as follows:

(SEQ ID NO: 83)

mvfnlailfgtlvamegvgtlahkyimhgwgwwlhrshhephlgmletnd
lylvalgliatalvalgksgyaplqwvgggvagygalyvlahdgffhrhw
prkprpvnrylkrlhrahrlhhavkgrtgsvsfgffyapplkvlkqqlrs
rrsqs

### Example 2

### Engineering Yarrowia lipolytica for Increased Carotenoid Production

### 2A. Production of Y. lipolytica Expressing Geranylgeranylpyrophosphate Synthase and Phytoene Dehydrogenase

MF350 (MATB ura2-21 leu2-35 ade1) was transformed with pMB4591 (tef1p-GGS1) that had been cleaved upstream of URA5 with Sspl; a Ura⁺ transformant carrying the plasmid at the ura2 locus was identified and named MF364. It was subsequently transformed with pMB4638 (tef1p-carB) that had been cleaved at ADE1 with Sspl and a prototrophic transformant was chosen that harbored the plasmid at the ade1 locus. This strain was named MF502.

### 2B. Production of Y. lipolytica Expressing Geranylgeranylpyrophosphate Synthase, Phytoene Dehydrogenase and Phytoene Synthase/Lycopene Cyclase

MF502 was transformed with pMB4705 (tef1p-carRP[i⁻]) that had been treated with SspI. Ninety percent of the prototrophic transformants were very orange on YPD agar plates, and one, MF719, produced greater than 10 mg carotene per g dry cell weight (DCW) after four days of growth in YPD at 30° C.

### 2C. Production of Y. lipolytica Expressing Phytoene Synthase/Lycopene Cyclase and Phytoene Dehydrogenase

ATCC 201249 (MATA ura3-302 leu2-270 lys8-11) was transformed with SspI-cleaved pMB4628. Hundreds of Leu⁺ colonies were pooled, re-grown, and transformed with pMB4660 (tef1p-carB) that had been cleaved upstream of URA3 with SalI. One colony that was noticeably yellow after 5 days at 30° C. on YNBglut media (per liter: 1.7 g yeast nitrogen base, 1 g monosodium glutamate, 1% glucose) plus 0.6 mM lysine was selected, named MF447, and found to produce 0.2 mg carotene per gram dry cell weight after 4 days of growth in YPD.

MF447 was challenged with 1 g/L 5-fluoroorotic acid and Ura⁻ segregants selected. Surprisingly, they were all found to retain the identical yellow appearance of their parent, implying that the loss of a functional URA3 gene did not coincide with the loss of a functional CarB enzyme. Southern analysis demonstrates that two fragments from a KpnI-HindIII digest of MF447 DNA contain URA3p-hybridizing sequences, only one of which also hybridizes to carB. The other is absent in MF578, the Ura3⁻ segregant chosen for further manipulation. Plasmid rescue and analysis of the DNA sequence surrounding tef-carB in MF578 confirmed the absence of nearby URA3 sequences. Plasmid rescue and analysis of the DNA sequence encompassing the carRP intron in MF447 revealed that exons 1 and 2 were contiguous and were each separated by an intron sequence that lacked the original internal SspI site (present in pMB4628). The sequence of this region shows a seven-base pair deletion (AATATTA) that would restore the proper frame to an unspliced message. Partial intron sequence comprising the sequence where the deletion occurred is shown as follows:

(SEQ ID NO: 84)

ACAAACAAATGATGTGCCGCATCGCATTTTAATATTAACCATTGCATACA
CAG.

Predicted partial amino acid sequence comprising this intron, if unspliced, is as follows:

KAWVSKQTNDVPHRILIPLHTQHLA...(SEQ ID NO: 85).
(VSKQTNDVPHRILIPLHTQ (SEQ ID NO: 86) is intron
encoded)

### 2D. Production of Y. lipolytica Expressing Phytoene Synthase/Lycopene Cyclase, Phytoene Dehydrogenase and Geranylgeranylpyrophosphate Synthase

MF578 was transformed with pMB4683 (tef1p-GGS1) that had been cleaved with SalI (upstream of URA3) or with StuI (within the GGS1 ORF). Ura⁺ Leu⁺ colonies in both cases appeared bright orange on YNBglut+Lysine and on YPD, and several produced greater than 4 mg carotene per gram of dry cell weight when grown as above. One, MF633, contained a single copy of the plasmid at the GGS1 locus, as inferred from Southern analysis. The others arose by non-homologous or more complex integrations.

### 2E. Production of Y. lipolytica Expressing Phytoene Synthase/Lycopene Cyclase, Phytoene Dehydrogenase and Geranylgeranylpyrophosphate Synthase

MF364 was crossed with MF578, and spores from the resulting diploid were plated on YPD for two to three days at 30° C. White Leu⁻ Ade⁻ Ura⁻ colonies were screened for the presence of tefp-carB and tefp-GGS1 and for the absence of tefp-carRP by PCR. Thirteen colonies meeting these criteria, as well as displaying resistance to 5-fluorootic acid, an indication that they harbor the ura3-302 allele, were chosen as hosts for further modifications.

One such strain, MF731, was transformed with pMB4705 cut with BbvCI, and one Leu⁺ orange colony, MF740, produced 6 mg of β-carotene per g DCW after four days of growth in YPD at 30° C.

Another tefp-carB tefp-GGS1 strain from the same cross, MF739, was transformed with pMB4705 cut with BbvCI, and one Leu⁺ orange colony, MF746, produced 8 mg of β-carotene per g DCW after four days of growth in YPD at 30° C. When this strain was transformed with pMB4812 (expressing N. crassa a1-2 protein) treated with SspI, the Leu⁺ transformants were less orange than parallel pMB4705 Leu⁺ transformants, and after 4 days of growth in YPD, produced about half the amount of β-carotene as pMB4705 transformants. In addition, the pMB4812 transformants produced significant amounts of γ-carotene (˜40% of total carotene.).

### 2F. Expression of a Truncated Form of HMG-CoA Reductase Results in Increased Carotenoid Production in Y. lipolytica Expressing Phytoene Synthase/Lycopene Cyclase, Phytoene Dehydrogenase, and Geranylgeranylpyrophosphate Synthase

In order to increase carotenoid production, carbon flow through the isoprenoid pathway is enhanced by introducing a truncated variant of the HMG-CoA reductase gene.

MF740 was transformed with pMB4637 treated with SnaBI, and Ade⁺ colonies were selected. One such colony, MF760, was shown to produce about 20 mg β-carotene per g DCW after four days of growth in YPD at 30° C. This strain was also the subject of several fermentor studies outlined in Example 5. In addition, MF740 was also transformed with MB4714 treated with AflII and Ura⁺ colonies, were selected. One such colony was designated MF779 (see Example 2G). MF746 was also transformed with pMB4637 treated with SnaBI, and Ade⁺ colonies were selected. One such colony, MF946, was shown to produce greater than 35 mg β-carotene per g DCW after four days of growth in YPD at 30° C.

MF760 was also transformed with pMB4691 (empty vector) cut with SalI, creating the prototroph MF858.

### 2G. Production of Y. lipolytica Expressing Carotene Ketolase, a Truncated form of HMG-CoA Reductase, Phytoene Synthase/Lycopene Cyclase, Phytoene Dehydrogenase and Geranylgeranylpyrophosphate Synthase

MF779 was transformed with either pMB4735 or pMB4741 cleaved with SnaBI, and a red prototrophic colony was chosen from each transformation: MF838 (pMB4735) and MF840 (pMB4741). After 4 days of growth in YPD, MF838 produced 25 mg canthaxanthin per g DCW, and MF840 produced 14 mg canthaxanthin and 30 mg echinenone per g DCW. Only trace levels of β-carotene were produced. These strains are the subject of fermentor studies described in Example 5.

In addition, MF740 was transformed with pMB4735 cleaved with SnaBI, and a red Ade⁺ colony was chosen for further manipulation and designated MF889 (See Example 2J).

### 2H. Manipulation of the Y. lipolytica ERG9 Gene to Enhance Carotenoid Production

In order to decrease the expression of Erg9 (squalene synthase) in a carotenoid-producing yeast, pMB4789, containing the following cassette, was constructed using standard molecular techniques. The 4.8 kb fragment contains the Y. lipolytica URA3 gene flanked by the ERG9 ORF and the ERG9 terminator.

Thus this fragment comprises the sequence: GATCtcgttctgctcgggtagatc (SEQ ID NO:87)----ERG9 (promoter and ORF)----gtgctctgcggtaagatcgACTAGTggtgtgttctgtggagcattc (SEQ ID NO:88)------URA3 (promoter, ORF, and terminator)------------ccaccactgcactaccactacacCTCGAGCATGCATcaggaaggactctccctgtggt (SEQ ID NO:89)----ERG9terminator---gtgttatggctctacgtgaagGGGCCC (SEQ ID NO:90). (Capital letters: restriction sites [engineered for assembly]) In addition, it was found that a mutation was generated during cloning that changed the coding sequence of ERG9 as follows: (cccgacgttAtccagaagaac (SEQ ID NO:91); F317I in the encoded protein).

Two overlapping fragments from this cassette, a 2.4 kb AlwNI-SmaI fragment and a 1.9 kb AlwNI-AflII fragment, were cotransformed into MF760 and Ura⁺ colonies were selected. PCR analysis showed that one, designated MF921, contains the erg9::URA3 cassette replacing the wild type ERG9 gene. MF921 produced greater than 30 mg β-carotene per g DCW after 4 days of growth at 30° C. in YPD.

### 2I. Production of Y. lipolytica Expressing Carotene Hydroxylase, Phytoene Synthase/Lycopene Cyclase, Phytoene Dehydrogenase and Geranylgeranylpyrophosphate Synthase

MF740 was transformed with pMB4837 cleaved with SalI, and a Ura⁺ colony was selected and designated MF1011. MF1011 produced 6 mg of zeaxanthin and 1.5 mg of β-carotene per g DCW after 4 days of growth at 30° C. in YPD.

### 2J. Production of Y. lipolytica Expressing Carotene Hydroxylase, Carotene Ketolase, Phytoene Synthase/Lycopene Cyclase, Phytoene Dehydrogenase and Geranylgeranylpyrophosphate Synthase

MF889 was transformed with pMB4837 cleaved with SalI, and a prototrophic colony was selected and designated MF1016. MF1016 produced 1.5 mg of astaxanthin and 3 mg of canthaxanthin per g DCW after 4 days of growth at 30° C. in YPD.

### Example 3

### Extraction of Carotenoids

### 3A: Total Extraction of Carotenoids from Yarrowia lipolytica Cells

Yarrowia lipolytica cultures to be tested for carotenoid production were grown in 20 ml YPD medium (1% yeast extract, 2% peptone, 2% glucose) in 125 flasks at 30° C. Following incubation for 72-96 hr, the cultures were harvested by centrifugation and the solvent extractions were performed to determine carotenoid form and quantity. Dry cell weights were determined by transferring 1.8 ml of each culture to an Eppendorf tube, which was then centrifuged to pellet the cells, and then the pellet washed twice with 1 ml water. After the second wash, the cells were resuspended in water and transferred to a pre-weighed snap-cap tube with a hole poked in the top, frozen, and then lyophilized overnight. After drying to constant weight, the tube was weighed in order to calculate dry cell weight (mg dry cell weight/ml).

The carotenoid content of the culture was calculated by solvent extraction from 0.25 ml of culture from the same shake flask culture. This 0.25 ml culture sample was transferred to a 2 ml screw-cap tube, the cells pelleted, and the supernatant aspirated. Such pelleted cells may be extracted immediately or frozen at −80° C. and stored.

An equal volume of cubic zirconia beads was added to cell pellets, along with 1 ml ice-cold extraction solvent (a 50/50 v/v mix of hexane and ethyl acetate containing 0.01% butylhydroxytoluene (BHT)). The mixture was then agitated (Mini-BeadBeater-8, BioSpec Products, Inc.) at maximum speed for 5 minutes at 4° C. The mixture was then spun at maximum speed for 1 minute, and the supernatant was collected and deposited in a cold 16 ml glass vial.

The remaining cell debris was re-extracted at least three times, without the addition of zirconia beads; all supernatants were pooled in the 16 ml glass vial. Following extraction, the glass vial was spun for 5 minutes at 2000 rpm at 4° C. in a Sorvall tabletop centrifuge, and the supernatant was transferred to a new cold 16 ml glass vial. A Speed Vac was used to concentrate the supernatant (room temperature in dark), and the samples were stored at −20° C. or −80° C. until immediately before HPLC analysis. Prior to HPLC analysis, the samples were resuspended in 1 ml ice-cold solvent and then transferred to a cold amber vial. Throughout the protocol, care was taken to avoid contact with oxygen, light, heat, and acids.

The use of a hexane:ethyl acetate (50:50) mixture to extract carotenoids efficiently extracted all carotenoids analyzed from Yarrowia even though the carotenoids possessed different polarity levels. For instance, in a strain containing β-carotene, γ-carotene, echinenone, and canthaxanthin, a hexane:ethyl acetate (50:50) mixture efficiently extracted all carotenoids even though echinenone and canthaxanthin, respectively, are progressively more polar than either β-carotene or γ-carotene. Given the high efficiency of extraction observed for all carotenoids with 50:50 hexane:ethyl acetate, these conditions were chosen as a “100%” standard against which the extraction efficiency of other conditions could be compared.

### 3B. Extraction of β-Carotene from Y. lipolytica MF858

Y. lipolytica strain MF858 was grown as described in Example 3a and found to contain β-carotene as the dominant carotenoid. Extraction and breakage with hexane yielded an equal amount of β-carotene as was observed with a 50:50 hexane:ethyl acetate mixture.

### 3C. Extraction of Mixed Carotenoids from Y. lipolytica MF838

Y. lipolytica strain MF838 (Example 2g) had previously been found to contain the following types carotenoids when extracted as described in Example 3a: β-carotene, γ-carotene, echinenone, and canthaxanthin. Extraction with 750 μl of hexane resulted in the following extraction efficiencies for each of the carotenoids (extraction efficiency is reported independently for each of the carotenoid species based on the total amount found by hexane:ethyl acetate extraction): β-carotene (79.3%), γ-carotene (82.4%), echinenone (42.6%), and canthaxanthin (8.0%).

When an identical aliquot of MF838 was extracted with 1 ml of ethanol (95%), the extraction efficiency of the same four carotenoids was as follows: β-carotene (53.6%), γ-carotene (71.3%), echinenone (39.9%), and canthaxanthin (28.0%). Thus ethanol can be used to extract both polar and nonpolar carotenoids from fungi (e.g., Y. lipolytica).

### Example 4

### Quantification of Carotenoid Production by HPLC

For carotenoid analysis, samples were resuspended in ice-cold extraction solvent (a 50/50 v/v mix of hexane and ethyl acetate containing 0.01% butylhydroxytoluene (BHT)). An Alliance 2795 HPLC (Waters) equipped with a Waters XBridge C18 column (3.5 μm, 2.1×50 mm) and Thermo Basic 8 guard column (2.1×10 mm) was used to resolve carotenoid at 25° C.; authentic carotenoid samples were used as standards. The mobile phases and flow rates are shown below (Solvent A=Ethyl Acetate; Solvent B=Water; Solvent C=Methanol; Solvent D=Acetonitrile). The injection volume was 10 μL. The detector is a Waters 996 photodiode array detector. The retention times for lipophilic molecules include astaxanthin (1.159 min), zeaxanthin (1.335 min), β-apo-8′-carotenal (2.86 min), ergosterol (3.11 min), lycopene (3.69 min), β-carotene (4.02 min), canthaxanthin (2.50 min), echinenone (3.38 min), and phytoene (4.13 min). Astaxanthin, zeaxanthin, β-apo-8′-carotenal, lycopene, β-carotene, canthaxanthin, and echinenone are detected at 475 nm, whereas ergosterol and phytoene were detected at 286 nm.

TABLE 28
HPLC Solvent Gradient Table

| Time (min) | Flow (mL/min) | % A | % B | % C | % D | Curve |
|---|---|---|---|---|---|---|
| | 0.50 | 0.0 | 20.0 | 0.0 | 80.0 | |
| 3.00 | 1.00 | 20.0 | 0.0 | 0.0 | 80.0 | 6 |
| 4.50 | 1.00 | 80.0 | 0.0 | 20.0 | 0.0 | 6 |
| 5.00 | 1.00 | 0.0 | 0.0 | 100.0 | 0.0 | 6 |
| 6.00 | 1.00 | 0.0 | 0.0 | 100.0 | 0.0 | 6 |
| 6.50 | 1.00 | 0.0 | 20.0 | 0.0 | 80.0 | 6 |
| 7.00 | 0.50 | 0.0 | 20 | 0.0 | 80.0 | 6 |

### Example 5A

### 2 Liter Fed-Batch Fermentation of β-Carotene Producing Strain MF760

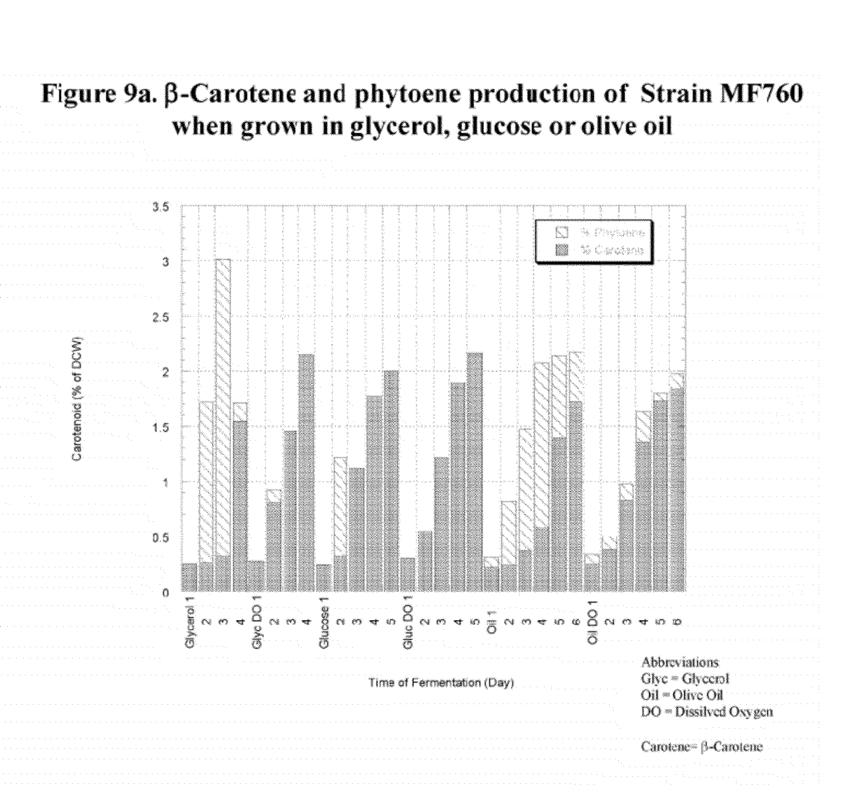

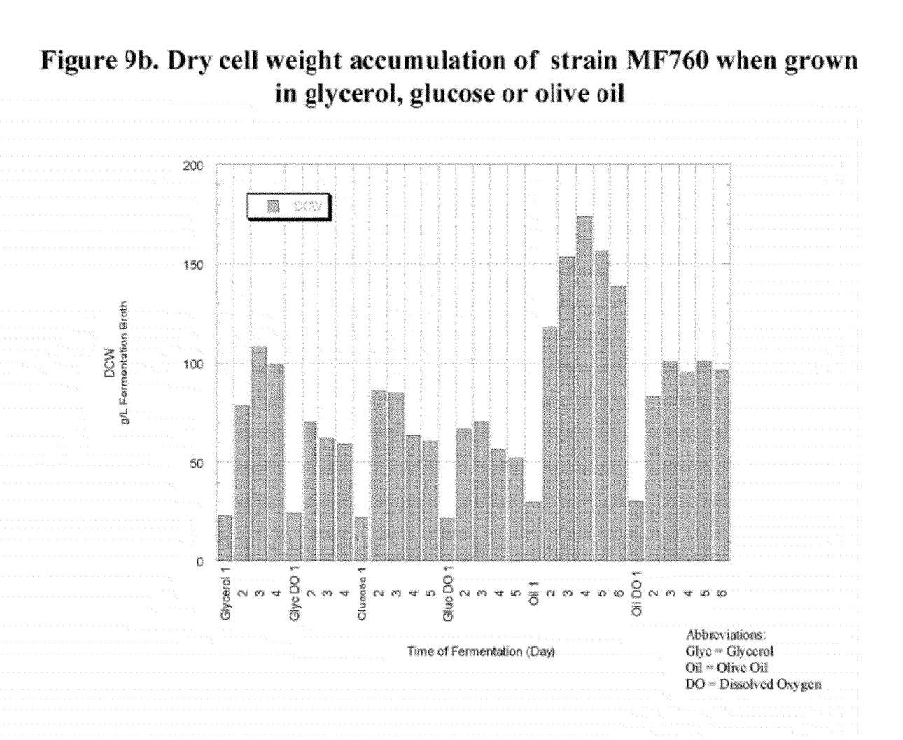

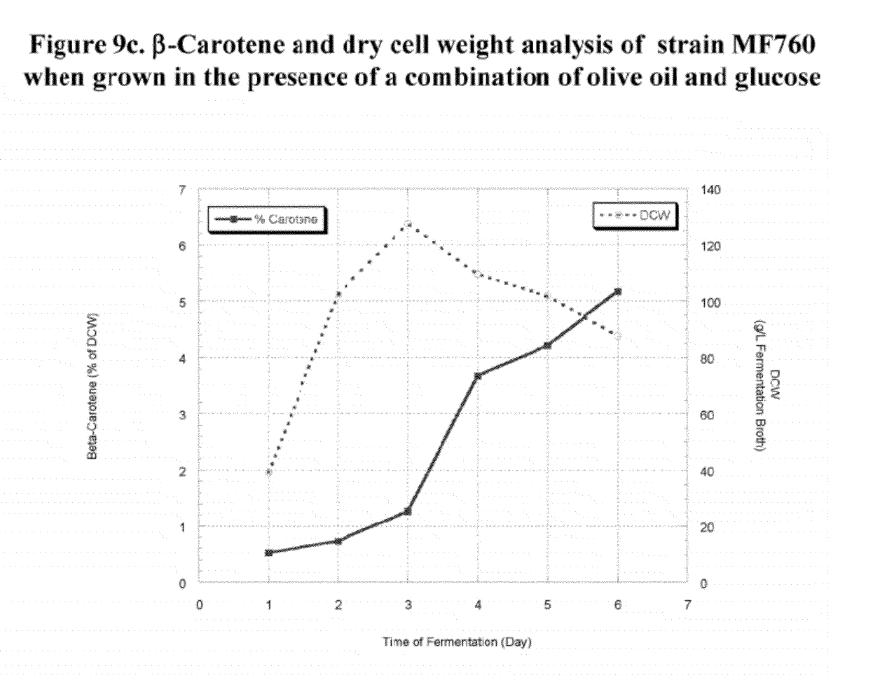

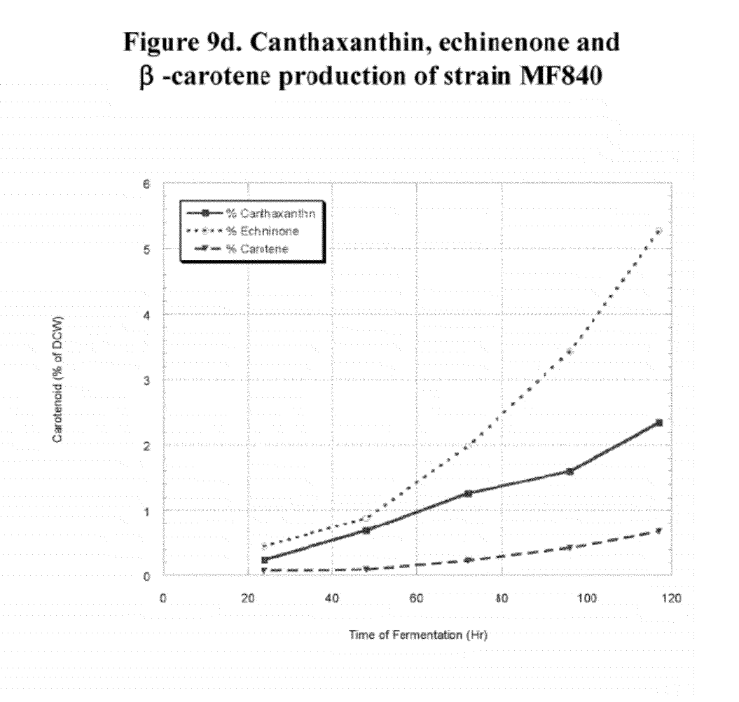

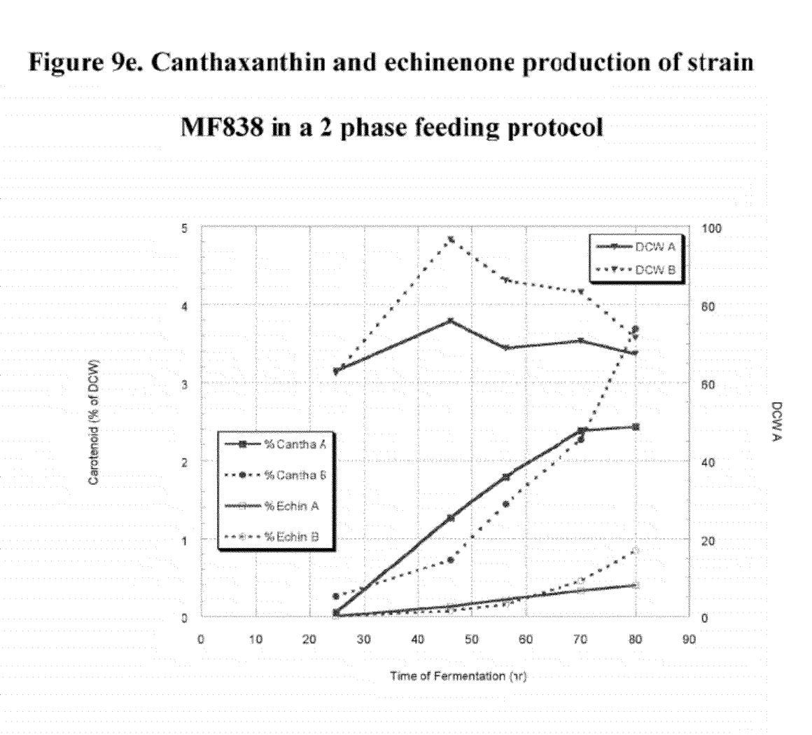

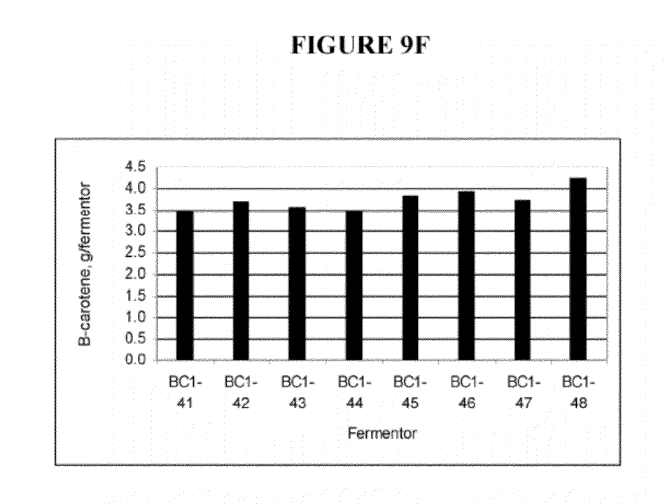

FIG. 9A depicts the production and intracellular accumulation of phytoene and β-carotene by strain MF760 (Example 2F) when grown in fed-batch fermentation on various carbon sources. Fermentation medium and process parameters are described below. Carbon sources used were glucose, glycerol, or olive oil. Feeding was initiated during the early exponential growth phase at a rate of 15.2 ml/hr. This feed rate either continued until feed exhaustion or, when the dissolved oxygen (dO2) level of the culture reached 20% saturation, feed was added to maintain the dO2 at 20% (DO controlled feed). As seen in FIG. 9a, β-carotene accumulates in all fermentations from 1.5 to 2.2% of DCW weight. Substantial phytoene accumulation was observed in the constant feed fermentations but not in the DO controlled feed fermentations.

FIG. 9B depicts dry cell weight accumulation during the course of the fermentations. For each carbon source examined, constant feeding resulted in greater biomass production relative to the DO controlled feed fermentation. This was especially true for the olive oil fed-batch fermentations where the constant feed fermentation reached greater than 150 g/L DCW. This was expected as Y. lipolytica has been reported to accumulate greater than 40% its biomass as lipid when grown on oils under conditions of excess carbon and oxygen limitation (Pananikolaou et al., Appl. Microbiol. Biotechnol. 58:308, 2002) and was independent of nitrogen concentration.

Batch Medium—1 L

| Carbon Source (one of the following): | Glucose | 60 | g |
|---|---|---|---|
| | Glycerol | 75 | g |
| | Olive Oil | 50 | ml |
| Yeast Nitrogen Base | | 4.1 | g |
| w/o Amino Acids and (NH₄)₂SO₄ | | | |
| (NH₄)₂SO₄ | | 6 | g |
| Uracil | | 72 | mg |
| Antifoam 204 (Sigma catalog A6426) | | 5 | ml |

Feed Medium—1 L

| | | | |
|---|---|---|---|
| Carbon Source (one of the following): | Glucose | 500 | g |
| | Glycerol | 500 | g |
| | Olive Oil | 500 | ml |
| (NH₄)₂SO₄ | | 72 | g |
| KH₂PO₄ | | 13.5 | g |
| MgSO₄ | | 5 | g |
| Inositol | | 70 | mg |
| Thiamine | | 10 | mg |
| Uracil | | 900 | mg |
| Trace Metal Solution | | 40 | ml |
| FeCl₃•6H₂O | | 2.7 | g/L |
| ZnCl₂•4H₂O | | 2.0 | g/L |
| CaCl₂•2H₂O | | 2.0 | g/L |
| Na₂MoO₄•2H₂O | | 2.0 | g/L |
| CuSO₄•5H₂O | | 1.9 | g/L |
| H₃BO₃ | | 0.5 | g/L |
| MnSO₄•H₂O | | 2.23 | g/L |
| Concentrated HCl | | 10 | ml/L |
| Vitamins Solution | | 40 | ml |
| Pantothenic acid | | 5.4 | g/L |
| Pyridoxine | | 1.4 | g/L |
| Niacin | | 6.1 | g/L |
| Folic acid | | 0.04 | g/L |
| Biotin | | 0.06 | g/L |

| | |
|---|---|
| Fermentation Parameters: | pH 5.5, controlled |
| | Temp—30° C. |
| | Air Flow—1.4 lpm |
| | Agitation—1200 rpm |

Inoculum—200 ml overnight culture grown in YEP + 5% glucose

### Example 5B

### 2 Liter Fed-Batch of β-Carotene Producing Strain MF760

FIG. 9c depicts the production and intracellular accumulation of β-carotene by strain MF760 when grown in fed-batch fermentation. In this fermentation, additions of olive oil were combined with a glucose feeding protocol. Medium and process parameters are described below. Both glucose and olive oil were present in the batch medium. Feeding of the glucose containing feed medium was initiated during the early exponential growth phase at a rate of 15.2 ml/hr. This feed rate continued until feed exhaustion. 25 ml of olive oil was added at 24, 48, and 72 hr.

As shown in FIG. 9c, this combined glucose and oil feeding protocol resulted in substantially higher DCW production when compared to glucose as the sole carbon source (Example 5a). In addition, β-carotene accumulated to over 5% of the DCW at the end of the fermentation, higher then either the glucose or oil fermentations of Example 5a.

Batch Medium - 1 L

| Glucose | 40 | g |
|---|---|---|
| Olive Oil | 50 | ml |
| Yeast Nitrogen Base | 4.1 | g |
| w/o Amino Acids and (NH₄)₂SO₄ | | |
| (NH₄)₂SO₄ | 6 | g |
| Uracil | 72 | mg |
| Antifoam 204 | 5 | ml |

Feed Medium - 1 L

| | | |
|---|---|---|
| Glucose | 500 | g |
| (NH₄)₂SO₄ | 72 | g |
| KH₂PO₄ | 13.5 | g |
| MgSO₄ | 5 | g |
| Inositol | 70 | mg |
| Thiamine | 10 | mg |
| Uracil | 900 | mg |
| Trace Metal Solution | 40 | ml |
| Vitamins Solution | 40 | ml |

Olive Oil Additions - 25 ml at 24, 48, and 72 hr

| | |
|---|---|
| Fermentation Parameters: | pH 5.5, controlled |
| | Temp - 30° C. |
| | Air Flow - 1.4 lpm |
| | Agitation - 1150 rpm |

Inoculum - 200 ml overnight culture grown in YEP + 5%
glucose 2.5% olive oil

### Example 5C

### 2 Liter Fed-Batch of Canthaxanthin Producing Strain MF840

FIG. 9d depicts the production and intracellular accumulation of canthaxanthin, echinenone and β-carotene by strain MF840 (Example 2g) when grown in fed-batch fermentation. Medium and process parameters are described below. Both glucose and olive oil were present in the batch medium. Feeding of the glucose containing feed medium was initiated during the early exponential growth phase at a rate of 15.2 ml/hr; this feed rate continued until the dissolved oxygen reached 20%, at which time feed was added to maintain the dO2 at 20% (DO controlled feed) for the remainder of the fermentation.

As seen in FIG. 9d, the combined total amount of canthaxanthin, echinenone and β-carotene represented over 8% of the DCW at the end of the fermentation and demonstrates the ability of genetically engineered Y. lipolytica to produce and accumulate significant amounts of carotenoids.

| Batch Medium - 1 L | | |
|---|---|---|
| Glucose | 40 | g |
| Olive Oil | 50 | ml |
| Yeast Nitrogen Base | 4.1 | g |
| w/o Amino Acids and (NH₄)₂SO₄ | | |
| (NH₄)₂SO₄ | 6 | g |
| Antifoam 204 | 5 | ml |
| Feed Medium - 1 L | | |
| Glucose | 500 | g |
| (NH₄)₂SO₄ | 72 | g |
| KH₂PO₄ | 13.5 | g |
| MgSO₄ | 5 | g |
| Inositol | 70 | mg |
| Thiamine | 10 | mg |
| Trace Metal Solution | 40 | ml |
| Vitamins Solution | 40 | ml |

| | |
|---|---|
| Fermentation Parameters: | pH 5.5, controlled |
| | Temp - 30° C. |
| | Air Flow - 1.4 lpm |
| | Agitation - 1150 rpm |

Inoculum - 200 ml overnight culture grown in YEP + 5%
glucose + 2.5% olive oil.

### Example 5D

### 2 Liter Fed-Batch of Canthaxanthin Producing Strain MF838

FIG. 9e depicts the production and intracellular accumulation of canthaxanthin and echinenone by strain MF838 (Example 2g) in fed-batch fermentation together with DCW levels. This example demonstrates the advantage of a two phase feeding protocol in which the first phase of feeding is designed to maintain excess carbon and oxygen limited conditions, while the second phase of feeding results in oxygen excess conditions via carbon limitation.

Fermentations A and B are depicted in FIG. 9e. Medium and process parameters are described below. In both fermentations, feeding of the glucose containing feed medium was initiated during the early exponential growth phase at a rate of 22.8 ml/hr. In fermentation A, this feed rate continued until the dissolved oxygen reached 20%, at which time feed was added to maintain the dO2 at 20% (DO controlled feed) for the remainder of the fermentation. In fermentation B, the constant feed rate was maintained such that glucose was in excess, and dO2 level was essentially zero, until approximately hour 40 of the fermentation. At that time, feed was added to maintain the dO2 at 20% (DO controlled feed) for the remainder of the fermentation. As seen in FIG. 9e, the extended period of carbon excess and oxygen limitation resulted in higher peak DCW, altered kinetics of canthaxanthin production, and produced a higher final level canthaxanthin—over 3.5% of DCW.

Batch Medium - 1 L

| | | |
|---|---|---|
| Glucose | 40 | g |
| Yeast Nitrogen Base | 8.2 | g |
| w/o Amino Acids and (NH₄)₂SO₄ | | |
| (NH₄)₂SO₄ | 6 | g |
| Antifoam 204 | 5 | ml |

Feed Medium - 1 L

| | | |
|---|---|---|
| Glucose | 500 | g |
| (NH₄)₂SO₄ | 72 | g |
| KH₂PO₄ | 13.5 | g |
| MgSO₄ | 5 | g |
| Inositol | 70 | mg |
| Thiamine | 10 | mg |
| Trace Metal Solution | 40 | ml |
| Vitamins Solution | 40 | ml |

| | |
|---|---|
| Fermentation Parameters: | pH 5.5, controlled |
| | Temp - 30 C. |
| | Air Flow - 1.4 lpm |
| | Agitation - 1150 rpm |

Inoculum - 200 ml overnight culture grown in YEP + 5% glucose

### Example 5E

### Reducing Levels of Certain Trace Metals Enhances Carotenoid Production in Fed-Batch Fermentation

The reduction of certain trace metals in the fermentation medium resulted in a significant increase in production and intracellular accumulation of β-carotene by a Y. lipolytica strain in fed-batch fermentation. The Yeast Nitrogen Base (YNB) in the batch medium contains low levels of a number of trace metals. The final concentration of trace elements in YNB batch medium lacking supplements, for YNB used at 4 g/L, is shown in Table 70 below.

TABLE 70
Concentration of Trace Element Compounds
in YNB Batch Medium

| Compound | Conc. (ug/L) |
|---|---|
| Boric Acid | 1176.47 |
| Copper Sulfate | 94.12 |
| Potassium Iodide | 235.29 |
| Ferric Chloride | 470.59 |
| Manganese Sulfate | 941.18 |
| Sodium Moybdate | 470.59 |
| Zinc Sulfate | 941.18 |

Additional amounts of trace metals are added as a trace metals solution. The table below outlines conditions for an experiment where each component of the trace metal solution was deleted and compared to the complete solution in 1 L fed-batch fermentations.

TABLE 71
Trace element deletion

| | Missing | Missing |
|---|---|---|
| Ferm. | Trace | Trace |
| No. | Compound | Element |
| BC1-41 | Complete | None |
| BC1-42 | H₃BO₃ | boron |
| BC1-43 | CaCl₂•2H₂O | calcium |
| BC1-44 | CuSO₄•5H₂O | copper |
| BC1-45 | FeCl₃•6H₂O | iron |
| BC1-46 | MnSO₄•H₂O | manganese |
| BC1-47 | Na₂MoO₄•2H₂O | molybdenum |
| BC1-48 | ZnCl₂ | zinc |

Medium and process parameters are described below. In all fermentations, feeding of the feed medium was initiated at 5 hr at a rate of 4.8 ml/hr and continued until hour 30, at which time the dissolved oxygen level was essentially zero and the feed was stopped. When the dissolved oxygen rose back to 20%, feeding was resumed and feed added to maintain the dO2 at 20% (DO controlled feed) for the remainder of the fermentation.

As seen in FIG. 9F, the reduction of zinc, and to a lesser extent, manganese or iron, resulted in a significant increase in β-carotene production.

Batch Medium - 500 ml

| Soybean Oil | 50 | ml |
|---|---|---|
| Yeast Nitrogen Base | 2 | g |
| w/o Amino Acids and (NH₄)₂SO₄ | | |
| Trace Elements Solution (Example 5A) | 20 | ml |
| Antifoam 204 | 0.25 | ml |

Feed Medium - 500 ml

| | | |
|---|---|---|
| Glucose | 325 | g |
| (NH₄)₂SO₄ | 36 | g |
| KH₂PO₄ | 2.5 | g |

| | |
|---|---|
| Fermentation Parameters: | pH 5.5, controlled |
| | Temp - 30 C. |
| | Air Flow - 0.7 lpm |
| | Agitation - 1000 rpm |

Inoculum - 100 ml overnight culture grown in YEP + 5% soybean oil

### Example 5F

### Production of Canthaxanthin by a Y. lipolytica Strain in a Two-Phase, Two Carbon Source Fed-Batch Fermentation

This example demonstrates the advantage of a two phase feeding protocol as described in Example 5D, further enhanced by utilizing two carbon sources in the fermentation. During the first phase of the fermentation, an oil is utilized as the primary carbon source under oxygen limited conditions. Under these conditions, a substantial amount of the cell dry cell weight accumulates as an intracellular lipid body. In the second phase of the fermentation, glucose is fed as the primary carbon source, with feeding controlled to maintain conditions of oxygen excess. Fermentation medium and operational parameters for 1 L fed-batch fermentations are described below.

Feeding of the feed medium was initiated at 5 hr at a rate of 4.8 ml/hr and continued until hour 30, at which time the dissolved oxygen level was essentially zero and the feed was stopped. When the dissolved oxygen rose back to 20%, feeding was resumed and feed added to maintain the dO2 at 20% (DO controlled feed) for the remainder of the fermentation.

Batch Medium - 500 ml

| Soybean Oil | 50 | ml |
|---|---|---|
| Yeast Nitrogen Base | 2 | g |
| w/o Amino Acids and (NH₄)₂SO₄ | | |
| (NH₄)₂SO₄ | 5 | g |
| Trace Elements Solution w/o Zinc (Example 5E) | 20 | ml |
| Antifoam 204 | 0.25 | ml |

Feed Medium - 500 ml

| | | |
|---|---|---|
| Glucose | 325 | g |
| KH₂PO₄ | 2.5 | g |

| | |
|---|---|
| Fermentation Parameters: | pH 5.5, controlled |
| | Temp - 30 C. |
| | Air Flow - 0.7 lpm |
| | Agitation - 1000 rpm |

Inoculum - 100 ml overnight culture grown in YEP + 5% soybean oil

In Example 5D, the use of a two phase fed-batch fermentation process resulted in increased carotenoid production by a canthaxanthin producing strain. However, a substantial amount of echinenone, an intermediate in the canthaxanthin biosynthetic pathway, also accumulated. The use of a two phase fed-batch fermentation process where an oil is the main carbon source in the first phase for the production of canthaxanthin resulted in essentially all of the carotenoid produced as only canthaxanthin. Table 72 shows results of HPLC analysis of the Y. lipolytica strain grown in a two phase, two carbon source fermentation process with soybean oil and glucose as carbon sources. Greater than 95% of the measured carotenoid HPLC peak was canthaxanthin, with other carotenoid intermediates less than 1.4%.

TABLE 72
HPLC analysis of cells grown in two phase,
two carbon source fermentation process

| Name | Retention Time | % Area |
|---|---|---|
| trans-Canthaxanthin | 2.271 | 95.51 |
| β-Cryptoxanthin | 3.325 | 1.39 |
| Echinenone | 3.377 | 0.33 |
| Lycopene | 3.594 | 0.75 |
| γ-Carotene | 3.767 | 0.70 |
| β-Carotene | 3.944 | 0.28 |

### Example 5G

### Production of Astaxanthin by a Y. lipolytica Strain in a Two-Phase Fed-Batch Fermentation

This example demonstrates the advantage of a two phase feeding protocol as described in Example 5D, but using an oil as the primary carbon source in both phases of the fermentation. During the first phase of the fermentation, an oil is utilized as the primary carbon source under oxygen limited conditions. Under these conditions, a substantial amount of the cell dry cell weight accumulates as an intracellular lipid body. In the second phase of the fermentation, an oil is fed as the primary carbon source, with feeding controlled to maintain conditions of oxygen excess. Fermentation medium and operational parameters for 1.8 L fed-batch fermentations are described below.

During the first phase of the fermentation, the dissolved oxygen level rapidly decreased to essentially zero as cell biomass accumulates during this growth phase. When the initial amount of oil in the batch medium was consumed, the dissolved level rises in the fermentor. When the dissolved oxygen rose back to 20%, feeding was initiated and feed added to maintain the dO2 at 20% (DO controlled feed) for the remainder of the fermentation.

| Batch Medium - 1 L | g/L |
|---|---|
| Soybean Oil | 138 |
| Yeast extract | 2.5 |
| Soy peptone | 5 |
| MgSO₄-anhydrous | 1.18 |
| (NH₄)₂SO₄ | 10 |
| KH₂PO₄ | 10.35 |
| Antifoam - Mazu DF204 | 5 |
| NaCl | 0.24 |
| Thiamine HCl | 0.001 |
| CuSO₄—5H₂O | 0.076 |
| FeCl₃—6H₂O | 0.108 |
| MnSO₄—H₂O | 0.089 |
| CaCl₂—2H₂O | 0.4 |
| Boric acid | 0.02 |
| Sodium molybdate | 0.08 |
| Feed Medium - 600 ml | g/L |
| Soybean Oil | 552 g |
| Fermentation Parameters: | pH 5.5, controlled |
| | Temp - 30 C. |
| | Air Flow - 1.4 lpm |
| | Agitation - 950 rpm |

Inoculum - 200 ml overnight shake flask culture grown in:

| Seed Culture Medium | g/L |
|---|---|
| Soy peptone | 20 |
| Yeast extract | 10 |
| Soybean Oil | 23 |

In this example, the use of a of a two phase fed-batch fermentation process where an oil was the main carbon source in both phases of the fermentation resulted in essentially all of the carotenoid produced being astaxanthin. Table 73 shows results of HPLC analysis of an astaxanthin-producing Y. lipolytica strain (constructed according to the methods described herein) grown in a two phase, soybean oil as carbon source, fermentation process. Greater than 90% of the measured carotenoid HPLC peak was astaxanthin, with other carotenoid intermediates less than 5%.

TABLE 73
HPLC analysis of cells grown in two phase,
oil fed-batch fermentation process

| Compound | Retention Time - min | % of Total Peak Area |
|---|---|---|
| Astaxanthin | 0.875 | 90.30 |
| Adonirubin | 1.171 | 4.77 |
| Trans-Canthaxanthin | 2.068 | 2.15 |
| Echinenone | 3.168 | 0.31 |
| Lycopene | 3.434 | 0.62 |
| γ-Carotene | 3.605 | 0.18 |
| β-Carotene | 3.769 | 0.19 |

### Example 5H

### Production of β-Carotene by a Y. lipolytica Strain in a Two-Phase Fed-Batch Fermentation where an Oil is the Primary Carbon Source in Both Phases

This example demonstrates the advantage of a two phase feeding protocol as described in Example 5D for β-carotene production by Y. lipolytica, but using an oil as the primary carbon source in both phases of the fermentation. During the first phase of the fermentation, an oil is utilized as the primary carbon source under oxygen limited conditions. Under these conditions, a substantial amount of the cell dry cell weight accumulates as an intracellular lipid body. In the second phase of the fermentation, an oil is fed as the primary carbon source, with feeding controlled to maintain conditions of oxygen excess. Fermentation medium and operational parameters for 1.8 L fed-batch fermentations are described below.

During the first phase of the fermentation, the dissolved oxygen level rapidly decreased to essentially zero as cell biomass accumulated during this growth phase. When the initial amount of oil in the batch medium was consumed, the dissolved level rose in the fermentor. When the dissolved oxygen rose back to 20%, feeding was initiated and feed added to maintain the dO2 at 20% (DO controlled feed) for the remainder of the fermentation.

| Batch Medium - 1 L | g/L |
|---|---|
| Soybean Oil | 138 |
| Yeast extract | 2.5 |
| Soy peptone | 5 |
| MgSO4-anhydrous | 1.18 |
| (NH₄)₂SO₄ | 10 |
| KH₂PO₄ | 10.35 |
| Antifoam - Mazu DF204 | 5 |
| NaCl | 0.24 |
| Thiamine HCl | 0.001 |
| CuSO₄—5H₂O | 0.076 |
| FeCl₃—6H₂O | 0.108 |
| MnSO₄—H₂O | 0.089 |
| CaCl₂—2H₂O | 0.4 |
| Boric acid | 0.02 |
| Sodium molybdate | 0.08 |
| Feed Medium - 600 ml | g/L |
| Soybean Oil | 552 g |
| Fermentation Parameters: | pH 5.5, controlled |
| | Temp - 30 C. |
| | Air Flow - 1.4 lpm |
| | Agitation - 950 rpm |

Inoculum - 200 ml overnight shake flask culture grown in:

| Seed Culture Medium | g/L |
|---|---|
| Soy peptone | 20 |
| Yeast extract | 10 |
| Soybean Oil | 23 |

In this example, the growth of Y. lipolytica β-carotene producing strain (constructed according to the methods described herein) in a two phase fed-batch fermentation process where an oil was the main carbon source in both phases of the fermentation resulted in essentially all (>97%) of the carotenoid produced being β-carotene as determined by HPLC analysis. When compared to the two phase, two carbon source, fermentation process of Example 5D, the use of an oil as the major carbon source in both phases of the fermentation described in this example resulted in an approximately 2-fold increase in β-carotene production on a grams of β-carotene per liter of fermentation broth basis. However, the fermentation process run time was increased by approx. 1.7 fold.

### Example 6

### Introduction of Heterologous Carotene Hydroxylase and Carotene Ketolase Genes into Y. lipolytica Strains Producing Carotenoid for Production of Astaxanthin

For introduction of carotene hydroxylase and carotene ketolase into carotenoid producing Y. lipolytica, pMB4692 and pMB4698 (described as in Example 1E and 1F above) can be sequentially introduced into MF740 or MF746 (described in Example 2E). For the introduction of pMB4692, the plasmid may be cleaved with SalI or BsrGI to direct integration at the ura3 locus, or with XbaI to promote random integration, selecting for uracil prototrophy. Ura⁺ transformants from MF740 or MF746 harboring pMB4692 are screened for zeaxanthin production in YPD. Zeaxanthin-producing cells are transformed with pMB4698 (which can be cleaved with PpuMI, SspI or BbvCI to direct integration at the ade1 locus, or with EcoRV to promote random integration) and prototrophic colonies are screened for astaxanthin production.

Alternatively, the order of plasmid transformation may be reversed wherein pMB4698 is transformed first and transformants are selected for adenine prototrophy. Ade⁺ transformants from MF740 or MF746 harboring pMB4698 are screened for canthaxanthin production. Canthaxanthin-producing MF740 [pMB4698] or MF746 [pMB4698] cells are transformed with pMB4692 and prototrophic colonies are screened for astaxanthin production.

In another approach, the carotenoid ketolase and carotenoid hydroxylase genes from P. marcusii can be introduced into a Leu2⁻ version of MF740 or MF746, in order to convert β-carotene into astaxanthin. P. marcusii genomic DNA is amplified with two primers.

CrtZfwd:

(SEQ ID NO: 92)

5′ CACACCGTCTCAAatgaccaatttcctgatcgtcgtc
CrtZrev:

(SEQ ID NO: 93)

5′ CACACAGATCtcacgtgcgctcctgcgcc,

and the resulting fragment is cleaved with BsmBI, modified with the Klenow fragment of DNA polymerase, and cleaved with BglII. This fragment is inserted into PmlI- and BamHI-cleaved pINA1269 (J. Mol. Microbiol. Biotechnol. 2 (2000): 207-216), containing the hp4d promoter, the XPR2 terminator, the selectable LEU2 gene, and sequences necessary for selection and propagation in E. coli. The resulting plasmid “pA” contains sequences encoding carotene hydroxylase from P. marcusii (crtZ gene)(Genbank accession: CAB56060.1) under the control of the hp4d promoter.

“pYEG1TEF” is modified by substituting the LIP2 terminator for the XPR2 terminator as follows. pINA1291 is digested with AvrII, modified with the Klenow fragment of DNA polymerase, and cleaved with EcoRI, and the small LIP2t containing fragment is ligated to “pYEG1TEF” that has been digested with SacII, modified with T4 DNA polymerase in the presence of dNTP, and cleaved with EcoRI. The resulting plasmid is named “pYEG1TEF-LIP2t”.

In order to amplify the carotenoid ketolase gene, P. marcusii genomic DNA is amplified with two primers.

CrtWfwd:

(SEQ ID NO: 94)

5′ CACACCCTAGGCCatgagcgcacatgccctgc
CrtWrev:

(SEQ ID NO: 95)

5′ CACACAAGCTTtcatgcggtgtcccccttg,

and the resulting fragment is cleaved with AvrII and HindIII, and inserted into AvrII- and HindIII-cleaved “pYEG1TEF-LIP2t”. The resulting plasmid, “pBt”, contains sequences encoding the carotene ketolase (crtW gene)(Genbank accession: CAB56059.1) under the control of the constitutive TEF1 promoter.

In order to combine the two expression cassettes into a single plasmid, “pBt” is cleaved with ClaI, modified with the Klenow fragment of DNA polymerase, and cleaved with EcoRI, and the crtW-containing fragment is isolated, mixed with the phosphorylated oligonucleotide adaptor pair:

| | |
|---|---|
| 5′ AATTCGCGGCCGCT | (SEQ ID NO: 96) |
| and | |
| 5′ AGCGGCCGCG, | (SEQ ID NO: 97) |

cleaved with NotI, and ligated to NotI-digested “pA”. The resulting plasmid, “pABt”, contains both the TEF1p/crtW/LIP2t cassette and the hp4d/crtZ/XPR2t cassette as well as the selectable LEU2 gene.

“pABt” can be introduced into MF740 or MF746 and transformants selected for leucine prototrophy.

### Example 7

### Partial Inactivation of Y. lipolytica ERG9 Gene Encoding Squalene Synthase Results in Increased Carotenoid Production

7A. In order to partially inactivate the ERG9 gene encoding squalene synthase, the neighboring FOL3 gene is disrupted, resulting in a folinic acid requirement. This strain is then transformed with a mutagenized fragment of DNA partially spanning the two genes, and For transformants are screened for decreased squalene synthase activity.

The following oligonucleotides are synthesized:

PRIMER K
(SEQ ID NO: 98)
5′-CCTTCTAGTCGTACGTAGTCAGC;
PRIMER L
(SEQ ID NO: 99)
5′-CCACTGATCTAGAATCTCTTTCTGG

and used to amplify a 2.3 kb fragment from Y. lipolytica genomic DNA spanning most of the FOL3 gene, using Pfu polymerase. The resulting fragment is cleaved with XbaI and phosphorylated, then ligated into pBluescriptSK⁻ that has been cleaved with KpnI, treated with T4 DNA polymerase (T4pol) in the presence of dNTPs, and subsequently cleaved with XbaI. The resultant plasmid, designated pBS-fol3, is then cleaved with Acc65I and EcoRI, treated with T4pol as above, and ligated to the 3.4 kb EcoRV-SpeI ADE1 fragment (treated with T4pol) from pMB4529.

The resulting plasmid, pBSfol3Δade, can be cleaved with BsiWI and XbaI to liberate a 5.5 kb fragment that is used to transform MF740 or MF746 to adenine prototrophy. Resulting Ade⁺ transformants are screened for a folinic acid requirement, and for homologous integration by PCR analysis.

Strains that harbor the resultant fol3 ΔADE1 allele can be transformed with a 3.5 kb DNA fragment generated by mutagenic PCR amplification using the primers:

PRIMER M
(SEQ ID NO: 100)
5′-GGCTCATTGCGCATGCTAACATCG;
PRIMER N
(SEQ ID NO: 101)
5′-CGACGATGCTATGAGCTTCTAGACG,

and Y. lipolytica genomic DNA as template. The resulting fragment containing the N-terminal three-quarters of the FOL3 ORF and the C-terminal nine-tenths of the ERG9 ORF is used to transform strains. The resulting Fol⁺ Ade⁻ transformants are screened for decreased squalene synthase activity by sensitivity to agents such as zaragozic acid, itraconazole, or fluconazole. Additionally, the resulting transformants are screened for increased carotenoid production.

7B. Alternatively, the PCR fragment produced in 7A could be cloned and altered in such a way as to remove the 3′-untranslated region of ERG9 gene. Replacement of the fol3ΔADE1 disruption by this fragment results in decreased expression of squalene synthase [Schuldiner et al. (2005), Cell 123:507-519][Muhlrad and Parker (1999), RNA 5:1299-1307], which can be confirmed as in 7A. This approach may also be used in a Fol⁺ Ade⁻ strain, using the ADE1 marker to disrupt the ERG9 3′-UTR.

7C. In still another approach, partially defective ERG9 alleles can be identified in S. cerevisiae using plasmid shuffling techniques [Boeke et al. (1987), Methods Enzymol. 154:164-175], and using drug sensitivities as a phenotype. Defective genes can be transferred to Y. lipolytica using standard molecular genetic techniques.

### Example 8

### Treatment of Y. lipolytica Strains Producing Carotenoid with Inhibitor of an Isoprenoid Biosynthesis Competitor Polypeptide Results in Increased Carotenoid Production

Cultures produced in Example 2 are treated with the squalene synthase inhibitor, zaragozic acid (zaragozic acid at 0.5 μM) and monitored for β-carotene production, as described above.

### Example 9

### Constructing an Oleaginous Strain of Saccharomyces cerevisiae

The genes encoding the two subunits of ATP-citrate lyase from N. crassa, the AMP deaminase from Saccharomyces cerevisiae, and the cytosolic malic enzyme from M. circinelloides are overexpressed in S. cerevisiae strains in order to increase the total lipid content. Similar approaches to enhance lipid production could be employed in other host organisms such as Xanthophyllomyces dendrorhous (Phaffia rhodozyma), using the same, homologous, or functionally similar oleaginic polypeptides.

Qiagen RNAEasy kits (Qiagen, Valencia, Calif.) are used to prepare messenger RNA from lyophilized biomass prepared from cultures of N. crassa. Subsequently, RT-PCR is performed in two reactions containing the mRNA template and either of the following primer pairs.

acl1:
(SEQ ID NO: 102)
1fwd: 5′ CACACGGATCCTATAatgccttccgcaacgaccg
(SEQ ID NO: 103)
1rev: 5′ CACACACTAGttaaatttggacctcaacacgaccc
acl2:
(SEQ ID NO: 104)
2fwd: 5′ CACACGGATCCAATATAAatgtctgcgaagagcatcctcg
(SEQ ID NO: 105)
2rev: 5′ CACACGCATGCttaagcttggaactccaccgcac

The resulting fragment from the acl1 reaction is cleaved with SpeI and BamHI, and that from the acl2 reaction is cleaved with BamHI and SphI, and both are ligated together into YEp24 that has been digested with NheI and SphI, creating the plasmid “p12”. The bi-directional GAL1-10 promoter is amplified from S. cerevisiae genomic DNA using the primers.

gal10:
(SEQ ID NO: 106)
5′ CACACGGATCCaattttcaaaaattcttactttttttttggatgga
c
gal1:
(SEQ ID NO: 107)
5′ CACACGGATCCttttttctccttgacgttaaagtatagagg,

and the resulting 0.67 kb fragment is cleaved with BamHI and ligated in either orientation to BamHI-digested “p12” to create “p1ga12” and “p2gal1”, containing GAL1-acl1/GAL10-acl2 and GAL10-acl1/GAL1-acl2, respectively (Genbank accession: acl1: CAB91740.2; acl2: CAB91741.2).

In order to amplify the S. cerevisiae gene encoding AMP deaminase and a promoter suitable for expressing this gene, S. cerevisiae genomic DNA is amplified using two primer pairs in separate reactions:

AMD1 ORF:
AMD1FWD:
(SEQ ID NO: 108)
5′ CACACGAGCTCAAAAatggacaatcaggctacacagag
AMD1rev:
(SEQ ID NO: 109)
5′ CACACCCTAGGtcacttttcttcaatggttctcttgaaattg
GAL7p:
gal7prox:
(SEQ ID NO: 110)
5′ CACACGAGCTCggaatattcaactgtttttttttatcatgttgatg
gal7dist:
(SEQ ID NO: 111)
5′ CACACGGAtccttcttgaaaatatgcactctatatcttttag,

and the resulting fragment from the AMD1 reaction (2.4 kb) is cleaved with SacI and AvrII, and that from the GAL7 reaction (0.7 kb) is cleaved with BamHI and SphI, and both are ligated together into YEp 13 that has been digested with NheI and BamHI, creating the plasmid “pAMPD”. This plasmid carries the S. cerevisiae gene, AMD1, encoding AMP deaminase, under the control of the galactose-inducible GAL7 promoter.

Messenger RNA is prepared from lyophilized biomass of M. circinelloides, as described above, and the mRNA template is used in a RT-PCR reaction with two primers:

MAEfwd:
(SEQ ID NO: 112)
5′ CACACGCTAGCTACAAAatgttgtcactcaaacgcatagcaac
MAErev:
(SEQ ID NO: 113)
5′ CACACGTCGACttaatgatctcggtatacgagaggaac,

and the resulting fragment is cleaved with NheI and SalI, and ligated to XhoI- and XhoI-digested pRS413TEF (Mumberg, D. et al. (1995) Gene, 156:119-122), creating the plasmid “pTEFMAE”, which contains sequences encoding the cytosolic NADP⁺-dependant malic enzyme from M. circinelloides (E.C. 1.1.1.40; mce gene; Genbank accession: AY209191) under the control of the constitutive TEF1 promoter.

The plasmids “p1gal2”, “pAMPD”, and “pTEFMAE” are sequentially transformed into a strain of S. cerevisiae to restore prototrophy for uracil (“p1gal2”), leucine (“pAMPD”), and histidine (“pTEFMAE”) (Guthrie and Fink Methods in Enzymology 194:1-933, 1991). The resulting transformants are tested for total lipid content following shake flask testing in either synthetic complete (SC) medium lacking uracil, leucine and histidine, as described in Example 3, or in a 2-step fermentation process. In the 2-step process, 1.5 ml of cells from an overnight 2 ml roll tube culture containing SC medium lacking uracil, leucine and histidine are centrifuged, washed in distilled water, and resuspended in 20 ml of a nitrogen-limiting medium suitable for lipid accumulation (30 g/L glucose, 1.5 g/L yeast extract, 0.5 g/L NH₄Cl, 7 g/L KH₂PO₄, 5 g/L Na₂HPO₄-12H₂O, 1.5 g/L MgSO₄-7H₂O, 0.08 g/L FeCl₃-6H₂O, 0.01 g/L ZnSO₄-7H₂O, 0.1 g/L CaCl₂-2H₂O, 0.1 mg/L MnSO₄-5H₂O, 0.1 mg/L CuSO₄-5H₂O, 0.1 mg/L Co(NO₃)₂-6H₂O; pH 5.5 (J Am Oil Chem Soc 70:891-894 (1993)).

Intracellular lipid content of the modified and control S. cerevisiae strains is analyzed using the fluorescent probe, Nile Red (J Microbiol Meth (2004) 56:331-338). In brief, cells diluted in buffer are stained with Nile Red, excited at 488 nm, and the fluorescent emission spectra in the wavelength region of 400-700 nm are acquired and compared to the corresponding spectra from cells not stained with Nile Red. To confirm results from the rapid estimation method, the total lipid content is determined by gas chromatographic analysis of the total fatty acids directly transmethylesterified from dried cells, as described (Appl Microbiol Biotechnol. (2002) 60:275-80). Non-transformed S. cerevisiae strains produce 6% and 10% total lipid (dry cell weight basis) after growth in YPD and lipid accumulation medium, respectively. Yeast strains expressing the multiple oleaginic polypeptides produce 17% and 25% total lipid following growth in YPD and lipid accumulation medium, respectively.

### Example 10

### Introduction of Heterologous Carotene Hydroxylase into Y. lipolytica Strains Producing Carotenoid for Production of Zeaxanthin

MF578 (tef-carRP tef-carB) was transformed with pMB4692 that had been cleaved with SalI. Several Ura⁺ colonies inferred to contain tef-crtZ by PCR analysis were able to produce zeaxanthin in YPD shake flasks, and in one case, all of the β-carotene was depleted.

### Example 11

### Regulatory Sequences

Sequences which consist of, consist essentially of, and comprise the following regulatory sequences (e.g., promoters and terminator sequences, including functional fragments thereof) may be useful to control expression of endogenous and heterologous genes in engineered host cells, and particularly in engineered fungal cells described herein.

Met2 promoter
(SEQ ID NO: 114)
5′cctctcactttgtgaatcgtgaaacatgaatcttcaagccaagaatgttaggcaggggaagctttctttcagactttttggaattggtcctcttttggac
attattgacgatattattattttttccccgtccaatgttgacccttgtaagccattccggttctggagcgcatctcgtctgaaggagtcttcgtgtggctata
actacaagcgttgtatggtggatcctatgaccgtctatatagggcaacttttgctcttgttcttccccctccttgagggacgtatggcaatggctatgaca
actatcgtagtgagcctctataacccattgaagtacaagtcctccaccttgctgccaaactcgcgagaaaaaaagtccaccaactccgccgggaaat
actggagaacacctctaagacgtgggcttctgcacctgtgtggcttgggtctgggttttgcgagctctgagccacaacctaaggacggtgtgattgg
gagataagtagtcgttggttttctaatcgcacgtgatatgcaagccacacttataacacaatgaagacaggccgatgaactgcatgtcattgtacaggt
gcggagagcaagaaactctggggcggaggtgaaagatgagacaaaaagcctcaggtgcaaggtagggagttgatcaacgtcaaacacaaataa
tctaggttgttaggcagctaaacatgtatataactgggctgccaccgagtgttacttgtcattaacgtcgcattttcgcctacacaaaatttgggttactcg
ccactacactgctcaaatctttcagctgtgcaacaagctttcaggtcacacatagactcgcataaggacccgggtcatctgttattctccactggtaaac
caatagtcctagctgatttgggtacagaagctcactttcacatcttttcatcttcttctacaaccatc
Met3 promoter
(SEQ ID NO: 115)
5′atctgtgaggagcccctggcgtcactgtcgactgtgccggcatttctgatggtatttccagccccgcagttctcgagacccccgaacaaatgtgcc
acacccttgccaaaatgacgaatacacggcgtcgcggccgggaatcgaactcttggcaccgccacaggagtgaaatttgaaatttgaaatttgaaa
aataattcacattttgagtttcaataatatatcgatgaccctcccaaaagacccaagtcgagacgcaaaaaaacacccagacgacatggatgcggtc
acgtgaccgcaaaaaccgccccggaaatccgtttgtgacgtgttcaattccatctctatgtttttctgcggtttctacgatgccgcaatggtggccaatg
tgcgtttcactgccgtagtggctggaacaagccacagggggtcgtcgggccaatcagacggtccctgacatggttctgcgccctaacccgggaac
tctaacccccgtggtggcgcaatcgctgtcttcatgtgctttatctcacgtgacggctggaatctggcagaagacggagtatgtacattttgtcgttggt
cacgttatccctaaaacgtggtgtttaaactggtcgaatgcttggcccagaacacaagaagaaaaaaacgagacaacttgatcagtttcaacgccac
agcaagcttgtcttcactgtggttggtcttctccacgccacaagcaacacgtacatgtcaattacgtcagggtcttttaagttctgtggcttttgaaccagt
tataaagaaccaaccacccttttttcaaagctaatcaagacggggaaattttttttttgatatttttcgaca
Met6 promoter
(SEQ ID NO: 116)
5′gatactgcagacggtgcattacttacccgtgtcgactgagagtctacttggtacttggccctgtggctaagcagtatttgagcaacaatgcaatgca
gttgctgactcggttccagatccccttgccccgatgtgtggaagcgttgtttttggggcaagggcatgtgggggctgcatcatactgtggctggggcc
gttggaagagccgtcggcagcgagcctgagtcgcttctcggggccttattccccccgcctctaggtcagcggcggccgaagtgtcgtactcagctc
gcctgtacagtatgacgtgaccgaatagcctctggaaggttggagaagtacagtgcaaaaaaaagttgcaaaatttcattttagcgttcgatccgacg
tggcagttggacaatgaatcgatggagacatgatcatgggcagaaatagaaggtctccatgttcaatggcagtaccaattgagcaacagacgggtc
gacaggcggcgggcacaccatccgccctccacatggcgcaatcgtcagtgcagcgattcgtactcggattgcatcatgttgcaccgaaagttggg
gcccgcacgttggagaggcgaggagccagggttagctttggtggggtcctttgttgtcacgtggcatcagcgaatggcgtcctccaatcagggcc
gtcagcgaagtcggcgtgtgatagtgcgtggggagcgaatagagtttctgggggggggcggcccaaaacgtgaaatccgagtacgcatgtaga
gtgtaaattgggtgtatagtgacattgtttgactctgaccctgagagtaatatataatgtgtacgtgtccccctccgttggtcttctttttttctcctttct
cctaaccaacacccaaactaatcaatc
Met25 promoter
(SEQ ID NO: 117)
5′aagtcgtattaacataactttccttacatttttttaaagcacgtcactatccacgtgacctagccacgcgataccaagtattcatccataatgacacact
catgacgtccggaggacgtcatcatcgtccagtcacgtgccaaggcacatgactaatcataacaccttatgactagcttctgaatcgctacacagttc
caattcgcaaataaactcgaaatgacgaaatgccataataaaaatgacgaaactcgagattgagagcagcacatgcactgaagtggtggacaacc
agcgtatccggagacacgacggatccagcaccatggaagctggccgaaaaagagatccccagcacattgagcaaccaagtcagctcaattgagt
aacatcacacactcagatcgagtctgatggtggtccccttttgttccttcacttgaaaaataattgaaaataacaataacaataaaaataaaaacaaaat
aaaaataaaaataaaaataaaaataaaaaaataaaaaaaccttgccgcatttagcgtcagccaccccccgcattgacctgagtacgttggattgacc
ccgatcctgcacgtcgagcgtggtcggccaaaaagcgcccgtggctggtgagtcagaaatagcagggttgcaagagagagctgcgcaacgagc
aataaacggtgtttttttcgcttctgtgctgcttagagtggagagccgaccctcgccatgctcacgtgaccattcacgtggttgcaaactccaccttagt
atagccgtgtccctctcgctacccattatcgcatcgtactccagccacatttttttgttccccgctaaatccggaaccttatctgggtcacgtgaaattgc
aatctcgacaggaggttatacttatagagtgagacactccacgcaaggtgttgcaagtcaattgacaccacctcacctcagactaacatccaca
Pox2 promoter
(SEQ ID NO: 118)
5′gaatctgcccccacattttatctccgcttttgactgtttttctcccccctttcacactctgcttttggctacataaaccccgcaccgtttggaactctgtt
ggtccggggaagccgccgttaggtgtgtcagatggagagcgccagacgagcagaaccgagggacagcggatcgggggagggctgtcacgtga
cgaagggcactgttgacgtggtgaatgtcgcccgttctcacgtgacccgtctcctctatatgtgtatccgcctctttgtttggttttttttctgcttcccccc
ccccccccccaccccaatcacatgctcagaaagtagacatctgcatcgtcctgcatgccatcccacaagacgaacaagtgataggccgagagccg
aggacgaggtggagtgcacaaggggtaggcgaatggtacgattccgccaagtgagactggcgatcgggagaagggttggtggtcatgggggat
agaatttgtacaagtggaaaaaccactacgagtagcggatttgataccacaagtagcagagatatacagcaatggtgggagtgcaagtatcggaat
gtactgtacctcctgtactcgtactcgtacggcactcgtagaaacggggcaatacgggggagaagcgatcgcccgtctgttcaatcgccacaagtc
cgagtaatgctcgagtatcgaagtcttgtacctccctgtcaatcatggcaccactggtcttgacttgtctattcatactggacaagcgccagagttaagc
ttgtagcgaatttcgccctcggacatcaccccatacgacggacacacatgcccgacaaacagcctctcttattgtagctgaaagtatattgaatgtgaa
cgtgtacaatatcaggtaccagcgggaggttacggccaaggtgataccggaataaccctggcttggagatggtcggtccattgtactgaagtgtcc
gtgtcgtttccgtcactgccccaattggacatgtttgtttttccgatctttcgggcgccctctccttgtctccttgtctgtctcctggactgttgctacccc
atttctttggcctccattggttcctccccgtctttcacgtcgtctatggttgcatggtttcccttatacttttccccacagtcacatgttatggaggggtct
agatggaggcctaattttgacgtgcaaggggcgaattggggcgagaaacacgtcgtggacatggtgcaaggcccgcagggttgattcgacgcttttccgc
gaaaaaaacaagtccaaatacccccgtttattctccctcggctctcggtatttcacatgaaaactataacctagactacacgggcaaccttaaccccag
agtatacttatataccaaagggatgggtcctcaaaaatcacacaagcaacg
Yef3 (YALI0E13277g) promoter
(SEQ ID NO: 119)
5′cgccattcggttccttccagaccattccagatcaatccacctcttcttatctcaggtgggtgtgctgacatcagaccccgtagcccttctcccagtgg
cgaacagcaggcataaaacagggccattgagcagagcaaacaaggtcggtgaaatcgtcgaaaaagtcggaaaacggttgcaagaaattggag
cgtcacctgccaccctccaggctctatataaagcattgccccaattgctaacgcttcatatttacacctttggcaccccagtccatccctccaataaaat
gtactacatgggacacaacaagagaggatgcgcgcccaaaccctaacctagcacatgcacgatgattctctttgtctgtgaaaaaatttttccaccaa
aatttccccattgggatgaaaccctaaccgcaaccaaaagtttttaactatcatcttgtacgtcacggtttccgattcttctcttctctttcatcatcatcac
ttgtgacc
Cam1 (YALI0C24420g) promoter
(SEQ ID NO: 120)
5′aactaccataaagtaccgagaaatataggcaattgtacaaattgtccacctccttcacttacattaccgaaccatggccatatcaccaaaatacccc
gagtgctaaaacacctccctccaaatgttctcttaccttccaccgaaaaccgatcttattatcccaacgcttgttgtggcttgacgcgccgcacccgctg
ggcttgccatttcgataccaatccaagaggaaaagctcatgagaaacaatcggaatatcacgagaacggcctggcgaaccaacaggatatttttga
atataattacccctcgaatctagtcatatctatgtctactgtagacttgggcggcatcatgatgtacattattttagcgtctggaaccctaaagttcacgta
caatcatgtgacaaacgaggctaaaaaatgtcaatttcgtatattagtgttattacgtggctcacatttccgaatcatctaccaccccccacctaaaaa
YALI0D16467g promoter
(SEQ ID NO: 121)
5′tttttttaattttcatatttattttcatatttattttcatatttattttcatttatttattcatgtatttatttattactttttaagtattttaaactcc
tcactaaaccgtcgattgcacaatattaaccttcattacacctgcagcgtggtttttgtggtcgttagccgaagtcttccaacgtgggtataagtaggaaca
attgggccgattttttgagccgtctaaatctctcgactcaattgatctgctgtcgaaaatccggctctctagctccttttccccgtccgctggagctcctctt
cattgtgccgtttttccaacatttaactttgccacccaccaccacccccactaccatcacccactcgatctctgttcgtgtcaccacgactttgtcttctca
cacatactctgtttgtgcaccacacattgcgaa
Tef4 (YALI0B12562g) promoter
(SEQ ID NO: 122)
5′gctacaatagctttattggccctattgagcacgctacaattcggtccagtatgtacaacgtctatgcgcactaacggccatacagtgagttacagca
cacccaaaagtaaccctgcctgacctgtctgcctgagacaggaagattaactcttgtagtgaccgagctcgataagactcaagccacacaattttttta
tagccttgcttcaagagtcgccaaaatgacattacacaactccacggaccgtcggttccatgtccacacccttggatgggtaagcgctccacgcacg
taccacgtgcattgagtttaaccacaaacataggtctgtgtcccagagttaccctgctgcatcagccaagtcttgaaagcaaaatttcttgcacaattttt
cctcttcttttcttcactgatcgcagtccaaacacaaaca
YALI0D12903g promoter
(SEQ ID NO: 123)
5′gcgctctgatccacttgtatggctccaagttcagtgtaccaagtagttggtgatgcagggagggatgtctctatccaccaataatgaactcatgggc
gaaattgtttctgttaaacactccaactgtcgttttaaatctcattctctttgcatttggactccattcgcttccgttgggccaatataatccatcgtaacgt
actttagatggaaatttagttacctgctacttgtctcaacaccccaacaggggctgttcgacagaggtaatagagcgtcaatgggttaataaaaacacact
gtcgattttcactcattgtctttatgatattacctgttttccgctgttatcaatgccgagcatcgtgttatatcttccaccccaactacttgcatttacttaa
ctattacctcaactatttacaccccgaattgttacctcccaataagtaactttatttcaaccaatgggacgagagcatctctgagaacatcgatctatctct
gtcaatattgcccagaatcgttcgaaaaaaaacaccaaaaggtttacagcgccattataaatataaattcgttgtcaattcccccgcaatgtctgttgaaa
tctcattttgagaccttccaacattaccctctctcccgtctggtcacatgacgtgactgcttcttcccaaaacgaacactcccaactcttcccccccgtc
agtgaaaagtatacatccgacctccaaatcttttcttcactcaac
Tef1 (YALI0C09141g) promoter
(SEQ ID NO: 124)
5′agagacgggttggcggcgtatttgtgtcccaaaaaacagccccaattgccccaattgaccccaaattgacccagtagcgggcccaaccccggc
gagagcccccttcaccccacatatcaaacctcccccggttcccacacttgccgttaagggcgtagggtactgcagtctggaatctacgcttgttcaga
ctttgtactagtttctttgtctggccatccgggtaacccatgccggacgcaaaatagactactgaaaatttttttgctttgtggttgggactttagccaagg
gtataaaagaccaccgtccccgaattacctttcctcttcttttctctctctccttgtcaactcacacccgaaatcgttaagcatttccttctgagtataaga
atcattc
Fba1 (YALI0E26004g) promoter
(SEQ ID NO: 125)
5′gctgcgctgatctggacaccacagaggttccgagcactttaggttgcaccaaatgtcccaccaggtgcaggcagaaaacgctggaacagcgtg
tacagtttgtcttagcaaaaagtgaaggcgctgaggtcgagcagggtggtgtgacttgttatagcctttagagctgcgaaagcgcgtatggatttggc
tcatcaggccagattgagggtctgtggacacatgtcatgttagtgtacttcaatcgccccctggatatagccccgacaataggccgtggcctcatttttt
tgccttccgcacatttccattgctcggtacccacaccttgcttctcctgcacttgccaaccttaatactggtttacattgaccaacatcttacaagcgggg
ggcttgtctagggtatatataaacagtggctctcccaatcggttgccagtctcttttttcctttctttccccacagattcgaaatctaaactacacatc
Pox2 terminator
(SEQ ID NO: 126)
5′gatgaggaatagacaagcgggtatttattgtatgaataaagattatgtattgattgcaaaaaagtgcatttgtagatgtggtttattgtagagagtacg
gtatgtactgtacgaacattaggagctacttctacaagtagattttcttaacaagggtgaaatttactaggaagtacatgcatatttcgttagtagaatcac
aaaagaaatgtacaagcacgtactacttgtactccacaatgtggagtgggagcaaaaaaattggacgacaccggaatcgaaccggggacctcgc
gcatgctaagcgcatgtgataaccaactacaccagacgcccaagaactttcttggtgattatggaatacgtggtctgctatatctcaattttgctgtaatg
aatcattagaattaaaaaaaaaaccccatttttgtgtgattgtcggccaagagatggaacaggaagaatacgtgaacaagcgagcacgaatgccata
tgctcttctgaacaaccgagtccgaatccgatttgtgggtatcacatgtctcaagtagctgaaatgtatttcgctagaataaaataaatgagattaagaat
taaaaatattggaatatattttcctagaatagaaactttggattttttttcggctattacagtctgaactggacaaacggctgactatatataaatattatt
gggtctgttttcttgtttatgtcgaaattatctgggttttactactgtgtcgtcgagtatagagtggcctgactggagaaaatgcagtagtatggacagtag
gtactgccagccagagaagtttttggaattgatacttgagtcatttttccattccccattccccattccaacacaatcaactgtttctgaacattttccaaaa
cgcggagatgtatgtcacttggcactgcaagtctcgattcaaaatgcatctctttcagaccaaagtgtcatcagctttgtttggccccaaattaccgcaaat
acttgtcgaaattgaagtgcaatacggcctcgtctgccatgaaacctgcctattctcttcaaattggcgtcaggtttcacgtccagcattcctcgcccag
acagagttgctatggttgaatcgtgtactgttaatatatgtatgtattatactcgtactacgatatactgttcaatagagtctcttataatcgtacgacgatt
ctgggca

### Example 12

### Y. lipolytica Oleaginic and Isoprenoid Biosynthesis Genes

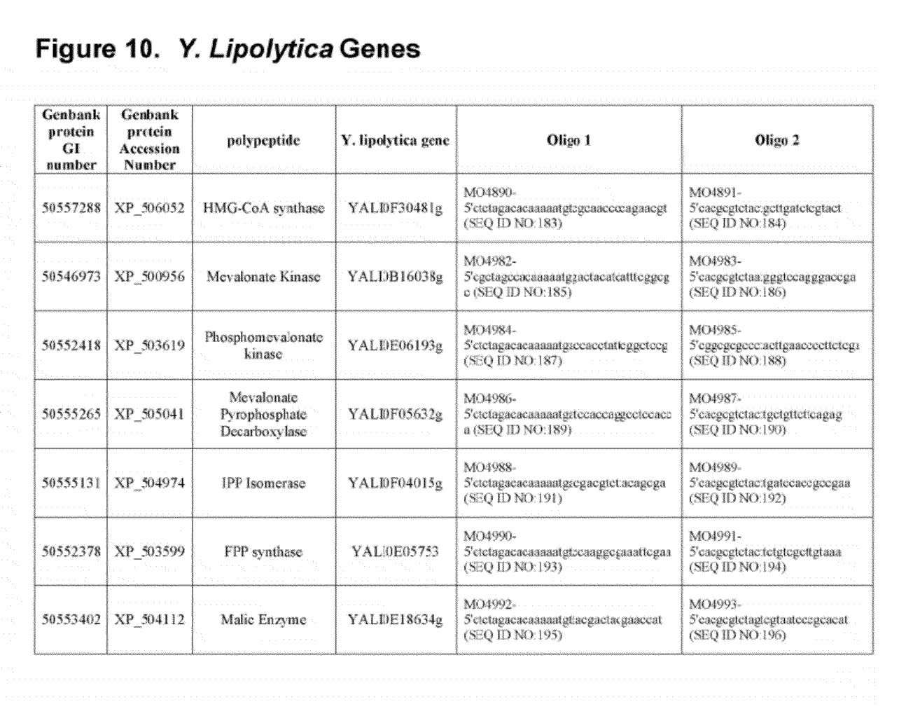

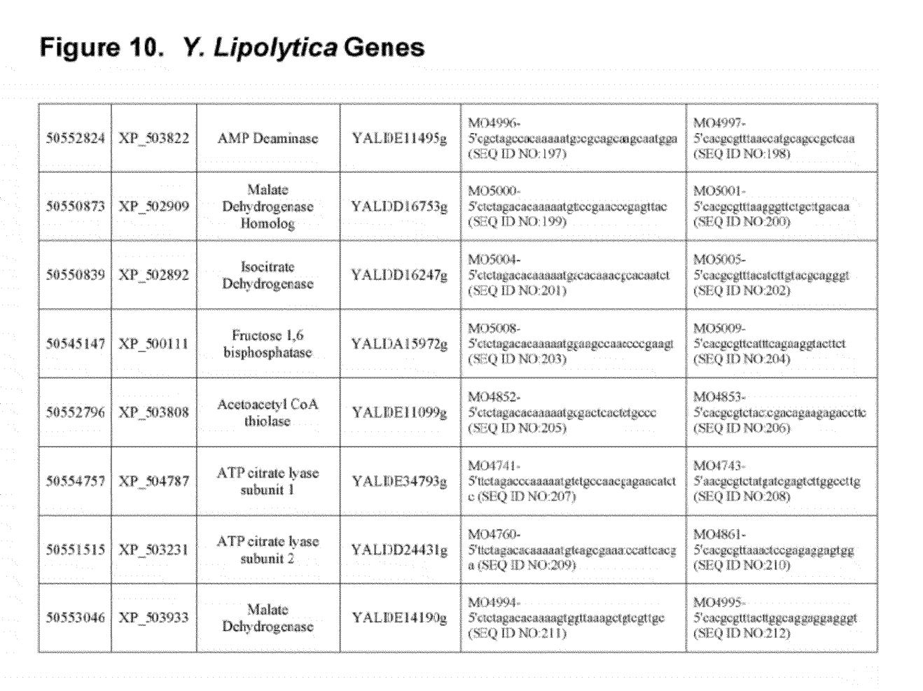

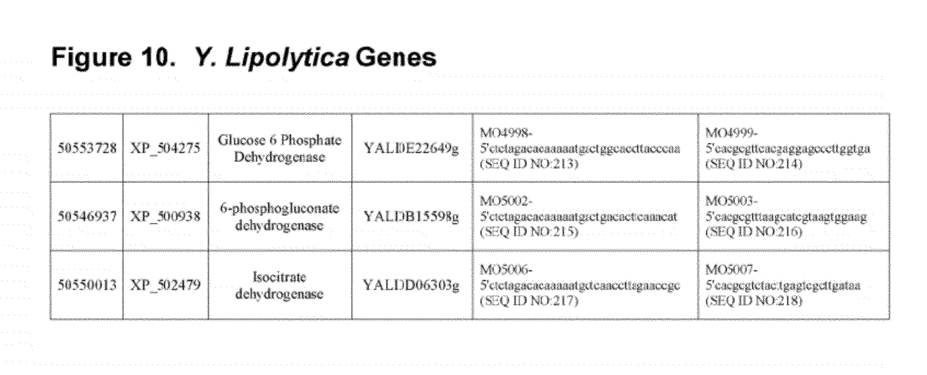

FIGS. 10 (10A, 10B and 10C) is a list of Y. lipolytica genes representing various polypeptides (e.g., oleaginic and isoprenoid biosynthesis peptides) useful in the fungal strains and methods described herein. The Genbank accession number and GI number is given for each polypeptide in addition to oligo pairs which can be used to amplify the coding region for each gene from Y. lipolytica genomic DNA or cDNA. Resulting PCR fragments can be cleaved with restriction enzyme pairs (e.g., depending on what site is present within the oligo sequence, XbaI/MluI or NheI/MluI or XbaI/AscI or NheI/AscI) and inserted into expression vectors (e.g., fungal expression vectors including Y. lipolytica expression vectors such as MB4629 and MB4691 described herein).

The DNA and proteins they encode of the Y. lipolytica genes represented in FIG. 10 are as follows (intron sequence is underlined):

YALI0F30481g
DNA:
(SEQ ID NO: 127)
atgtcgcaaccccagaacgttggaatcaaagccctcgagatctacgtgccttctcgaattgtcaaccaggctgagctcgagaagcacgacggtgtc
gctgctggcaagtacaccattggtcttggtcagaccaacatggcctttgtcgacgacagagaggacatctattcctttgccctgaccgccgtctctcg
actgctcaagaacaacaacatcgaccctgcatctattggtcgaatcgaggttggtactgaaacccttctggacaagtccaagtccgtcaagtctgtgc
tcatgcagctctttggcgagaacagcaacattgagggtgtggacaacgtcaacgcctgctacggaggaaccaacgccctgttcaacgctatcaact
gggttgagggtcgatcttgggacggccgaaacgccatcgtcgttgccggtgacattgccctctacgcaaagggcgctgcccgacccaccggagg
tgccggctgtgttgccatgctcattggccccgacgctcccctggttcttgacaacgtccacggatcttacttcgagcatgcctacgatttctacaagcct
gatctgacctccgagtacccctatgttgatggccactactccctgacctgttacacaaaggccctcgacaaggcctacgctgcctacaacgcccgag
ccgagaaggtcggtctgttcaaggactccgacaagaagggtgctgaccgatttgactactctgccttccacgtgcccacctgcaagcttgtcaccaa
gtcttacgctcgacttctctacaacgactacctcaacgacaagagcctgtacgagggccaggtccccgaggaggttgctgccgtctcctacgatgc
ctctctcaccgacaagaccgtcgagaagaccttccttggtattgccaaggctcagtccgccgagcgaatggctccttctctccagggacccaccaa
caccggtaacatgtacaccgcctctgtgtacgcttctctcatctctctgctgacttttgtccccgctgagcagctgcagggcaagcgaatctctctcttct
cttacggatctggtcttgcttccactcttttctctctgaccgtcaagggagacatttctcccatcgtcaaggcctgcgacttcaaggctaagctcgatgac
cgatccaccgagactcccgtcgactacgaggctgccaccgatctccgagagaaggcccacctcaagaagaactttgagccccagggagacatca
agcacatcaagtctggcgtctactacctcaccaacatcgatgacatgttccgacgaaagtacgagatcaagcagtag
Protein:
(SEQ ID NO: 128)
Msqpqnvgikaleiyvpsrivnqaelekhdgvaagkytiglgqtnmafvddrediysfaltavsrllknnnidpasigrievgtetlldksksvk
svlmqlfgensniegvdnvnacyggtnalfnainwvegrswdgrnaivvagdialyakgaarptggagcvamligpdaplvldnvhgsyfe
haydfykpdltseypyvdghysltcytkaldkayaaynaraekvglfkdsdkkgadrfdysafhvptcklvtksyarllyndylndkslyegq
vpeevaavsydasltdktvektflgiakaqsaermapslqgptntgnmytasvyaslislltfvpaeqlqgkrislfsygsglastlfsltvkgdisp
ivkacdfkaklddrstetpvdyeaatdlrekahlkknfepqgdikhiksgvyyltniddmfrrkyeikq
YALI0B16038g
DNA:
(SEQ ID NO: 129)
atggactacatcatttcggcgccaggcaaagtgattctatttggtgaacatgccgctgtgtttggtaagcctgcgattgcagcagccatcgacttgcga
acatacctgcttgtcgaaaccacaacatccgacaccccgacagtcacgttggagtttccagacatccacttgaacttcaaggtccaggtggacaagc
tggcatctctcacagcccagaccaaggccgaccatctcaattggtcgactcccaaaactctggataagcacattttcgacagcttgtctagcttggcg
cttctggaagaacctgggctcactaaggtccagcaggccgctgttgtgtcgttcttgtacctctacatccacctatgtcccccttctgtgtgcgaagatt
catcaaactgggtagttcgatcaacgctgcctatcggcgcgggcctgggctcttccgcatccatttgtgtctgtttggctgcaggtcttctggttctcaa
cggccagctgagcattgaccaggcaagagatttcaagtccctgaccgagaagcagctgtctctggtggacgactggtccttcgtcggtgaaatgtg
cattcacggcaacccgtcgggcatcgacaatgctgtggctactcagggaggtgctctgttgttccagcgacctaacaaccgagtccctcttgttgac
attcccgagatgaagctgctgcttaccaatacgaagcatcctcgatctaccgcagacctggttggtggagtcggagttctcactaaagagtttggctc
catcatggatcccatcatgacttcagtaggcgagatttccaaccaggccatggagatcatttctagaggcaagaagatggtggaccagtctaaccttg
agattgagcagggtatcttgcctcaacccacctctgaggatgcctgcaacgtgatggaagatggagctactcttcaaaagttgagagatatcggttcg
gaaatgcagcatctagtgagaatcaatcacggcctgcttatcgctatgggtgtttcccacccgaagctcgaaatcattcgaactgcctccattgtccac
aacctgggtgagaccaagctcactggtgctggaggaggaggttgcgccatcactctagtcacttctaaagacaagactgcgacccagctggagga
aaatgtcattgctttcacagaggagatggctacccatggctttcgaggtgcacgagactactattggtgccagaggagttggtatgtgcattgaccatc
cctctctcaagactgttgaagccttcaagaaggtggagcgggcggatctcaaaaacatcggtccctggacccattag
Protein:
(SEQ ID NO: 130)
mdyiisapgkvilfgehaavfgkpaiaaaidlrtyllvetttsdtptvtlefpdihlnfkvqvdklasltaqtkadhlnwstpktldkhifdslsslall
eepgltkvqqaavvsflylyihlcppsvcedssnwvvrstlpigaglgssasicvclaagllvlngqlsidqardfksltekqlslvddwsfvgem
cihgnpsgidnavatqggallfqrpnnrvplvdipemkllltntkhprstadlvggvgvltkefgsimdpimtsvgeisnqameiisrgkkmv
dqsnleieqgilpqptsedacnvmedgatlqklrdigsemqhlvrinhglliamgvshpkleiirtasivhnlgetkltgaggggcaitlvtskdk
tatqleenviafteemathgfevhettigargvgmcidhpslktveafkkveradlknigpwth
YALI0E06193g
DNA:
(SEQ ID NO: 131)
atgaccacctattcggctccgggaaaggccctcctttgcggcggttatttggttattgatccggcgtattcagcatacgtcgtgggcctctcggcgcgt
atttacgcgacagtttcggcttccgaggcctccaccacctctgtccatgtcgtctctccgcagtttgacaagggtgaatggacctacaactacacgaa
cggccagctgacggccatcggacacaacccatttgctcacgcggccgtcaacaccgttctgcattacgttcctcctcgaaacctccacatcaacatc
agcatcaaaagtgacaacgcgtaccactcgcaaattgacagcacgcagagaggccagtttgcataccacaaaaaggcgatccacgaggtgccta
aaacgggcctcggtagctccgctgctcttaccaccgttcttgtggcagctttgctcaagtcatacggcattgatcccttgcataacacccacctcgttca
caacctgtcccaggttgcacactgctcggcacagaagaagattgggtctggatttgacgtggcttcggccgtttgtggctctctagtctatagacgttt
cccggcggagtccgtgaacatggtcattgcagctgaagggacctccgaatacggggctctgttgagaactaccgttaatcaaaagtggaaggtga
ctctggaaccatccttcttgccgccgggaatcagcctgcttatgggagacgtccagggaggatctgagactccaggtatggtggccaaggtgatgg
catggcgaaaagcaaagccccgagaagccgagatggtgtggagagatctcaacgctgccaacatgctcatggtcaagttgttcaacgacctgcgc
aagctctctctcactaacaacgaggcctacgaacaacttttggccgaggctgctcctctcaacgctctaaagatgataatgttgcagaaccctctcgg
agaactagcacgatgcattatcactattcgaaagcatctcaagaagatgacacgggagactggtgctgctattgagccggatgagcagtctgcattg
ctcaacaagtgcaacacttatagtggagtcattggaggtgttgtgcctggagcaggaggctacgatgctatttctcttctggtgatcagctctacggtg
aacaatgtcaagcgagagagccagggagtccaatggatggagctcaaggaggagaacgagggtctgcggctcgagaaggggttcaagtag
Protein:
(SEQ ID NO: 132)
mttysapgkallcggylvidpaysayvvglsariyatvsaseasttsvhvvspqfdkgewtynytngqltaighnpfahaavntvlhyvpprnl
hinisiksdnayhsqidstqrgqfayhkkaihevpktglgssaalttvlvaallksygidplhnthlvhnlsqvahcsaqkkigsgfdvasavcgs
lvyrrfpaesvnmviaaegtseygallrttvnqkwkvtlepsflppgisllmgdvqggsetpgmvakvmawrkakpreaemvwrdlnaan
mlmvklfndlrklsltnneayeqllaeaaplnalkmimlqnplgelarciitirkhlkkmtretgaaiepdeqsallnkcntysgviggvvpgag
gydaisllvisstvnnvkresqgvqwmelkeeneglrlekgfk
YALI0F05632g
DNA:
(SEQ ID NO: 133)
atgatccaccaggcctccaccaccgctccggtgaacattgcgacactcaagtactggggcaagcgagaccctgctctcaatctgcccactaacaac
tccatctccgtgactttgtcgcaggatgatctgcggaccctcaccacagcctcgtgttcccctgatttcacccaggacgagctgtggctcaatggcaa
gcaggaggacgtgagcggcaaacgtctggttgcgtgtttccgagagctgcgggctctgcgacacaaaatggaggactccgactcttctctgcctaa
gctggccgatcagaagctcaagatcgtgtccgagaacaacttccccaccgccgctggtctcgcctcatcggctgctggctttgccgccctgatccg
agccgttgcaaatctctacgagctccaggagacccccgagcagctgtccattgtggctcgacagggctctggatccgcctgtcgatctctctacgga
ggctacgtggcatgggaaatgggcaccgagtctgacggaagcgactcgcgagcggtccagatcgccaccgccgaccactggcccgagatgcg
agccgccatcctcgttgtctctgccgacaagaaggacacgtcgtccactaccggtatgcaggtgactgtgcacacttctcccctcttcaaggagcga
gtcaccactgtggttcccgagcggtttgcccagatgaagaagtcgattctggaccgagacttccccacctttgccgagctcaccatgcgagactcaa
accagttccacgccacctgtctggactcgtatcctcccattttctacctcaacgacgtgtcgcgagcctccattcgggtagttgaggccatcaacaag
gctgccggagccaccattgccgcctacacctttgatgctggacccaactgtgtcatctactacgaggacaagaacgaggagctggttctgggtgct
ctcaaggccattctgggccgtgtggagggatgggagaagcaccagtctgtggacgccaagaagattgatgttgacgagcggtgggagtccgagc
tggccaacggaattcagcgggtgatccttaccaaggttggaggagatcccgtgaagaccgctgagtcgcttatcaacgaggatggttctctgaaga
acagcaagtag
Protein:
(SEQ ID NO: 134)
mihqasttapvniatlkywgkrdpalnlptnnsisvtlsqddlrtlttascspdftqdelwlngkqedvsgkrlvacfrelralrhkmedsdsslpk
ladqklkivsennfptaaglassaagfaaliravanlyelqetpeqlsivarqgsgsacrslyggyvawemgtesdgsdsravqiatadhwpem
raailvvsadkkdtssttgmqvtvhtsplfkervttvvperfaqmkksildrdfptfaeltmrdsnqfhatcldsyppifylndvsrasirvveain
kaagatiaaytfdagpncviyyedkneelvlgalkailgrvegwekhqsvdakkidvderweselangiqrviltkvggdpvktaeslinedg
slknsk
YALI0F04015g
DNA:
(SEQ ID NO: 135)
Atgacgacgtcttacagcgacaaaatcaagagtatcagcgtgagctctgtggctcagcagtttcctgaggtggcgccgattgcggacgtgtccaag
gctagccggcccagcacggagtcgtcggactcgtcggccaagctatttgatggccacgacgaggagcagatcaagctgatggacgagatctgtg
tggtgctggactgggacgacaagccgattggcggcgcgtccaaaaagtgctgtcatctgatggacaacatcaacgacggactggtgcatcgggc
cttttccgtgttcatgttcaacgaccgcggtgagctgcttctgcagcagcgggcggcggaaaaaatcacctttgccaacatgtggaccaacacgtgc
tgctcgcatcctctggcggtgcccagcgagatgggcgggctggatctggagtcccggatccagggcgccaaaaacgccgcggtccggaagctt
gagcacgagctgggaatcgaccccaaggccgttccggcagacaagttccatttcctcacccggatccactacgccgcgccctcctcgggcccctg
gggcgagcacgagattgactacattctgtttgtccggggcgaccccgagctcaaggtggtggccaacgaggtccgcgataccgtgtgggtgtcgc
agcagggactcaaggacatgatggccgatcccaagctggttttcaccccttggttccggctcatttgtgagcaggcgctgtttccctggtgggacca
gttggacaatctgcccgcgggcgatgacgagattcggcggtggatcaagtag
Protein:
(SEQ ID NO: 136)
mttsysdkiksisvssvaqqfpevapiadvskasrpstessdssaklfdghdeeqiklmdeicvvldwddkpiggaskkcchlmdnindglv
hrafsvfmfndrgelllqqraaekitfanmwtntccshplavpsemggldlesriqgaknaavrklehelgidpkavpadkfhfltrihyaapss
gpwgeheidyilfvrgdpelkvvanevrdtvwvsqqglkdmmadpklvftpwfrliceqalfpwwdqldnlpagddeirrwik
YALI0E05753
DNA:
(SEQ ID NO: 137)
atgtccaaggcgaaattcgaaagcgtgttcccccgaatctccgaggagctggtgcagctgctgcgagacgagggtctgccccaggatgccgtgc
agtggttttccgactcacttcagtacaactgtgtgggtggaaagctcaaccgaggcctgtctgtggtcgacacctaccagctactgaccggcaagaa
ggagctcgatgacgaggagtactaccgactcgcgctgctcggctggctgattgagctgctgcaggcgtttttcctcgtgtcggacgacattatggat
gagtccaagacccgacgaggccagccctgctggtacctcaagcccaaggtcggcatgattgccatcaacgatgctttcatgctagagagtggcat
ctacattctgcttaagaagcatttccgacaggagaagtactacattgaccttgtcgagctgttccacgacatttcgttcaagaccgagctgggccagct
ggtggatcttctgactgcccccgaggatgaggttgatctcaaccggttctctctggacaagcactcctttattgtgcgatacaagactgcttactactcc
ttctacctgcccgttgttctagccatgtacgtggccggcattaccaaccccaaggacctgcagcaggccatggatgtgctgatccctctcggagagt
acttccaggtccaggacgactaccttgacaactttggagaccccgagttcattggtaagatcggcaccgacatccaggacaacaagtgctcctggct
cgttaacaaagcccttcagaaggccacccccgagcagcgacagatcctcgaggacaactacggcgtcaaggacaagtccaaggagctcgtcatc
aagaaactgtatgatgacatgaagattgagcaggactaccttgactacgaggaggaggttgttggcgacatcaagaagaagatcgagcaggttga
cgagagccgaggcttcaagaaggaggtgctcaacgctttcctcgccaagatttacaagcgacagaagtag
Protein:
(SEQ ID NO: 138)
mskakfesvfpriseelvqllrdeglpqdavqwfsdslqyncvggklnrglsvvdtyqlltgkkelddeeyyrlallgwliellqafflvsddim
desktrrgqpcwylkpkvgmiaindafmlesgiyillkkhfrqekyyidlvelfhdisfktelgqlvdlltapedevdlnrfsldkhsfivrykta
yysfylpvvlamyvagitnpkdlqqamdvliplgeyfqvqddyldnfgdpefigkigtdiqdnkcswlvnkalqkatpeqrqilednygvk
dkskelvikklyddmkieqdyldyeeevvgdikkkieqvdesrgfkkevlnaflakiykrqk
YALI0E18634g
DNA:
(SEQ ID NO: 139)
atgttacgactacgaaccatgcgacccacacagaccagcgtcagggcggcgcttgggcccaccgccgcggcccgaaacatgtcctcctccagc
ccctccagcttcgaatactcgtcctacgtcaagggcacgcgggaaatcggccaccgaaaggcgcccacaacccgtctgtcggttgagggcccca
tctacgtgggcttcgacggcattcgtcttctcaacctgccgcatctcaacaagggctcgggattccccctcaacgagcgacgggaattcagactcag
tggtcttctgccctctgccgaagccaccctggaggaacaggtcgaccgagcataccaacaattcaaaaagtgtggcactcccttagccaaaaacgg
gttctgcacctcgctcaagttccaaaacgaggtgctctactacgccctgctgctcaagcacgttaaggaggtcttccccatcatctatacaccgactca
gggagaagccattgaacagtactcgcggctgttccggcggcccgaaggctgcttcctcgacatcaccagtccctacgacgtggaggagcgtctg
ggagcgtttggagaccatgacgacattgactacattgtcgtgactgactccgagggtattctcggaattggagaccaaggagtgggcggtattggta
tttccatcgccaagctggctctcatgactctatgtgctggagtcaacccctcacgagtcattcctgtggttctggatacgggaaccaacaaccaggag
ctgctgcacgaccccctgtatctcggccgacgaatgccccgagtgcgaggaaagcagtacgacgacttcatcgacaactttgtgcagtctgcccga
aggctgtatcccaaggcggtgatccatttcgaggactttgggctcgctaacgcacacaagatcctcgacaagtatcgaccggagatcccctgcttca
acgacgacatccagggcactggagccgtcactttggcctccatcacggccgctctcaaggtgctgggcaaaaatatcacagatactcgaattctcgt
gtacggagctggttcggccggcatgggtattgctgaacaggtctatgataacctggttgcccagggtctcgacgacaagactgcgcgacaaaacat
ctttctcatggaccgaccgggtctactgaccaccgcacttaccgacgagcagatgagcgacgtgcagaagccgtttgccaaggacaaggccaatt
acgagggagtggacaccaagactctggagcacgtggttgctgccgtcaagccccatattctcattggatgttccactcagcccggcgcctttaacga
gaaggtcgtcaaggagatgctcaaacacacccctcgacccatcattctccctctttccaaccccacacgtcttcatgaggctgtccctgcagatctgt
acaagtggaccgacggcaaggctctggttgccaccggctcgccctttgacccagtcaacggcaaggagacgtctgagaacaataactgctttgtttt
ccccggaatcgggctgggagccattctgtctcgatcaaagctcatcaccaacaccatgattgctgctgccatcgagtgcctcgccgaacaggcccc
cattctcaagaaccacgacgagggagtacttcccgacgtagctctcatccagatcatttcggcccgggtggccactgccgtggttcttcaggccaag
gctgagggcctagccactgtcgaggaagagctcaagcccggcaccaaggaacatgtgcagattcccgacaactttgacgagtgtctcgcctgggt
cgagactcagatgtggcggcccgtctaccggcctctcatccatgtgcgggattacgactag
Protein:
(SEQ ID NO: 140)
mlrlrtmrptqtsvraalgptaaarnmsssspssfeyssyvkgtreighrkapttrlsvegpiyvgfdgirllnlphlnkgsgfplnerrefrlsgllp
saeatleeqvdrayqqfkkcgtplakngfctslkfqnevlyyalllkhvkevfpiiytptqgeaieqysrlfrrpegcflditspydveerlgafgdh
ddidyivvtdsegilgigdqgvggigisiaklalmtlcagvnpsrvipvvldtgrtnnqellhdplylgrrmprvrgkqydfidnfvqsarrlyp
kavihfedfglanahkildkyrpeipcfnddiqgtgavtlasitaalkvlgknitdtrilvygagsagmgiaeqvydnlvaqglddktarqniflm
drpgllttaltdeqmsdvqkpfakdkanyegvdtktlehvvaavkphiligestqpgafnekvvkemlkhtprpiilplsnptrlheavpadly
kwtdgkalvatgspfdpvngketsennnefvfpgiglgailsrsklitntmiaaaieclaeqapilknhdegvlpdvaliqiisarvatavvlqak
aeglatveeelkpgtkehvqipdnfdeclawvetqmwrpvyrplihvrdyd
YALI0E11495g
DNA:
(SEQ ID NO: 141)
atgccgcagcaagcaatggatatcaagggcaaggccaagtctgtgcccatgcccgaagaagacgacctggactcgcattttgtgggtcccatctct
ccccgacctcacggagcagacgagattgctggctacgtgggctgcgaagacgacgaagacgagcttgaagaactgggaatgctgggccgatct
gcgtccacccacttctcttacgcggaagaacgccacctcatcgaggttgatgccaagtacagagctcttcatggccatctgcctcatcagcactctca
gagtcccgtgtccagatcttcgtcatttgtgcgggccgaaatgaaccacccccctcccccaccctccagccacacccaccaacagccagaggacg
atgacgcatcttccactcgatctcgatcgtcgtctcgagcttctggacgcaagttcaacagaaacagaaccaagtctggatcttcgctgagcaagggt
ctccagcagctcaacatgaccggatcgctcgaagaagagccctacgagagcgatgacgatgcccgactatctgcggaagacgacattgtctatga
tgctacccagaaagacacctgcaagcccatatctcctactctcaaacgcacccgcaccaaggacgacatgaagaacatgtccatcaacgacgtca
aaatcaccaccaccacagaagatcctcttgtggcccaggagctgtccatgatgttcgaaaaggtgcagtactgccgagacctccgagacaagtacc
aaaccgtgtcgctacagaaggacggagacaaccccaaggatgacaagacacactggaaaatttaccccgagcctccaccaccctcctggcacga
gaccgaaaagcgattccgaggctcgtccaaaaaggagcaccaaaagaaagacccgacaatggatgaattcaaattcgaggactgcgaaatcccc
ggacccaacgacatggtcttcaagcgagatcctacctgtgtctatcaggtctatgaggatgaaagctcttctcaacgaaaataagccgtttgttgccatc
ccctcaatccgagattactacatggatctggaggatctcattgtggcttcgtctgacggacctgccaagtcttttgctttccgacgactgcaatatctag
aagccaagtggaacctctactacctgctcaacgagtacacggagacaaccgagtccaagaccaacccccatcgagacttttacaaacgtacgaaag
gtcgacacccacgttcaccactctgcctgcatgaaccagaagcatctgctgcgattcatcaaatacaagatgaagaactgccctgatgaagttgtcat
ccaccgagacggtcgggagctgacactctcccaggtgtttgagtcacttaacttgactgcctacgacctgtctatcgatacccttgatatgcatgctca
caaggactcgttccatcgatttgacaagttcaacctcaagtacaaccctgtcggtgagtctcgactgcgagaaatcttcctaaagaccgacaactaca
tccagggtcgatacctagctgagatcacaaaggaggtgttccaggatctcgagaactcgaagtaccagatggcggagtaccgtatttccatctacg
gtcggtccaaggacgagtgggacaagctggctgcctgggtgctggacaacaaactgttttcgcccaatgttcggtggttgatccaggtgcctcgact
gtacgacatttacaagaaggctggtctggttaacacctttgccgacattgtgcagaacgtctttgagcctcttttcgaggtcaccaaggatcccagtac
ccatcccaagctgcacgtgttcctgcagcgagttgtgggctttgactctgtcgatgacgagtcgaagctggaccgacgtttccaccgaaagttccca
actgcagcatactgggacagcgcacagaaccctccctactcgtactcgtactggcagtactatctatacgccaacatggcctccatcaacacctggagacag
cgtttgggctataatacttttgagttgcgaccccatgctggagaggctggtgacccagagcatcttctgtgcacttatctggttgctcagggtatcaacc
acggtattctgttgcgaaaggtgcccttcattcagtacctttactacctggaccagatccccattgccatgtctcctgtgtccaacaatgcgctgttcctc
acgttcgacaagaaccccttctactcatacttcaagcggggtctcaacgtgtccttgtcatcggatgatcctctgcagttgcttacactaaggaggctc
tgattgaggagtactgtggctgcgctcatttacaagctttccaacgtggatatgtgtgagcttgctcgaaactcggtactgcaatctggctttgagcg
aatcatcaaggagcattggatcggcgaaaactacgagatccatggccccgagggcaacaccatccagaagacaaacgtgcccaatgtgcgtctg
gccttccgagacgagactttgacccacgagcttgctctggtggacaagtacaccaatcttgaggagtttgagcggctgcatggttaa
Protein:
(SEQ ID NO: 142)
mpqqamdikgkaksvpmpeeddldshfvgpisprphgadeiagyvgceddedeleelgmlgrsasthfsyaeerhlievdakyralhghl
phqhsqspvsrsssfvraemnhpppppsshthqqpedddasstrsrsssrasgrkfnrnrtksgsslskglqqlnmtgsleeepyesdddarls
aeddivydatqkdtckpisptlkrtrtkddmknmsindvkittttedplvaqelsmmfekvqycrdlrdkyqtvslqkdgdnpkddkthwki
ypeppppswhetekrfrgsskkehqkkdptmdefkfedceipgpndmvfkrdptcvyqvyedesslnenkpfvaipsirdyymdledliv
assdgpaksfafrrlqyleakwnlyyllneytettesktnphrdfynvrkvdthvhhsacmnqkhllrfikykmkncpdevvihrdgreltlsq
vfeslnltaydlsidtldmhahkdsfhrfdkfnlkynpvgesrlreiflktdnyiqgrylaeitkevfqdlenskyqmaeyrisiygrskdewdkl
aawvldnklfspnvrwliqvprlydiykkaglvntfadivqnvfeplfevtkdpsthpklhvflqrvvgfdsvddeskldrrfhrkfptaaywd
saqnppysywqyylyanmasintwrqrlgyntfelrphageagdpehllctylvaqginhgillrkvpfiqylyyldqipiamspvsnnalflt
fdknpfysyfkrglnvslssddplqfaytkealieeysvaaliyklsnvdmcelarnsvlqsgferiikehwigenyeihgpegntiqktnvpnv
rlafrdetlthelalvdkytnleeferlhg
YALI0D16753g
DNA:
(SEQ ID NO: 143)
atgttccgaacccgagttaccggctccaccctgcgatccttctccacctccgctgcccgacagcacaaggttgtcgtccttggcgccaacggaggc
attggccagcccctgtctctgctgctcaagctcaacaagaacgtgaccgacctcggtctgtacgatctgcgaggcgcccccggcgttgctgccgat
gtctcccacatccccaccaactccaccgtggccggctactctcccgacaacaacggcattgccgaggccctcaagggcgccaagctggtgctgat
ccccgccggtgtcccccgaaagcccggcatgacccgagacgatctgttcaacaccaacgcctccattgtgcgagacctggccaaggccgtcggt
gagcacgcccccgacgcctttgtcggagtcattgctaaccccgtcaactccaccgtccccattgtcgccgaggtgctcaagtccaagggcaagtac
gaccccaagaagctcttcggtgtcaccaccctcgacgtcatccgagccgagcgattcgtctcccagctcgagcacacccaaccccaccaaggagta
cttccccgttgttggcggccactccggtgtcaccattgtccccctcgtgtcccagtccgaccaccccgacattgccggtgaggctcgagacaagctt
gtccaccgaatccagtttggcggtgacgaggttgtcaaggccaaggacggtgccggatccgccaccctttccatggcccaggctgccgcccgatt
cgccgactctctcctccgaggtgtcaacggcgagaaggacgttgttgagcccactttcgtcgactctcctctgttcaagggtgagggcatcgacttct
tctccaccaaggtcactcttggccctaacggtgttgaggagatccaccccatcggaaaggtcaacgagtacgaggagaagctcatcgaggctgcc
aaggccgatctcaagaagaacattgagaagggtgtcaactttgtcaagcagaacccttaa
Protein:
(SEQ ID NO: 144)
mfrtrvtgstlrsfstsaarqhkvvvlganggigqplslllklnknvtdlglydlrgapgvaadvshiptnstvagyspdnngiaealkgaklvlip
agvprkpgmtrddlfntnasivrdlakavgehapdafvgvianpvnstvpivaevlkskgkydpkklfgvttldviraerfvsqlehtnptkey
fpvvgghsgvtivplvsqsdhpdiageardklvhriqfggdevvkakdgagsatlsmaqaaarfadsllrgvngekdvveptfvdsplfkgeg
idffstkvtlgpngveeihpigkvneyeeklieaakadlkkniekgvnfvkqnp
YALI0D16247g
DNA:
(SEQ ID NO: 145)
atgacacaaacgcacaatctgttttcgccaatcaaagtgggctcttcggagctccagaaccggatcgttctcgcacccttgactcgaaccagagctct
gcccggaaacgtgccctcggatcttgccacagagtactacgcacaaagagcagcatctccaggcactctcctcatcaccgaggccacatacatctc
ccccggatctgctggagtgcccattccaggagacggaatcgttccgggcatctggagtgacgagcagctgaagcatggaaaaaggtgttcaag
gccgtgcacgaccgaggatccaaaatctacgtccagctgtgggacattggacgtgtcgcatggtaccacaagctgcaggaactgggcaactactt
ccctacaggcccctcagctatccccatgaagggagaggagagcgagcatctcaaggctctgactcactgggagatcaagggcaaggtggccctc
tacgtcaacgctgccaagaacgccattgccgcaggcgctgatggcgtcgagatccactcggccaacggctaccttcccgacacatttctgagaag
cgcctccaaccaacgaacagacgaatatggaggaagcatcgagaaccgggcccgattctcgctggagattgtcgacgctatcaccgaggccatt
ggagcagacaaaaccgccatccgtctgtctccctggtccactttccaggacattgaggtgaatgacaccgagacccccgcacagttcacatacctg
tttgagcagctgcagaagcgagccgacgagggaaagcagctggcctacgtgcatgtagttgagccccgactgtttggtccccccgagccctggg
ccaccaatgagcctttcagaaaaatttggaagggtaacttcattagagcaggtggatacgatagagagactgctcttgaggatgcagacaagtcaga
caacaccctgattgcctttggtcgagacttcattgccaatcctgatctcgtccaacgcctcaagaataacgagcctttggccaagtacgacagaacaa
ccttctacgttccaggtgccaagggctacactgattaccctgcgtacaagatgtaa
Protein:
(SEQ ID NO: 146)
mtqthnlfspikvgsselqnrivlapltrtralpgnvpsdlateyyaqraaspgtlliteatyispgsagvpipgdgivpgiwsdeqleawkkvfk
avhdrgskiyvqlwdigrvawyhklqelgnyfptgpsaipmkgeesehlkalthweikgkvalyvnaaknaiaagadgveihsangylpdt
flrsasnqrtdeyggsienrarfsleivdaiteaigadktairlspwstfqdievndtetpaqftylfeqlqkradegkqlayvhvveprlfgppep
watnepfrkiwkgnfiraggydretaledadksdntliafgrdfianpdlvqrlknneplakydrttfyvpgakgytdypaykm
YALI0A15972g
DNA:
(SEQ ID NO: 147)
atggaagccaaccccgaagtccagaccgatatcatcacgctgacccggttcattctgcaggaacagaacaaggtgggcgcgtcgtccgcaatccc
caccggagacttcactctgctgctcaactcgctgcagtttgccttcaagttcattgcccacaacatccgacgatcgaccctggtcaacctgattggcct
gtcgggaaccgccaactccaccggcgacgaccagaagaagctggacgtgatcggagacgagatcttcatcaacgccatgaaggcctccggtaa
ggtcaagctggtggtgtccgaggagcaggaggacctcattgtgtttgagggcgacggccgatacgccgtggtctgcgaccccatcgacggatcct
ccaacctcgacgccggcgtctccgtcggcaccattttcggcgtctacaagctccccgagggctcctccggatccatcaaggacgtgctccgaccc
ggaaaggagatggttgccgccggctacaccatgtacggtgcctccgccaacctggtgctgtccaccggaaacggctgcaacggcttcactctcga
tgaccctctgggagagttcatcctgacccaccccgatctcaagctccccgatctgcgatccatctactccgtcaacgagggtaactcctccctgtggt
ccgacaacgtcaaggactacttcaaggccctcaagttccccgaggacggctccaagccctactcggcccgatacattggctccatggtcgccgac
gtgcaccgaaccattctctacggaggtatgtttgcctaccccgccgactccaagtccaagaagggcaagctccgacttttgtacgagggtttccccat
ggcctacatcattgagcaggccggcggtcttgccatcaacgacaacggcgagcgaatcctcgatctggtccccaccgagatccacgagcgatcc
ggcgtctggctgggctccaagggcgagattgagaaggccaagaagtaccttctgaaatga
Protein:
(SEQ ID NO: 148)
meanpevqtdiitltrfilqeqnkvgassaiptgdftlllnslqfafkfiahnirrstlvnliglsgtanstgddqkkldvigdeifinamkasgkvkl
vvseeqedlivfegdgryavvcdpidgssnldagvsvgtifgvyklpegsspsikdvlrpgkemvaagytmygasanlvlstgngcngftld
dplgefilthpdlklpdlrsiysvnegnsslwsdnvkdyfkalkfpedgskpysaryigsmvadvhrtilyggmfaypadskskkgklrllye
gfpmayiieqagglaindngerildlvpteihersgvwlgskgeiekakkyllk
YALI0E11099g
DNA:
(SEQ ID NO: 149)
atgcgactcactctgccccgacttaacgccgcctacattgtaggagccgcccgaactcctgtcggcaagttcaacggagccctcaagtccgtgtctg
ccattgacctcggtatcaccgctgccaaggccgctgtccagcgatccaaggtccccgccgaccagattgacgagtttctgtttggccaggtgctgac
cgccaactccggccaggcccccgcccgacaggtggttatcaagggtggtttccccgagtccgtcgaggccaccaccatcaacaaggtgtgctctt
ccggcctcaagaccgtggctctggctgcccaggccatcaaggccggcgaccgaaacgttatcgtggccggtggaatggagtccatgtccaacac
cccctactactccggtcgaggtcttgttttcggcaaccagaagctcgaggactccactcgtcaaggacggtctctgggacccctacaacaacatccac
atgggcaactgctgcgagaacaccaacaagcgagacggcatcacccgagagcagcaggacgagtacgccatcgagtcctaccgacgggccaa
cgagtccatcaagaacggcgccttcaaggatgagattgtccccgttgagatcaagacccgaaagggcaccgtgactgtctccgaggacgaggag
cccaagggagccaacgccgagaagctcaagggcctcaagcctgtctttgacaagcagggctccgtcactgccggtaacgcctcccccatcaacg
atggtgcttctgccgttgtcgttgcctctggcaccaaggccaaggagctcggtacccccgtgctcgccaagattgtctcttacgcagacgccgccac
cgcccccattgactttaccattgctccctctctggccattcccgccgccctcaagaaggcttggccttaccaaggacgacattgccctctgggagatca
acgaggccttctccggtgtcgctctcgccaacctcatgcgactcggaattgacaagtccaaggtcaacgtcaagggtggagctgttgctctcggcc
accccattggtgcctccggtaaccgaatctttgtgactttggtcaacgccctcaaggagggcgagtacggagttgccgccatctgcaacggtggag
gagcttccaccgccatcgtcatcaagaaggtctcttctgtcgagtag
Protein:
(SEQ ID NO: 150)
mrltlprlnaayivgaartpvgkfngalksvsaidlgitaakaavqrskvpadqideflfgqvltansgqaparqvvikggfpesveattinkvcs
sglktvalaaqaikagdrnvivaggmesmsntpyysgrglvfgnqkledsivkdglwdpynnihmgnccentnkrdgitreqqdeyaiesy
rranesikngafkdeivpeiktrkgtvtvsedeepkganaeklkglkpvfdkqgsvtagnaspindgasavvvasgtkakelgtpvlakivsy
adaatapidftiapslaipaalkkagltkddialweineafsgvalanlmrlgidkskvnvkggavalghpigasgnrifvtlvnalkegeygva
aicngggastaivkkvssve
YALI0E34793g
DNA:
(SEQ ID NO: 151)
atgtctgccaacgagaacatctcccgattcgacgcccctgtgggcaaggagcaccccgcctacgagctcttccataaccacacacgatctttcgtct
atggtctccagcctcgagcctgccagggtatgctggacttcgacttcatctgtaagcgagagaacccctccgtggccggtgtcatctatcccttcggc
ggccagttcgtcaccaagatgtactggggcaccaaggagactcttctccctgtctaccagcaggtcgagaaggccgctgccaagcaccccgaggt
cgatgtcgtggtcaactttgcctcctctcgatccgtctactcctctaccatggagctgctcgagtacccccagttccgaaccatcgccattattgccgag
ggtgtccccgagcgacgagcccgagagatcctccacaaggcccagaagaagggtgtgaccatcattggtcccgctaccgtcggaggtatcaagc
ccggttgcttcaaggttggaaacaccggaggtatgatggacaacattgtcgcctccaagctctaccgacccggctccgttgcctacgtctccaagtc
cggaggaatgtccaacgagctgaacaacattatctctcacaccaccgacggtgtctacgagggtattgctattggtggtgaccgataccctggtact
accttcattgaccatatcctgcgatacgaggccgaccccaagtgtaagatcatcgtcctccttggtgaggttggtggtgttgaggagtaccgagtcat
cgaggctgttaagaacggccagatcaagaagcccatcgtcgcttgggccattggtacttgtgcctccatgttcaagactgaggttcagttcggccac
gccggctccatggccaactccgacctggagactgccaaggctaagaacgccgccatgaagtctgctggcttctacgtccccgataccttcgagga
catgcccgaggtccttgccgagctctacgagaagatggtcgccaagggcgagctgtctcgaatctctgagcctgaggtccccaagatccccattga
ctactcttgggcccaggagcttggtcttatccgaaagcccgctgctttcatctccactatttccgatgaccgaggccaggagcttctgtacgctggcat
gcccatttccgaggttttcaaggaggacattggtatcggcggtgtcatgtctctgctgtggttccgacgacgactccccgactacgcctccaagtttctt
gagatggttctcatgcttactgctgaccacggtcccgccgtatccggtgccatgaacaccattatcaccacccgagctggtaaggatctcatttcttcc
ctggttgctggtctcctgaccattggtacccgattcggaggtgctcttgacggtgctgccaccgagttcaccactgcctacgacaagggtctgtcccc
ccgacagttcgttgataccatgcgaaagcagaacaagctgattcctggtattggccatcgagtcaagtctgaaacaaccccgatttccgagtcgag
cttgtcaaggactttgttaagaagaacttcccctccacccagctgctcgactacgcccttgctgtcgaggaggtcaccacctccaagaaggacaacc
tgattctgaacgttgacggtgctattgctgtttcttttgtcgatctcatgcgatcttgcggtgcctttactgtggaggagactgaggactacctcaagaac
ggtgttctcaacggtctgttcgttctcggtcgatccattggtctcattgcccaccatctcgatcagaagcgactcaagaccggtctgtaccgacatcctt
gggacgatatcacctacctggttggccaggaggctatccagaagaagcgagtcgagatcagcgccggcgacgtttccaaggccaagactcgatc
atag
Protein:
(SEQ ID NO: 152)
msanenisrfdapvgkehpayelfhnhtrsfvyglqpracqgmldfdfickrenpsvagviypfggqfvtkmywgtketllpvyqqvekaa
akhpevdvvvnfassrsvysstmelleypqfrtiaiiaegvperrareilhkaqkkgvtiigpatvggikpgcfkvgntggmmdnivasklyr
pgsvayvsksggmsnelnniishttdgvyegiaiggdrypgttfidhilryeadpkckiivllgevggveeyrvieavkngqikkpivawaigt
casmfktevqfghagsmansdletakaknaamksagfyvpdtfedmpevlaelyekmvakgelsrisepevpkipidyswaqelglirkp
aafistisddrgqellyagmpisevfkedigiggvmsllwfrrrlpdyaskflemvlmltadhgpavsgamntiittragkdlisslvaglltigtrf
ggaldgaatefttaydkglsprqfvdtmrkqnklipgighrvksrnnpdfrvelvkdfvkknfpstqlldyalaveevttskkdnlilnvdgaia
vsfvdlmrscgaftveetedylkngvlnglfvlgrsigliahhldqkrlktglyrhpwdditylvgqeaiqkkrveisagdvskaktrs
YALI0D24431g
DNA:
(SEQ ID NO: 153)
atgtcagcgaaatccattcacgaggccgacggcaaggccctgctcgcacactttctgtccaaggcgcccgtgtgggccgagcagcagcccatca
acacgtttgaaatgggcacacccaagctggcgtctctgacgttcgaggacggcgtggcccccgagcagatcttcgccgccgctgaaaagacctac
ccctggctgctggagtccggcgccaagtttgtggccaagcccgaccagctcatcaagcgacgaggcaaggccggcctgctggtactcaacaagt
cgtgggaggagtgcaagccctggatcgccgagcgggccgccaagcccatcaacgtggagggcattgacggagtgctgcgaacgttcctggtcg
agccctttgtgccccacgaccagaagcacgagtactacatcaacatccactccgtgcgagagggcgactggatcctcttctaccacgagggagga
gtcgacgtcggcgacgtggacgccaaggccgccaagatcctcatccccgttgacattgagaacgagtacccctccaacgccacgctcaccaagg
agctgctggcacacgtgcccgaggaccagcaccagaccctgctcgacttcatcaaccggctctacgccgtctacgtcgatctgcagtttacgtatct
ggagatcaaccccctggtcgtgatccccaccgcccagggcgtcgaggtccactacctggatcttgccggcaagctcgaccagaccgcagagtttg
agtgcggccccaagtgggctgctgcgcggtcccccgccgctctgggccaggtcgtcaccattgacgccggctccaccaaggtgtccatcgacgc
cggccccgccatggtcttccccgctcctttcggtcgagagctgtccaaggaggaggcgtacattgcggagctcgattccaagaccggagcttctct
gaagctgactgttctcaatgccaagggccgaatctggacccttgtggctggtggaggagcctccgtcgtctacgccgacgccattgcgtctgccgg
ctttgctgacgagctcgccaactacggcgagtactctggcgctcccaacgagacccagacctacgagtacgccaaaaccgtactggatctcatgac
ccggggcgacgctcaccccgagggcaaggtactgttcattggcggaggaatcgccaacttcacccaggttggatccaccttcaagggcatcatcc
gggccttccgggactaccagtcttctctgcacaaccacaaggtgaagatttacgtgcgacgaggcggtcccaactggcaggagggtctgcggttg
atcaagtcggctggcgacgagctgaatctgcccatggagatttacggccccgacatgcacgtgtcgggtattgttcctttggctctgcttggaaagcg
gcccaagaatgtcaagccttttggcaccggaccttctactgaggcttccactcctctcggagtttaa
Protein:
(SEQ ID NO: 154)
Msaksiheadgkallahflskapvwaeqqpintfemgtpklasltfedgvapeqifaaaektypwllesgakfvakpdqlikrrgkagllvlnk
sweeckpwiaeraakpinvegidgvlrtflvepfvphdqkheyyinihsvregdwilfyheggvdvgdvdakaakilipvdieneypsnatl
tkellahvpedqhqtlldfinrlyavyvdlqftyleinplvviptaqgvevhyldlagkldqtaefecgpkwaaarspaalgqvvtidagstkvsi
dagpamvfpapfgrelskeeayiaeldsktgaslkltvlnakgriwtlvagggasvvyadaiasagfadelanygeyspapnetqtyeyaktvl
dlmtrgdahpegkvlfigggianftqvgstfkgiirafrdyqsslhnhkvkiyvrrggpnwqeglrliksagdelnlpmeiygpdmhvsgivp
lallgkrpknvkpfgtgpsteastplgv
YALI0E14190g
DNA:
(SEQ ID NO: 155)
atggttattatgtgrtgtgggacctcagcacacgcatcatcccaacacagggtgcagtatatatagacagacgtgttccttcgcaccgttcttcacatatc
aaaacactaacaaattcaaaagtgagtatcatggtgggagtcaattgattgctcggggagttgaacaggcaacaatggcatgcacagggccagtga
aggcagactgcagtcgctgcacatggatcgtggttctgaggcgttgctatcaaaagggtcaattacctcacgaaacacagctggatgttgtgcaatc
gtcaattgaaaaacccgacacaatgcaagatctctttgcgcgcattgccatcgctgttgccatcgctgtcgccatcgccaatgccgctgcggattatta
tccctaccttgttccccgcttccgcacaaccggcgatgtctttgtatcatgaactctcgaaactaactcagtggttaaagctgtcgttgccggagccgct
ggtggtattggccagcccctttctcttctcctcaaactctctccttacgtgaccgagcttgctctctacgatgtcgtcaactcccccggtgttgccgctga
cctctcccacatctccaccaaggctaaggtcactggctacctccccaaggatgacggtctcaagaacgctctgaccggcgccaacattgtcgttatc
cccgccggtatcccccgaaagcccggtatgacccgagacgatctgttcaagatcaacgctggtatcgtccgagatctcgtcaccggtgtcgcccag
tacgcccctgacgcctttgtgctcatcatctccaaccccgtcaactctaccgtccctattgctgccgaggtcctcaagaagcacaacgtcttcaaccct
aagaagctcttcggtgtcaccacccttgacgttgtccgagcccagaccttcaccgccgctgttgttggcgagtctgaccccaccaagctcaacatcc
ccgtcgttggtggccactccggagacaccattgtccctctcctgtctctgaccaagcctaaggtcgagatccccgccgacaagctcgacgacctcgt
caagcgaatccagtttggtggtgacgaggttgtccaggctaaggacggtcttggatccgctaccctctccatggcccaggctggtttccgattgccg
aggctgtcctcaagggtgccgctggtgagaagggcatcatcgagcccgcctacatctaccttgacggtattgatggcacctccgacatcaagcgag
aggtcggtgtcgccttcttctctgtccctgtcgagttcggccctgagggtgccgctaaggcttacaacatccttcccgaggccaacgactacgagaa
gaagcttctcaaggtctccatcgacggtctttacggcaacattgccaagggcgaggagttcattgttaaccctcctcctgccaagtaa
Protein:
(SEQ ID NO: 156)
vvkavvagaaggigqplslllklspyvtelalydvvnspgvaadlshistkakvtgylpkddglknaltganivvipagiprkpgmtrddlfkin
agivrdlvtgvaqyapdafvliisnpvnstvpiaaevlkkhnvfnpkklfgvttldvvraqtftaavvgesdptklnipvvgghsgdtivpllsltk
pkveipadklddlvkriqfggdevvqakdglgsatlsmaqagfrfaeavlkgaagekgiiepayiyldgidgtsdikrevgvaffsvpvefgpe
gaakaynilpeandyekkllkvsidglygniakgeefivnpppak
YALI0E22649g
DNA:
(SEQ ID NO: 157)
atgactggcaccttacccaagttcggcgacggaaccaccattgtggttcttggagcctccggcgacctcgctaagaagaagaccgtgagtattgaa
ccagactgaggtcaattgaagagtaggagagtctgagaacattcgacggacctgattgtgctctggaccactcaattgactcgttgagagccccaat
gggtcttggctagccgagtcgttgacttgttgacttgttgagcccagaacccccaacttttgccaccatacaccgccatcaccatgacacccagatgt
gcgtgcgtatgtgagagtcaattgttccgtggcaaggcacagcttattccaccgtgttccttgcacaggtggtctttacgctctcccactctatccgagc
aataaaagcggaaaaacagcagaccatcccaacagacttctgctccgaataaggcgtctagcaagtgtgcccaaaactcaattcaaaaatgtcaga
aacctgatatcaacccgtcttcaaaagctaaccccagttccccgccctcttcggcctttaccgaaaacggcctgctgcccaaaaatgttgaaatcatcg
gctacgcacggtcgaaaatgactcaggaggagtaccacgagcgaatcagccactacttcaagacccccgacgaccagtccaaggagcaggcca
agaagttccttgagaacacctgctacgtccagggcccttacgacggtgccgagggctaccagcgactgaatgaaaagattgaggagtttgagaag
aagaagcccgagccccactaccgtcttttctacctggctctgccccccagcgtcttccttgaggctgccaacggtctgaagaagtatgtctaccccg
gcgagggcaaggcccgaatcatcatcgagaagccctttggccacgacctggcctcgtcacgagagctccaggacggccttgctcctctctggaa
ggagtctgagatcttccgaatcgaccactacctcggaaaggagatggtcaagaacctcaacattctgcgatttggcaaccagttcctgtccgccgtgt
gggacaagaacaccatttccaacgtccagatctccttcaaggagccctttggcactgagggccgaggtggatacttcaacgacattggaatcatcc
gagacgttattcagaaccatctgttgcaggttctgtccattctagccatggagcgacccgtcactttcggcgccgaggacattcgagatgagaaggtc
aaggtgctccgatgtgtcgacattctcaacattgacgacgtcattctcggccagtacggcccctctgaagacggaaagaagcccggatacaccgat
gacgatggcgttcccgatgactcccgagctgtgacctttgctgctctccatctccagatccacaacgacagatgggagggtgttcctttcatcctccg
agccggtaaggctctggacgagggcaaggtcgagatccgagtgcagttccgagacgtgaccaagggcgttgtggaccatctgcctcgaaatgag
ctcgtcatccgaatccagccctccgagtccatctacatgaagatgaactccaagctgcctggccttactgccaagaacattgtcaccgacctggatct
gacctacaaccgacgatactcggacgtgcgaatccctgaggcttacgagtctctcattctggactgcctcaagggtgaccacaccaactttgtgcga
aacgacgagctggacatttcctggaagattttcaccgatctgctgcacaagattgacgaggacaagagcattgtgcccgagaagtacgcctacggc
tctcgtggccccgagcgactcaagcagtggctccgagaccgaggctacgtgcgaaacggcaccgagctgtaccaatggcctgtcaccaagggct
cctcgtga
Protein:
(SEQ ID NO: 158)
mtgtlpkfgdgttivvlgasgdlakkktfpalfglyrngllpknveiigyarskmtqeeyherishyfktpddqskeqakkflentcyvqgpyd
gaegyqrlnekieefekkkpephyrlfylalppsvfleaanglkkyvypgegkariiiekpfghdlassrelqdglaplwkeseifridhylgke
mvknlnilrfgnqflsavwdkntisnvqisfkepfgtegrggyfndigiirdviqnhllqvlsilamerpvtfgaedirdekvkvlrcvdilnidd
vilgqygpsedgkkpgytdddgvpddsravtfaalhlqihndrwegvpfilragkaldegkveirvqfrdvtkgvvdhlprnelviriqpsesi
ymkmnsklpgltaknivtdldltynrrysdvripeayeslildclkgdhtnfvrndeldiswkiftdllhkidedksivpekyaygsrgperlkq
wlrdrgyvrngtelyqwpvtkgss
YALI0B15598g
DNA:
(SEQ ID NO: 159)
atgactgacacttcaaacatcaagtgagtattgccgcacacaattgcaatcaccgccgggctctacctcctcagctctcgacgtcaatgggccagca
gccgccatttgaccccaattacactggttgtgtaaaaccctcaaccacaatcgcttatgctcaccacagactacgacttaaccaagtcatgtcacaggt
caaagtaaagtcagcgcaacaccccctcaatctcaacacacttttgctaactcaggcctgtcgctgacattgccctcatcggtctcgccgtcatgggc
cagaacctgatcctcaacatggccgaccacggtaagtatcaattgactcaagacgcaccagcaagatacagagcatacccagcaatcgctcctctg
ataatcgccattgtaacactacgttggttagattgatctaaggtcgttgctggttccatgcacttccacttgctcatatgaagggagtcaaactctattttg
atagtgtcctctcccatccccgaaatgtcgcattgttgctaacaataggctacgaggttgttgcctacaaccgaaccacctccaaggtcgaccacttcc
tcgagaacgaggccaagggtgagtatccgtccagctatgctgtttacagccattgaccccaccttcccccacaattgctacgtcaccattaaaaaaca
aaattaccggtatcggcaagctagactttcatgcaacctacgcagggtaacaagttgagtttcagccgtgcaccttacaggaaaaccagtcatacgc
cgaggcagtgtgaaagcgaaagcacacagcctacggtgattgattgcatttttttgacataggagggaaacacgtgacatggcaagtgcccaacac
gaatactaacaaacaggaaagtccattattggtgctcactctatcaaggagctgtgtgctctgctgaagcgaccccgacgaatcattctgctcgttaag
gccggtgctgctgtcgattctttcatcgaacagctcctgccctatctcgataagggtgatatcatcattgacggtggtaactcccacttccccgactcca
accgacgatacgaggagcttaacgagaagggaatcctctttgttggttccggtgtttccggcggtgaggagggtgcccgatacggtccctccatcat
gcccggtggaaacaaggaggcctggccccacattaagaagattttccaggacatctctgctaaggctgatggtgagccctgctgtgactgggtcgg
tgacgctggtgccggccactttgtcaagatggttcacaacggtattgagtatggtgacatgcagcttatctgcgaggcttacgacctcatgaagcgag
gtgctggtttcaccaatgaggagattggagacgttttcgccaagtggaacaacggtatcctcgactccttcctcattgagatcacccgagacatcttca
agtacgacgacggctctggaactcctctcgttgagaagatctccgacactgctggccagaagggtactggaaagtggaccgctatcaacgctcttg
accttggtatgcccgtcaccctgatcggtgaggccgtcttcgctcgatgcctttctgccctcaagcaggagcgtgtccgagcttccaaggttcttgatg
gccccgagcccgtcaagttcactggtgacaagaaggagtttgtcgaccagctcgagcaggccctttacgcctccaagatcatctcttacgcccagg
gtttcatgcttatccgagaggccgccaagacctacggctgggagctcaacaacgccggtattgccctcatgtggcgaggtggttgcatcatccgatc
cgtcttccttgctgacatcaccaaggcttaccgacaggaccccaacctcgagaacctgctgttcaacgacttcttcaagaacgccatctccaaggcc
aacccctcttggcgagctaccgtggccaaggctgtcacctggggtgttcccactcccgcctttgcctcggctctggctttctacgacggttaccgatct
gccaagctccccgctaacctgctccaggcccagcgagactacttcggcgcccacacctaccagctcctcgatggtgatggaaagtggatccacac
caactggaccggccgaggtggtgaggtttcttcttccacttacgatgcttaa
Protein:
(SEQ ID NO: 160)
mtdtsnikpvadialiglavmgqnlilnmadhgyevvaynrttskvdhfleneakgksiigahsikelcallkrprriillvkagaavdsfieqll
pyldkgdiiidggnshfpdsnrryeelnekgilfvgsgvsggeegarygpsimpggnkeawphikkifqdisakadgepccdwvgdagag
hfvkmvhngieygdmqliceaydlmkrgagftneeigdvfakwnngildsflieitrdifkyddgsgtplvekisdtagqkgtgkwtainald
lgmpvtligeavfarclsalkqervraskvldgpepvkftgdkkefvdqleqalyaskiisyaqgfmlireaaktygwelnnagialmwrggci
irsvfladitkayrqdpnlenllfndffknaiskanpswratvakavtwgvptpafasalafydgyrsaklpanllqaqrdyfgahtyqlldgdgk
wihtnwtgrggevssstyda
YALI0D06303g
DNA:
(SEQ ID NO: 161)
atgctcaaccttagaaccgcccttcgagctgtgcgacccgtcactctggtgagtatctcggagcccgggacggctaccaacacacaagcaagatg
caacagaaaccggactttttaaatgcggattgcggaaaatttgcatggcggcaacgactcggagaaggagcgggacaattgcaatggcaggatgc
cattgacgaactgagggtgatgagagaccgggcctccgatgacgtggtggtgacgacagcccggctggtgttgccgggactgtctctgaaaagc
aatttctctatctccggtctcaacagactccccttctctagctcaattggcattgtcttcagaaggtgtcttagtggtatccccattgttatcttcttttcccca
atgtcaatgtcaatgtcaatggctccgacctctttcacattaacacggcgcaaacacagataccacggaaccgactcaaacaaatccaaagagacg
cagcggaataattggcatcaacgaacgatttgggatactctggcgagaatgccgaaatatttcgcttgtcttgttgtttctcttgagtgagttgtttgtgaa
gtcgtttggaagaaggttcccaatgtcacaaaccataccaactcgttacagccagcttgtaatcccccacctcttcaatacatactaacgcagacccg
atcctacgccacttccgtggcctctttcaccggccagaagaactccaacggcaagtacactgtgtctctgattgagggagacggtatcggaaccga
gatctccaaggctgtcaaggacatctaccatgccgccaaggtccccatcgactgggaggttgtcgacgtcacccccactctggtcaacggcaaga
ccaccatccccgacagcgccattgagtccatcaaccgaaacaaggttgccctcaagggtcccctcgccacccccatcggtaagggccacgtttcc
atgaacctgactctgcgacgaaccttcaacctgttcgccaacgtccgaccttgcaagtccgtcgtgggctacaagaccccttacgagaacgtcgac
accctgctcatccgagagaacactgagggtgagtactccggtatcgagcacaccgtcgtccccggtgtcgttcagtccatcaagctgatcacccga
gaggcttccgagcgagtcatccggtacgcttacgagtacgccctgtcccgaggcatgaagaaggtccttgttgtccacaaggcctctattatgaagg
tctccgatggtcttttccttgaggttgctcgagagctcgccaaggagtacccctccattgacctttccgtcgagctgatcgacaacacctgtctgcgaat
ggtccaggaccccgctctctaccgagatgtcgtcatggtcatgcccaacctttacggtgacattctgtccgatcttgcctccggtcttatcggtggtctt
ggtctgaccccctccggtaacatgggtgacgaggtctccatcttcgaggccgtccacggatccgctcccgacattgctggcaagggtcttgctaac
cccactgctctgctgctctcctccgtgatgatgctgcgacacatgggtctcaacgacaacgccaccaacatcgagcaggccgtctttggcaccattg
cttccggccccgagaaccgaaccaaggatcttaagggtaccgccaccacttctcactttgctgagcagattatcaagcgactcaagtag
Protein:
(SEQ ID NO: 162)
mlnlrtalravrpvtltrsyatsvasftgqknsngkytvsliegdgigteiskavkdiyhaakvpidwevvdvtptlvngkttipdsaiesinrnkv
alkgplatpigkghvsmnltlrrtfnlfanvrpcksvvgyktpyenvdtllirentegeysgiehtvvpgvvqsiklitreaserviryayeyalsrg
mkkvlvvhkasimkvsdglflevarelakeypsidlsvelidntclrmvqdpalyrdvvmvmpnlygdilsdlasgligglgltpsgnmgde
vsifeavhgsapdiagkglanptalllssvmmlrhmglndnatnieqavfgtiasgpenrtkdlkgtattshfaeqiikrlk

### Example 13

### Determination of Lipid Levels of Y. lipolytica

### 13A. Determination of Lipid Levels of Y. lipolytica in Various Growth Conditions of Varying Carbon to Nitrogen Ratios

Shake flask testing was conducted using carbon to nitrogen (C/N) ratios of 160, 80, 60, 40, 30, 20, and 10 with yeast nitrogen base being the base medium providing vitamins, trace elements and salts Ammonium sulfate (which contains 21% nitrogen) was used as the nitrogen source and glucose (which contains 40% carbon) was used as the carbon source at a concentration of 30 g/L. The concentrations of ammonium sulfate corresponding to these ratios are: 0.36, 0.71, 0.95, 1.43, 1.91, 2.86, and 4.6 g/L, respectively. Uracil was supplemented at 0.2 mM. As controls, strains were also grown in yeast extract-peptone with 50 g/L of glucose (media in which lipids do not accumulate at high levels) and yeast extract-peptone with 5% olive oil (v/v) (media in which lipids accumulate at high levels).

Strain MF760 (10-14 ml of culture) was harvested after 4 days of growth at 30° C., during which time the cultures were shaking at 250 rpm. Following harvesting, cells were washed three times with water, with the exception of the oil-grown cells which were washed three times in 0.5% BSA and one time with water before lipid extractions. Lipids were extracted as described in Folch J, Lees, M, and Stanley, G.H.S. J. Biol. Chem. 226: 497-509, 1957. In brief, cell pellets were resuspended in 6 ml of water. A 1 ml aliquot was transferred to a pre-weighed tube with a hole on the lid, spun down and the cell pellet lyophilized overnight to determine the dry cell weight. The remaining 5 ml were placed in a 15 ml Falcon tube and spun down. Cell pellets were frozen at −20° C. until extractions were performed.

Two to three volumes of a Zymolyase solution (2 mg/ml Zymolyase 100T in 1M Sorbitol, 50 mM EDTA and 0.01% β-mercaptoethanol) was added to each cell pellet and placed at 37° C. with constant agiatation for 1 hr. Two volumes of cubic zirconia beads were added to each tube and vortexed for 15-20 min. Samples were viewed under a microscope to ensure cell breakage before continuing with extractions. After cell breakage was complete, 6 ml of extraction solvent was added (a 2:1 mix of chloroform and methanol) and mixed. The mixture was spun down for 5 min at 3000 rpm and the organic layer was transferred to a clean tube. NaCl was added to the remaining aqueous layer to make it a 0.29% NaCl solution. 6 ml of extraction solvent was added and mixed, and the mixture was spun down for 5 min. The organic layers were pooled and filtered through a 0.2 μm filter to get rid of any cell debris. The extract was washed with 0.2 volumes of 0.29% NaCl solution and another 6 ml of extraction solvent added and mixed. Mixtures were spun and the organic layer was placed in a pre-weighed glass vial, the solvent was evaporated under a flow on nitrogen and the vial was weighed again to determine the weight of the lipid extracted. The dry cell weight is used to determine the percentage of lipid per dry cell weight. The lipid accumulation results are in the Table 48 below:

TABLE 48
Lipid accumulation under various carbon:nitrogen
ratio growth conditions

| C/N Ratio | | % lipid |
|---|---|---|
| YNB | 160 | 61 |
| 3% Glucose | 80 | 49 |
| | 60 | 34 |
| | 40 | 17 |
| | 30 | 16 |
| | 20 | 14 |
| | 10 | 15 |
| YEP | 5% Glucose | 22 |
| | 5% olive oil | 38 |

Other nitrogen sources tested were proline (12% nitrogen), sodium glutamate (7% nitrogen), soy acid hydrolysate (12% nitrogen), and yeast extract-peptone (26.8% nitrogen). All nitrogen sources tested at C/N ratios of 80 (with glucose as a carbon source), had significantly larger lipid bodies than at C/N ratios of 10 (also with glucose as a carbon source).

Strains MF858 and MF921 (Examples 2F and 2H) were harvested after 4 days of growth at 30° C. (3% glucose was used as the carbon source). Cells were washed three times with water and lipids extracted as described above. Lipid accumulation data for soy hydrolysate, yeast extract-peptone and yeast nitrogen base, used as a control, are listed in Table 49 below.

TABLE 49
Lipid accumulation under different carbon and
nitrogen conditions with various nitrogen sources

| | | % lipid | |
|---|---|---|---|
| | C/N Ratio | MF858 | MF921 |
| Soy hydrolysate | 80 | 36 | 36 |
| | 60 | 36 | 35 |
| | 10 | 14 | 15 |
| Yeast Extract- | 80 | 37 | 37 |
| Peptone | 10 | 15 | 14 |
| Yeast Nitrogen | 80 | 37 | 38 |
| Base | 10 | 13 | 11 |

### 13b. Determination of Lipid Levels Under High Carbon and Phosphate or Magnesium Limiting Conditions

To test whether other nutrient limitations, under high carbon conditions, will allow for higher lipid accumulation, phosphate or magnesium limiting conditions were tested. For phosphate limiting conditions, yeast nitrogen base medium without phosphate was prepared. Shake flask testing was performed using carbon to phosphate ratios ranging from 5376 down to 42. This range corresponds to 7.8 mg/L up to 1 g/L, respectively, and the latter concentration corresponds to that commonly used in yeast nitrogen base medium. Glucose, at 30 g/L, was used at the carbon source. Potassium phosphate monobasic (containing 28.7% phosphate) was used as the phosphate source.

For magnesium limiting conditions, yeast nitrogen base medium without magnesium was prepared. Shake flask testing was conducted using carbon to magnesium ratios ranging from 31360 down to 245. This range corresponds to 0.375 mg/L up to 0.5 g/L, and the latter magnesium concentration corresponds to that commonly used in yeast nitrogen base. Glucose, at 30 g/L, was used as the carbon source. Magnesium sulfate (containing 9.8% magnesium) was used as the magnesium source.

Strains MF858 and MF921 were harvested after 4 days of growth at 30° C., during which time the cultures were shaking at 250 rpm. Cells were washed three times with water before lipid extraction. Lipids were extracted as described above. Lipid accumulation data is listed in Table 50 below:

TABLE 50
Lipid accumulation in phosphate or
magnesium limiting growth conditions

| | | % Lipid | |
|---|---|---|---|
| | g/L | MF858 | MF921 |
| phosphate | 1 | 14 | 14 |
| | 0.0625 | 18 | 20 |
| | 0.0313 | 34 | 41 |
| | 0.0156 | 62 | 63 |
| | 0.0078 | 83 | 76 |
| magnesium | 0.5 | 12 | 11 |
| | 0.0313 | NA | 16 |
| | 0.0156 | NA | 25 |
| | 0.0078 | NA | 42 |
| | 0.0039 | 48 | 48 |

### Example 14

### Effect of Temperature on Carotenoid Production

MF740 was transformed with pMB4719 with SalI, and a Ura⁺ colony was designated ML878. MF740 was transformed with pMB4629 cleaved with SalI, an Ade⁺ colony was designated ML857, and subsequently transformed with pMB4719 cleaved with SalI, to create ML836. ML878 and ML836 were grown for 4 days in YPD at 20° C., 24° C., and 28° C., and carotenoids were extracted and analyzed by HPLC. β-carotene or zeaxanthin yield (% dry cell weight) at 20° C. was chosen as a standard against which yields at other temperatures were compared. In addition, the ratio of zeaxanthin/carotenoid (% dry cell weight) was calculated for each temperature. Whereas the β-carotene levels fell with decreasing temperatures, the ratio of zeaxanthin to β-carotene increased with lower temperatures (Table 51).

TABLE 51
Effect of Temperature on carotenoid production

| | | | | Zeaxanthin/β-carotene |
|---|---|---|---|---|
| | Temperature | β-carotene | Zeaxanthin | ratio (% dry cell |
| Strain | (° C.) | yield* | yield** | weight)*** |
| MF878 | 20 | 1.0 | 1.0 | 2.4 |
| MF878 | 24 | 1.3 | 1.0 | 1.9 |
| MF878 | 28 | 3.2 | 1.5 | 1.1 |
| MF836 | 20 | 1.0 | 1.0 | 1.0 |
| MF836 | 24 | 1.9 | 1.2 | 0.7 |
| MF836 | 28 | 3.4 | 1.1 | 0.4 |

*β-carotene yield is calculated as % DCW β-carotene at 20° C. divided by % DCW β-carotene at each temperature
**Zeaxanthin yield is calculated as % DCW zeaxanthin at 20° C. divided by % DCW zeaxanthin at each temperature
***Zeaxanthin/β-carotene ratio is calculated as % DCW zeaxanthin divided by % DCW β-carotene

### Example 15

### Construction of a Recyclable URA3 Marker Plasmid pMB5082

To create a selectively excisable (“recyclable”) URA3 marker, an 860 bp SpeI-SacI (blunt ended with T4 DNA ligase) fragment (containing the URA3 promoter and the first 121 nucleotides of the URA3 gene) from plasmid pMB4691 was inserted into the SpeI-NotI sites of plasmid pMB4534 to create pMB5055.

The URA3 promoter was excised from pMB5055 as an 878 bp fragment by XbaI-SpeI digest, and was ligated into XbaI-cleaved pMB4691. Orientation of the promoter was verified by restriction digest. The resulting plasmid, designated pMB5082, contained the URA3 promoter both upstream of the URA3 gene and downstream of its terminator. This cassette, once integrated into the Yarrowia genome, permits excision of the URA3 marker by homologous recombination between the two copies of the URA3 promoter. Colonies containing the excision may be selected on 5-FOA.

### Example 16

### Effects of Mutations in the Transcriptional Regulator, SPT8 on Carotenoid Production

Y. lipolytica strain ML1018 was isolated by plasmid insertion mutagenesis. ML1018 was darker in hue, shiny, exclusively yeast-form rather than partial mycelial morphology and exhibited increased carotenoid levels when compared to its sibling transformants. Sequence analysis identified the site of ML1018 plasmid insertion between base pairs 701 and 702 of the SPT8 coding sequence. Experiments were undertaken to examine carotenoid levels in a targeted SPT8 disruption strain.

A 2.5 kb fragment containing the SPT8 gene (YALI0E23804g) with its endogenous promoter and terminator was amplified from genomic DNA isolated from Y. lipolytica strain NRRL Y-1095 using primers: MO5651 (5′-CACAAACTAGTGTCAGGAATATGAAACCAGG-3′) (SEQ ID NO:163) and MO5652 (5′-CACAAACTAGTGCATGTGATAGGAAGGAGGA-3′) (SEQ ID NO:164). Plasmid pMB5083 was constructed by phosphorylating the 2.5 kb SPT8 fragment with T4 polynucleotide kinase and ligating the phosphorylated fragment with desphosphorylated, EcoRV-digested pBluescriptSK-.

A 3.4 kb fragment containing the TEF1 promoter, XPR terminator, and a recyclable URA3 marker was isolated from plasmid pMB5082 by Acc65I and XbaI (subsequently made blunt with Klenow) digestion. This fragment was cloned into the BsiWI and SmaI sites of pMB5083 to create pMB5086. BamHI-XbaI digestion of pMB5086 yields a 5.6 kb Y. lipolytica SPT8 disruption fragment containing the TEF1 promoter and XPR terminators followed by a recyclable URA3 marker between base pairs 752 and 753 of the SPT8 coding sequence (SPT8:: URA3 disruption cassette).

A 3.6 kb fragment containing the XPR terminator and ADE1 gene was excised from plasmid pMB4629 by MluI and EcoRV digest and subsequently cloned into MluI-PmlI-digested pMB5086. The resulting plasmid, pMB5124, contains a 5.8 kb BamHI-XbaI SPT8 disruption cassette similar to that in pMB5086, with the distinction that the recyclable URA3 marker is replaced with a non-recyclable ADE1 marker (SPT8::ADE1 disruption cassette).

Y. lipolytica strains MF740 and MF746 (both ade1 ura3) are transformed with a 5.8 kb BamHI-XbaI fragment from pMB5124 (spt8::ADE1). spt8 disruptants are distinguished from ectopic integrants by colony morphology, as spt8 strains are shinier, darker in hue, and less mycelial than SPT8 strains. Correct integration may be assayed by PCR or by Southern blotting. Carotenoid yield is assayed in spt8 disrupted and SPT8⁺ strains by harvesting carotenoids after a four-day fermentation in YPD shake flasks at 30° C.

### Example 17

### Construction Plasmid pMB4844 Encoding a Chimeric β-Carotene Hydroxylase-Ketolase (crtZW)

A β-carotene hydroxylase: β-carotene ketolase chimera is constructed as follows. First, a 0.5 kb fragment containing crtZ from Erythrobacter litoralis is amplified from pMB4715, a plasmid containing a copy of the crtZ gene, using primers MO4814: 5′-CACAACGTCTCTCTAGACACAAAAATGAGCT-3′ (SEQ ID NO:165) and MO4816: 5′-CACAACGTCTCAGCCGGCACCTGCTCCCATAGAATCTCG-3′ (SEQ ID NO:166) and the resulting fragment is digested with XbaI and BsmBI. Similarly, a 0.8 kb fragment containing crtW from Parvularcula bermudensis is amplified from pMB4731, a plasmid containing a copy of the crtW gene, with primers MO5060: 5′-CACAAGAAGACAACGGCGCAGGAGCCATGGACCCTACCGGAGACG-3′ (SEQ ID NO:167) and MO5061: 5′-CACAAGAAGACAACGCGTTTAAGGGCCGGTTCTCTTTC-3′ (SEQ ID NO:168) and the resulting fragment is digested with Bbsl and MluI. The digested fragments containing the crtZ and crtW genes are then ligated in a three-piece reaction into NheI-MluI cleaved vector pMB4691 to create pMB4844. Sequence analysis confirms the creation of an in-frame fusion of crtZ and crtW placed under control of the TEF1 promoter and the XPR terminator. The chimeric sequence is designated crtZW. The amino acid sequence of crtZW is:

(SEQ ID NO: 169)
mswwaialivfgavvgmeffawfahkyimhgwgwswhrdhhephdntlekndlfavvfgsvaallfvigalwsd
plwwaavgitlygviytlvhdglvhqrywrwtpkrgyakrlvqahrlhhatvgkeggvsfgfvfardpaklkaelkqqreqglavvrdsmg
agagagamdptgdvtasprpqttipvrqalwglslagaiiaawvfmhigfvffapldpivlalapviillqswlsvglfiishdaihgslapgrpa
fnramgrlcmtlyagfdfdrmaaahhrhhrspgtaadpdfsvdspdrplpwfgaffrryfgwrpfltvnavvftywlvlganpvnivlfygv
pallsagqlfyfgtflphrherqgfadhhrarsvrspymlslvtcyhfggyhhehhlfphepwwrlpqrggwerdrrkrtgp

### Example 18

### pH Effects on Total Carotenoid Yield And Hydroxylation of Beta-Carotene

The effect of altering pH on total carotenoid yield and relative amount of individual carotenoids was investigated. Strain ML1011 (MF740 transformed with multiple integrated copies of the X. autotrophicus crtZ gene) which accumulates a mixture of carotenoids comprising beta-carotene, beta-cryptoxanthin, and zeaxanthin was fermented under the following parameters.

Batch medium: YPD

Temperature setpoint: 30° C.

Initial volume: 210 ml

Vessel volume: 400 ml

Agitation rate: 1000 rpm

Feed: 40% glucose

Feed rate: 2 ml/hour, starting at 24th hour after inoculation

Four separate fermentor units were setup and the pH was controlled as follows:
Unit 1: pH 5.5
Unit 2: pH 7.0
Unit 3: pH 7.0 at inoculation, continuously rising to a setpoint of pH 8.0 at 48 hours (change of 0.021 pH units/hour)
Unit 4: pH 7.0 at inoculation, continuously rising to a setpoint of pH 9.0 at 48 hours (change of 0.042 pH units/hour

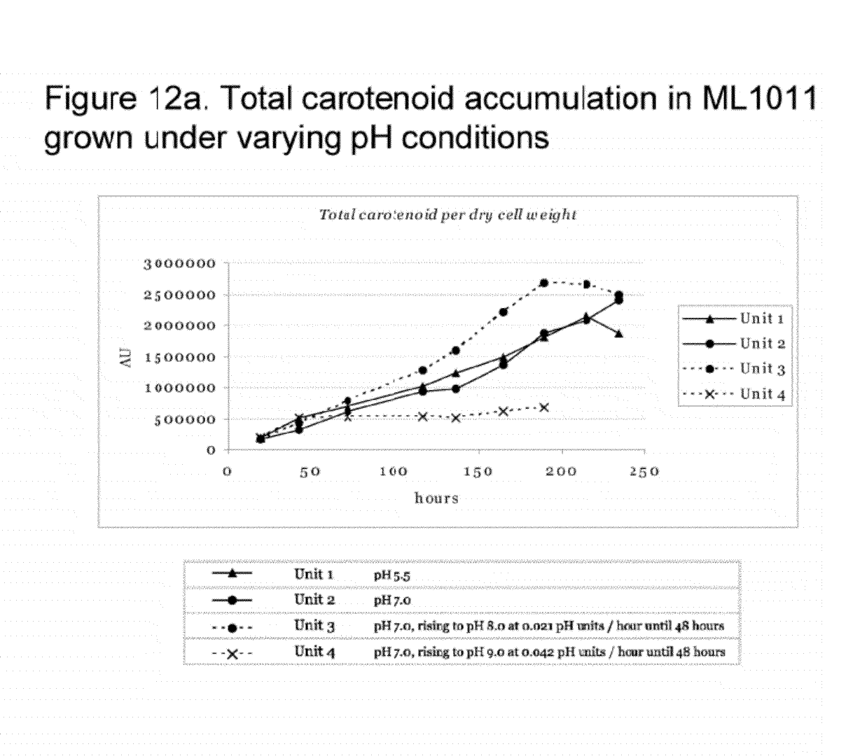

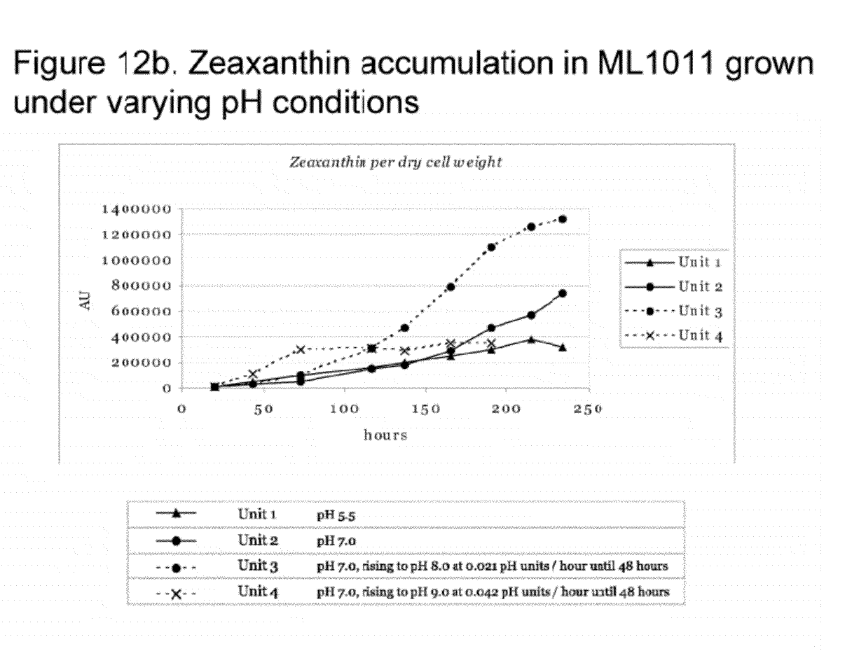

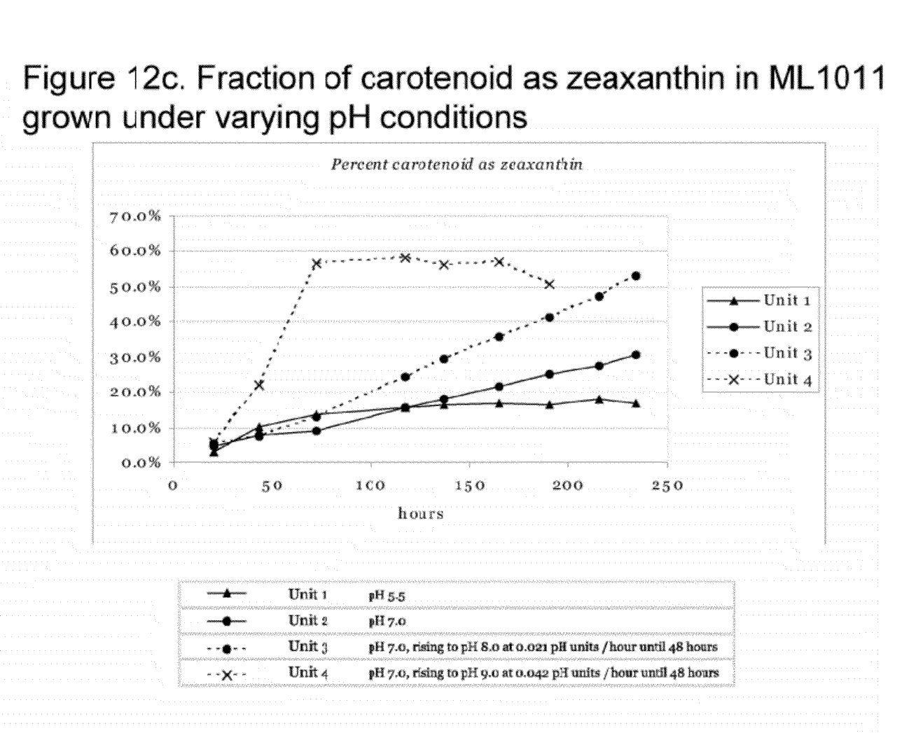

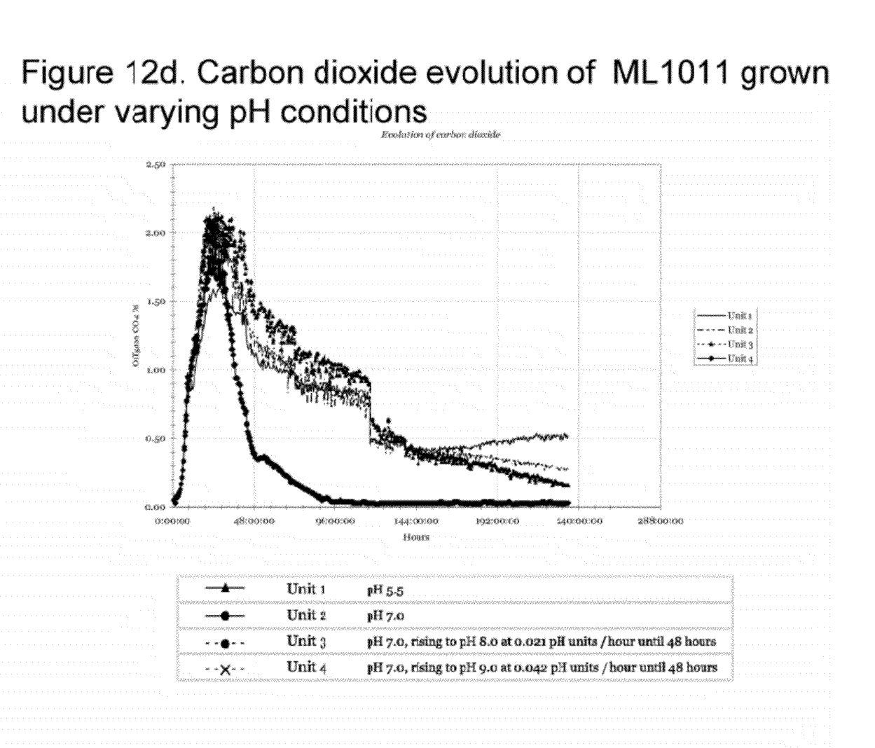

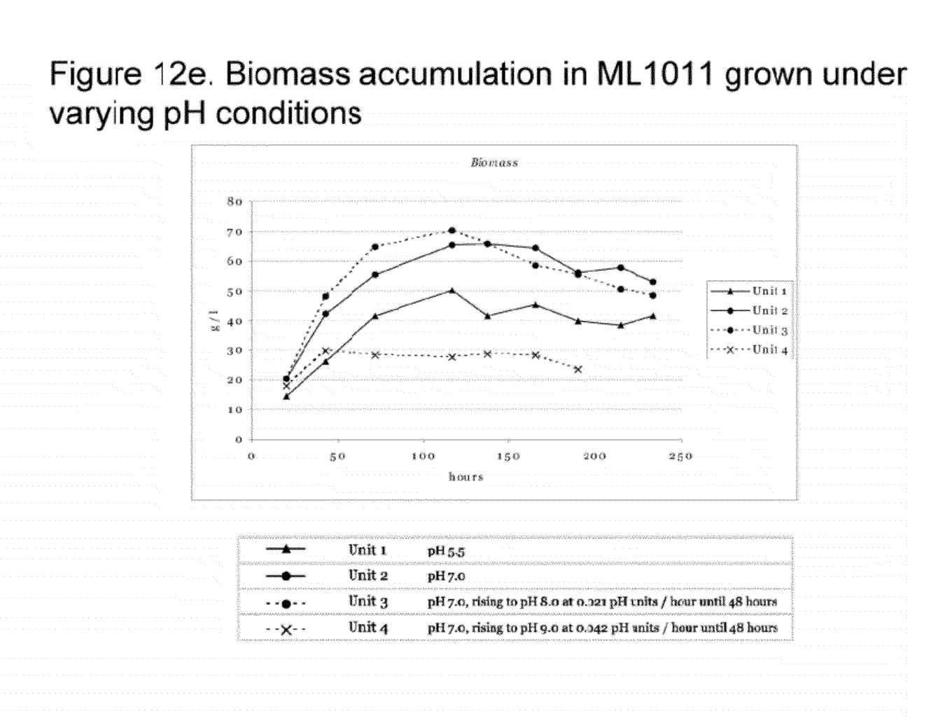

Additionally, the glucose feed of unit 4 was halted at 64 hours (see below). FIG. 12a depicts accumulation of total carotenoid (absorbance units per unit dry cell weight) throughout the fermentation. Excluding the last timepoint, units 1 and 2 accumulated similar amounts of carotenoid throughout the run. This result, consistent with previous experiments, suggests that varying the pH in the range of 5.5-7.0 does not affect total carotenoid yield. During this same period, fermentor unit 3 accumulated more carotenoid than units 1 and 2, suggesting setting pH to be within the range of 7.0-8.0 improves the rate of carotenoid biosynthesis. In unit 4, carbon dioxide evolution (indicating metabolic activity) and carotenoid accumulation started to fall precipitously when the unit reached pH 8.3 at approximately 31 hours (FIG. 12d), suggesting toxicity due to high pH. The feed to this unit was therefore stopped at 64 hours. Together, these results suggest that carotenoid yield may be maximized by maintaining pH within the range 7.0-8.0, while pH levels below or above this range were ineffective or toxic, respectively.

FIG. 12b depicts accumulation of zeaxanthin (absorbance units per dry cell weight; AU) over the course of the fermentation. As seen in FIG. 12b, zeaxanthin accumulation improves with increasing pH. Unit 4, at highest pH, shows superior zeaxanthin accumulation compared to all other units until hour 64, when its feed was terminated. Likewise, unit 3, maintained at pH 8.0 after hour 48, shows significantly improved zeaxanthin accumulation over units 1 and 2, maintained at pH 5.5 and 7.0, respectively. These results indicate that hydroxylation of beta carotene to zeaxanthin is favored by higher pH.

FIG. 12c depicts the fraction of carotenoid as zeaxanthin (AU zeaxanthin/AU total carotenoid) throughout the course of the fermentation. Unit 3 hydroxylated a greater fraction of beta-carotene than units 1 and 2, in addition to producing more total carotenoid (FIG. 12a) and more total zeaxanthin (FIG. 12b). This result demonstrates that at pH 8.0, zeaxanthin accumulation outpaced the global increase in carotenoid biosynthesis also seen at this pH

As seen in FIG. 12e, biomass accumulated fastest in unit 3 and remained above all other units until the hundred-thirtieth or so hour of fermentation. This unit similarly was the most metabolically active, as shown by its increased rate of carbon dioxide evolution compared to the other units over the same time period (FIG. 12d). The subsequent decline in biomass in this unit may be attributed to accelerated metabolism of carbon stored as intracellular oil, relative to the other three units. Thus, it appears that the pH range of 7.0-8.0 enables Yarrowia lipolytica both to accumulate biomass and metabolize stored carbon at rates faster than it is able at lower pH.

Together, these results indicate that total biomass accumulation, percentage of biomass representing carotenoid accumulation, and the hydroxylation of beta-carotene to zeaxanthin may be manipulated by maintaining fermentation pH in the approximate range of 7.0-8.0. Moreover, these results suggest that within this same range, an optimum pH may be selected at which to maximize production of both non-oxygenated carotenoids and xanthophylls (e.g., hydroxylation of β-carotene to zeaxanthin and total carotenoid production).

### Example 19

### Lycopene Epsilon Cyclase Sequences

The DNA and proteins they encode of the certain lycopene epsilon cyclase sequences are provided below. Corresponding Genbank Accession and GI numbers are found in Table 23.

Ostreococcus lucimarinus sequence XP_{—}001422490

DNA
(SEQ ID NO: 170)
ATGAAGGATGATCGCGAATGGATTGCGTTTCAACAGCGCAAGGTGTTTAGTGAGCAAAA
GCAAATCAAAGAGTACCTCAGTGCTTTGAACGACCGCGACAAGGTCGACGTTCTCGTTGT
CGGTGCGGGCCCCGCAGGTCTGGCGATCGCAGCGGAGACGGCGAAGAAGGGTCTTTCTG
TTGGTCTCGTCGCACCAGACACCCCGTTCGTGAACAACTACGGAGTATGGCTCGACGAGT
TCAAAGATCTAGGGCTCGAACACTGCTTGCTTCATAAGTATGACGACGCATTGGTTTGGT
TCGATGATTCTGATCCTGCGAGTGGAACTGAACTCGGTCGACCTTACGGTCAAGTGTGCC
GCAGGCGTCTTCGCGACCATTTGTTGAAGGAGTGCGCGGCGGCTGGCGTCAAGTATTTAC
CAGGCCTGGTAGATTTTGTGCGTCACGGTGACGTCGAAAAGAACGAGTTAGCCGAAGCA
AACAGAGGCCAGCAATTCACGTTGAATTCGCGTCTCGTCGTTGCCGGCACCGGTCACAAC
CGCGACATGCTCAGCTACGAAGAGGGTGCGCCGCCGGGCTGGCAGACTGCGTATGGCGT
TGAGGTGCGCATTCCGAACCACGGTTTTCCCGTGAACAAGGCCGTGTTCATGGATTTTCG
TCAAAGCGATCCGGAGGCGATGAAAGAGGAACAAGACGAGGGCGTTTGGCGCGTGCCG
TCTTTCCTTTACGTGTTACCCGTGGACAAGGATGTGGTGTTCGTCGAGGAGACGTGCCTC
GTCGCGCGCGTACAAGTGCCGTTCGATGAACTCAAACGGCGATTGTATCGTCGTATGAAG
CGGATGGGTATGGAAATCGTCGAAGAAGACATCTTGGAAGTCGAGGCGAGTTGGATTCC
ACTGGGCGGTACCCCGCCGGTTGCCCCGCAACGCACCATCGCGTACGGTGCAGCAGCCG
GCATGGTCCACCCTGCGTCTGGCTACTCCGTCGTAAACAGTATTAGCAAAGCTCCGCGTG
TTGCGACGGCCATGGCCGAAGGCTTGAAGGAGGGTGGCGAGATTGAGGCGAGCCGAAG
AGCGTGGGAAATCCTTTGGGGTGCGGAGCCACGAAGACAAATCGGTTTCTACCAGTTCG
GTATGGAGCTTCTCATGTCGCTTCGCATCGAGCAGATGCGCAACTTCTTTAGTACCTTCTT
TGCGCTTCCAACAAATCTGAGCAGAGGATTTTTGGGTAACAGATTGTCGAGCTCAGAGTT
GATCATGTTTGCTCTCACTACGTTCGCAATTGGTAACAACGAACTTCGTGGGTTGTTGCTC
GCTCACCTGGTTTCA
Protein
(SEQ ID NO: 171)
MKDDREWIAFQQRKVFSEQKQIKEYLSALNDRDKVDVLVVGAGPAGLAIAAETAKKGLSVG
LVAPDTPFVNNYGVWLDEFKDLGLEHCLLHKYDDALVWFDDSDPASGTELGRPYGQVCRR
RLRDHLLKECAAAGVKYLPGLVDFVRHGDVEKNELAEANRGQQFTLNSRLVVAGTGHNRD
MLSYEEGAPPGWQTAYGVEVRIPNHGFPVNKAVFMDFRQSDPEAMKEEQDEGVWRVPSFL
YVLPVDKDVVFVEETCLVARVQVPFDELKRRLYRRMKRMGMEIVEEDILEVEASWIPLGGTP
PVAPQRTIAYGAAAGMVHPASGYSVVNSISKAPRVATAMAEGLKEGGEIEASRRAWEILWG
AEPRRQIGFYQFGMELLMSLRIEQMRNFFSTFFALPTNLSRGFLGNRLSSSELIMFALTTFAIGN
NELRGLLLAHLVS

lycopene epsilon cyclase (Diospyros kaki) sequence BAE94036

DNA
(SEQ ID NO: 172)
ACTACGGCGTATGGGAGGATGAATTTAGAGATCTTGGACTTGAAAGGTG
TATTGAACATGTTTGGAGAGACACAATTGTATATCTTGATGACAATGAT
CCCATTCTGATTGGTCGTGCTTATGGACGAGTTAGTCGTCACTTGCTCC
ACGAGGAGCTATTAAGAAGGTGTGTGGAGTCAGGTGTTTCATATTTGAG
CTCAAAAGTGGAAAGAATTATTGAAACTACGAATGGGCAGAGTCTCATA
GAGTGCGGAACTGATGTTGTTGTCCCATGCAGGCTTGCTACTGTTGCTT
CGGGAGCAGCTTCTGGGAAACTTTTGAAGTTTGAGGTGGGAGGACCCAG
AGTTTCTGTTCAAACAGCTTATGGTGTGGAGGTTGAGGTGGAAAACAAT
CCATATGACCCCAACTTGATGGTTTTCATGGATTACAGAGACTATGCCA
AACAAAAAGTTCAGCCTTTGGAAGCACAATATCCAACATTTCTTTATGC
CATGCCTATGTCCCCTACAAGAGTCTTCTTTGAGGAAACTTGTTTGGCT
TCAAAGGATGCCATGCCTTTTGATCTATTAAAGAGGAAACTCATGGACA
GATTAGAGACAATGGGAGTCCATGTTCTAAAAACGTATGAGGAGGAATG
GTCTT
Protein
(SEQ ID NO: 173)
YGVWEDEFRDLGLERCIEHVWRDTIVYLDDNDPILIGRAYGRVSRHLLH
EELLRRCVESGVSYLSSKVERIIETTNGQSLIECGTDVVVPCRLATVAS
GAASGKLLKFEVGGPRVSVQTAYGVEVEVENNPYDPNLMVFMDYRDYAK
QKVQPLEAQYPTFLYAMPMSPTRVFFEETCLASKDAMPFDLLKRKLMDR
LETMGVHVLKTYEEEWS

### Example 20

### Construction of a Lutein Producing Strain

The following sequence, optimized for Y. lipolytica codon bias and encoding a putative lycopene epsilon cyclase from Ostreococcus lucimarinus CCE9901, is synthesized de novo:

(SEQ ID NO: 174)
TTCTAGAACAAAATGAAGGACGACCGAGAGTGGATCGCCTTCCAGCAGCGAAAGGTGTT
CTCTGAGCAGAAGCAGATCAAGGAGTACCTGTCTGCCCTGAACGACCGAGACAAGGTGG
ACGTGCTGGTGGTGGGCGCCGGCCCCGCCGGCCTGGCCATCGCCGCCGAGACCGCCAAG
AAGGGCCTGTCTGTGGGCCTGGTGGCCCCCGACACCCCCTTCGTGAACAACTACGGCGTG
TGGCTGGACGAGTTCAAGGACCTGGGCCTGGAGCACTGTCTGCTGCACAAGTACGACGA
CGCCCTGGTGTGGTTCGACGACTCTGACCCCGCCTCTGGCACCGAGCTGGGCCGACCCTA
CGGCCAGGTGTGTCGACGACGACTGCGAGACCACCTGCTGAAGGAGTGTGCCGCCGCCG
GCGTGAAGTACCTGCCCGGCCTGGTGGACTTCGTGCGACACGGCGACGTGGAGAAGAAC
GAGCTGGCCGAGGCCAACCGAGGCCAGCAGTTCACCCTGAACTCTCGACTGGTGGTGGC
CGGCACCGGCCACAACCGAGACATGCTGTCTTACGAGGAGGGCGCCCCCCCCGGCTGGC
AGACCGCCTACGGCGTGGAGGTGCGAATCCCCAACCACGGCTTCCCCGTGAACAAGGCC
GTGTTCATGGACTTCCGACAGTCTGACCCCGAGGCCATGAAGGAGGAGCAGGACGAGGG
CGTGTGGCGAGTGCCCTCTTTCCTGTACGTGCTGCCCGTGGACAAGGACGTGGTGTTCGT
GGAGGAGACCTGTCTGGTGGCCCGAGTGCAGGTGCCCTTCGACGAGCTGAAGCGACGAC
TGTACCGACGAATGAAGCGAATGGGCATGGAGATCGTGGAGGAGGACATCCTGGAGGTG
GAGGCCTCTTGGATCCCCCTGGGCGGCACCCCCCCCGTGGCCCCCCAGCGAACCATCGCC
TACGGCGCCGCCGCCGGCATGGTGCACCCCGCCTCTGGCTACTCTGTGGTGAACTCTATC
TCTAAGGCCCCCCGAGTGGCCACCGCCATGGCCGAGGGCCTGAAGGAGGGCGGCGAGAT
CGAGGCCTCTCGACGAGCCTGGGAGATCCTGTGGGGCGCCGAGCCCCGACGACAGATCG
GCTTCTACCAGTTCGGCATGGAGCTGCTGATGTCTCTGCGAATCGAGCAGATGCGAAACT
TCTTCTCTACCTTCTTCGCCCTGCCCACCAACCTGTCTCGAGGCTTCCTGGGCAACCGACT
GTCTTCTTCTGAGCTGATCATGTTCGCCCTGACCACCTTCGCCATCGGCAACAACGAGCT
GCGAGGCCTGCTGCTGGCCCACCTGGTGTCTTAAACGCGT

This fragment, liberated with XbaI and MluI, is cloned into NheI- and MluI-cleaved pMB5082 to produce pEpCyOs1.

A second putative lycopene epsilon cyclase from Ostreococcus lucimarinus CCE9901 is similarly codon-optimized and synthesized de novo:

(SEQ ID NO: 175)
TTCTAGAACAAAATGCGAGCCCGACGAGCCCCCGCCGCCCGAGTGACCCGAGCCATCCG
AGCCCGAGGCGACGCCGGCACCCGAGCCCGAGACGTGGCCCCCGGCGCCACCCGACGAG
GCGCCTCTGCCACCCCCCGAGCCACCCGACGACCCTCTGCCCGAGAGACCCGACCCGAG
CTGTACGGCCTGGACGCCTCTTGGGACCCCCTGACCTCTGGCGACCGACGAGAGTCTGAG
GAGTCTCGAACCCCCCTGCCCGAGACCCTGCCCAACGTGCGATGGGGCACCTCTGCCTCT
GAGGCCTACGACCTGGTGATCGTGGGCTGTGGCCCCGCCGGCCTGACCGCCGCCGACGA
GGCCTCTAAGCGAGGCCTGCGAGTGGCCCTGATGGACCCCTCTCCCCTGGCCCCCTGGAT
GAACAACTACGGCGTGTGGTGTGACGAGTTCAAGTCTCTGGGCTTCGACGACTGTTACCG
AGCCGTGTGGAACAAGGCCCGAGTGATCATCGACGACGGCGACGCCGACGGCAAGATGC
TGGACCGAGCCTACGCCCAGGTGGACCGAAAGAAGCTGAAGCAGAAGCTGATCGCCCGA
TCTGTGACCCAGGGCGTGGAGTTCGGCATCGCCGCCGTGGACTCTTGTGACAACTCTGAC
CCCAACCACTCTGTGGTGACCCTGTCTGACGGCCGAAAGGTGTACGCCAAGATGGTGCTG
GACGCCACCGGCCACTCTCGAAAGCTGGTGGACTTCGACCGAGACTTCACCCCCGGCTAC
CAGGCCGCCTTCGGCATCGTGTGTACCGTGGAGAAGCACGACTTCCCCCTGGACACCATG
CTGTTCATGGACTGGCGAGACGAGCACCTGTCTCCCGAGTTCAAGCGAGCCAACGACCG
ACTGCCCACCTTCCTGTACGCCATGCCCTTCTCTGAGACCGAGGTGTTCCTGGAGGAGAC
CTCTCTGGTGGCCCGACCCGGCCTGGAGTTCGACGACCTGAAGCTGAAGCTGAAGGAGC
GACTGGACTACCTGGGCGTGAAGGTGACCAAGGTGCACGAGGAGGAGTACTGTCTGATC
CCCATGGGCGGCGTGCTGCCCACCTTCCCCCAGCGAACCCTGGGCATCGGCGGCACCGCC
GGCATGGTGCACCCCTCTACCGGCTTCATGGTGGCCAAGACCATGCTGTGTGTGCGAACC
CTGGTGGGCACCCTGGACGAGGCCCTGAAGGCCGGCAAGCGAGGCGACATCACCGGCGC
CCTGGAGGCCGCCGAGGCCGCCCAGATGAACAACGGCAAGTTCGACGCCGACGCCACCG
CCGCCCTGGTGTGGAACTCTATCTGGCCCGAGAACGACCTGCGAATGCGAACCTTCATGT
GTTTCGGCATGGAGACCCTGATGCAGCTGGACATCGACGGCACCCGACAGTTCTTCGACA
CCTTCTTCGACCTGCCCAAGGACGTGTGGGCCGGCTTCCTGTCTTGGCGAATCCAGCCCG
TGGGCCTGCTGTCTCTGGGCGTGAACCTGTTCGCCCTGTTCTCTAACTACATGCGAGTGA
ACTTCGTGAAGTCTGCCCTGCCCTTCATGGGCTCTTTCTTCGCCAACTAAACGCGT

This fragment, liberated with XbaI and MluI, is cloned into NheI- and MluI-cleaved pMB5082 to produce pEpCyOs2.

The following sequence, optimized for Y. lipolytica codon bias and encoding a putative carotene epsilon hydroxylase from Ostreococcus tauri, is synthesized de novo:

(SEQ ID NO: 176)
TTCTAGAACAAAATGAAGGACGGCCAGGACGAGGACTCTGACGAGATCTGGGGCGGCCA
GCGACACGCCTCTGAGATGAAGACCCCCACCCGACGAAAGGCCCGAACCAAGGCCGAGC
GAGAGGCCTCTGCCGCCTCTTACGAGTGGTCTGCCTGGGCCTCTTCTTGTGGCGTGATCTC
TGTGGCCATCACCGCCACCTACTTCCGAATCCTGCGAGAGGTGGACGTGAACGGCGGCG
TGTTCCCCGTGGCCGAGCTGGTGGCCCAGCTGGCCCTGATCGCCGGCGCCGCCGTGGGCA
TGGAGTTCTACGCCCGATACGCCCACAAGCACCTGTGGCACGGCTCTTGGTGGACCATGT
CTAACAAGTACCGACAGGAGTGGAACCGACCCATCTGGCTGCTGCACGAGTCTCACCAC
CTGCCCCGAGAGGGCGCCTTCGAGGCCAACGACGTGTTCGCCCTGATGAACGGCGTGCC
CGCCTTCGCCCTGTGTGCCTTCGGCTTCTTCACCCCCGGCGTGTTCGGCGGCCTGTGTTTC
GGCGCCGGCCTGGGCATCACCCTGTTCGGCATCGCCTACATGTACGTGCACGACGGCCTG
GTGCACAAGCGATTCCCCACCGGCCCCCTGGGCAAGCTGCCCGTGATGCGACGAATCGC
CGCCGGCCACACCATCCACCACACCGAGGCCTTCGAGGGCGTGCCCTGGGGCCTGTTCCT
GGGCATCCAGGAGCTGGCCGCCGTGCCCGGCGGCCTGGAGGAGCTGGAGAAGGTGGTGA
TCGCCGCCGAGCGAAAGGAGAAGCGAGACGAGCTGGAGCTGGCCCGACGAGCCTCTGTG
GGCCTGGTGACCGAGGGCGCCCACATCCCCTCTATGAAGGAGGCCCCCCAGTGTAAGCT
GCCCGAGGACCCCTAAACGCGT

This fragment, liberated with XbaI and MluI, is cloned into NheI- and MluI-cleaved pMB5082 to produce pEpHyOs1.

The 1.9 kb KpnI-SacI TEF1p-crtZ fragment from pMB4837 (Example 1O) is cloned into KpnI- and SacI-cleaved pMB5082 to create pCrtZ-Ub.

A strain expressing carRP, carB, GGS, and HMG1_{trunc} and auxotrophic for ura3 (MF946; Example 2F) is transformed successively, in any order, with the URA3 plasmids pEpCyOs1 (or pEpCyOs2), pEpHyOs1, and pCrtZ-Ub, with the recycling of the ura3 marker between each step, as described in Example 15. Such a strain is expected to produce >1 mg/g DCW lutein. This strain may be further modified by transformation with pMB4789 (erg9[F3171]-3′UTR:: URA3), as described in Example 2H.

The following tables are referenced throughout the description. Each reference and information designated by each of the Genbank Accession and GI numbers are hereby incorporated by reference in their entirety. The order of genes, polypeptides and sequences presented in the tables is not indicative of their relative importance and/or suitability to any of the embodiments disclosed herein.

Lengthy table referenced here
US20120149886A1-20120614-T00001
Please refer to the end of the specification for access instructions.

Lengthy table referenced here
US20120149886A1-20120614-T00002
Please refer to the end of the specification for access instructions.

Lengthy table referenced here
US20120149886A1-20120614-T00003
Please refer to the end of the specification for access instructions.

Lengthy table referenced here
US20120149886A1-20120614-T00004
Please refer to the end of the specification for access instructions.

Lengthy table referenced here
US20120149886A1-20120614-T00005
Please refer to the end of the specification for access instructions.

Lengthy table referenced here
US20120149886A1-20120614-T00006
Please refer to the end of the specification for access instructions.

Lengthy table referenced here
US20120149886A1-20120614-T00007
Please refer to the end of the specification for access instructions.

Lengthy table referenced here
US20120149886A1-20120614-T00008
Please refer to the end of the specification for access instructions.

Lengthy table referenced here
US20120149886A1-20120614-T00009
Please refer to the end of the specification for access instructions.

Lengthy table referenced here
US20120149886A1-20120614-T00010
Please refer to the end of the specification for access instructions.

Lengthy table referenced here
US20120149886A1-20120614-T00011
Please refer to the end of the specification for access instructions.

Lengthy table referenced here
US20120149886A1-20120614-T00012
Please refer to the end of the specification for access instructions.

Lengthy table referenced here
US20120149886A1-20120614-T00013
Please refer to the end of the specification for access instructions.

Lengthy table referenced here
US20120149886A1-20120614-T00014
Please refer to the end of the specification for access instructions.

Lengthy table referenced here
US20120149886A1-20120614-T00015
Please refer to the end of the specification for access instructions.

Lengthy table referenced here
US20120149886A1-20120614-T00016
Please refer to the end of the specification for access instructions.

Lengthy table referenced here
US20120149886A1-20120614-T00017
Please refer to the end of the specification for access instructions.

Lengthy table referenced here
US20120149886A1-20120614-T00018
Please refer to the end of the specification for access instructions.

Lengthy table referenced here
US20120149886A1-20120614-T00019
Please refer to the end of the specification for access instructions.

Lengthy table referenced here
US20120149886A1-20120614-T00020
Please refer to the end of the specification for access instructions.

Lengthy table referenced here
US20120149886A1-20120614-T00021
Please refer to the end of the specification for access instructions.

Lengthy table referenced here
US20120149886A1-20120614-T00022
Please refer to the end of the specification for access instructions.

Lengthy table referenced here
US20120149886A1-20120614-T00023
Please refer to the end of the specification for access instructions.

Lengthy table referenced here
US20120149886A1-20120614-T00024
Please refer to the end of the specification for access instructions.

Lengthy table referenced here
US20120149886A1-20120614-T00025
Please refer to the end of the specification for access instructions.

Lengthy table referenced here
US20120149886A1-20120614-T00026
Please refer to the end of the specification for access instructions.

Lengthy table referenced here
US20120149886A1-20120614-T00027
Please refer to the end of the specification for access instructions.

Lengthy table referenced here
US20120149886A1-20120614-T00028
Please refer to the end of the specification for access instructions.

Lengthy table referenced here
US20120149886A1-20120614-T00029
Please refer to the end of the specification for access instructions.

Lengthy table referenced here
US20120149886A1-20120614-T00030
Please refer to the end of the specification for access instructions.

Lengthy table referenced here
US20120149886A1-20120614-T00031
Please refer to the end of the specification for access instructions.

Lengthy table referenced here
US20120149886A1-20120614-T00032
Please refer to the end of the specification for access instructions.

Lengthy table referenced here
US20120149886A1-20120614-T00033
Please refer to the end of the specification for access instructions.

Lengthy table referenced here
US20120149886A1-20120614-T00034
Please refer to the end of the specification for access instructions.

Lengthy table referenced here
US20120149886A1-20120614-T00035
Please refer to the end of the specification for access instructions.

Lengthy table referenced here
US20120149886A1-20120614-T00036
Please refer to the end of the specification for access instructions.

Lengthy table referenced here
US20120149886A1-20120614-T00037
Please refer to the end of the specification for access instructions.

Lengthy table referenced here
US20120149886A1-20120614-T00038
Please refer to the end of the specification for access instructions.

Lengthy table referenced here
US20120149886A1-20120614-T00039
Please refer to the end of the specification for access instructions.

Lengthy table referenced here
US20120149886A1-20120614-T00040
Please refer to the end of the specification for access instructions.

Lengthy table referenced here
US20120149886A1-20120614-T00041
Please refer to the end of the specification for access instructions.

Lengthy table referenced here
US20120149886A1-20120614-T00042
Please refer to the end of the specification for access instructions.

Lengthy table referenced here
US20120149886A1-20120614-T00043
Please refer to the end of the specification for access instructions.

Lengthy table referenced here
US20120149886A1-20120614-T00044
Please refer to the end of the specification for access instructions.

Lengthy table referenced here
US20120149886A1-20120614-T00045
Please refer to the end of the specification for access instructions.

Lengthy table referenced here
US20120149886A1-20120614-T00046
Please refer to the end of the specification for access instructions.

Lengthy table referenced here
US20120149886A1-20120614-T00047
Please refer to the end of the specification for access instructions.

Lengthy table referenced here
US20120149886A1-20120614-T00048
Please refer to the end of the specification for access instructions.

Lengthy table referenced here
US20120149886A1-20120614-T00049
Please refer to the end of the specification for access instructions.

Lengthy table referenced here
US20120149886A1-20120614-T00050
Please refer to the end of the specification for access instructions.

Lengthy table referenced here
US20120149886A1-20120614-T00051
Please refer to the end of the specification for access instructions.

Lengthy table referenced here
US20120149886A1-20120614-T00052
Please refer to the end of the specification for access instructions.

Lengthy table referenced here
US20120149886A1-20120614-T00053
Please refer to the end of the specification for access instructions.

Lengthy table referenced here
US20120149886A1-20120614-T00054
Please refer to the end of the specification for access instructions.

Lengthy table referenced here
US20120149886A1-20120614-T00055
Please refer to the end of the specification for access instructions.

Lengthy table referenced here
US20120149886A1-20120614-T00056
Please refer to the end of the specification for access instructions.

Lengthy table referenced here
US20120149886A1-20120614-T00057
Please refer to the end of the specification for access instructions.

Lengthy table referenced here
US20120149886A1-20120614-T00058
Please refer to the end of the specification for access instructions.

Lengthy table referenced here
US20120149886A1-20120614-T00059
Please refer to the end of the specification for access instructions.

Lengthy table referenced here
US20120149886A1-20120614-T00060
Please refer to the end of the specification for access instructions.

Lengthy table referenced here
US20120149886A1-20120614-T00061
Please refer to the end of the specification for access instructions.

Lengthy table referenced here
US20120149886A1-20120614-T00062
Please refer to the end of the specification for access instructions.

### EQUIVALENTS

Those skilled in the art will recognize, or be able to ascertain using no more than routine experimentation, many equivalents to the specific embodiments of the disclosure described herein. The scope of the present disclosure is not intended to be limited to the above Description, but rather is as set forth in the following claims:

LENGTHY TABLES
The patent application contains a lengthy table section. A copy of the table is available in electronic form from the USPTO web site (http://seqdata.uspto.gov/?pageRequest=docDetail&DocID=US20120149886A1). An electronic copy of the table will also be available from the USPTO upon request and payment of the fee set forth in 37 CFR 1.19(b)(3).

1.-864. (canceled)

865. A method of producing a carotenoid, the method comprising cultivating host cells of a recombinant fungus under conditions that allow production of the carotenoid, wherein the cultivating comprises growing cells under conditions in which one or more trace metals is limiting; and isolating the produced carotenoid wherein the fungus:
a. is oleaginous in that it can accumulate lipid to at least about 20% of its dry cell weight; and
b. produces at least one carotenoid, and can accumulate the produced carotenoid to at least about 1% of its dry cell weight;
wherein the recombinant fungus comprises at least one modification as compared with a parental fungus, which parental fungus both is not oleaginous and does not accumulate the carotenoid to at least about 1% of its dry cell weight, the at least one modification being selected from the group consisting of carotenogenic modifications, oleaginic modifications, and combinations thereof, and
wherein the at least one modification alters oleaginicity of the recombinant fungus, confers to the recombinant fungus oleaginy, confers to the recombinant fungus the ability to produce the at least one carotenoid to a level at least about 1% of its dry cell weight, or confers to the recombinant fungus the ability to produce at least one carotenoid which the parental fungus does not produce.

866. The method as recited in claim 865 wherein the limiting trace metal comprises zinc.

867. The method as recited in claim 865 wherein the limiting trace metal comprises manganese, iron, or a combination thereof.

868. The method as recited in claim 865 wherein the produced carotenoid comprises a carotenoid selected from the group consisting of antheraxanthin, adonirubin, adonixanthin, astaxanthin, canthaxanthin, capsorubrin, β-cryptoxanthin, α-carotene, β-carotene, β,ψ-carotene, δ-carotene, ε-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, phytofluene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, a C30 carotenoid, and combinations thereof.

869. The method as recited in claim 868, wherein the produced carotenoid comprises O-carotene.

870. A method of producing a carotenoid, the method comprising cultivating host cells of a recombinant fungus under conditions that allow production of the carotenoid, wherein the cultivating comprises a first phase of growing cells in medium comprising a first carbon source, wherein the first carbon source comprises an oil, followed by a second phase of growing cells in medium comprising a second carbon source; and isolating the produced carotenoid wherein the recombinant fungus:
a. is oleaginous in that it can accumulate lipid to at least about 20% of its dry cell weight; and
b. produces at least one carotenoid, and can accumulate the produced carotenoid to at least about 1% of its dry cell weight;
wherein the recombinant fungus comprises at least one modification as compared with a parental fungus, which parental fungus both is not oleaginous and does not accumulate the carotenoid to at least about 1% of its dry cell weight, the at least one modification being selected from the group consisting of carotenogenic modifications, oleaginic modifications, and combinations thereof, and
wherein the at least one modification alters oleaginicity of the recombinant fungus, confers to the recombinant fungus oleaginy, confers to the recombinant fungus the ability to produce the at least one carotenoid to a level at least about 1% of its dry cell weight, or confers to the recombinant fungus the ability to produce at least one carotenoid which the parental fungus does not produce.

871. The method as recited in claim 870 wherein the first carbon source comprises soybean oil.

872. The method as recited in claim 870 wherein the first phase comprises growing cells under conditions of limiting oxygen.

873. The method as recited in claim 870 wherein the second carbon source comprises glucose.

874. The method as recited in claim 870 wherein the second phase comprises growing cells under conditions of excess oxygen.

875. The method as recited in claim 870 wherein the cultivating comprises growing cells under conditions in which one or more trace metals is limiting.

876. The method as recited in claim 875 wherein the limiting trace metal comprises zinc.

877. The method as recited in claim 875 wherein the limiting trace metal comprises manganese, iron, or a combination thereof.

878. The method as recited in claim 870 wherein the produced carotenoid comprises a carotenoid selected from the group consisting of antheraxanthin, adonirubin, adonixanthin, astaxanthin, canthaxanthin, capsorubrin, β-cryptoxanthin, α-carotene, β-carotene, β,ψ-carotene, δ-carotene, ε-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, phytofluene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, a C30 carotenoid, and combinations thereof.

879. The method as recited in claim 878 wherein the produced carotenoid comprises O-carotene.

880. The method as recited in claim 878 wherein the produced carotenoid comprises canthaxanthin.

881. The method as recited in claim 878 wherein the produced carotenoid comprises astaxanthin.

882. An isolated carotenoid composition produced by a method comprising cultivating host cells of a recombinant fungus under conditions that allow production of a carotenoid, wherein the cultivating comprises growing cells under conditions in which one or more trace metals is limiting; and isolating the produced carotenoid wherein the recombinant fungus:
a. is oleaginous in that it can accumulate lipid to at least about 20% of its dry cell weight; and
b. produces at least one carotenoid, and can accumulate the produced carotenoid to at least about 1% of its dry cell weight;
wherein the recombinant fungus comprises at least one modification as compared with a parental fungus, which parental fungus both is not oleaginous and does not accumulate the carotenoid to at least about 1% of its dry cell weight, the at least one modification being selected from the group consisting of carotenogenic modifications, oleaginic modifications, and combinations thereof, and
wherein the at least one modification alters oleaginicity of the recombinant fungus, confers to the recombinant fungus oleaginy, confers to the recombinant fungus the ability to produce the at least one carotenoid to a level at least about 1% of its dry cell weight, or confers to the recombinant fungus the ability to produce at least one carotenoid which the parental fungus does not produce.

883. The composition as recited in claim 882 wherein the limiting trace metal comprises zinc.

884. The composition as recited in claim 882, wherein the limiting trace metal comprises manganese, iron, or a combination thereof.

885. The composition as recited in claim 882 wherein the produced carotenoid comprises a carotenoid selected from the group consisting of antheraxanthin, adonirubin, adonixanthin, astaxanthin, canthaxanthin, capsorubrin, β-cryptoxanthin, α-carotene, β-carotene, β,ψ-carotene, δ-carotene, ε-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, phytofluene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, a C30 carotenoid, and combinations thereof.

886. The composition as recited in claim 885 wherein the produced carotenoid comprises β-carotene.

887. An isolated carotenoid composition produced by a method comprising cultivating host cells of a fungus wherein the cultivating comprises a first phase of growing cells in medium comprising a first carbon source, wherein the first carbon source comprises an oil, followed by a second phase of growing cells in medium comprising a second carbon source; and isolating the produced carotenoid.

888. The composition as recited in claim 887 wherein the first carbon source comprises soybean oil.

889. The composition as recited in claim 887 wherein the first phase comprises growing cells under conditions of limiting oxygen.

890. The composition as recited in claim 887 wherein the second carbon source comprises glucose.

891. The composition as recited in claim 887 wherein the second phase comprises growing cells under conditions of excess oxygen.

892. The composition as recited in claim 887 wherein the cultivating comprises growing cells under conditions in which one or more trace metals is limiting.

893. The composition as recited in claim 892 wherein the limiting trace metal comprises zinc.

894. The composition as recited in claim 892 wherein the limiting trace metal comprises manganese, iron, or a combination thereof.

895. The composition as recited in claim 887 wherein the produced carotenoid comprises a carotenoid selected from the group consisting of antheraxanthin, adonirubin, adonixanthin, astaxanthin, canthaxanthin, capsorubrin, β-cryptoxanthin, α-carotene, β-carotene, β,ψ-carotene, δ-carotene, ε-carotene, echinenone, 3-hydroxyechinenone, 3′-hydroxyechinenone, γ-carotene, ψ-carotene, 4-keto-γ-carotene, ζ-carotene, α-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, phytofluene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, a C30 carotenoid, and combinations thereof.

896. The composition as recited in claim 895 wherein the produced carotenoid comprises β-carotene.

897. The composition as recited in claim 895 wherein the produced carotenoid comprises canthaxanthin.

898. The composition as recited in claim 895 wherein the produced carotenoid comprises astaxanthin.

899. An engineered yeast or fungus strain, wherein the strain produces at least one carotenoid to a level at least about 1%, 2%, 3%, 5%, or 10% of its dry weight when grown by a method, the method comprising:
cultivating cells of the strain under conditions that allow production of the carotenoid, wherein the cultivating comprises a first phase of growing cells in medium comprising a first carbon source, wherein the first carbon source comprises an oil, followed by a second phase of growing cells in medium comprising a second carbon source.

900. The strain as recited in claim 899 wherein the first carbon source comprises soybean oil.

901. The strain as recited in claim 899 wherein the first phase comprises growing cells under conditions of limiting oxygen.

902. The strain as recited in claim 899 wherein the second carbon source comprises glucose.

903. The strain as recited in claim 899 wherein the second phase comprises growing cells under conditions of excess oxygen.

904. The strain as recited in claim 899 wherein the cultivating comprises growing cells under conditions in which one or more trace metals is limiting.

905. The strain as recited in claim 899 wherein the limiting trace metal comprises zinc.

906. The strain as recited in claim 899 wherein the limiting trace metal comprises manganese, iron, or a combination thereof.

907. The strain as recited in claim 899 wherein the at least one carotenoid comprises β-carotene.

908. The strain as recited in claim 899 wherein the at least one carotenoid comprises canthaxanthin.

909. The strain as recited in claim 899 wherein the at least one carotenoid comprises astaxanthin.


SEQUENCE LISTING (218 sequences)

SEQ ID NO: 1 - protein, 34 aa, Yarrowia lipolytica
MLSRNLSKFARAGLIRPATTSTHTRLFSVSARRL

SEQ ID NO: 2 - protein, 32 aa, Yarrowia lipolytica
MLRLIRPRLAALARPTTRAPQALNARTHIVSV

SEQ ID NO: 3 - protein, 53 aa, Saccharomyces cerevisiae
MFQRSGAAHHIKLISSRRCRFKSSFAVALNAASKLVTPKILWNNPISLVSKEM

SEQ ID NO: 4 - protein, 77 aa, Yarrowia lipolytica
MLRVGRIGTKTLASSSLRFVAGARPKSTLTEAVLETTGLLKTTPQNPEWSGAVKQASRLVETDTPIRDPFSIVSQEM

SEQ ID NO: 5 - DNA, 984 bp, Yarrowia lipolytica
atggattataacagcgcggatttcaaggagatatggggcaaggccgccgacaccgcgctgctgggaccgtacaactacctcgccaacaaccggggccacaacatcagagaacacttgatcgcagcgttcggagcggttatcaaggtggacaagagcgatctcgagaccatttcgcacatcaccaagattttgcataactcgtcgctgcttgttgatgacgtggaagacaactcgatgctccgacgaggcctgccggcagcccattgtctgtttggagtcccccaaaccatcaactccgccaactacatgtactttgtggctctgcaggaggtgctcaagctcaagtcttatgatgccgtctccattttcaccgaggaaatgatcaacttgcatagaggtcagggtatggatctctactggagagaaacactcacttgcccctcggaagacgagtatctggagatggtggtgcacaagaccggtggactgtttcggctggctctgagacttatgctgtcggtggcatcgaaacaggaggaccatgaaaagatcaactttgatctcacacaccttaccgacacactgggagtcatttaccagattctggatgattacctcaacctgcagtccacggaattgaccgagaacaagggattctgcgaagatatcagcgaaggaaagttttcgtttccgctgattcacagcatacgcaccaacccggataaccacgagattctcaacattctcaaacagcgaacaagcgacgcttcactcaaaaagtacgccgtggactacatgagaacagaaaccaagagtttcgactactgcctcaagaggatacaggccatgtcactcaaggcaagttcgtacattgatgatctagcagcagctggccacgatgtctccaagctacgagccattttgcattattttgtgtccacctctgactgtgaggagagaaagtactttgaggatgcgcagtga

SEQ ID NO: 6 - protein, 327 aa, Yarrowia lipolytica
MDYNSADFKEIWGKAADTALLGPYNYLANNRGHNIREHLIAAFGAVIKVDKSDLETISHITKILHNSSLLVDDVEDNSMLRRGLPAAHCLFGVPQTINSANYMYFVALQEVLKLKSYDAVSIFTEEMINLHRGQGMDLYWRETLTCPSEDEYLEMVVHKTGGLFRLALRLMLSVASKQEDHEKINFDLTHLTDTLGVIYQILDDYLNLQSTELTENKGFCEDISEGKFSFPLIHSIRTNPDNHEILNILKQRTSDASLKKYAVDYMRTETKSFDYCLKRIQAMSLKASSYIDDLAAAGHDVSKLRAILHYFVSTSDCEERKYFEDAQ

SEQ ID NO: 7 - DNA, 20 bp, Artificial Sequence
  synthetic oligonucleotide MO4471
ctgggtgacctggaagcctt

SEQ ID NO: 8 - DNA, 23 bp, Artificial Sequence
  synthetic oligonucleotide MO4472
aagatcaatccgtagaagttcag

SEQ ID NO: 9 - DNA, 23 bp, Artificial Sequence
  synthetic oligonucleotide MO4475
aagcgattacaatcttcctttgg

SEQ ID NO: 10 - DNA, 22 bp, Artificial Sequence
  synthetic oligonucleotide MO4476
ccagtccatcaactcagtctca

SEQ ID NO: 11 - DNA, 23 bp, Artificial Sequence
  synthetic oligonucleotide MO4477
gcattgcttattacgaagactac

SEQ ID NO: 12 - DNA, 23 bp, Artificial Sequence
  synthetic oligonucleotide MO4478
ccactgtcctccactacaaacac

SEQ ID NO: 13 - DNA, 31 bp, Artificial Sequence
  synthetic oligonucleotide MO4534
cacaaacgcgttcactgcgcatcctcaaagt

SEQ ID NO: 14 - DNA, 37 bp, Artificial Sequence
  synthetic oligonucleotide MO4544
cacaatctagacacaaatggattataacagcgcggat

SEQ ID NO: 15 - DNA, 31 bp, Artificial Sequence
  synthetic oligonucleotide MO4566
cacaaactagtttgccacctacaagccagat

SEQ ID NO: 16 - DNA, 31 bp, Artificial Sequence
  synthetic oligonucleotide MO4568
cacaaggtaccaatgtgaaagtgcgcgtgat

SEQ ID NO: 17 - DNA, 28 bp, Artificial Sequence
  synthetic oligonucleotide MO4571
cacaaggtaccagagaccgggttggcgg

SEQ ID NO: 18 - DNA, 40 bp, Artificial Sequence
  synthetic oligonucleotide MO4591
cacaagcggccgcgctagcatggggatcgatctcttatat

SEQ ID NO: 19 - DNA, 41 bp, Artificial Sequence
  synthetic oligonucleotide MO4592
cacaagcggccgcgctagcgaatgattcttatactcagaag

SEQ ID NO: 20 - DNA, 40 bp, Artificial Sequence
  synthetic oligonucleotide MO4593
cacaagcggccgcacgcgtgcaattaacagatagtttgcc

SEQ ID NO: 21 - DNA, 33 bp, Artificial Sequence
  synthetic oligonucleotide MO4659
cacaagctagctggggatgcgatctcttatatc

SEQ ID NO: 22 - DNA, 36 bp, Artificial Sequence
  synthetic oligonucleotide MO4525
cacaaacgcgtttaaatggtatttagatttctcatt

SEQ ID NO: 23 - DNA, 36 bp, Artificial Sequence
  synthetic oligonucleotide MO4541
cacaatctagacacaaatgctgctcacctacatgga

SEQ ID NO: 24 - DNA, 1906 bp, Mucor circinelloides
atgctgctcacctacatggaagtccacctctactacacgctgcctgtgctgggcgtcctgtcctggctgtcgcggccgtactacacagccaccgatgcgctcaaattcaaatttctgacactggttgccttcacgaccgcctccgcctgggacaactacattgtctaccacaaggcgtggtcctactgccccacctgcgtcaccgctgtcattggctacgtgcccttggaggagtacatgttcttcatcatcatgactctgttgaccgtggcattcaccaatctggtgatgcgctggcacctgcacagcttctttatcaggcctgaaacgcccgtcatgcagtccgtcctggtccgtcttgtccccataacagccttattaatcactgcatacaaggcttgggtaagcaaacaaacaaatgatgtgccgcatcgcattttaatattaaccattgcatacacagcatttggcggtccctggaaagccactgttctacggatcatgcattttgtggtacgcctgtccggttttggccttattgtggtttggtgctggcgagtacatgatgcgtcgtccgctggcggtgctcgtctccattgcgctgcccacgctgtttctctgctgggtcgatgtcgtcgctattggcgccggcacatgggacatttcgctggccacaagcaccggcaagttcgtcgtgccccacctgcccgtggaggaattcatgttctttgcgctaattaataccgttttggtatttggtacgtgtgcgatcgatcgcacgatggcgatcctccacctgttcaaaaacaagagtccttatcagcgcccataccagcacagcaagtcgttcctccaccagatcctcgagatgacctgggccttctgtttacccgaccaagtgctgcattcagacacattccacgacctgtccgtcagctgggacatcctgcgcaaggcctccaagtccttttacacggcctctgctgtctttcccggcgacgtgcgccaagagctcggtgtgctatacgccttttgcagagccacggacgatctctgcgacaacgagcaggtccctgtgcagacgcgaaaggagcagctgatactgacacatcagttcgtcagcgatctgtttggccaaaagacaagcgcgccgactgccattgactgggacttttacaacgaccaactgcctgcctcgtgcatctctgccttcaagtcgttcacccgtttgcgccatgtgctggaagctggagccatcaaggaactgctcgacgggtacaagtgggatttggagcgtcgctccatcagggatcaggaggatctcagatattactcagcttgtgtcgccagcagtgttggtgaaatgtgcactcgcatcatactggcccacgccgacaagcccgcctcccgccagcaaacacagtggatcattcagcgtgcgcgtgaaatgggtctggtactccaatatacaaacattgcaagagacattgtcaccgacagcgaggaactgggcagatgctacctgcctcaggattggcttaccgagaaggaggtggcgctgattcaaggcggccttgcccgagaaattggcgaggagcgattgctctcactgtcgcatcgcctcatctaccaggcagacgagctcatggtggttgccaacaagggcatcgacaagctgcccagccattgtcaaggcggcgtgcgtgcggcctgcaacgtctatgcttccattggcaccaagctcaagtcttacaagcaccactatcccagcagagcacatgtcggcaattcgaaacgagtggaaattgctcttcttagcgtatacaacctttacaccgcgccaattgcgactagtagtaccacacattgcagacagggaaaaatgagaaatctaaataccatttaa

SEQ ID NO: 25 - protein, 614 aa, Mucor circinelloides
MLLTYMEVHLYYTLPVLGVLSWLSRPYYTATDALKFKFLTLVAFTTASAWDNYIVYHKAWSYCPTCVTAVIGYVPLEEYMFFIIMTLLTVAFTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLPVEEFMFFALINTVLVFGTCAIDRTMAILHLFKNKSPYQRPYQHSKSFLHQILEMTWAFCLPDQVLHSDTFHDLSVSWDILRKASKSFYTASAVFPGDVRQELGVLYAFCRATDDLCDNEQVPVQTRKEQLILTHQFVSDLFGQKTSAPTAIDWDFYNDQLPASCISAFKSFTRLRHVLEAGAIKELLDGYKWDLERRSIRDQEDLRYYSACVASSVGEMCTRIILAHADKPASRQQTQWIIQRAREMGLVLQYTNIARDIVTDSEELGRCYLPQDWLTEKEVALIQGGLAREIGEERLLSLSHRLIYQADELMVVANKGIDKLPSHCQGGVRAACNVYASIGTKLKSYKHHYPSRAHVGNSKRVEIALLSVYNLYTAPIATSSTTHCRQGKMRNLNTI

SEQ ID NO: 26 - DNA, 17 bp, Artificial Sequence
  synthetic oligonucleotide MO4318
gtaaaacgacggccagt

SEQ ID NO: 27 - DNA, 38 bp, Artificial Sequence
  synthetic oligonucleotide MO4643
cacacggtctcatgccaagccttgtatgcagtgattaa

SEQ ID NO: 28 - DNA, 19 bp, Artificial Sequence
  synthetic oligonucleotide MO4639
ccactgtgtttgctggcgg

SEQ ID NO: 29 - DNA, 34 bp, Artificial Sequence
  synthetic oligonucleotide MO4644
cacacggtctctggcatttggcggtccctggaaa

SEQ ID NO: 30 - DNA, 1845 bp, Artificial Sequence
  synthetic plasmid sequence pMB4705
atgctgctcacctacatggaagtccacctctactacacgctgcctgtgctgggcgtcctgtcctggctgtcgcggccgtactacacagccaccgatgcgctcaaattcaaatttctgacactggttgccttcacgaccgcctccgcctgggacaactacattgtctaccacaaggcgtggtcctactgccccacctgcgtcaccgctgtcattggctacgtgcccttggaggagtacatgttcttcatcatcatgactctgttgaccgtggcattcaccaatctggtgatgcgctggcacctgcacagcttctttatcaggcctgaaacgcccgtcatgcagtccgtcctggtccgtcttgtccccataacagccttattaatcactgcatacaaggcttggcatttggcggtccctggaaagccactgttctacggatcatgcattttgtggtacgcctgtccggttttggccttattgtggtttggtgctggcgagtacatgatgcgtcgtccgctggcggtgctcgtctccattgcgctgcccacgctgtttctctgctgggtcgatgtcgtcgctattggcgccggcacatgggacatttcgctggccacaagcaccggcaagttcgtcgtgccccacctgcccgtggaggaattcatgttctttgcgctaattaataccgttttggtatttggtacgtgtgcgatcgatcgcacgatggcgatcctccacctgttcaaaaacaagagtccttatcagcgcccataccagcacagcaagtcgttcctccaccagatcctcgagatgacctgggccttctgtttacccgaccaagtgctgcattcagacacattccacgacctgtccgtcagctgggacatcctgcgcaaggcctccaagtccttttacacggcctctgctgtctttcccggcgacgtgcgccaagagctcggtgtgctatacgccttttgcagagccacggacgatctctgcgacaacgagcaggtccctgtgcagacgcgaaaggagcagctgatactgacacatcagttcgtcagcgatctgtttggccaaaagacaagcgcgccgactgccattgactgggacttttacaacgaccaactgcctgcctcgtgcatctctgccttcaagtcgttcacccgtttgcgccatgtgctggaagctggagccatcaaggaactgctcgacgggtacaagtgggatttggagcgtcgctccatcagggatcaggaggatctcagatattactcagcttgtgtcgccagcagtgttggtgaaatgtgcactcgcatcatactggcccacgccgacaagcccgcctcccgccagcaaacacagtggatcattcagcgtgcgcgtgaaatgggtctggtactccaatatacaaacattgcaagagacattgtcaccgacagcgaggaactgggcagatgctacctgcctcaggattggcttaccgagaaggaggtggcgctgattcaaggcggccttgcccgagaaattggcgaggagcgattgctctcactgtcgcatcgcctcatctaccaggcagacgagctcatggtggttgccaacaagggcatcgacaagctgcccagccattgtcaaggcggcgtgcgtgcggcctgcaacgtctatgcttccattggcaccaagctcaagtcttacaagcaccactatcccagcagagcacatgtcggcaattcgaaacgagtggaaattgctcttcttagcgtatacaacctttacaccgcgccaattgcgactagtagtaccacacattgcagacagggaaaaatgagaaatctaaataccatttaa

SEQ ID NO: 31 - DNA, 35 bp, Artificial Sequence
  synthetic oligonucleotide MO4530
cacaaacgcgtttaaatgacattagagttatgaac

SEQ ID NO: 32 - DNA, 37 bp, Artificial Sequence
  synthetic oligonucleotide MO4542
cacaatctagacacaaatgtccaagaaacacattgtc

SEQ ID NO: 33 - DNA, 17 bp, Artificial Sequence
  synthetic oligonucleotide MO4318
gtaaaacgacggccagt

SEQ ID NO: 34 - DNA, 31 bp, Artificial Sequence
  synthetic oligonucleotide MO4648
cacaaggtctcaagcacgcatcccggaactg

SEQ ID NO: 35 - DNA, 32 bp, Artificial Sequence
  synthetic oligonucleotide MO4646
cacacggtctcaggcatgtcgccctacgatgc

SEQ ID NO: 36 - DNA, 35 bp, Artificial Sequence
  synthetic oligonucleotide MO4647
cacacggtctcatgcttgcacccacaaagaatagg

SEQ ID NO: 37 - DNA, 17 bp, Artificial Sequence
  synthetic oligonucleotide MO4343
caggaaacagctatgac

SEQ ID NO: 38 - DNA, 36 bp, Artificial Sequence
  synthetic oligonucleotide MO4645
cacacggtctcttgcccatatacatggtctgaaacg

SEQ ID NO: 39 - DNA, 1740 bp, Artificial Sequence
  synthetic plasmid - CarB protein of pMB4638
atgtccaagaaacacattgtcattatcggtgctggcgtgggtggcacggctacagctgctcgtttggcccgcgaaggcttcaaggtcactgtggtggagaaaaacgactttggtggcggccgctgctccttgatccatcaccagggccatcgctttgatcagggcccgtcgctctacctgatgcccaagtactttgaggacgcctttgccgatctggacgagcgcattcaagaccacctggagctgctgcgatgcgacaacaactacaaggtgcactttgacgacggtgagtcgatccagctgtcgtctgacttgacacgcatgaaggctgaattggaccgcgtggagggcccccttggttttggccgattcctggatttcatgaaagagacacacatccactacgaaagcggcaccctgattgcgctcaagaagaatttcgaatccatctgggacctgattcgcatcaagtacgctccagagatctttcgcttgcacctgtttggcaagatctacgaccgcgcttccaagtacttcaagaccaagaagatgcgcatggcattcacgtttcagaccatgtatatgggcatgtcgccctacgatgcgcctgctgtctacagcctgttgcagtacaccgagttcgctgaaggcatctggtatccccgtggcggcttcaacatggtggttcagaagctagaggcgattgcaaagcaaaagtacgatgccgagtttatctacaatgcgcctgttgccaagattaacaccgatgatgccaccaaacaagtgacaggtgtaaccttggaaaatggccacatcatcgatgccgatgcggttgtgtgtaacgcagatctggtctatgcttatcacaatctgttgcctccctgccgatggacgcaaaacacactggcttccaagaaattgacgtcttcttccatttccttctactggtccatgtccaccaaggtgcctcaattggacgtgcacaacatctttttggccgaggcttatcaggagagctttgacgaaatcttcaaggactttggcctgccttctgaagcctccttctacgtcaatgtgccctctcgcatcgatccttctgctgctcccgacggcaaggactctgtcattgtcttggtgcctattggtcatatgaagagcaagacgggcgatgcttccaccgagaactacccggccatggtggacaaggcacgcaagatggtgctggctgtgattgagcgtcgtctgggcatgtcgaatttcgccgacttgattgagcatgagcaagtcaatgatcccgctgtatggcagagcaagttcaatctgtggagaggctcaattctgggtttgtctcatgatgtgcttcaggtgctgtggttccgtcccagcacaaaggattctaccggtcgttatgataacctattctttgtgggtgcaagcacgcatcccggaactggtgttcccattgtccttgcaggaagcaagctcacctctgaccaagttgtcaagagctttggaaagacgcccaagccaagaaagatcgagatggagaacacgcaagcacctttggaggagcctgatgctgaatcgacattccctgtgtggttctggttgcgcgctgccttttgggtcatgtttatgttcttttacttcttccctcaatccaatggccaaacgcccgcatcttttatcaataatttgttacctgaagtattccgcgttcataactctaatgtcatttaa

SEQ ID NO: 40 - protein, 579 aa, Artificial Sequence
  synthetic plasmid - CarB protein of pMB4638
MSKKHIVIIGAGVGGTATAARLAREGFKVTVVEKNDFGGGRCSLIHHQGHRFDQGPSLYLMPKYFEDAFADLDERIQDHLELLRCDNNYKVHFDDGESIQLSSDLTRMKAELDRVEGPLGFGRFLDFMKETHIHYESGTLIALKKNFESIWDLIRIKYAPEIFRLHLFGKIYDRASKYFKTKKMRMAFTFQTMYMGMSPYDAPAVYSLLQYTEFAEGIWYPRGGFNMVVQKLEAIAKQKYDAEFIYNAPVAKINTDDATKQVTGVTLENGHIIDADAVVCNADLVYAYHNLLPPCRWTQNTLASKKLTSSSISFYWSMSTKVPQLDVHNIFLAEAYQESFDEIFKDFGLPSEASFYVNVPSRIDPSAAPDGKDSVIVLVPIGHMKSKTGDASTENYPAMVDKARKMVLAVIERRLGMSNFADLIEHEQVNDPAVWQSKFNLWRGSILGLSHDVLQVLWFRPSTKDSTGRYDNLFFVGASTHPGTGVPIVLAGSKLTSDQVVKSFGKTPKPRKIEMENTQAPLEEPDAESTFPVWFWLRAAFWVMFMFFYFFPQSNGQTPASFINNLLPEVFRVHNSNVI

SEQ ID NO: 41 - DNA, 32 bp, Artificial Sequence
  synthetic oligonucleotide MO4684
cattcactagtggtgtgttctgtggagcattc

SEQ ID NO: 42 - DNA, 36 bp, Artificial Sequence
  synthetic oligonucleotide MO4685
cacacggtctcatcgaggtgtagtggtagtgcagtg

SEQ ID NO: 43 - DNA, 1740 bp, Artificial Sequence; identical to SEQ ID NO: 39
  synthetic plasmid - CarB(i) protein of pMB4660

SEQ ID NO: 44 - protein, 579 aa, Artificial Sequence; identical to SEQ ID NO: 40
  synthetic plasmid - CarB(i) protein of pMB4660

SEQ ID NO: 45 - DNA, 36 bp, Artificial Sequence
  synthetic oligonucleotide - Primer O
ttctagacacaaaaatggctgcagaccaattggtga

SEQ ID NO: 46 - DNA, 33 bp, Artificial Sequence
  synthetic oligonucleotide - Primer P
cattaattcttctaaaggacgtattttcttatc

SEQ ID NO: 47 - DNA, 21 bp, Artificial Sequence
  synthetic oligonucleotide - Primer Q
gttctctggacgacctagagg

SEQ ID NO: 48 - DNA, 33 bp, Artificial Sequence
  synthetic oligonucleotide MO4658
cacacacgcgtacacctatgaccgtatgcaaat

SEQ ID NO: 49 - DNA, 40 bp, Artificial Sequence
  synthetic oligonucleotide MO4657
cacactctagacacaaaaatgacccagtctgtgaaggtgg

SEQ ID NO: 50 - DNA, 1503 bp, Artificial Sequence
  synthetic plasmid - Hmg1trunc protein of pMB4637 and pMB4714
atgacccagtctgtgaaggtggttgagaagcacgttcctatcgtcattgagaagcccagcgagaaggaggaggacacctcttctgaagactccattgagctgactgtcggaaagcagcccaagcccgtgaccgagacccgttctctggacgacctagaggctatcatgaaggcaggtaagaccaagcttctggaggaccacgaggttgtcaagctctctctcgagggcaagcttcctttgtatgctcttgagaagcagcttggtgacaacacccgagctgttggcatccgacgatctatcatctcccagcagtctaataccaagactttagagacctcaaagcttccttacctgcactacgactacgaccgtgtttttggagcctgttgcgagaacgttattggttacatgcctctccccgttggtgttgctggccccatgaacattgatggcaagaactaccacattcctatggccaccactgagggttgtcttgttgcctcaaccatgcgaggttgcaaggccatcaacgccggtggcggtgttaccactgtgcttactcaggacggtatgacacgaggtccttgtgtttccttcccctctctcaagcgggctggagccgctaagatctggcttgattccgaggagggtctcaagtccatgcgaaaggccttcaactccacctctcgatttgctcgtctccagtctcttcactctacccttgctggtaacctgctgtttattcgattccgaaccaccactggtgatgccatgggcatgaacatgatctccaagggcgtcgaacactctctggccgtcatggtcaaggagtacggcttccctgatatggacattgtgtctgtctcgggtaactactgcactgacaagaagcccgcagcgatcaactggatcgaaggccgaggcaagagtgttgttgccgaagccaccatccctgctcacattgtcaagtctgttctcaaaagtgaggttgacgctcttgttgagctcaacatcagcaagaatctgatcggtagtgccatggctggctctgtgggaggtttcaatgcacacgccgcaaacctggtgaccgccatctaccttgccactggccaggatcctgctcagaatgtcgagtcttccaactgcatcacgctgatgagcaacgtcgacggtaacctgctcatctccgtttccatgccttctatcgaggtcggtaccattggtggaggtactattttggagccccagggggctatgctggagatgcttggcgtgcgaggtcctcacatcgagacccccggtgccaacgcccaacagcttgctcgcatcattgcttctggagttcttgcagcggagctttcgctgtgttctgctcttgctgccggccatcttgtgcaaagtcatatgacccacaaccggtcccaggctcctactccggccaagcagtctcaggccgatctgcagcgtctacaaaacggttcgaatatttgcatacggtcatag

SEQ ID NO: 51 - protein, 500 aa, Artificial Sequence
  synthetic plasmid - Hmg1trunc protein of pMB4637 and pMB4714
MTQSVKVVEKHVPIVIEKPSEKEEDTSSEDSIELTVGKQPKPVTETRSLDDLEAIMKAGKTKLLEDHEVVKLSLEGKLPLYALEKQLGDNTRAVGIRRSIISQQSNTKTLETSKLPYLHYDYDRVFGACCENVIGYMPLPVGVAGPMNIDGKNYHIPMATTEGCLVASTMRGCKAINAGGGVTTVLTQDGMTRGPCVSFPSLKRAGAAKIWLDSEEGLKSMRKAFNSTSRFARLQSLHSTLAGNLLFIRFRTTTGDAMGMNMISKGVEHSLAVMVKEYGFPDMDIVSVSGNYCTDKKPAAINWIEGRGKSVVAEATIPAHIVKSVLKSEVDALVELNISKNLIGSAMAGSVGGFNAHAANLVTAIYLATGQDPAQNVESSNCITLMSNVDGNLLISVSMPSIEVGTIGGGTILEPQGAMLEMLGVRGPHIETPGANAQQLARIIASGVLAAELSLCSALAAGHLVQSHMTHNRSQAPTPAKQSQADLQRLQNGSNICIRS

SEQ ID NO: 52 - DNA, 551 bp, Artificial Sequence
  synthetic CrtZ based on protein sequence of Novosphingobium aromaticivorans, using Y. lipolytica codon bias
ttctagacacaaaaatgggtggagccatgcagaccctcgctgctatcctgatcgtcctcggtacagtgctcgctatggagtttgtcgcttggtcttctcataagtatatcatgcatggcttcggatggggatggcatagagaccatcacgagccccatgagggatttcttgagaagaatgacttatacgccatcgttggcgctgccctctcgatactcatgtttgccctcggctctcccatgatcatgggcgctgacgcctggtggcccggaacctggatcggactcggtgtcctcttctatggtgtcatctataccctcgtgcacgacggtctggtgcaccaacgatggtttagatgggtgcctaaacgaggttacgccaaacgactcgtgcaggcccataagctgcaccacgccaccattggcaaggaaggaggcgtctcattcggtttcgtgttcgcccgagatcccgccgttctgaagcaggagcttcgagctcaacgagaagcaggtatcgccgtgctgcgagaggctgtggacggctagacgcgt

SEQ ID NO: 53 - protein, 176 aa, Artificial Sequence
  CrtZ protein of pMB4692
MGGAMQTLAAILIVLGTVLAMEFVAWSSHKYIMHGFGWGWHRDHHEPHEGFLEKNDLYAIVGAALSILMFALGSPMIMGADAWWPGTWIGLGVLFYGVIYTLVHDGLVHQRWFRWVPKRGYAKRLVQAHKLHHATIGKEGGVSFGFVFARDPAVLKQELRAQREAGIAVLREAVDG

SEQ ID NO: 54 - DNA, 815 bp, Artificial Sequence
  Synthetic CrtW ORF based on protein sequence of an environmental sequence isolated from the Sargasso Sea
ttctagacacaaaaatgactcgatctatttcctggccttccacctactggcacctccagccctcctgttcttcttgggtcgcaaacgaattctctcctcaagcccgaaaaggtctcgtcctcgctggtctcattggttccgcttggctgcttactctcggacttggcttttcccttcccctccatcaaacgagctggcttctcatcggttgtctcgttctccttagatctttcctgcacaccggactttttatcgttgcccatgacgctatgcacgcttctcttgttcctgaccaccctggccttaaccgttggattggacgtgtctgtcttctcatgtatgctggactctcctacaaaagatgctgccgaaatcaccgtcgacaccaccaagcccctgaaacagttgaagaccctgactaccaacgatgcactaacaacaatatcctcgactggtacgttcactttatgggaaattacctcggatggcaacaattgcttaatctctcttgcgtttggctcgctctcaccttccgtgtttctgactactctgctcaattcttccacctgctccttttctctgtccttcctctcatcgtctcctcctgtcaactcttcctcgtgggaacctggctgccacaccgacgaggcgctactactcgacccggcgttaccactcgatccctgaacttccaccctgctctttccttcgctgcttgctaccacttcggttaccaccgtgaacaccatgaatctccctctactccttggttccaacttcctaaactccgagaaggttctctcatctaaacgcgt

SEQ ID NO: 55 - protein, 264 aa, Artificial Sequence
  CrtW protein of pMB4698
MTRSISWPSTYWHLQPSCSSWVANEFSPQARKGLVLAGLIGSAWLLTLGLGFSLPLHQTSWLLIGCLVLLRSFLHTGLFIVAHDAMHASLVPDHPGLNRWIGRVCLLMYAGLSYKRCCRNHRRHHQAPETVEDPDYQRCTNNNILDWYVHFMGNYLGWQQLLNLSCVWLALTFRVSDYSAQFFHLLLFSVLPLIVSSCQLFLVGTWLPHRRGATTRPGVTTRSLNFHPALSFAACYHFGYHREHHESPSTPWFQLPKLREGSLI

SEQ ID NO: 56 - DNA, 968 bp, Artificial Sequence
  Synthetic CrtW ORF based on protein sequence of Aurantimonas sp. SI85-9A1, using Y. lipolytica codon bias
ctctagacacaaaaatgtcttcctttgcccctatgaatgatgttgctattcctgccggtcaagctcctttctctgcctgtactagaaaacctgtcctgagaccttttcaagctgccatcggtcttacactcgccggatgtgttatctctgcttggattgcaatccacgttggagctgtctttttcctcgatgtcggttggcgaacccttcctgttgttcctgtcctcattgccgttcagtgctggctcacggtcggtctttttattgtcgcacacgatgctatgcacggctccctcgctcctggttggccacgacttaacgctcgaattggtgccttcatcctcaccatctacgctggattcgcttggagacgtgtccgaggagctcacatggcccatcacgacgcccctggtactgccgatgaccctgacttctttgttgatgaacctgaccgattttggccttggtttcgagctttcttccttagatattttggacgtcgatctattctctttgtttgcacagttgtcaccgtttacattctggtccttggagcccctgttcttaatgttgttctcttttacggtcttccttcccttctgtcttctcttcaactcttttactttggaacttttcgtcctcaccgtcatgaagaagatgatttcgttgacgcccataatgcccgatctaatgaatttggttacatcgcctccctcctttcttgctttcactttggataccatcacgaacatcatgccgagccgtgggtcccttggtggggtcttccttctcaatggcgccagagacaagcctcttcttcccgacaggtcccgggcggccgagacgctgctgacgccgctggagcatctcgacaacctgccggacgataccgatctgtttcttctcgaggtcgaaatcaggcccgttctcccgcttctggtcgaaacgaacaaatgagataaacgcgt

SEQ ID NO: 57 - protein, 315 aa, Artificial Sequence
  CrtW protein of pMB4741
MSSFAPMNDVAIPAGQAPFSACTRKPVLRPFQAAIGLTLAGCVISAWIAIHVGAVFFLDVGWRTLPVVPVLIAVQCWLTVGLFIVAHDAMHGSLAPGWPRLNARIGAFILTIYAGFAWRRVRGAHMAHHDAPGTADDPDFFVDEPDRFWPWFRAFFLRYFGRRSILFVCTVVTVYILVLGAPVLNVVLFYGLPSLLSSLQLFYFGTFRPHRHEEDDFVDAHNARSNEFGYIASLLSCFHFGYHHEHHAEPWVPWWGLPSQWRQRQASSSRQVPGGRDAADAAGASRQPAGRYRSVSSRGRNQARSPASGRNEQMR

SEQ ID NO: 58 - DNA, 811 bp, Artificial Sequence
  Synthetic CrtW ORF based on protein sequence of Parvularcula bermudensis, using Y. lipolytica codon bias
ctctagacacaaaaatggaccctaccggagacgttactgctagccctcgacctcaaaccaccattcctgtccgacaagcactctggggacttagccttgctggagccatcatcgccgcatgggtttttatgcacattggtttcgttttttttgccccccttgatcctatcgttctcgccctcgccccagttattattcttcttcaatcctggctttctgttggtctttttattatttctcacgacgcaattcacggttccctcgcccctggacgacccgcctttaatagagccatgggacgactctgcatgacactttacgccggtttcgactttgaccgtatggccgctgcacatcaccgacatcacagatcccctggaaccgccgctgaccccgatttttctgttgactcccctgatcgacctctcccttggtttggagctttcttccgacgttactttggctggagaccttttcttaccgttaacgctgtcgtctttacctactggcttgttcttggagctaaccctgttaatattgttctcttttatggcgttcctgcactcctttccgccggacagctcttttactttggtacatttctccctcaccgacacgaacgacaaggctttgctgatcaccaccgagcacgatccgtccgatccccttacatgctttctcttgttacttgttaccactttggaggctatcatcacgaacatcatctctttccacacgaaccctggtggcgcctgcctcaacgaggaggttgggaacgtgacagacgaaagagaaccggcccttaacgcgt

SEQ ID NO: 59 - protein, 263 aa, Artificial Sequence
  CrtW protein of pMB4735
MDPTGDVTASPRPQTTIPVRQALWGLSLAGAIIAAWVFMHIGFVFFAPLDPIVLALAPVIILLQSWLSVGLFIISHDAIHGSLAPGRPAFNRAMGRLCMTLYAGFDFDRMAAAHHRHHRSPGTAADPDFSVDSPDRPLPWFGAFFRRYFGWRPFLTVNAVVFTYWLVLGANPVNIVLFYGVPALLSAGQLFYFGTFLPHRHERQGFADHHRARSVRSPYMLSLVTCYHFGGYHHEHHLFPHEPWWRLPQRGGWERDRRKRTGP

SEQ ID NO: 60 - DNA, 575 bp, Artificial Sequence
  (CrtZ) ORF based on protein sequence of Parvularcula bermudensis, using Y. lipolytica codon bias
ctctagacacaaaaatgactctcgctctctggcaaaagatcaccctcgtccttggttccgctgctctgatggaaggatttgcttggtgggcccatagatatattatgcacggttggggatgggcttggcatagagatcatcatgaacctcacgacaaagtttttgaaaaaaatgacctgtttgctgtggtttttggctcgttcgcatttggtttgttcatcgtcggttacctttattggccacctgtttggtacgttgctgctggcatcactctttacggacttctttacgcatttgttcatgacggtttggttcatcaacgttggccctggcatttcatgcctaaacgaggatacctccgaagactggttcaagctcacaaacttcatcatgctgttacaacacaaggcggaaatgtttcgtttggattcgtccttgcccctgaccctagacatcttagagaaaaacttagacaatttcgtgctgaaagacatcgtgcccttgccgccgaaggtgcttcctcctctgaccctcgtgttcccccttttcgaaaagttcaagacgtttaaacgcgt

SEQ ID NO: 61 - protein, 184 aa, Artificial Sequence
  CrtZ protein of pMB4778
MTLALWQKITLVLGSAALMEGFAWWAHRYIMHGWGWAWHRDHHEPHDKVFEKNDLFAVVFGSFAFGLFIVGYLYWPPVWYVAAGITLYGLLYAFVHDGLVHQRWPWHFMPKRGYLRRLVQAHKLHHAVTTQGGNVSFGFVLAPDPRHLREKLRQFRAERHRALAAEGASSSDPRVPPFRKVQDV

SEQ ID NO: 62 - DNA, 521 bp, Artificial Sequence
  (CrtZ) ORF based on protein sequence of Erythrobacter litoralis, using Y. lipolytica codon bias
ctctagacacaaaaatgagctggtgggctatcgctcttattgtctttggtgctgtcgttggaatggaattttttgcttggttcgctcataagtacattatgcatggttggggatggagctggcaccgagatcatcacgaacctcacgataatactcttgaaaaaaacgaccttttcgccgttgtctttggctcggttgccgcacttctgtttgttattggagctctctggtctgatcctctctggtgggcagcagttggtattacattgtatggcgtcatttacactctggttcacgacggacttgttcatcaacgttactggcgttggacccctaagcgaggttatgctaagagacttgtccaggcccatcgacttcatcacgctactgttggaaaggaaggaggtgtttcttttggttttgtgttcgcccgagatcctgctaagttgaaagccgaattgaaacaacaaagagaacagggacttgccgtcgttcgagattctatgggagcataaacgcgt

SEQ ID NO: 63 - protein, 166 aa, Artificial Sequence
  CrtZ protein of pMB4719
MSWWAIALIVFGAVVGMEFFAWFAHKYIMHGWGWSWHRDHHEPHDNTLEKNDLFAVVFGSVAALLFVIGALWSDPLWWAAVGITLYGVIYTLVHDGLVHQRYWRWTPKRGYAKRLVQAHRLHHATVGKEGGVSFGFVFARDPAKLKAELKQQREQGLAVVRDSMGA

SEQ ID NO: 64 - DNA, 32 bp, Artificial Sequence
  synthetic oligonucleotide MO5017
ctagacacaaaaatgtacgactacgccttcgt

SEQ ID NO: 65 - DNA, 32 bp, Artificial Sequence
  synthetic oligonucleotide MO5018
gcacctgaagttcaccgtgcccgcggttccaa

SEQ ID NO: 66 - DNA, 32 bp, Artificial Sequence
  synthetic oligonucleotide MO5019
gtgcacgaaggcgtagtcgtacatttttgtgt

SEQ ID NO: 67 - DNA, 32 bp, Artificial Sequence
  synthetic oligonucleotide MO5020
cgcgttggaaccgcgggcacggtgaacttcag

SEQ ID NO: 68 - DNA, 17 bp, Artificial Sequence
  synthetic oligonucleotide MO5016
cccgcggcggtacttct

SEQ ID NO: 69 - DNA, 29 bp, Artificial Sequence
  synthetic oligonucleotide MO5013
ccgtctctacagcaggatcaggtcaatgc

SEQ ID NO: 70 - DNA, 29 bp, Artificial Sequence
  synthetic oligonucleotide MO5014
ccgtctcactgtactccttctgtcgcctg

SEQ ID NO: 71 - DNA, 28 bp, Artificial Sequence
  synthetic oligonucleotide MO5015
cacgcgtctactgctcatacaacgccct

SEQ ID NO: 72 - DNA, 1809 bp, Artificial Sequence
  al-2 protein of pMB4812
atgtacgactacgccttcgtgcacctgaagttcaccgtgcccgcggcggtacttctcaccgctatcgcctaccccattctcaacaggatacatctcatccaaacaggcttcctcgtcgtcgtcgcctttaccgccgctctgccatgggatgcctacttgattaagcacaaagtatggtcttacccaccagaagccattgttgggccgcgtttgcttggaattccctttgaagagctgttcttctttgtgatacagacttacatcacggcgctcgtatacatcctcttcaacaagccggtgctgcacgcgttgcacctcaacaatcaacaaaacccgccagcatggatgagggttgtcaaggttaccggccaggtagtcctcgtagccttgtcggtatggggatggaatgccgctcaggttcatcaggaaacaagctatctcggcttgatccttgtttgggcttgtccgttcttactggctatctggaccctcgctgggcgcttcattctcagcctaccctggtacgcgacggtgctcccgatgttcctacccaccttctatctttgggcggtagacgagtttgccttgcacaggggtacttggtccatcggatcggggacgaagctcgatttttgtctgtttggcaagttggacattgaagaagccacgttcttcctggtgaccaacatgctcatcgttggcggtatggccgcgttcgatcaatatctggccgtcatttacgctttcccaactctgttccccaaggtcaaccggtatccgacaactcatatgcttcttcaaagccgtcttatcaacacttccaggtacgatcttgagcgcattgagggcctgagagaagcggtcgagagactgcgcctgaagagcaggagtttttacctggccaattcgctcttttctggtcgactccgcattgacctgatcctgctgtactccttctgtcgcctggctgatgatctagtcgacgacgccaaatctcgccgtgaggtcttgtcctggaccgcgaagctgaaccacttccttgatctgcactacaaggacgcggacgccaccgaggaccccaagaaaaaggcggagcgaatcgacgcctacatcaagacagcgttccctccctgtgcctaccaagccctccacctcctgcccactcacattcttcctcccaagcctctttacgatctcatcaagggtttcgagatggactctcaattcaccttccacggtacttccgactctacggatctccaataccccatcgccgacgacaaggaccttgagaactacgctatctatgtcgccggtaccgtcggcgagctctgcatcgccctcatcatctaccactgcctgccagacatgtcggacactcagaagcgcgagctcgagaccgccgcgtgccggatgggcatcgcgctgcagtacgtcaacatcgctcgtgacatcgtcgtcgacgcacgtatcgggcgcgtttacttgcctaccacctggctcaagaaggaagggttgacgcacaagatggtcttggagaaccccgagggtcccgaggtcattgagcggatgagaagacggcttttggaaaatgcgtttgagctgtatgggggcgcgaggcctgagatgcaacggataccgagcgaggctaggggcccgatgattggtgccgttgaaaattacatggcgattggaagggtgttgagggagaggaaggaggggacggtgtttgtgaggatggaggggagggctacggtcccgaagcgaaggaggttgagcacgctgttgagggcgttgtatgagcagtag

SEQ ID NO: 73 - protein, 602 aa, Artificial Sequence
  al-2 protein of pMB4812
MYDYAFVHLKFTVPAAVLLTAIAYPILNRIHLIQTGFLVVVAFTAALPWDAYLIKHKVWSYPPEAIVGPRLLGIPFEELFFFVIQTYITALVYILFNKPVLHALHLNNQQNPPAWMRVVKVTGQVVLVALSVWGWNAAQVHQETSYLGLILVWACPFLLAIWTLAGRFILSLPWYATVLPMFLPTFYLWAVDEFALHRGTWSIGSGTKLDFCLFGKLDIEEATFFLVTNMLIVGGMAAFDQYLAVIYAFPTLFPKVNRYPTTHMLLQSRLINTSRYDLERIEGLREAVERLRLKSRSFYLANSLFSGRLRIDLILLYSFCRLADDLVDDAKSRREVLSWTAKLNHFLDLHYKDADATEDPKKKAERIDAYIKTAFPPCAYQALHLLPTHILPPKPLYDLIKGFEMDSQFTFHGTSDSTDLQYPIADDKDLENYAIYVAGTVGELCIALIIYHCLPDMSDTQKRELETAACRMGIALQYVNIARDIVVDARIGRVYLPTTWLKKEGLTHKMVLENPEGPEVIERMRRRLLENAFELYGGARPEMQRIPSEARGPMIGAVENYMAIGRVLRERKEGTVFVRMEGRATVPKRRRLSTLLRALYEQ

SEQ ID NO: 74 - DNA, 521 bp, Artificial Sequence
  (CrtZ) ORF based on protein sequence of Erythrobacter sp. NAP1, using Y. lipolytica codon bias
ctctagacacaaaaatgtcttggcctgccgctattgcagttacacttggtgcccttatttttatggaattctttgcttggtacgctcacaaatacattatgcatggatggggatggggttggcacagagaccatcacgaacctcacgacaacaaactggaaaaaaatgacctgttcgctgtggttttcggaacaattaacgctggtatgtatatttttggtgctctttattgggatgctttgtggtgggctgcacttggagttaatctttacggagtgatttacgcccttgttcatgacggactggttcatcaaagatttggaagatacgtccctaaaaacgcatacgctaaacgacttgttcaagcacacagattgcatcacgctactatcggtaaagaaggaggagtgtccttcggattcgttcttgctcgagaccctgctaaacttaaagccgaacttaaacgacaatctcaatccggagaagctattgttcgagaatccgccggagcctaaacgcgt

SEQ ID NO: 75 - protein, 166 aa, Artificial Sequence
  CrtZ protein of pMB4846
MSWPAAIAVTLGALIFMEFFAWYAHKYIMHGWGWGWHRDHHEPHDNKLEKNDLFAVVFGTINAGMYIFGALYWDALWWAALGVNLYGVIYALVHDGLVHQRFGRYVPKNAYAKRLVQAHRLHHATIGKEGGVSFGFVLARDPAKLKAELKRQSQSGEAIVRESAGA

SEQ ID NO: 76 - DNA, 623 bp, Artificial Sequence
  (CrtZ) ORF based on protein sequence of Sphingopyxis alaskensis, using Y. lipolytica codon bias
ctctagacacaaaaatgagccaccgaagagatccaggacttagaagagacgacgcacgatctatggcctcctgtctcagacgagcttacaacccccacatgtccctgcctgcaattttgtttttggttcttgctactgtcattgcaatggaaggagtcgcctgggcatcccacaaatacatcatgcacggatttggatgggcctggcacagagaccaccatgaaccccacgacaatcgactcgagaaaaacgacctgtttgccctgttcggagccgctatgtctatttctgccttcgctattggttctcctatgattatgggtgcagctgcctggaagcctggaacttggattggacttggtattcttctttacggtattatctacacactcgttcacgacggccttgtgcaccaaagatactttcgatgggtcccacgacgaggttacgcaaaacgacttgttcaagcacacaaacttcatcacgctacaatcggaaaagagggaggagtttctttcggatttgtttttgctcgtgaccctgctaaacttaaagccgaactgaaagcacaacgagaagctggtattgcagtcgtcagagaagcccttgctgactaaacgcgt

SEQ ID NO: 77 - protein, 200 aa, Artificial Sequence
  CrtZ protein of pMB4835
MSHRRDPGLRRDDARSMASCLRRAYNPHMSLPAILFLVLATVIAMEGVAWASHKYIMHGFGWAWHRDHHEPHDNRLEKNDLFALFGAAMSISAFAIGSPMIMGAAAWKPGTWIGLGILLYGIIYTLVHDGLVHQRYFRWVPRRGYAKRLVQAHKLHHATIGKEGGVSFGFVFARDPAKLKAELKAQREAGIAVVREALAD

SEQ ID NO: 78 - DNA, 482 bp, Robiginitalea biformata
cacaatctagacacaaaaatgacagtcttgatttggatcgcaattttcctggccaccttctgcttcatggaattcatggcctggtttacgcataaatatatcatgcacggtttcctctggagccttcataaggaccaccataaaaaggaccacgacagttggtttgagcgaaacgacgccttctttctattttatgcgatagtctccatgtcctttatcggggccgccgtgaacacgggattctggcaggggtggcccatcggcctgggcatcctcgcttacgggattgcctactttatcgtacacgatatctttatccatcagcggttcaagctctttcgcaatgcgaataactggtacgcgcggggtatccgcagggcccataaaatccaccacaagcacctgggaaaagaggaaggggaatgcttcgggatgctgtttgtcccatttaagtacttccggaagacctgaacgcgtttgtg

SEQ ID NO: 79 - protein, 150 aa, Artificial Sequence
  CrtZ protein of pMB4845
MTVLIWIAIFLATFCFMEFMAWFTHKYIMHGFLWSLHKDHHKKDHDSWFERNDAFFLFYAIVSMSFIGAAVNTGFWQGWPIGLGILAYGIAYFIVHDIFIHQRFKLFRNANNWYARGIRRAHKIHHKHLGKEEGECFGMLFVPFKYFRKT

SEQ ID NO: 80 - DNA, 536 bp, Xanthobacter autotrophicus
cacaatctagacacaaaaatgtccaccagcctcgccttcctcgtcaacgcgctcatcgtgatcgccacggtcgccgccatggaaggggtggcctgggccgcgcacaaatatgtcatgcacggcttcggctggggctggcacaagtcccaccacgagccgcgcgagggcgtgttcgagcgcaacgacctttatgcgctgctgttcgcaggcatcgccatcgccctcatctacgcgttccgcaatggcggcgcgctgctgtgggtgggcgtggggatgacggtctacggcttcctttatttcttcgtgcacgacggcatcacccaccagcgctggccgttccgctacgtgccgcgcaacggctatctcaagcgcctggtgcaggcccaccggctgcaccatgcggtggatggcaaggagggctgcgtctccttcggcttcatctatgccccgccgcctgccgacctgaaggccaagctgaagaagctgcacggcggcagcctgaagcagaacgaggcggcggaatagacgcgtttgtg

SEQ ID NO: 81 - protein, 168 aa, Artificial Sequence
  CrtZ protein of pMB4837
MSTSLAFLVNALIVIATVAAMEGVAWAAHKYVMHGFGWGWHKSHHEPREGVFERNDLYALLFAGIAIALIYAFRNGGALLWVGVGMTVYGFLYFFVHDGITHQRWPFRYVPRNGYLKRLVQAHRLHHAVDGKEGCVSFGFIYAPPPADLKAKLKKLHGGSLKQNEAAE

SEQ ID NO: 82 - DNA, 502 bp, Pseudomonas putida
tctctctagacacaaaaatggtgttcaatctcgccatattgttcggcaccctggtggccatggagggcgttggtacgctggctcacaagtacatcatgcatggctggggctggtggctgcaccgatcgcaccatgagccacacctgggcatgctcgaaaccaacgacctgtacctggtggccctggggctgatcgccacggcgctggtggcgctgggcaaaagtggttatgcgcctttgcagtgggtgggcggtggtgtggcaggctatggagcactgtatgtactggcccacgacggtttctttcaccggcactggccgcgcaagccgcggccggtcaaccgctacctgaaacgcttgcaccgcgcgcaccgcttgcaccatgcggtgaaggggcgcacggggagcgtgtcgttcgggttcttctatgcgccgccgctgaaggtgttgaagcagcaattgcgcagcaggcgcagccaatcgtgaacgcgtgagacgttgtg

SEQ ID NO: 83 - protein, 155 aa, Artificial Sequence
  CrtZ protein of pMB4850
MVFNLAILFGTLVAMEGVGTLAHKYIMHGWGWWLHRSHHEPHLGMLETNDLYLVALGLIATALVALGKSGYAPLQWVGGGVAGYGALYVLAHDGFFHRHWPRKPRPVNRYLKRLHRAHRLHHAVKGRTGSVSFGFFYAPPLKVLKQQLRSRRSQS

SEQ ID NO: 84 - DNA, 53 bp, Artificial Sequence
  Partial synthetic intron sequence
acaaacaaatgatgtgccgcatcgcattttaatattaaccattgcatacacag

SEQ ID NO: 85 - protein, 25 aa, Artificial Sequence
  partal synthetic intron sequence
KAWVSKQTNDVPHRILIPLHTQHLA

SEQ ID NO: 86 - protein, 19 aa, Artificial Sequence
  partial synthetic intron sequence
VSKQTNDVPHRILIPLHTQ

SEQ ID NO: 87 - DNA, 24 bp, Yarrowia lipolytica
gatctcgttctgctcgggtagatc

SEQ ID NO: 88 - DNA, 46 bp, Yarrowia lipolytica
gtgctctgcggtaagatcgactagtggtgtgttctgtggagcattc

SEQ ID NO: 89 - DNA, 58 bp, Yarrowia lipolytica
ccaccactgcactaccactacacctcgagcatgcatcaggaaggactctccctgtggt

SEQ ID NO: 90 - DNA, 27 bp, Yarrowia lipolytica
gtgttatggctctacgtgaaggggccc

SEQ ID NO: 91 - DNA, 21 bp, Yarrowia lipolytica
cccgacgttatccagaagaac

SEQ ID NO: 92 - DNA, 37 bp, Artificial Sequence
  synthetic oligonucleotide primer CrtZfwd
cacaccgtctcaaatgaccaatttcctgatcgtcgtc

SEQ ID NO: 93 - DNA, 29 bp, Artificial Sequence
  synthetic oligonucleotide primer CrtZrev
cacacagatctcacgtgcgctcctgcgcc

SEQ ID NO: 94 - DNA, 32 bp, Artificial Sequence
  synthetic oligonucleotide primer CrtWfwd
cacaccctaggccatgagcgcacatgccctgc

SEQ ID NO: 95 - DNA, 30 bp, Artificial Sequence
  synthetic oligonucleotide primer CrtWrev
cacacaagctttcatgcggtgtcccccttg

SEQ ID NO: 96 - DNA, 14 bp, Artificial Sequence
  synthetic phosphorylated oligonucleotide adaptor
aattcgcggccgct

SEQ ID NO: 97 - DNA, 10 bp, Artificial Sequence
  synthetic phosphorylated oligonucleotide adaptor
agcggccgcg

SEQ ID NO: 98 - DNA, 23 bp, Artificial Sequence
  synthetic oligonucleotide primer K
ccttctagtcgtacgtagtcagc

SEQ ID NO: 99 - DNA, 25 bp, Artificial Sequence
  synthetic oligonucleotide primer L
ccactgatctagaatctctttctgg

SEQ ID NO: 100 - DNA, 24 bp, Artificial Sequence
  synthetic oligonucleotide primer M
ggctcattgcgcatgctaacatcg

SEQ ID NO: 101 - DNA, 25 bp, Artificial Sequence
  synthetic oligonucleotide primer N
cgacgatgctatgagcttctagacg

SEQ ID NO: 102 - DNA, 34 bp, Artificial Sequence
  synthetic oligonucleotide primer 1fwd
cacacggatcctataatgccttccgcaacgaccg

SEQ ID NO: 103 - DNA, 35 bp, Artificial Sequence
  synthetic oligonucleotide primer 1rev
cacacactagttaaatttggacctcaacacgaccc

SEQ ID NO: 104 - DNA, 40 bp, Artificial Sequence
  synthetic oligonucleotide primer 2fwd
cacacggatccaatataaatgtctgcgaagagcatcctcg

SEQ ID NO: 105 - DNA, 34 bp, Artificial Sequence
  synthetic oligonucleotide primer 2rev
cacacgcatgcttaagcttggaactccaccgcac

SEQ ID NO: 106 - DNA, 47 bp, Artificial Sequence
  synthetic oligonucleotide primer gal10
cacacggatccaattttcaaaaattcttactttttttttggatggac

SEQ ID NO: 107 - DNA, 41 bp, Artificial Sequence
  synthetic oligonucleotide primer gal1
cacacggatccttttttctccttgacgttaaagtatagagg

SEQ ID NO: 108 - DNA, 38 bp, Artificial Sequence
  synthetic oligonucleotide primer AMD1fwd
cacacgagctcaaaaatggacaatcaggctacacagag

SEQ ID NO: 109 - DNA, 42 bp, Artificial Sequence
  synthetic oligonucleotide primer AMD1rev
cacaccctaggtcacttttcttcaatggttctcttgaaattg

SEQ ID NO: 110 - DNA, 46 bp, Artificial Sequence
  synthetic oligonucleotide primer gal7prox
cacacgagctcggaatattcaactgtttttttttatcatgttgatg

SEQ ID NO: 111 - DNA, 42 bp, Artificial Sequence
  synthetic oligonucleotide primer gal7dist
cacacggatccttcttgaaaatatgcactctatatcttttag

SEQ ID NO: 112 - DNA, 43 bp, Artificial Sequence
  synthetic oligonucleotide primer MAEfwd
cacacgctagctacaaaatgttgtcactcaaacgcatagcaac

SEQ ID NO: 113 - DNA, 38 bp, Artificial Sequence
  synthetic oligonucleotide primer MAErev
cacacgtcgacttaatgatctcggtatacgagaggaac

SEQ ID NO: 114 - DNA, 960 bp, Artificial Sequence
  synthetic Met2 promoter sequence
cctctcactttgtgaatcgtgaaacatgaatcttcaagccaagaatgttaggcaggggaagctttctttcagactttttggaattggtcctcttttggacattattgacgatattattattttttccccgtccaatgttgacccttgtaagccattccggttctggagcgcatctcgtctgaaggagtcttcgtgtggctataactacaagcgttgtatggtggatcctatgaccgtctatatagggcaacttttgctcttgttcttccccctccttgagggacgtatggcaatggctatgacaactatcgtagtgagcctctataacccattgaagtacaagtcctccaccttgctgccaaactcgcgagaaaaaaagtccaccaactccgccgggaaatactggagaacacctctaagacgtgggcttctgcacctgtgtggcttgggtctgggttttgcgagctctgagccacaacctaaggacggtgtgattgggagataagtagtcgttggttttctaatcgcacgtgatatgcaagccacacttataacacaatgaagacaggccgatgaactgcatgtcattgtacaggtgcggagagcaagaaactctggggcggaggtgaaagatgagacaaaaagcctcaggtgcaaggtagggagttgatcaacgtcaaacacaaataatctaggttgttaggcagctaaacatgtatataactgggctgccaccgagtgttacttgtcattaacgtcgcattttcgcctacacaaaatttgggttactcgccactacactgctcaaatctttcagctgtgcaacaagctttcaggtcacacatagactcgcataaggacccgggtcatctgttattctccactggtaaaccaatagtcctagctgatttgggtacagaagctcactttcacatcttttcatcttcttctacaaccatc

SEQ ID NO: 115 - DNA, 855 bp, Artificial Sequence
  synthetic Met3 promoter sequence
atctgtgaggagcccctggcgtcactgtcgactgtgccggcatttctgatggtatttccagccccgcagttctcgagacccccgaacaaatgtgccacacccttgccaaaatgacgaatacacggcgtcgcggccgggaatcgaactcttggcaccgccacaggagtgaaatttgaaatttgaaatttgaaaaataattcacattttgagtttcaataatatatcgatgaccctcccaaaagacccaagtcgagacgcaaaaaaacacccagacgacatggatgcggtcacgtgaccgcaaaaaccgccccggaaatccgtttgtgacgtgttcaattccatctctatgtttttctgcggtttctacgatgccgcaatggtggccaatgtgcgtttcactgccgtagtggctggaacaagccacagggggtcgtcgggccaatcagacggtccctgacatggttctgcgccctaacccgggaactctaacccccgtggtggcgcaatcgctgtcttcatgtgctttatctcacgtgacggctggaatctggcagaagacggagtatgtacattttgtcgttggtcacgttatccctaaaacgtggtgtttaaactggtcgaatgcttggcccagaacacaagaagaaaaaaacgagacaacttgatcagtttcaacgccacagcaagcttgtcttcactgtggttggtcttctccacgccacaagcaacacgtacatgtcaattacgtcagggtcttttaagttctgtggcttttgaaccagttataaagaaccaaccacccttttttcaaagctaatcaagacggggaaattttttttttgatatttttcgaca

SEQ ID NO: 116 - DNA, 897 bp, Artificial Sequence
  synthetic Met6 promoter sequence
gatactgcagacggtgcattacttacccgtgtcgactgagagtctacttggtacttggccctgtggctaagcagtatttgagcaacaatgcaatgcagttgctgactcggttccagatccccttgccccgatgtgtggaagcgttgtttttggggcaagggcatgtgggggctgcatcatactgtggctggggccgttggaagagccgtcggcagcgagcctgagtcgcttctcggggccttattccccccgcctctaggtcagcggcggccgaagtgtcgtactcagctcgcctgtacagtatgacgtgaccgaatagcctctggaaggttggagaagtacagtgcaaaaaaaagttgcaaaatttcattttagcgttcgatccgacgtggcagttggacaatgaatcgatggagacatgatcatgggcagaaatagaaggtctccatgttcaatggcagtaccaattgagcaacagacgggtcgacaggcggcgggcacaccatccgccctccacatggcgcaatcgtcagtgcagcgattcgtactcggattgcatcatgttgcaccgaaagttggggcccgcacgttggagaggcgaggagccagggttagctttggtggggtcctttgttgtcacgtggcatcagcgaatggcgtcctccaatcagggccgtcagcgaagtcggcgtgtgatagtgcgtggggagcgaatagagtttctgggggggggcggcccaaaacgtgaaatccgagtacgcatgtagagtgtaaattgggtgtatagtgacattgtttgactctgaccctgagagtaatatataatgtgtacgtgtccccctccgttggtcttctttttttctcctttctcctaaccaacacccaaactaatcaatc

SEQ ID NO: 117 - DNA, 972 bp, Artificial Sequence
  synthetic Met25 promoter sequence
aagtcgtattaacataactttccttacatttttttaaagcacgtcactatccacgtgacctagccacgcgataccaagtattcatccataatgacacactcatgacgtccggaggacgtcatcatcgtccagtcacgtgccaaggcacatgactaatcataacaccttatgactagcttctgaatcgctacacagttccaattcgcaaataaactcgaaatgacgaaatgccataataaaaatgacgaaactcgagattgagagcagcacatgcactgaagtggtggacaaccagcgtatccggagacacgacggatccagcaccatggaagctggccgaaaaagagatccccagcacattgagcaaccaagtcagctcaattgagtaacatcacacactcagatcgagtctgatggtggtccccttttgttccttcacttgaaaaataattgaaaataacaataacaataaaaataaaaacaaaataaaaataaaaataaaaataaaaataaaaaaataaaaaaaccttgccgcatttagcgtcagccaccccccgcattgacctgagtacgttggattgaccccgatcctgcacgtcgagcgtggtcggccaaaaagcgcccgtggctggtgagtcagaaatagcagggttgcaagagagagctgcgcaacgagcaataaacggtgtttttttcgcttctgtgctgcttagagtggagagccgaccctcgccatgctcacgtgaccattcacgtggttgcaaactccaccttagtatagccgtgtccctctcgctacccattatcgcatcgtactccagccacatttttttgttccccgctaaatccggaaccttatctgggtcacgtgaaattgcaatctcgacaggaggttatacttatagagtgagacactccacgcaaggtgttgcaagtcaattgacaccacctcacctcagactaacatccaca

SEQ ID NO: 118 - DNA, 1425 bp, Artificial Sequence
  synthetic Pox2 promoter sequence
gaatctgcccccacattttatctccgcttttgactgtttttctcccccctttcacactctgcttttggctacataaaccccgcaccgtttggaactctgttggtccggggaagccgccgttaggtgtgtcagatggagagcgccagacgagcagaaccgagggacagcggatcgggggagggctgtcacgtgacgaagggcactgttgacgtggtgaatgtcgcccgttctcacgtgacccgtctcctctatatgtgtatccgcctctttgtttggttttttttctgcttccccccccccccccccaccccaatcacatgctcagaaagtagacatctgcatcgtcctgcatgccatcccacaagacgaacaagtgataggccgagagccgaggacgaggtggagtgcacaaggggtaggcgaatggtacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacg

SEQ ID NO: 119 - DNA, 500 bp, Artificial Sequence
  synthetic Yef3 promoter sequence
cgccattcggttccttccagaccattccagatcaatccacctcttcttatctcaggtgggtgtgctgacatcagaccccgtagcccttctcccagtggcgaacagcaggcataaaacagggccattgagcagagcaaacaaggtcggtgaaatcgtcgaaaaagtcggaaaacggttgcaagaaattggagcgtcacctgccaccctccaggctctatataaagcattgccccaattgctaacgcttcatatttacacctttggcaccccagtccatccctccaataaaatgtactacatgggacacaacaagagaggatgcgcgcccaaaccctaacctagcacatgcacgatgattctctttgtctgtgaaaaaatttttccaccaaaatttccccattgggatgaaaccctaaccgcaaccaaaagtttttaactatcatcttgtacgtcacggtttccgattcttctcttctctttcatcatcatcacttgtgacc

SEQ ID NO: 120 - DNA, 494 bp, Artificial Sequence
  synthetic Can1 promoter sequence
aactaccataaagtaccgagaaatataggcaattgtacaaattgtccacctccttcacttacattaccgaaccatggccatatcaccaaaataccccgagtgctaaaacacctccctccaaatgttctcttaccttccaccgaaaaccgatcttattatcccaacgcttgttgtggcttgacgcgccgcacccgctgggcttgccatttcgataccaatccaagaggaaaagctcatgagaaacaatcggaatatcacgagaacggcctggcgaaccaacaggatatttttgaatataattacccctcgaatctagtcatatctatgtctactgtagacttgggcggcatcatgatgtacattattttagcgtctggaaccctaaagttcacgtacaatcatgtgacaaacgaggctaaaaaatgtcaatttcgtatattagtgttattacgtggctcacatttccgaatcatctaccaccccccacctaaaaa

SEQ ID NO: 121 - DNA, 440 bp, Artificial Sequence
  synthetic YALI0D16467g promoter sequence
tttttttaattttcatatttattttcatatttattttcatatttattttcatttatttattcatgtatttatttattactttttaagtattttaaactcctcactaaaccgtcgattgcacaatattaaccttcattacacctgcagcgtggtttttgtggtcgttagccgaagtcttccaacgtgggtataagtaggaacaattgggccgattttttgagccgtctaaatctctcgactcaattgatctgctgtcgaaaatccggctctctagctccttttccccgtccgctggagctcctcttcattgtgccgtttttccaacatttaactttgccacccaccaccacccccactaccatcacccactcgatctctgttcgtgtcaccacgactttgtcttctcacacatactctgtttgtgcaccacacattgcgaa

SEQ ID NO: 122 - DNA, 434 bp, Artificial Sequence
  synthetic Tef4 (YALI0B12562g) promoter sequence
gctacaatagctttattggccctattgagcacgctacaattcggtccagtatgtacaacgtctatgcgcactaacggccatacagtgagttacagcacacccaaaagtaaccctgcctgacctgtctgcctgagacaggaagattaactcttgtagtgaccgagctcgataagactcaagccacacaatttttttatagccttgcttcaagagtcgccaaaatgacattacacaactccacggaccgtcggttccatgtccacacccttggatgggtaagcgctccacgcacgtaccacgtgcattgagtttaaccacaaacataggtctgtgtcccagagttaccctgctgcatcagccaagtcttgaaagcaaaatttcttgcacaatttttcctcttcttttcttcactgatcgcagtccaaacacaaaca

SEQ ID NO: 123 - DNA, 752 bp, Artificial Sequence
  synthetic YALI0D12903g promoter sequence
gcgctctgatccacttgtatggctccaagttcagtgtaccaagtagttggtgatgcagggagggatgtctctatccaccaataatgaactcatgggcgaaattgtttctgttaaacactccaactgtcgttttaaatctcattctctttgcatttggactccattcgcttccgttgggccaatataatccatcgtaacgtactttagatggaaatttagttacctgctacttgtctcaacaccccaacaggggctgttcgacagaggtaatagagcgtcaatgggttaataaaaacacactgtcgattttcactcattgtctttatgatattacctgttttccgctgttatcaatgccgagcatcgtgttatatcttccaccccaactacttgcatttacttaactattacctcaactatttacaccccgaattgttacctcccaataagtaactttatttcaaccaatgggacgagagcatctctgagaacatcgatctatctctgtcaatattgcccagaatcgttcgaaaaaaaacaccaaaaggtttacagcgccattataaatataaattcgttgtcaattcccccgcaatgtctgttgaaatctcattttgagaccttccaacattaccctctctcccgtctggtcacatgacgtgactgcttcttcccaaaacgaacactcccaactcttcccccccgtcagtgaaaagtatacatccgacctccaaatcttttcttcactcaac

SEQ ID NO: 124 - DNA, 402 bp, Artificial Sequence
  synthetic Tef1 (YALI0C09141g) promoter sequence
agagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattc

SEQ ID NO: 125 - DNA, 491 bp, Artificial Sequence
  synthetic Fba1 (YALI0E26004g) promoter sequence
gctgcgctgatctggacaccacagaggttccgagcactttaggttgcaccaaatgtcccaccaggtgcaggcagaaaacgctggaacagcgtgtacagtttgtcttagcaaaaagtgaaggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatc

SEQ ID NO: 126 - DNA, 1213 bp, Artificial Sequence
  synthetic Pox2 terminator
gatgaggaatagacaagcgggtatttattgtatgaataaagattatgtattgattgcaaaaaagtgcatttgtagatgtggtttattgtagagagtacggtatgtactgtacgaacattaggagctacttctacaagtagattttcttaacaagggtgaaatttactaggaagtacatgcatatttcgttagtagaatcacaaaagaaatgtacaagcacgtactacttgtactccacaatgtggagtgggagcaaaaaaattggacgacaccggaatcgaaccggggacctcgcgcatgctaagcgcatgtgataaccaactacaccagacgcccaagaactttcttggtgattatggaatacgtggtctgctatatctcaattttgctgtaatgaatcattagaattaaaaaaaaaaccccatttttgtgtgattgtcggccaagagatggaacaggaagaatacgtgaacaagcgagcacgaatgccatatgctcttctgaacaaccgagtccgaatccgatttgtgggtatcacatgtctcaagtagctgaaatgtatttcgctagaataaaataaatgagattaagaattaaaaatattggaatatattttcctagaatagaaactttggattttttttcggctattacagtctgaactggacaaacggctgactatatataaatattattgggtctgttttcttgtttatgtcgaaattatctgggttttactactgtgtcgtcgagtatagagtggcctgactggagaaaatgcagtagtatggacagtaggtactgccagccagagaagtttttggaattgatacttgagtcatttttccattccccattccccattccaacacaatcaactgtttctgaacattttccaaaacgcggagatgtatgtcacttggcactgcaagtctcgattcaaaatgcatctctttcagaccaaagtgtcatcagctttgtttggccccaaattaccgcaaatacttgtcgaaattgaagtgcaatacggcctcgtctgccatgaaacctgcctattctcttcaaattggcgtcaggtttcacgtccagcattcctcgcccagacagagttgctatggttgaatcgtgtactgttaatatatgtatgtattatactcgtactacgatatactgttcaatagagtctcttataatcgtacgacgattctgggca

SEQ ID NO: 127 - DNA, 1341 bp, Yarrowia lipolytica
atgtcgcaaccccagaacgttggaatcaaagccctcgagatctacgtgccttctcgaattgtcaaccaggctgagctcgagaagcacgacggtgtcgctgctggcaagtacaccattggtcttggtcagaccaacatggcctttgtcgacgacagagaggacatctattcctttgccctgaccgccgtctctcgactgctcaagaacaacaacatcgaccctgcatctattggtcgaatcgaggttggtactgaaacccttctggacaagtccaagtccgtcaagtctgtgctcatgcagctctttggcgagaacagcaacattgagggtgtggacaacgtcaacgcctgctacggaggaaccaacgccctgttcaacgctatcaactgggttgagggtcgatcttgggacggccgaaacgccatcgtcgttgccggtgacattgccctctacgcaaagggcgctgcccgacccaccggaggtgccggctgtgttgccatgctcattggccccgacgctcccctggttcttgacaacgtccacggatcttacttcgagcatgcctacgatttctacaagcctgatctgacctccgagtacccctatgttgatggccactactccctgacctgttacacaaaggccctcgacaaggcctacgctgcctacaacgcccgagccgagaaggtcggtctgttcaaggactccgacaagaagggtgctgaccgatttgactactctgccttccacgtgcccacctgcaagcttgtcaccaagtcttacgctcgacttctctacaacgactacctcaacgacaagagcctgtacgagggccaggtccccgaggaggttgctgccgtctcctacgatgcctctctcaccgacaagaccgtcgagaagaccttccttggtattgccaaggctcagtccgccgagcgaatggctccttctctccagggacccaccaacaccggtaacatgtacaccgcctctgtgtacgcttctctcatctctctgctgacttttgtccccgctgagcagctgcagggcaagcgaatctctctcttctcttacggatctggtcttgcttccactcttttctctctgaccgtcaagggagacatttctcccatcgtcaaggcctgcgacttcaaggctaagctcgatgaccgatccaccgagactcccgtcgactacgaggctgccaccgatctccgagagaaggcccacctcaagaagaactttgagccccagggagacatcaagcacatcaagtctggcgtctactacctcaccaacatcgatgacatgttccgacgaaagtacgagatcaagcagtag

SEQ ID NO: 128 - protein, 446 aa, Yarrowia lipolytica
MSQPQNVGIKALEIYVPSRIVNQAELEKHDGVAAGKYTIGLGQTNMAFVDDREDIYSFALTAVSRLLKNNNIDPASIGRIEVGTETLLDKSKSVKSVLMQLFGENSNIEGVDNVNACYGGTNALFNAINWVEGRSWDGRNAIVVAGDIALYAKGAARPTGGAGCVAMLIGPDAPLVLDNVHGSYFEHAYDFYKPDLTSEYPYVDGHYSLTCYTKALDKAYAAYNARAEKVGLFKDSDKKGADRFDYSAFHVPTCKLVTKSYARLLYNDYLNDKSLYEGQVPEEVAAVSYDASLTDKTVEKTFLGIAKAQSAERMAPSLQGPTNTGNMYTASVYASLISLLTFVPAEQLQGKRISLFSYGSGLASTLFSLTVKGDISPIVKACDFKAKLDDRSTETPVDYEAATDLREKAHLKKNFEPQGDIKHIKSGVYYLTNIDDMFRRKYEIKQ

SEQ ID NO: 129 - DNA, 1350 bp, Yarrowia lipolytica
atggactacatcatttcggcgccaggcaaagtgattctatttggtgaacatgccgctgtgtttggtaagcctgcgattgcagcagccatcgacttgcgaacatacctgcttgtcgaaaccacaacatccgacaccccgacagtcacgttggagtttccagacatccacttgaacttcaaggtccaggtggacaagctggcatctctcacagcccagaccaaggccgaccatctcaattggtcgactcccaaaactctggataagcacattttcgacagcttgtctagcttggcgcttctggaagaacctgggctcactaaggtccagcaggccgctgttgtgtcgttcttgtacctctacatccacctatgtcccccttctgtgtgcgaagattcatcaaactgggtagttcgatcaacgctgcctatcggcgcgggcctgggctcttccgcatccatttgtgtctgtttggctgcaggtcttctggttctcaacggccagctgagcattgaccaggcaagagatttcaagtccctgaccgagaagcagctgtctctggtggacgactggtccttcgtcggtgaaatgtgcattcacggcaacccgtcgggcatcgacaatgctgtggctactcagggaggtgctctgttgttccagcgacctaacaaccgagtccctcttgttgacattcccgagatgaagctgctgcttaccaatacgaagcatcctcgatctaccgcagacctggttggtggagtcggagttctcactaaagagtttggctccatcatggatcccatcatgacttcagtaggcgagatttccaaccaggccatggagatcatttctagaggcaagaagatggtggaccagtctaaccttgagattgagcagggtatcttgcctcaacccacctctgaggatgcctgcaacgtgatggaagatggagctactcttcaaaagttgagagatatcggttcggaaatgcagcatctagtgagaatcaatcacggcctgcttatcgctatgggtgtttcccacccgaagctcgaaatcattcgaactgcctccattgtccacaacctgggtgagaccaagctcactggtgctggaggaggaggttgcgccatcactctagtcacttctaaagacaagactgcgacccagctggaggaaaatgtcattgctttcacagaggagatggctacccatggcttcgaggtgcacgagactactattggtgccagaggagttggtatgtgcattgaccatccctctctcaagactgttgaagccttcaagaaggtggagcgggcggatctcaaaaacatcggtccctggacccattag

SEQ ID NO: 130 - protein, 449 aa, Yarrowia lipolytica
MDYIISAPGKVILFGEHAAVFGKPAIAAAIDLRTYLLVETTTSDTPTVTLEFPDIHLNFKVQVDKLASLTAQTKADHLNWSTPKTLDKHIFDSLSSLALLEEPGLTKVQQAAVVSFLYLYIHLCPPSVCEDSSNWVVRSTLPIGAGLGSSASICVCLAAGLLVLNGQLSIDQARDFKSLTEKQLSLVDDWSFVGEMCIHGNPSGIDNAVATQGGALLFQRPNNRVPLVDIPEMKLLLTNTKHPRSTADLVGGVGVLTKEFGSIMDPIMTSVGEISNQAMEIISRGKKMVDQSNLEIEQGILPQPTSEDACNVMEDGATLQKLRDIGSEMQHLVRINHGLLIAMGVSHPKLEIIRTASIVHNLGETKLTGAGGGGCAITLVTSKDKTATQLEENVIAFTEEMATHGFEVHETTIGARGVGMCIDHPSLKTVEAFKKVERADLKNIGPWTH

SEQ ID NO: 131 - DNA, 1257 bp, Yarrowia lipolytica
atgaccacctattcggctccgggaaaggccctcctttgcggcggttatttggttattgatccggcgtattcagcatacgtcgtgggcctctcggcgcgtatttacgcgacagtttcggcttccgaggcctccaccacctctgtccatgtcgtctctccgcagtttgacaagggtgaatggacctacaactacacgaacggccagctgacggccatcggacacaacccatttgctcacgcggccgtcaacaccgttctgcattacgttcctcctcgaaacctccacatcaacatcagcatcaaaagtgacaacgcgtaccactcgcaaattgacagcacgcagagaggccagtttgcataccacaaaaaggcgatccacgaggtgcctaaaacgggcctcggtagctccgctgctcttaccaccgttcttgtggcagctttgctcaagtcatacggcattgatcccttgcataacacccacctcgttcacaacctgtcccaggttgcacactgctcggcacagaagaagattgggtctggatttgacgtggcttcggccgtttgtggctctctagtctatagacgtttcccggcggagtccgtgaacatggtcattgcagctgaagggacctccgaatacggggctctgttgagaactaccgttaatcaaaagtggaaggtgactctggaaccatccttcttgccgccgggaatcagcctgcttatgggagacgtccagggaggatctgagactccaggtatggtggccaaggtgatggcatggcgaaaagcaaagccccgagaagccgagatggtgtggagagatctcaacgctgccaacatgctcatggtcaagttgttcaacgacctgcgcaagctctctctcactaacaacgaggcctacgaacaacttttggccgaggctgctcctctcaacgctctaaagatgataatgttgcagaaccctctcggagaactagcacgatgcattatcactattcgaaagcatctcaagaagatgacacgggagactggtgctgctattgagccggatgagcagtctgcattgctcaacaagtgcaacacttatagtggagtcattggaggtgttgtgcctggagcaggaggctacgatgctatttctcttctggtgatcagctctacggtgaacaatgtcaagcgagagagccagggagtccaatggatggagctcaaggaggagaacgagggtctgcggctcgagaaggggttcaagtag

SEQ ID NO: 132 - protein, 418 aa, Yarrowia lipolytica
MTTYSAPGKALLCGGYLVIDPAYSAYVVGLSARIYATVSASEASTTSVHVVSPQFDKGEWTYNYTNGQLTAIGHNPFAHAAVNTVLHYVPPRNLHINISIKSDNAYHSQIDSTQRGQFAYHKKAIHEVPKTGLGSSAALTTVLVAALLKSYGIDPLHNTHLVHNLSQVAHCSAQKKIGSGFDVASAVCGSLVYRRFPAESVNMVIAAEGTSEYGALLRTTVNQKWKVTLEPSFLPPGISLLMGDVQGGSETPGMVAKVMAWRKAKPREAEMVWRDLNAANMLMVKLFNDLRKLSLTNNEAYEQLLAEAAPLNALKMIMLQNPLGELARCIITIRKHLKKMTRETGAAIEPDEQSALLNKCNTYSGVIGGVVPGAGGYDAISLLVISSTVNNVKRESQGVQWMELKEENEGLRLEKGFK

SEQ ID NO: 133 - DNA, 1164 bp, Yarrowia lipolytica
atgatccaccaggcctccaccaccgctccggtgaacattgcgacactcaagtactggggcaagcgagaccctgctctcaatctgcccactaacaactccatctccgtgactttgtcgcaggatgatctgcggaccctcaccacagcctcgtgttcccctgatttcacccaggacgagctgtggctcaatggcaagcaggaggacgtgagcggcaaacgtctggttgcgtgtttccgagagctgcgggctctgcgacacaaaatggaggactccgactcttctctgcctaagctggccgatcagaagctcaagatcgtgtccgagaacaacttccccaccgccgctggtctcgcctcatcggctgctggctttgccgccctgatccgagccgttgcaaatctctacgagctccaggagacccccgagcagctgtccattgtggctcgacagggctctggatccgcctgtcgatctctctacggaggctacgtggcatgggaaatgggcaccgagtctgacggaagcgactcgcgagcggtccagatcgccaccgccgaccactggcccgagatgcgagccgccatcctcgttgtctctgccgacaagaaggacacgtcgtccactaccggtatgcaggtgactgtgcacacttctcccctcttcaaggagcgagtcaccactgtggttcccgagcggtttgcccagatgaagaagtcgattctggaccgagacttccccacctttgccgagctcaccatgcgagactcaaaccagttccacgccacctgtctggactcgtatcctcccattttctacctcaacgacgtgtcgcgagcctccattcgggtagttgaggccatcaacaaggctgccggagccaccattgccgcctacacctttgatgctggacccaactgtgtcatctactacgaggacaagaacgaggagctggttctgggtgctctcaaggccattctgggccgtgtggagggatgggagaagcaccagtctgtggacgccaagaagattgatgttgacgagcggtgggagtccgagctggccaacggaattcagcgggtgatccttaccaaggttggaggagatcccgtgaagaccgctgagtcgcttatcaacgaggatggttctctgaagaacagcaagtag

SEQ ID NO: 134 - protein, 387 aa, Yarrowia lipolytica
MIHQASTTAPVNIATLKYWGKRDPALNLPTNNSISVTLSQDDLRTLTTASCSPDFTQDELWLNGKQEDVSGKRLVACFRELRALRHKMEDSDSSLPKLADQKLKIVSENNFPTAAGLASSAAGFAALIRAVANLYELQETPEQLSIVARQGSGSACRSLYGGYVAWEMGTESDGSDSRAVQIATADHWPEMRAAILVVSADKKDTSSTTGMQVTVHTSPLFKERVTTVVPERFAQMKKSILDRDFPTFAELTMRDSNQFHATCLDSYPPIFYLNDVSRASIRVVEAINKAAGATIAAYTFDAGPNCVIYYEDKNEELVLGALKAILGRVEGWEKHQSVDAKKIDVDERWESELANGIQRVILTKVGGDPVKTAESLINEDGSLKNSK

SEQ ID NO: 135 - DNA, 813 bp, Yarrowia lipolytica
atgacgacgtcttacagcgacaaaatcaagagtatcagcgtgagctctgtggctcagcagtttcctgaggtggcgccgattgcggacgtgtccaaggctagccggcccagcacggagtcgtcggactcgtcggccaagctatttgatggccacgacgaggagcagatcaagctgatggacgagatctgtgtggtgctggactgggacgacaagccgattggcggcgcgtccaaaaagtgctgtcatctgatggacaacatcaacgacggactggtgcatcgggccttttccgtgttcatgttcaacgaccgcggtgagctgcttctgcagcagcgggcggcggaaaaaatcacctttgccaacatgtggaccaacacgtgctgctcgcatcctctggcggtgcccagcgagatgggcgggctggatctggagtcccggatccagggcgccaaaaacgccgcggtccggaagcttgagcacgagctgggaatcgaccccaaggccgttccggcagacaagttccatttcctcacccggatccactacgccgcgccctcctcgggcccctggggcgagcacgagattgactacattctgtttgtccggggcgaccccgagctcaaggtggtggccaacgaggtccgcgataccgtgtgggtgtcgcagcagggactcaaggacatgatggccgatcccaagctggttttcaccccttggttccggctcatttgtgagcaggcgctgtttccctggtgggaccagttggacaatctgcccgcgggcgatgacgagattcggcggtggatcaagtag

SEQ ID NO: 136 - protein, 270 aa, Yarrowia lipolytica
MTTSYSDKIKSISVSSVAQQFPEVAPIADVSKASRPSTESSDSSAKLFDGHDEEQIKLMDEICVVLDWDDKPIGGASKKCCHLMDNINDGLVHRAFSVFMFNDRGELLLQQRAAEKITFANMWTNTCCSHPLAVPSEMGGLDLESRIQGAKNAAVRKLEHELGIDPKAVPADKFHFLTRIHYAAPSSGPWGEHEIDYILFVRGDPELKVVANEVRDTVWVSQQGLKDMMADPKLVFTPWFRLICEQALFPWWDQLDNLPAGDDEIRRWIK

SEQ ID NO: 137 - DNA, 1035 bp, Yarrowia lipolytica
atgtccaaggcgaaattcgaaagcgtgttcccccgaatctccgaggagctggtgcagctgctgcgagacgagggtctgccccaggatgccgtgcagtggttttccgactcacttcagtacaactgtgtgggtggaaagctcaaccgaggcctgtctgtggtcgacacctaccagctactgaccggcaagaaggagctcgatgacgaggagtactaccgactcgcgctgctcggctggctgattgagctgctgcaggcgtttttcctcgtgtcggacgacattatggatgagtccaagacccgacgaggccagccctgctggtacctcaagcccaaggtcggcatgattgccatcaacgatgctttcatgctagagagtggcatctacattctgcttaagaagcatttccgacaggagaagtactacattgaccttgtcgagctgttccacgacatttcgttcaagaccgagctgggccagctggtggatcttctgactgcccccgaggatgaggttgatctcaaccggttctctctggacaagcactcctttattgtgcgatacaagactgcttactactccttctacctgcccgttgttctagccatgtacgtggccggcattaccaaccccaaggacctgcagcaggccatggatgtgctgatccctctcggagagtacttccaggtccaggacgactaccttgacaactttggagaccccgagttcattggtaagatcggcaccgacatccaggacaacaagtgctcctggctcgttaacaaagcccttcagaaggccacccccgagcagcgacagatcctcgaggacaactacggcgtcaaggacaagtccaaggagctcgtcatcaagaaactgtatgatgacatgaagattgagcaggactaccttgactacgaggaggaggttgttggcgacatcaagaagaagatcgagcaggttgacgagagccgaggcttcaagaaggaggtgctcaacgctttcctcgccaagatttacaagcgacagaagtag

SEQ ID NO: 138 - protein, 344 aa, Yarrowia lipolytica
MSKAKFESVFPRISEELVQLLRDEGLPQDAVQWFSDSLQYNCVGGKLNRGLSVVDTYQLLTGKKELDDEEYYRLALLGWLIELLQAFFLVSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 139 - DNA, 1890 bp, Yarrowia lipolytica
atgttacgactacgaaccatgcgacccacacagaccagcgtcagggcggcgcttgggcccaccgccgcggcccgaaacatgtcctcctccagcccctccagcttcgaatactcgtcctacgtcaagggcacgcgggaaatcggccaccgaaaggcgcccacaacccgtctgtcggttgagggccccatctacgtgggcttcgacggcattcgtcttctcaacctgccgcatctcaacaagggctcgggattccccctcaacgagcgacgggaattcagactcagtggtcttctgccctctgccgaagccaccctggaggaacaggtcgaccgagcataccaacaattcaaaaagtgtggcactcccttagccaaaaacgggttctgcacctcgctcaagttccaaaacgaggtgctctactacgccctgctgctcaagcacgttaaggaggtcttccccatcatctatacaccgactcagggagaagccattgaacagtactcgcggctgttccggcggcccgaaggctgcttcctcgacatcaccagtccctacgacgtggaggagcgtctgggagcgtttggagaccatgacgacattgactacattgtcgtgactgactccgagggtattctcggaattggagaccaaggagtgggcggtattggtatttccatcgccaagctggctctcatgactctatgtgctggagtcaacccctcacgagtcattcctgtggttctggatacgggaaccaacaaccaggagctgctgcacgaccccctgtatctcggccgacgaatgccccgagtgcgaggaaagcagtacgacgacttcatcgacaactttgtgcagtctgcccgaaggctgtatcccaaggcggtgatccatttcgaggactttgggctcgctaacgcacacaagatcctcgacaagtatcgaccggagatcccctgcttcaacgacgacatccagggcactggagccgtcactttggcctccatcacggccgctctcaaggtgctgggcaaaaatatcacagatactcgaattctcgtgtacggagctggttcggccggcatgggtattgctgaacaggtctatgataacctggttgcccagggtctcgacgacaagactgcgcgacaaaacatctttctcatggaccgaccgggtctactgaccaccgcacttaccgacgagcagatgagcgacgtgcagaagccgtttgccaaggacaaggccaattacgagggagtggacaccaagactctggagcacgtggttgctgccgtcaagccccatattctcattggatgttccactcagcccggcgcctttaacgagaaggtcgtcaaggagatgctcaaacacacccctcgacccatcattctccctctttccaaccccacacgtcttcatgaggctgtccctgcagatctgtacaagtggaccgacggcaaggctctggttgccaccggctcgccctttgacccagtcaacggcaaggagacgtctgagaacaataactgctttgttttccccggaatcgggctgggagccattctgtctcgatcaaagctcatcaccaacaccatgattgctgctgccatcgagtgcctcgccgaacaggcccccattctcaagaaccacgacgagggagtacttcccgacgtagctctcatccagatcatttcggcccgggtggccactgccgtggttcttcaggccaaggctgagggcctagccactgtcgaggaagagctcaagcccggcaccaaggaacatgtgcagattcccgacaactttgacgagtgtctcgcctgggtcgagactcagatgtggcggcccgtctaccggcctctcatccatgtgcgggattacgactag

SEQ ID NO: 140 - protein, 629 aa, Yarrowia lipolytica
MLRLRTMRPTQTSVRAALGPTAAARNMSSSSPSSFEYSSYVKGTREIGHRKAPTTRLSVEGPIYVGFDGIRLLNLPHLNKGSGFPLNERREFRLSGLLPSAEATLEEQVDRAYQQFKKCGTPLAKNGFCTSLKFQNEVLYYALLLKHVKEVFPIIYTPTQGEAIEQYSRLFRRPEGCFLDITSPYDVEERLGAFGDHDDIDYIVVTDSEGILGIGDQGVGGIGISIAKLALMTLCAGVNPSRVIPVVLDTGTNNQELLHDPLYLGRRMPRVRGKQYDDFIDNFVQSARRLYPKAVIHFEDFGLANAHKILDKYRPEIPCFNDDIQGTGAVTLASITAALKVLGKNITDTRILVYGAGSAGMGIAEQVYDNLVAQGLDDKTARQNIFLMDRPGLLTTALTDEQMSDVQKPFAKDKANYEGVDTKTLEHVVAAVKPHILIGCSTQPGAFNEKVVKEMLKHTPRPIILPLSNPTRLHEAVPADLYKWTDGKALVATGSPFDPVNGKETSENNNCFVFPGIGLGAILSRSKLITNTMIAAAIECLAEQAPILKNHDEGVLPDVALIQIISARVATAVVLQAKAEGLATVEEELKPGTKEHVQIPDNFDECLAWVETQMWRPVYRPLIHVRDYD

SEQ ID NO: 141 - DNA, 2610 bp, Yarrowia lipolytica
atgccgcagcaagcaatggatatcaagggcaaggccaagtctgtgcccatgcccgaagaagacgacctggactcgcattttgtgggtcccatctctccccgacctcacggagcagacgagattgctggctacgtgggctgcgaagacgacgaagacgagcttgaagaactgggaatgctgggccgatctgcgtccacccacttctcttacgcggaagaacgccacctcatcgaggttgatgccaagtacagagctcttcatggccatctgcctcatcagcactctcagagtcccgtgtccagatcttcgtcatttgtgcgggccgaaatgaaccacccccctcccccaccctccagccacacccaccaacagccagaggacgatgacgcatcttccactcgatctcgatcgtcgtctcgagcttctggacgcaagttcaacagaaacagaaccaagtctggatcttcgctgagcaagggtctccagcagctcaacatgaccggatcgctcgaagaagagccctacgagagcgatgacgatgcccgactatctgcggaagacgacattgtctatgatgctacccagaaagacacctgcaagcccatatctcctactctcaaacgcacccgcaccaaggacgacatgaagaacatgtccatcaacgacgtcaaaatcaccaccaccacagaagatcctcttgtggcccaggagctgtccatgatgttcgaaaaggtgcagtactgccgagacctccgagacaagtaccaaaccgtgtcgctacagaaggacggagacaaccccaaggatgacaagacacactggaaaatttaccccgagcctccaccaccctcctggcacgagaccgaaaagcgattccgaggctcgtccaaaaaggagcaccaaaagaaagacccgacaatggatgaattcaaattcgaggactgcgaaatccccggacccaacgacatggtcttcaagcgagatcctacctgtgtctatcaggtctatgaggatgaaagctctctcaacgaaaataagccgtttgttgccatcccctcaatccgagattactacatggatctggaggatctcattgtggcttcgtctgacggacctgccaagtcttttgctttccgacgactgcaatatctagaagccaagtggaacctctactacctgctcaacgagtacacggagacaaccgagtccaagaccaacccccatcgagacttttacaacgtacgaaaggtcgacacccacgttcaccactctgcctgcatgaaccagaagcatctgctgcgattcatcaaatacaagatgaagaactgccctgatgaagttgtcatccaccgagacggtcgggagctgacactctcccaggtgtttgagtcacttaacttgactgcctacgacctgtctatcgatacccttgatatgcatgctcacaaggactcgttccatcgatttgacaagttcaacctcaagtacaaccctgtcggtgagtctcgactgcgagaaatcttcctaaagaccgacaactacatccagggtcgatacctagctgagatcacaaaggaggtgttccaggatctcgagaactcgaagtaccagatggcggagtaccgtatttccatctacggtcggtccaaggacgagtgggacaagctggctgcctgggtgctggacaacaaactgttttcgcccaatgttcggtggttgatccaggtgcctcgactgtacgacatttacaagaaggctggtctggttaacacctttgccgacattgtgcagaacgtctttgagcctcttttcgaggtcaccaaggatcccagtacccatcccaagctgcacgtgttcctgcagcgagttgtgggctttgactctgtcgatgacgagtcgaagctggaccgacgtttccaccgaaagttcccaactgcagcatactgggacagcgcacagaaccctccctactcgtactggcagtactatctatacgccaacatggcctccatcaacacctggagacagcgtttgggctataatacttttgagttgcgaccccatgctggagaggctggtgacccagagcatcttctgtgcacttatctggttgctcagggtatcaaccacggtattctgttgcgaaaggtgcccttcattcagtacctttactacctggaccagatccccattgccatgtctcctgtgtccaacaatgcgctgttcctcacgttcgacaagaaccccttctactcatacttcaagcggggtctcaacgtgtccttgtcatcggatgatcctctgcagtttgcttacactaaggaggctctgattgaggagtactctgtggctgcgctcatttacaagctttccaacgtggatatgtgtgagcttgctcgaaactcggtactgcaatctggctttgagcgaatcatcaaggagcattggatcggcgaaaactacgagatccatggccccgagggcaacaccatccagaagacaaacgtgcccaatgtgcgtctggccttccgagacgagactttgacccacgagcttgctctggtggacaagtacaccaatcttgaggagtttgagcggctgcatggttaa

SEQ ID NO: 142 - protein, 869 aa, Yarrowia lipolytica
MPQQAMDIKGKAKSVPMPEEDDLDSHFVGPISPRPHGADEIAGYVGCEDDEDELEELGMLGRSASTHFSYAEERHLIEVDAKYRALHGHLPHQHSQSPVSRSSSFVRAEMNHPPPPPSSHTHQQPEDDDASSTRSRSSSRASGRKFNRNRTKSGSSLSKGLQQLNMTGSLEEEPYESDDDARLSAEDDIVYDATQKDTCKPISPTLKRTRTKDDMKNMSINDVKITTTTEDPLVAQELSMMFEKVQYCRDLRDKYQTVSLQKDGDNPKDDKTHWKIYPEPPPPSWHETEKRFRGSSKKEHQKKDPTMDEFKFEDCEIPGPNDMVFKRDPTCVYQVYEDESSLNENKPFVAIPSIRDYYMDLEDLIVASSDGPAKSFAFRRLQYLEAKWNLYYLLNEYTETTESKTNPHRDFYNVRKVDTHVHHSACMNQKHLLRFIKYKMKNCPDEVVIHRDGRELTLSQVFESLNLTAYDLSIDTLDMHAHKDSFHRFDKFNLKYNPVGESRLREIFLKTDNYIQGRYLAEITKEVFQDLENSKYQMAEYRISIYGRSKDEWDKLAAWVLDNKLFSPNVRWLIQVPRLYDIYKKAGLVNTFADIVQNVFEPLFEVTKDPSTHPKLHVFLQRVVGFDSVDDESKLDRRFHRKFPTAAYWDSAQNPPYSYWQYYLYANMASINTWRQRLGYNTFELRPHAGEAGDPEHLLCTYLVAQGINHGILLRKVPFIQYLYYLDQIPIAMSPVSNNALFLTFDKNPFYSYFKRGLNVSLSSDDPLQFAYTKEALIEEYSVAALIYKLSNVDMCELARNSVLQSGFERIIKEHWIGENYEIHGPEGNTIQKTNVPNVRLAFRDETLTHELALVDKYTNLEEFERLHG

SEQ ID NO: 143 - DNA, 1017 bp, Yarrowia lipolytica
atgttccgaacccgagttaccggctccaccctgcgatccttctccacctccgctgcccgacagcacaaggttgtcgtccttggcgccaacggaggcattggccagcccctgtctctgctgctcaagctcaacaagaacgtgaccgacctcggtctgtacgatctgcgaggcgcccccggcgttgctgccgatgtctcccacatccccaccaactccaccgtggccggctactctcccgacaacaacggcattgccgaggccctcaagggcgccaagctggtgctgatccccgccggtgtcccccgaaagcccggcatgacccgagacgatctgttcaacaccaacgcctccattgtgcgagacctggccaaggccgtcggtgagcacgcccccgacgcctttgtcggagtcattgctaaccccgtcaactccaccgtccccattgtcgccgaggtgctcaagtccaagggcaagtacgaccccaagaagctcttcggtgtcaccaccctcgacgtcatccgagccgagcgattcgtctcccagctcgagcacaccaaccccaccaaggagtacttccccgttgttggcggccactccggtgtcaccattgtccccctcgtgtcccagtccgaccaccccgacattgccggtgaggctcgagacaagcttgtccaccgaatccagtttggcggtgacgaggttgtcaaggccaaggacggtgccggatccgccaccctttccatggcccaggctgccgcccgattcgccgactctctcctccgaggtgtcaacggcgagaaggacgttgttgagcccactttcgtcgactctcctctgttcaagggtgagggcatcgacttcttctccaccaaggtcactcttggccctaacggtgttgaggagatccaccccatcggaaaggtcaacgagtacgaggagaagctcatcgaggctgccaaggccgatctcaagaagaacattgagaagggtgtcaactttgtcaagcagaacccttaa

SEQ ID NO: 144 - protein, 338 aa, Yarrowia lipolytica
MFRTRVTGSTLRSFSTSAARQHKVVVLGANGGIGQPLSLLLKLNKNVTDLGLYDLRGAPGVAADVSHIPTNSTVAGYSPDNNGIAEALKGAKLVLIPAGVPRKPGMTRDDLFNTNASIVRDLAKAVGEHAPDAFVGVIANPVNSTVPIVAEVLKSKGKYDPKKLFGVTTLDVIRAERFVSQLEHTNPTKEYFPVVGGHSGVTIVPLVSQSDHPDIAGEARDKLVHRIQFGGDEVVKAKDGAGSATLSMAQAAARFADSLLRGVNGEKDVVEPTFVDSPLFKGEGIDFFSTKVTLGPNGVEEIHPIGKVNEYEEKLIEAAKADLKKNIEKGVNFVKQNP

SEQ ID NO: 145 - DNA, 1107 bp, Yarrowia lipolytica
atgacacaaacgcacaatctgttttcgccaatcaaagtgggctcttcggagctccagaaccggatcgttctcgcacccttgactcgaaccagagctctgcccggaaacgtgccctcggatcttgccacagagtactacgcacaaagagcagcatctccaggcactctcctcatcaccgaggccacatacatctcccccggatctgctggagtgcccattccaggagacggaatcgttccgggcatctggagtgacgagcagctcgaagcatggaaaaaggtgttcaaggccgtgcacgaccgaggatccaaaatctacgtccagctgtgggacattggacgtgtcgcatggtaccacaagctgcaggaactgggcaactacttccctacaggcccctcagctatccccatgaagggagaggagagcgagcatctcaaggctctgactcactgggagatcaagggcaaggtggccctctacgtcaacgctgccaagaacgccattgccgcaggcgctgatggcgtcgagatccactcggccaacggctaccttcccgacacatttctgagaagcgcctccaaccaacgaacagacgaatatggaggaagcatcgagaaccgggcccgattctcgctggagattgtcgacgctatcaccgaggccattggagcagacaaaaccgccatccgtctgtctccctggtccactttccaggacattgaggtgaatgacaccgagacccccgcacagttcacatacctgtttgagcagctgcagaagcgagccgacgagggaaagcagctggcctacgtgcatgtagttgagccccgactgtttggtccccccgagccctgggccaccaatgagcctttcagaaaaatttggaagggtaacttcattagagcaggtggatacgatagagagactgctcttgaggatgcagacaagtcagacaacaccctgattgcctttggtcgagacttcattgccaatcctgatctcgtccaacgcctcaagaataacgagcctttggccaagtacgacagaacaaccttctacgttccaggtgccaagggctacactgattaccctgcgtacaagatgtaa

SEQ ID NO: 146 - protein, 368 aa, Yarrowia lipolytica
MTQTHNLFSPIKVGSSELQNRIVLAPLTRTRALPGNVPSDLATEYYAQRAASPGTLLITEATYISPGSAGVPIPGDGIVPGIWSDEQLEAWKKVFKAVHDRGSKIYVQLWDIGRVAWYHKLQELGNYFPTGPSAIPMKGEESEHLKALTHWEIKGKVALYVNAAKNAIAAGADGVEIHSANGYLPDTFLRSASNQRTDEYGGSIENRARFSLEIVDAITEAIGADKTAIRLSPWSTFQDIEVNDTETPAQFTYLFEQLQKRADEGKQLAYVHVVEPRLFGPPEPWATNEPFRKIWKGNFIRAGGYDRETALEDADKSDNTLIAFGRDFIANPDLVQRLKNNEPLAKYDRTTFYVPGAKGYTDYPAYKM

SEQ ID NO: 147 - DNA, 1017 bp, Yarrowia lipolytica
atggaagccaaccccgaagtccagaccgatatcatcacgctgacccggttcattctgcaggaacagaacaaggtgggcgcgtcgtccgcaatccccaccggagacttcactctgctgctcaactcgctgcagtttgccttcaagttcattgcccacaacatccgacgatcgaccctggtcaacctgattggcctgtcgggaaccgccaactccaccggcgacgaccagaagaagctggacgtgatcggagacgagatcttcatcaacgccatgaaggcctccggtaaggtcaagctggtggtgtccgaggagcaggaggacctcattgtgtttgagggcgacggccgatacgccgtggtctgcgaccccatcgacggatcctccaacctcgacgccggcgtctccgtcggcaccattttcggcgtctacaagctccccgagggctcctccggatccatcaaggacgtgctccgacccggaaaggagatggttgccgccggctacaccatgtacggtgcctccgccaacctggtgctgtccaccggaaacggctgcaacggcttcactctcgatgaccctctgggagagttcatcctgacccaccccgatctcaagctccccgatctgcgatccatctactccgtcaacgagggtaactcctccctgtggtccgacaacgtcaaggactacttcaaggccctcaagttccccgaggacggctccaagccctactcggcccgatacattggctccatggtcgccgacgtgcaccgaaccattctctacggaggtatgtttgcctaccccgccgactccaagtccaagaagggcaagctccgacttttgtacgagggtttccccatggcctacatcattgagcaggccggcggtcttgccatcaacgacaacggcgagcgaatcctcgatctggtccccaccgagatccacgagcgatccggcgtctggctgggctccaagggcgagattgagaaggccaagaagtaccttctgaaatga

SEQ ID NO: 148 - protein, 338 aa, Yarrowia lipolytica
MEANPEVQTDIITLTRFILQEQNKVGASSAIPTGDFTLLLNSLQFAFKFIAHNIRRSTLVNLIGLSGTANSTGDDQKKLDVIGDEIFINAMKASGKVKLVVSEEQEDLIVFEGDGRYAVVCDPIDGSSNLDAGVSVGTIFGVYKLPEGSSGSIKDVLRPGKEMVAAGYTMYGASANLVLSTGNGCNGFTLDDPLGEFILTHPDLKLPDLRSIYSVNEGNSSLWSDNVKDYFKALKFPEDGSKPYSARYIGSMVADVHRTILYGGMFAYPADSKSKKGKLRLLYEGFPMAYIIEQAGGLAINDNGERILDLVPTEIHERSGVWLGSKGEIEKAKKYLLK

SEQ ID NO: 149 - DNA, 1194 bp, Yarrowia lipolytica
atgcgactcactctgccccgacttaacgccgcctacattgtaggagccgcccgaactcctgtcggcaagttcaacggagccctcaagtccgtgtctgccattgacctcggtatcaccgctgccaaggccgctgtccagcgatccaaggtccccgccgaccagattgacgagtttctgtttggccaggtgctgaccgccaactccggccaggcccccgcccgacaggtggttatcaagggtggtttccccgagtccgtcgaggccaccaccatcaacaaggtgtgctcttccggcctcaagaccgtggctctggctgcccaggccatcaaggccggcgaccgaaacgttatcgtggccggtggaatggagtccatgtccaacaccccctactactccggtcgaggtcttgttttcggcaaccagaagctcgaggactccatcgtcaaggacggtctctgggacccctacaacaacatccacatgggcaactgctgcgagaacaccaacaagcgagacggcatcacccgagagcagcaggacgagtacgccatcgagtcctaccgacgggccaacgagtccatcaagaacggcgccttcaaggatgagattgtccccgttgagatcaagacccgaaagggcaccgtgactgtctccgaggacgaggagcccaagggagccaacgccgagaagctcaagggcctcaagcctgtctttgacaagcagggctccgtcactgccggtaacgcctcccccatcaacgatggtgcttctgccgttgtcgttgcctctggcaccaaggccaaggagctcggtacccccgtgctcgccaagattgtctcttacgcagacgccgccaccgcccccattgactttaccattgctccctctctggccattcccgccgccctcaagaaggctggccttaccaaggacgacattgccctctgggagatcaacgaggccttctccggtgtcgctctcgccaacctcatgcgactcggaattgacaagtccaaggtcaacgtcaagggtggagctgttgctctcggccaccccattggtgcctccggtaaccgaatctttgtgactttggtcaacgccctcaaggagggcgagtacggagttgccgccatctgcaacggtggaggagcttccaccgccatcgtcatcaagaaggtctcttctgtcgagtag

SEQ ID NO: 150 - protein, 397 aa, Yarrowia lipolytica
MRLTLPRLNAAYIVGAARTPVGKFNGALKSVSAIDLGITAAKAAVQRSKVPADQIDEFLFGQVLTANSGQAPARQVVIKGGFPESVEATTINKVCSSGLKTVALAAQAIKAGDRNVIVAGGMESMSNTPYYSGRGLVFGNQKLEDSIVKDGLWDPYNNIHMGNCCENTNKRDGITREQQDEYAIESYRRANESIKNGAFKDEIVPVEIKTRKGTVTVSEDEEPKGANAEKLKGLKPVFDKQGSVTAGNASPINDGASAVVVASGTKAKELGTPVLAKIVSYADAATAPIDFTIAPSLAIPAALKKAGLTKDDIALWEINEAFSGVALANLMRLGIDKSKVNVKGGAVALGHPIGASGNRIFVTLVNALKEGEYGVAAICNGGGASTAIVIKKVSSVE

SEQ ID NO: 151 - DNA, 1953 bp, Yarrowia lipolytica
atgtctgccaacgagaacatctcccgattcgacgcccctgtgggcaaggagcaccccgcctacgagctcttccataaccacacacgatctttcgtctatggtctccagcctcgagcctgccagggtatgctggacttcgacttcatctgtaagcgagagaacccctccgtggccggtgtcatctatcccttcggcggccagttcgtcaccaagatgtactggggcaccaaggagactcttctccctgtctaccagcaggtcgagaaggccgctgccaagcaccccgaggtcgatgtcgtggtcaactttgcctcctctcgatccgtctactcctctaccatggagctgctcgagtacccccagttccgaaccatcgccattattgccgagggtgtccccgagcgacgagcccgagagatcctccacaaggcccagaagaagggtgtgaccatcattggtcccgctaccgtcggaggtatcaagcccggttgcttcaaggttggaaacaccggaggtatgatggacaacattgtcgcctccaagctctaccgacccggctccgttgcctacgtctccaagtccggaggaatgtccaacgagctgaacaacattatctctcacaccaccgacggtgtctacgagggtattgctattggtggtgaccgataccctggtactaccttcattgaccatatcctgcgatacgaggccgaccccaagtgtaagatcatcgtcctccttggtgaggttggtggtgttgaggagtaccgagtcatcgaggctgttaagaacggccagatcaagaagcccatcgtcgcttgggccattggtacttgtgcctccatgttcaagactgaggttcagttcggccacgccggctccatggccaactccgacctggagactgccaaggctaagaacgccgccatgaagtctgctggcttctacgtccccgataccttcgaggacatgcccgaggtccttgccgagctctacgagaagatggtcgccaagggcgagctgtctcgaatctctgagcctgaggtccccaagatccccattgactactcttgggcccaggagcttggtcttatccgaaagcccgctgctttcatctccactatttccgatgaccgaggccaggagcttctgtacgctggcatgcccatttccgaggttttcaaggaggacattggtatcggcggtgtcatgtctctgctgtggttccgacgacgactccccgactacgcctccaagtttcttgagatggttctcatgcttactgctgaccacggtcccgccgtatccggtgccatgaacaccattatcaccacccgagctggtaaggatctcatttcttccctggttgctggtctcctgaccattggtacccgattcggaggtgctcttgacggtgctgccaccgagttcaccactgcctacgacaagggtctgtccccccgacagttcgttgataccatgcgaaagcagaacaagctgattcctggtattggccatcgagtcaagtctcgaaacaaccccgatttccgagtcgagcttgtcaaggactttgttaagaagaacttcccctccacccagctgctcgactacgcccttgctgtcgaggaggtcaccacctccaagaaggacaacctgattctgaacgttgacggtgctattgctgtttcttttgtcgatctcatgcgatcttgcggtgcctttactgtggaggagactgaggactacctcaagaacggtgttctcaacggtctgttcgttctcggtcgatccattggtctcattgcccaccatctcgatcagaagcgactcaagaccggtctgtaccgacatccttgggacgatatcacctacctggttggccaggaggctatccagaagaagcgagtcgagatcagcgccggcgacgtttccaaggccaagactcgatcatag

SEQ ID NO: 152 - protein, 650 aa, Yarrowia lipolytica
MSANENISRFDAPVGKEHPAYELFHNHTRSFVYGLQPRACQGMLDFDFICKRENPSVAGVIYPFGGQFVTKMYWGTKETLLPVYQQVEKAAAKHPEVDVVVNFASSRSVYSSTMELLEYPQFRTIAIIAEGVPERRAREILHKAQKKGVTIIGPATVGGIKPGCFKVGNTGGMMDNIVASKLYRPGSVAYVSKSGGMSNELNNIISHTTDGVYEGIAIGGDRYPGTTFIDHILRYEADPKCKIIVLLGEVGGVEEYRVIEAVKNGQIKKPIVAWAIGTCASMFKTEVQFGHAGSMANSDLETAKAKNAAMKSAGFYVPDTFEDMPEVLAELYEKMVAKGELSRISEPEVPKIPIDYSWAQELGLIRKPAAFISTISDDRGQELLYAGMPISEVFKEDIGIGGVMSLLWFRRRLPDYASKFLEMVLMLTADHGPAVSGAMNTIITTRAGKDLISSLVAGLLTIGTRFGGALDGAATEFTTAYDKGLSPRQFVDTMRKQNKLIPGIGHRVKSRNNPDFRVELVKDFVKKNFPSTQLLDYALAVEEVTTSKKDNLILNVDGAIAVSFVDLMRSCGAFTVEETEDYLKNGVLNGLFVLGRSIGLIAHHLDQKRLKTGLYRHPWDDITYLVGQEAIQKKRVEISAGDVSKAKTRS

SEQ ID NO: 153 - DNA, 1494 bp, Yarrowia lipolytica
atgtcagcgaaatccattcacgaggccgacggcaaggccctgctcgcacactttctgtccaaggcgcccgtgtgggccgagcagcagcccatcaacacgtttgaaatgggcacacccaagctggcgtctctgacgttcgaggacggcgtggcccccgagcagatcttcgccgccgctgaaaagacctacccctggctgctggagtccggcgccaagtttgtggccaagcccgaccagctcatcaagcgacgaggcaaggccggcctgctggtactcaacaagtcgtgggaggagtgcaagccctggatcgccgagcgggccgccaagcccatcaacgtggagggcattgacggagtgctgcgaacgttcctggtcgagccctttgtgccccacgaccagaagcacgagtactacatcaacatccactccgtgcgagagggcgactggatcctcttctaccacgagggaggagtcgacgtcggcgacgtggacgccaaggccgccaagatcctcatccccgttgacattgagaacgagtacccctccaacgccacgctcaccaaggagctgctggcacacgtgcccgaggaccagcaccagaccctgctcgacttcatcaaccggctctacgccgtctacgtcgatctgcagtttacgtatctggagatcaaccccctggtcgtgatccccaccgcccagggcgtcgaggtccactacctggatcttgccggcaagctcgaccagaccgcagagtttgagtgcggccccaagtgggctgctgcgcggtcccccgccgctctgggccaggtcgtcaccattgacgccggctccaccaaggtgtccatcgacgccggccccgccatggtcttccccgctcctttcggtcgagagctgtccaaggaggaggcgtacattgcggagctcgattccaagaccggagcttctctgaagctgactgttctcaatgccaagggccgaatctggacccttgtggctggtggaggagcctccgtcgtctacgccgacgccattgcgtctgccggctttgctgacgagctcgccaactacggcgagtactctggcgctcccaacgagacccagacctacgagtacgccaaaaccgtactggatctcatgacccggggcgacgctcaccccgagggcaaggtactgttcattggcggaggaatcgccaacttcacccaggttggatccaccttcaagggcatcatccgggccttccgggactaccagtcttctctgcacaaccacaaggtgaagatttacgtgcgacgaggcggtcccaactggcaggagggtctgcggttgatcaagtcggctggcgacgagctgaatctgcccatggagatttacggccccgacatgcacgtgtcgggtattgttcctttggctctgcttggaaagcggcccaagaatgtcaagccttttggcaccggaccttctactgaggcttccactcctctcggagtttaa

SEQ ID NO: 154 - protein, 497 aa, Yarrowia lipolytica
MSAKSIHEADGKALLAHFLSKAPVWAEQQPINTFEMGTPKLASLTFEDGVAPEQIFAAAEKTYPWLLESGAKFVAKPDQLIKRRGKAGLLVLNKSWEECKPWIAERAAKPINVEGIDGVLRTFLVEPFVPHDQKHEYYINIHSVREGDWILFYHEGGVDVGDVDAKAAKILIPVDIENEYPSNATLTKELLAHVPEDQHQTLLDFINRLYAVYVDLQFTYLEINPLVVIPTAQGVEVHYLDLAGKLDQTAEFECGPKWAAARSPAALGQVVTIDAGSTKVSIDAGPAMVFPAPFGRELSKEEAYIAELDSKTGASLKLTVLNAKGRIWTLVAGGGASVVYADAIASAGFADELANYGEYSGAPNETQTYEYAKTVLDLMTRGDAHPEGKVLFIGGGIANFTQVGSTFKGIIRAFRDYQSSLHNHKVKIYVRRGGPNWQEGLRLIKSAGDELNLPMEIYGPDMHVSGIVPLALLGKRPKNVKPFGTGPSTEASTPLGV

SEQ ID NO: 155 - DNA, 1458 bp, Yarrowia lipolytica
atggttattatgtgtgtgggacctcagcacacgcatcatcccaacacagggtgcagtatatatagacagacgtgttccttcgcaccgttcttcacatatcaaaacactaacaaattcaaaagtgagtatcatggtgggagtcaattgattgctcggggagttgaacaggcaacaatggcatgcacagggccagtgaaggcagactgcagtcgctgcacatggatcgtggttctgaggcgttgctatcaaaagggtcaattacctcacgaaacacagctggatgttgtgcaatcgtcaattgaaaaacccgacacaatgcaagatctctttgcgcgcattgccatcgctgttgccatcgctgtcgccatcgccaatgccgctgcggattattatccctaccttgttccccgcttccgcacaaccggcgatgtctttgtatcatgaactctcgaaactaactcagtggttaaagctgtcgttgccggagccgctggtggtattggccagcccctttctcttctcctcaaactctctccttacgtgaccgagcttgctctctacgatgtcgtcaactcccccggtgttgccgctgacctctcccacatctccaccaaggctaaggtcactggctacctccccaaggatgacggtctcaagaacgctctgaccggcgccaacattgtcgttatccccgccggtatcccccgaaagcccggtatgacccgagacgatctgttcaagatcaacgctggtatcgtccgagatctcgtcaccggtgtcgcccagtacgcccctgacgcctttgtgctcatcatctccaaccccgtcaactctaccgtccctattgctgccgaggtcctcaagaagcacaacgtcttcaaccctaagaagctcttcggtgtcaccacccttgacgttgtccgagcccagaccttcaccgccgctgttgttggcgagtctgaccccaccaagctcaacatccccgtcgttggtggccactccggagacaccattgtccctctcctgtctctgaccaagcctaaggtcgagatccccgccgacaagctcgacgacctcgtcaagcgaatccagtttggtggtgacgaggttgtccaggctaaggacggtcttggatccgctaccctctccatggcccaggctggtttccgatttgccgaggctgtcctcaagggtgccgctggtgagaagggcatcatcgagcccgcctacatctaccttgacggtattgatggcacctccgacatcaagcgagaggtcggtgtcgccttcttctctgtccctgtcgagttcggccctgagggtgccgctaaggcttacaacatccttcccgaggccaacgactacgagaagaagcttctcaaggtctccatcgacggtctttacggcaacattgccaagggcgaggagttcattgttaaccctcctcctgccaagtaa

SEQ ID NO: 156 - protein, 331 aa, Yarrowia lipolytica
VVKAVVAGAAGGIGQPLSLLLKLSPYVTELALYDVVNSPGVAADLSHISTKAKVTGYLPKDDGLKNALTGANIVVIPAGIPRKPGMTRDDLFKINAGIVRDLVTGVAQYAPDAFVLIISNPVNSTVPIAAEVLKKHNVFNPKKLFGVTTLDVVRAQTFTAAVVGESDPTKLNIPVVGGHSGDTIVPLLSLTKPKVEIPADKLDDLVKRIQFGGDEVVQAKDGLGSATLSMAQAGFRFAEAVLKGAAGEKGIIEPAYIYLDGIDGTSDIKREVGVAFFSVPVEFGPEGAAKAYNILPEANDYEKKLLKVSIDGLYGNIAKGEEFIVNPPPAK

SEQ ID NO: 157 - DNA, 1937 bp, Yarrowia lipolytica
atgactggcaccttacccaagttcggcgacggaaccaccattgtggttcttggagcctccggcgacctcgctaagaagaagaccgtgagtattgaaccagactgaggtcaattgaagagtaggagagtctgagaacattcgacggacctgattgtgctctggaccactcaattgactcgttgagagccccaatgggtcttggctagccgagtcgttgacttgttgacttgttgagcccagaacccccaacttttgccaccatacaccgccatcaccatgacacccagatgtgcgtgcgtatgtgagagtcaattgttccgtggcaaggcacagcttattccaccgtgttccttgcacaggtggtctttacgctctcccactctatccgagcaataaaagcggaaaaacagcagcaagtcccaacagacttctgctccgaataaggcgtctagcaagtgtgcccaaaactcaattcaaaaatgtcagaaacctgatatcaacccgtcttcaaaagctaaccccagttccccgccctcttcggcctttaccgaaacggcctgctgcccaaaaatgttgaaatcatcggctacgcacggtcgaaaatgactcaggaggagtaccacgagcgaatcagccactacttcaagacccccgacgaccagtccaaggagcaggccaagaagttccttgagaacacctgctacgtccagggcccttacgacggtgccgagggctaccagcgactgaatgaaaagattgaggagtttgagaagaagaagcccgagccccactaccgtcttttctacctggctctgccccccagcgtcttccttgaggctgccaacggtctgaagaagtatgtctaccccggcgagggcaaggcccgaatcatcatcgagaagccctttggccacgacctggcctcgtcacgagagctccaggacggccttgctcctctctggaaggagtctgagatcttccgaatcgaccactacctcggaaaggagatggtcaagaacctcaacattctgcgatttggcaaccagttcctgtccgccgtgtgggacaagaacaccatttccaacgtccagatctccttcaaggagccctttggcactgagggccgaggtggatacttcaacgacattggaatcatccgagacgttattcagaaccatctgttgcaggttctgtccattctagccatggagcgacccgtcactttcggcgccgaggacattcgagatgagaaggtcaaggtgctccgatgtgtcgacattctcaacattgacgacgtcattctcggccagtacggcccctctgaagacggaaagaagcccggatacaccgatgacgatggcgttcccgatgactcccgagctgtgacctttgctgctctccatctccagatccacaacgacagatgggagggtgttcctttcatcctccgagccggtaaggctctggacgagggcaaggtcgagatccgagtgcagttccgagacgtgaccaagggcgttgtggaccatctgcctcgaaatgagctcgtcatccgaatccagccctccgagtccatctacatgaagatgaactccaagctgcctggccttactgccaagaacattgtcaccgacctggatctgacctacaaccgacgatactcggacgtgcgaatccctgaggcttacgagtctctcattctggactgcctcaagggtgaccacaccaactttgtgcgaaacgacgagctggacatttcctggaagattttcaccgatctgctgcacaagattgacgaggacaagagcattgtgcccgagaagtacgcctacggctctcgtggccccgagcgactcaagcagtggctccgagaccgaggctacgtgcgaaacggcaccgagctgtaccaatggcctgtcaccaagggctcctcgtga

SEQ ID NO: 158 - protein, 498 aa, Yarrowia lipolytica
MTGTLPKFGDGTTIVVLGASGDLAKKKTFPALFGLYRNGLLPKNVEIIGYARSKMTQEEYHERISHYFKTPDDQSKEQAKKFLENTCYVQGPYDGAEGYQRLNEKIEEFEKKKPEPHYRLFYLALPPSVFLEAANGLKKYVYPGEGKARIIIEKPFGHDLASSRELQDGLAPLWKESEIFRIDHYLGKEMVKNLNILRFGNQFLSAVWDKNTISNVQISFKEPFGTEGRGGYFNDIGIIRDVIQNHLLQVLSILAMERPVTFGAEDIRDEKVKVLRCVDILNIDDVILGQYGPSEDGKKPGYTDDDGVPDDSRAVTFAALHLQIHNDRWEGVPFILRAGKALDEGKVEIRVQFRDVTKGVVDHLPRNELVIRIQPSESIYMKMNSKLPGLTAKNIVTDLDLTYNRRYSDVRIPEAYESLILDCLKGDHTNFVRNDELDISWKIFTDLLHKIDEDKSIVPEKYAYGSRGPERLKQWLRDRGYVRNGTELYQWPVTKGSS

SEQ ID NO: 159 - DNA, 2202 bp, Yarrowia lipolytica
atgactgacacttcaaacatcaagtgagtattgccgcacacaattgcaatcaccgccgggctctacctcctcagctctcgacgtcaatgggccagcagccgccatttgaccccaattacactggttgtgtaaaaccctcaaccacaatcgcttatgctcaccacagactacgacttaaccaagtcatgtcacaggtcaaagtaaagtcagcgcaacaccccctcaatctcaacacacttttgctaactcaggcctgtcgctgacattgccctcatcggtctcgccgtcatgggccagaacctgatcctcaacatggccgaccacggtaagtatcaattgactcaagacgcaccagcaagatacagagcatacccagcaatcgctcctctgataatcgccattgtaacactacgttggttagattgatctaaggtcgttgctggttccatgcacttccacttgctcatatgaagggagtcaaactctattttgatagtgtcctctcccatccccgaaatgtcgcattgttgctaacaataggctacgaggttgttgcctacaaccgaaccacctccaaggtcgaccacttcctcgagaacgaggccaagggtgagtatccgtccagctatgctgtttacagccattgaccccaccttcccccacaattgctacgtcaccattaaaaaacaaaattaccggtatcggcaagctagactttcatgcaacctacgcagggtaacaagttgagtttcagccgtgcaccttacaggaaaaccagtcatacgccgaggcagtgtgaaagcgaaagcacacagcctacggtgattgattgcatttttttgacataggagggaaacacgtgacatggcaagtgcccaacacgaatactaacaaacaggaaagtccattattggtgctcactctatcaaggagctgtgtgctctgctgaagcgaccccgacgaatcattctgctcgttaaggccggtgctgctgtcgattctttcatcgaacagctcctgccctatctcgataagggtgatatcatcattgacggtggtaactcccacttccccgactccaaccgacgatacgaggagcttaacgagaagggaatcctctttgttggttccggtgtttccggcggtgaggagggtgcccgatacggtccctccatcatgcccggtggaaacaaggaggcctggccccacattaagaagattttccaggacatctctgctaaggctgatggtgagccctgctgtgactgggtcggtgacgctggtgccggccactttgtcaagatggttcacaacggtattgagtatggtgacatgcagcttatctgcgaggcttacgacctcatgaagcgaggtgctggtttcaccaatgaggagattggagacgttttcgccaagtggaacaacggtatcctcgactccttcctcattgagatcacccgagacatcttcaagtacgacgacggctctggaactcctctcgttgagaagatctccgacactgctggccagaagggtactggaaagtggaccgctatcaacgctcttgaccttggtatgcccgtcaccctgatcggtgaggccgtcttcgctcgatgcctttctgccctcaagcaggagcgtgtccgagcttccaaggttcttgatggccccgagcccgtcaagttcactggtgacaagaaggagtttgtcgaccagctcgagcaggccctttacgcctccaagatcatctcttacgcccagggtttcatgcttatccgagaggccgccaagacctacggctgggagctcaacaacgccggtattgccctcatgtggcgaggtggttgcatcatccgatccgtcttccttgctgacatcaccaaggcttaccgacaggaccccaacctcgagaacctgctgttcaacgacttcttcaagaacgccatctccaaggccaacccctcttggcgagctaccgtggccaaggctgtcacctggggtgttcccactcccgcctttgcctcggctctggctttctacgacggttaccgatctgccaagctccccgctaacctgctccaggcccagcgagactacttcggcgcccacacctaccagctcctcgatggtgatggaaagtggatccacaccaactggaccggccgaggtggtgaggtttcttcttccacttacgatgcttaa

SEQ ID NO: 160 - protein, 489 aa, Yarrowia lipolytica
MTDTSNIKPVADIALIGLAVMGQNLILNMADHGYEVVAYNRTTSKVDHFLENEAKGKSIIGAHSIKELCALLKRPRRIILLVKAGAAVDSFIEQLLPYLDKGDIIIDGGNSHFPDSNRRYEELNEKGILFVGSGVSGGEEGARYGPSIMPGGNKEAWPHIKKIFQDISAKADGEPCCDWVGDAGAGHFVKMVHNGIEYGDMQLICEAYDLMKRGAGFTNEEIGDVFAKWNNGILDSFLIEITRDIFKYDDGSGTPLVEKISDTAGQKGTGKWTAINALDLGMPVTLIGEAVFARCLSALKQERVRASKVLDGPEPVKFTGDKKEFVDQLEQALYASKIISYAQGFMLIREAAKTYGWELNNAGIALMWRGGCIIRSVFLADITKAYRQDPNLENLLFNDFFKNAISKANPSWRATVAKAVTWGVPTPAFASALAFYDGYRSAKLPANLLQAQRDYFGAHTYQLLDGDGKWIHTNWTGRGGEVSSSTYDA

SEQ ID NO: 161 - DNA, 1742 bp, Yarrowia lipolytica
atgctcaaccttagaaccgcccttcgagctgtgcgacccgtcactctggtgagtatctcggagcccgggacggctaccaacacacaagcaagatgcaacagaaaccggactttttaaatgcggattgcggaaaatttgcatggcggcaacgactcggagaaggagcgggacaattgcaatggcaggatgccattgacgaactgagggtgatgagagaccgggcctccgatgacgtggtggtgacgacagcccggctggtgttgccgggactgtctctgaaaagcaatttctctatctccggtctcaacagactccccttctctagctcaattggcattgtcttcagaaggtgtcttagtggtatccccattgttatcttcttttccccaatgtcaatgtcaatgtcaatggctccgacctctttcacattaacacggcgcaaacacagataccacggaaccgactcaaacaaatccaaagagacgcagcggaataattggcatcaacgaacgatttgggatactctggcgagaatgccgaaatatttcgcttgtcttgttgtttctcttgagtgagttgtttgtgaagtcgtttggaagaaggttcccaatgtcacaaaccataccaactcgttacagccagcttgtaatcccccacctcttcaatacatactaacgcagacccgatcctacgccacttccgtggcctctttcaccggccagaagaactccaacggcaagtacactgtgtctctgattgagggagacggtatcggaaccgagatctccaaggctgtcaaggacatctaccatgccgccaaggtccccatcgactgggaggttgtcgacgtcacccccactctggtcaacggcaagaccaccatccccgacagcgccattgagtccatcaaccgaaacaaggttgccctcaagggtcccctcgccacccccatcggtaagggccacgtttccatgaacctgactctgcgacgaaccttcaacctgttcgccaacgtccgaccttgcaagtccgtcgtgggctacaagaccccttacgagaacgtcgacaccctgctcatccgagagaacactgagggtgagtactccggtatcgagcacaccgtcgtccccggtgtcgttcagtccatcaagctgatcacccgagaggcttccgagcgagtcatccggtacgcttacgagtacgccctgtcccgaggcatgaagaaggtccttgttgtccacaaggcctctattatgaaggtctccgatggtcttttccttgaggttgctcgagagctcgccaaggagtacccctccattgacctttccgtcgagctgatcgacaacacctgtctgcgaatggtccaggaccccgctctctaccgagatgtcgtcatggtcatgcccaacctttacggtgacattctgtccgatcttgcctccggtcttatcggtggtcttggtctgaccccctccggtaacatgggtgacgaggtctccatcttcgaggccgtccacggatccgctcccgacattgctggcaagggtcttgctaaccccactgctctgctgctctcctccgtgatgatgctgcgacacatgggtctcaacgacaacgccaccaacatcgagcaggccgtctttggcaccattgcttccggccccgagaaccgaaccaaggatcttaagggtaccgccaccacttctcactttgctgagcagattatcaagcgactcaagtag

SEQ ID NO: 162 - protein, 369 aa, Yarrowia lipolytica
MLNLRTALRAVRPVTLTRSYATSVASFTGQKNSNGKYTVSLIEGDGIGTEISKAVKDIYHAAKVPIDWEVVDVTPTLVNGKTTIPDSAIESINRNKVALKGPLATPIGKGHVSMNLTLRRTFNLFANVRPCKSVVGYKTPYENVDTLLIRENTEGEYSGIEHTVVPGVVQSIKLITREASERVIRYAYEYALSRGMKKVLVVHKASIMKVSDGLFLEVARELAKEYPSIDLSVELIDNTCLRMVQDPALYRDVVMVMPNLYGDILSDLASGLIGGLGLTPSGNMGDEVSIFEAVHGSAPDIAGKGLANPTALLLSSVMMLRHMGLNDNATNIEQAVFGTIASGPENRTKDLKGTATTSHFAEQIIKRLK

SEQ ID NO: 163 - DNA, 31 bp, Artificial Sequence
  synthetic oligonucleotide primer MO5651
cacaaactagtgtcaggaatatgaaaccagg

SEQ ID NO: 164 - DNA, 31 bp, Artificial Sequence
  synthetic oligonucleotide primer MO5652
cacaaactagtgcatgtgataggaaggagga

SEQ ID NO: 165 - DNA, 31 bp, Artificial Sequence
  synthetic oligonucleotide primer MO4814
cacaacgtctctctagacacaaaaatgagct

SEQ ID NO: 166 - DNA, 39 bp, Artificial Sequence
  synthetic oligonucleotide primer MO4816
cacaacgtctcagccggcacctgctcccatagaatctcg

SEQ ID NO: 167 - DNA, 45 bp, Artificial Sequence
  synthetic oligonucleotide primer MO5060
cacaagaagacaacggcgcaggagccatggaccctaccggagacg

SEQ ID NO: 168 - DNA, 38 bp, Artificial Sequence
  synthetic oligonucleotide primer MO5061
cacaagaagacaacgcgtttaagggccggttctctttc

SEQ ID NO: 169 - protein, 435 aa, Artificial Sequence
  synthetic crtZW chimeric sequence
MSWWAIALIVFGAVVGMEFFAWFAHKYIMHGWGWSWHRDHHEPHDNTLEKNDLFAVVFGSVAALLFVIGALWSDPLWWAAVGITLYGVIYTLVHDGLVHQRYWRWTPKRGYAKRLVQAHRLHHATVGKEGGVSFGFVFARDPAKLKAELKQQREQGLAVVRDSMGAGAGAGAMDPTGDVTASPRPQTTIPVRQALWGLSLAGAIIAAWVFMHIGFVFFAPLDPIVLALAPVIILLQSWLSVGLFIISHDAIHGSLAPGRPAFNRAMGRLCMTLYAGFDFDRMAAAHHRHHRSPGTAADPDFSVDSPDRPLPWFGAFFRRYFGWRPFLTVNAVVFTYWLVLGANPVNIVLFYGVPALLSAGQLFYFGTFLPHRHERQGFADHHRARSVRSPYMLSLVTCYHFGGYHHEHHLFPHEPWWRLPQRGGWERDRRKRTGP

SEQ ID NO: 170 - DNA, 1326 bp, Ostreococcus lucimarinus
atgaaggatgatcgcgaatggattgcgtttcaacagcgcaaggtgtttagtgagcaaaagcaaatcaaagagtacctcagtgctttgaacgaccgcgacaaggtcgacgttctcgttgtcggtgcgggccccgcaggtctggcgatcgcagcggagacggcgaagaagggtctttctgttggtctcgtcgcaccagacaccccgttcgtgaacaactacggagtatggctcgacgagttcaaagatctagggctcgaacactgcttgcttcataagtatgacgacgcattggtttggttcgatgattctgatcctgcgagtggaactgaactcggtcgaccttacggtcaagtgtgccgcaggcgtcttcgcgaccatttgttgaaggagtgcgcggcggctggcgtcaagtatttaccaggcctggtagattttgtgcgtcacggtgacgtcgaaaagaacgagttagccgaagcaaacagaggccagcaattcacgttgaattcgcgtctcgtcgttgccggcaccggtcacaaccgcgacatgctcagctacgaagagggtgcgccgccgggctggcagactgcgtatggcgttgaggtgcgcattccgaaccacggttttcccgtgaacaaggccgtgttcatggattttcgtcaaagcgatccggaggcgatgaaagaggaacaagacgagggcgtttggcgcgtgccgtctttcctttacgtgttacccgtggacaaggatgtggtgttcgtcgaggagacgtgcctcgtcgcgcgcgtacaagtgccgttcgatgaactcaaacggcgattgtatcgtcgtatgaagcggatgggtatggaaatcgtcgaagaagacatcttggaagtcgaggcgagttggattccactgggcggtaccccgccggttgccccgcaacgcaccatcgcgtacggtgcagcagccggcatggtccaccctgcgtctggctactccgtcgtaaacagtattagcaaagctccgcgtgttgcgacggccatggccgaaggcttgaaggagggtggcgagattgaggcgagccgaagagcgtgggaaatcctttggggtgcggagccacgaagacaaatcggtttctaccagttcggtatggagcttctcatgtcgcttcgcatcgagcagatgcgcaacttctttagtaccttctttgcgcttccaacaaatctgagcagaggatttttgggtaacagattgtcgagctcagagttgatcatgtttgctctcactacgttcgcaattggtaacaacgaacttcgtgggttgttgctcgctcacctggtttca

SEQ ID NO: 171 - protein, 442 aa, Ostreococcus lucimarinus
MKDDREWIAFQQRKVFSEQKQIKEYLSALNDRDKVDVLVVGAGPAGLAIAAETAKKGLSVGLVAPDTPFVNNYGVWLDEFKDLGLEHCLLHKYDDALVWFDDSDPASGTELGRPYGQVCRRRLRDHLLKECAAAGVKYLPGLVDFVRHGDVEKNELAEANRGQQFTLNSRLVVAGTGHNRDMLSYEEGAPPGWQTAYGVEVRIPNHGFPVNKAVFMDFRQSDPEAMKEEQDEGVWRVPSFLYVLPVDKDVVFVEETCLVARVQVPFDELKRRLYRRMKRMGMEIVEEDILEVEASWIPLGGTPPVAPQRTIAYGAAAGMVHPASGYSVVNSISKAPRVATAMAEGLKEGGEIEASRRAWEILWGAEPRRQIGFYQFGMELLMSLRIEQMRNFFSTFFALPTNLSRGFLGNRLSSSELIMFALTTFAIGNNELRGLLLAHLVS

SEQ ID NO: 172 - DNA, 642 bp, Diospyros kaki
actacggcgtatgggaggatgaatttagagatcttggacttgaaaggtgtattgaacatgtttggagagacacaattgtatatcttgatgacaatgatcccattctgattggtcgtgcttatggacgagttagtcgtcacttgctccacgaggagctattaagaaggtgtgtggagtcaggtgtttcatatttgagctcaaaagtggaaagaattattgaaactacgaatgggcagagtctcatagagtgcggaactgatgttgttgtcccatgcaggcttgctactgttgcttcgggagcagcttctgggaaacttttgaagtttgaggtgggaggacccagagtttctgttcaaacagcttatggtgtggaggttgaggtggaaaacaatccatatgaccccaacttgatggttttcatggattacagagactatgccaaacaaaaagttcagcctttggaagcacaatatccaacatttctttatgccatgcctatgtcccctacaagagtcttctttgaggaaacttgtttggcttcaaaggatgccatgccttttgatctattaaagaggaaactcatggacagattagagacaatgggagtccatgttctaaaaacgtatgaggaggaatggtctt

SEQ ID NO: 173 - protein, 213 aa, Diospyros kaki
YGVWEDEFRDLGLERCIEHVWRDTIVYLDDNDPILIGRAYGRVSRHLLHEELLRRCVESGVSYLSSKVERIIETTNGQSLIECGTDVVVPCRLATVASGAASGKLLKFEVGGPRVSVQTAYGVEVEVENNPYDPNLMVFMDYRDYAKQKVQPLEAQYPTFLYAMPMSPTRVFFEETCLASKDAMPFDLLKRKLMDRLETMGVHVLKTYEEEWS

SEQ ID NO: 174 - DNA, 1347 bp, Artificial Sequence
  putative lycopene epsilon cyclase from Ostreococcus lucimarinus optimized for Y. lipolytica codon bias
ttctagaacaaaatgaaggacgaccgagagtggatcgccttccagcagcgaaaggtgttctctgagcagaagcagatcaaggagtacctgtctgccctgaacgaccgagacaaggtggacgtgctggtggtgggcgccggccccgccggcctggccatcgccgccgagaccgccaagaagggcctgtctgtgggcctggtggcccccgacacccccttcgtgaacaactacggcgtgtggctggacgagttcaaggacctgggcctggagcactgtctgctgcacaagtacgacgacgccctggtgtggttcgacgactctgaccccgcctctggcaccgagctgggccgaccctacggccaggtgtgtcgacgacgactgcgagaccacctgctgaaggagtgtgccgccgccggcgtgaagtacctgcccggcctggtggacttcgtgcgacacggcgacgtggagaagaacgagctggccgaggccaaccgaggccagcagttcaccctgaactctcgactggtggtggccggcaccggccacaaccgagacatgctgtcttacgaggagggcgccccccccggctggcagaccgcctacggcgtggaggtgcgaatccccaaccacggcttccccgtgaacaaggccgtgttcatggacttccgacagtctgaccccgaggccatgaaggaggagcaggacgagggcgtgtggcgagtgccctctttcctgtacgtgctgcccgtggacaaggacgtggtgttcgtggaggagacctgtctggtggcccgagtgcaggtgcccttcgacgagctgaagcgacgactgtaccgacgaatgaagcgaatgggcatggagatcgtggaggaggacatcctggaggtggaggcctcttggatccccctgggcggcaccccccccgtggccccccagcgaaccatcgcctacggcgccgccgccggcatggtgcaccccgcctctggctactctgtggtgaactctatctctaaggccccccgagtggccaccgccatggccgagggcctgaaggagggcggcgagatcgaggcctctcgacgagcctgggagatcctgtggggcgccgagccccgacgacagatcggcttctaccagttcggcatggagctgctgatgtctctgcgaatcgagcagatgcgaaacttcttctctaccttcttcgccctgcccaccaacctgtctcgaggcttcctgggcaaccgactgtcttcttctgagctgatcatgttcgccctgaccaccttcgccatcggcaacaacgagctgcgaggcctgctgctggcccacctggtgtcttaaacgcgt

SEQ ID NO: 175 - DNA, 1605 bp, Artificial Sequence
  putative lycopene epsilon cyclase from Ostreococcus lucimarinus optimized for Y. lipolytica codon bias
ttctagaacaaaatgcgagcccgacgagcccccgccgcccgagtgacccgagccatccgagcccgaggcgacgccggcacccgagcccgagacgtggcccccggcgccacccgacgaggcgcctctgccaccccccgagccacccgacgaccctctgcccgagagacccgacccgagctgtacggcctggacgcctcttgggaccccctgacctctggcgaccgacgagagtctgaggagtctcgaacccccctgcccgagaccctgcccaacgtgcgatggggcacctctgcctctgaggcctacgacctggtgatcgtgggctgtggccccgccggcctgaccgccgccgacgaggcctctaagcgaggcctgcgagtggccctgatggacccctctcccctggccccctggatgaacaactacggcgtgtggtgtgacgagttcaagtctctgggcttcgacgactgttaccgagccgtgtggaacaaggcccgagtgatcatcgacgacggcgacgccgacggcaagatgctggaccgagcctacgcccaggtggaccgaaagaagctgaagcagaagctgatcgcccgatctgtgacccagggcgtggagttcggcatcgccgccgtggactcttgtgacaactctgaccccaaccactctgtggtgaccctgtctgacggccgaaaggtgtacgccaagatggtgctggacgccaccggccactctcgaaagctggtggacttcgaccgagacttcacccccggctaccaggccgccttcggcatcgtgtgtaccgtggagaagcacgacttccccctggacaccatgctgttcatggactggcgagacgagcacctgtctcccgagttcaagcgagccaacgaccgactgcccaccttcctgtacgccatgcccttctctgagaccgaggtgttcctggaggagacctctctggtggcccgacccggcctggagttcgacgacctgaagctgaagctgaaggagcgactggactacctgggcgtgaaggtgaccaaggtgcacgaggaggagtactgtctgatccccatgggcggcgtgctgcccaccttcccccagcgaaccctgggcatcggcggcaccgccggcatggtgcacccctctaccggcttcatggtggccaagaccatgctgtgtgtgcgaaccctggtgggcaccctggacgaggccctgaaggccggcaagcgaggcgacatcaccggcgccctggaggccgccgaggccgcccagatgaacaacggcaagttcgacgccgacgccaccgccgccctggtgtggaactctatctggcccgagaacgacctgcgaatgcgaaccttcatgtgtttcggcatggagaccctgatgcagctggacatcgacggcacccgacagttcttcgacaccttcttcgacctgcccaaggacgtgtgggccggcttcctgtcttggcgaatccagcccgtgggcctgctgtctctgggcgtgaacctgttcgccctgttctctaactacatgcgagtgaacttcgtgaagtctgccctgcccttcatgggctctttcttcgccaactaaacgcgt

SEQ ID NO: 176 - DNA, 915 bp, Artificial Sequence
  putative carotene epsilon hydroxylase from Ostreococcus tauri optimized for Y. lipolytica codon bias
ttctagaacaaaatgaaggacggccaggacgaggactctgacgagatctggggcggccagcgacacgcctctgagatgaagacccccacccgacgaaaggcccgaaccaaggccgagcgagaggcctctgccgcctcttacgagtggtctgcctgggcctcttcttgtggcgtgatctctgtggccatcaccgccacctacttccgaatcctgcgagaggtggacgtgaacggcggcgtgttccccgtggccgagctggtggcccagctggccctgatcgccggcgccgccgtgggcatggagttctacgcccgatacgcccacaagcacctgtggcacggctcttggtggaccatgtctaacaagtaccgacaggagtggaaccgacccatctggctgctgcacgagtctcaccacctgccccgagagggcgccttcgaggccaacgacgtgttcgccctgatgaacggcgtgcccgccttcgccctgtgtgccttcggcttcttcacccccggcgtgttcggcggcctgtgtttcggcgccggcctgggcatcaccctgttcggcatcgcctacatgtacgtgcacgacggcctggtgcacaagcgattccccaccggccccctgggcaagctgcccgtgatgcgacgaatcgccgccggccacaccatccaccacaccgaggccttcgagggcgtgccctggggcctgttcctgggcatccaggagctggccgccgtgcccggcggcctggaggagctggagaaggtggtgatcgccgccgagcgaaaggagaagcgagacgagctggagctggcccgacgagcctctgtgggcctggtgaccgagggcgcccacatcccctctatgaaggaggccccccagtgtaagctgcccgaggacccctaaacgcgt

SEQ ID NO: 177 - protein, 1124 aa, Aspergillus nidulans
MASVLIRRKFGTEGGSDAEPSWLKRQVTGCLQSISRRACIHPIHTIVVIALLASTTYVGLLEGSLFDSFRNSNNVAGHVDVDSLLLGNRSLRLGEGTSWKWQVEDSLNQDDQKVGNPELKREVDQHLALTTLIFPDSISKSASTAPAADALPVPANASAQLLPHTPNLFSPFSHDSSLVFTLPFDQVPQFLRAVQELPDPTLEDDEGEQKRWIMRATRGPVSGPNGTISSWLSDAWSSFVDLIKHAETIDIIIMTLGYLAMYLSFASLEFSMKQLGSKFWLATTVLFSGMFAFLFGLLVTTKFGVPLNLLLLSEGLPELVTTIGFEKPIILTRAVLSASIDKKRQGSATSTPSSIQDSIQTAIREQGFEIIRDYCIEISILIAGAASGVQGGLQQFCFLAAWILFFDCILLFTFYTTILCIKLEITRIRRHVTLRKALEEDGTTQSVAEKVASSNDWFGAGSDNSDADDASVFGRKIKSNNVRRFKFLMVGGFVLVNVVNMTAIPFRNSSLSPLCNVFSPTPIDPFKVAENGLDATYVSAKSQKLELVTVVPPIKVKLEYPSVHYAKLGESQSIEIEYTDQLLDAVGGHVLNGVLKSIEDPVISKWITAVLTISIVLNGYLFNAARWSIKEPQAAPAPKEPAKPKVYPKTDLNAGPKRSMEECEAMLKAKKAAYLSDELLIELSLSGKLPGYALLKSLENEELMSRVDAFLRAVKLRRAVVSRTPATSAVTSSLETSKLPYKDYNYALVHGACCENVIGTLPLPLGVAGPLVTDGQSYFIPMATIEGVLVASASRGAKAINAGGGAVIVLTGDGMTRGPCVGFPTLARAAAAKVWLDSEEGKSVMTAAFNSTSRFARLQHLKTALAGTYLYIRFKTTTGDAMGMNMISKGVEKALHVMATECGFDDMATISVSGNFCTDKKAAALNWIDGRGKSVVAEAIIPGDVVRNVLKSDVDALVELNTSKNLIGSAMAGSLGGFNAHASNIVTAIFLATGDPAQNVESSSCITTMKNTNGNLQTAVSMPSIEVGTIGGGTILEAQGAMILDILGVRGSHPTNPGDNARQLARIVAAAVLAGFLSLCSALAAGHLVRAHMAHNRSAAPTRSATPVSAAVGATRGLSMTSSR

SEQ ID NO: 178 - protein, 1210 aa, Gibberella zeae
MASILLPKKFRGETAPAEKTTPSWASKRLTPIAQFISRLACSHPIHTVVLVAVLASTSYVGLLQESFFSTDLPTVGKADWSSLVEGSRVLRAGPETAWNWKAIEQDSIQHAGADADHLALLTLVFPDTHSAESSSTAPRSSHVPVPQNLSITPLPSTKNSFTAYSQDSILAYSLPYAEGPDVVQWANNAWTEFLDLLKNAETLDIVIMFLGYTAMHLTFVSLFLSMRKIGSKFWLGICTLFSSVFAFLFGLIVTTKLGVPISVILLSEGLPFLVVTIGFEKNIVLTRAVMSHAIEHRRQIQNSKSGKGSPERSMQNVIQYAVQSAIKEKGFEIMRDYAIEIVILALGAASGVQGGLQHFCFLAAWTLFFDFILLFTFYTAILSIKLEINRIKRHVDMRMALEDDGVSRRVAENVAKSDGDWTRVKGDSSLFGRKSSSVPTFKVLMILGFIFVNIVNICSIPFRNPRSLSTIRTWASSLGGVVAPLSVDPFKVASNGLDAILAAAKSNNRPTLVTVLTPIKYELEYPSIHYALGSAINGNNAEYTDAFHHHFQGYGVGGRMVGGILKSLEDPVLSKWIVIALALSVALNGYLFNVARWGIKDPNVPEHNIDRNELARAQQFNDTGSATLPLGEYVPPTPMRTEPSTPAITDDEAEGLQMTKARSDKLPNRPNEELEKLLAEKRVKEMSDEELVSLSMRCKIPGYALLKTLGDFTRAVKIRRSIIARNRATSDLTHSLERSKLPFEKYNWERVFCACCENVIGYMPLPVGVAGRLVTDGQSYFIPMATTEGVLVASASRGCKAINAGGGAVTVLTADGMTRGPCVAFETLERAGAAKLWIDSEAGSDIMKKAFNSTSRFARLQSMKTALAGTNLYIRFKTTTGDAMGMNIISKGVEHALSVMSNEAGFDDMQIVSVSGNYCTDKKAAALNWIDGRGKGVVAEAIIPGDVVRSVLKSDVDALVELNISKNIIGSAMAGSVGGFNAHAANIVAAIFLATGQDPAQVVESANCITLMKNLRGALQTSVSMPSLEVGTLGGGTILEPQSAMLDLLGVRGSHPTNPGDNSRRLARIIGASVLAGELSLCSALAAGHLVRAHMQHNRSAAPSRSTTPAPMTPVRSFDTKVRCQPNNKDIRNILLTQHPSKPTITYSKRVIKSTIHLNPLILALFDNSVQTRDVQLGDQVSTRGTLDAVGGPQGGGVAAGGVARRVVGS

SEQ ID NO: 179 - protein, 1173 aa, Neurospora crassa
MIASSLLPSKFRGEQPATQAATPSWINKKVTPPLQKLSKITSSNPIHTIVIVALLASSSYIGLLQNSLFNVTRSVRKAEWESLQAGSRMLRAGANTEWNWQNHDPEAPVPANANHLALLTLVFPDTAESGPVVAQTNTVPLPSNLSTTPLPSTAISFTYSQDSALAFSLPYSQAPEFLANAQEIPNAVSSQETIETERGHEKKMWIMKAARVQTRSSTVKWVQNAWVEFTDLLRNAETLDIIIMALGYISMHLTFVSLFLSMRRMGSNFWLATSVIFSSIFAFLFGLLVTTKLGVPMNMVLLSEGLPFLVVTIGFEKNIVLTRAVLSHAIDHRRPTEKSGKPSKQADSAHSIQSAIQLAIKEKGFDIVKDYAIEAGILVLGAASGVQGGLQQFCFLAAWILFFDCILLFSFYTAILCIKLFINRIKRHVQMRKALEEDGVSRRVAEKVAQSNDWPRADGKDQPGTTIFGRQLKSTHIPKFKVMMVTGFVLINVLNLCTIPFRSANSISSISSWARGLGGVVTPPPVDPFKVASNGLDIILEAARADGRETTVTVLTPIRYELEYPSTHYDLPQKSAEVEGGDYANLGGYGGRMVGSILKSLEDPTLSKWIVVALALSVALNGYLFNAARWGIKDPNVPDHPINPKELDEAQKFNDTASATLPLGEYMKPTAPSSPVAPLTPSSTDDENDAQAKENRAVTLAAQRATTIRSQGELDKMTAEKRTHELNDEETVHLSLKGKIPGYALEKTLKDFTRAVKVRRSIISRTKATTELTNILDRSKLPYQNVNWAQVHGACCENVIGYMPLPVGVAGPLVTDGQSFFVPMATTEGVLVASTSRGCKAINSGGGAVTVLTADGMTRGPCVQFETLERAGAAKLWLDSEKGQSIMKKAFNSTSRFRALETMKTAMAGTNLYIRFKITTGDAMGMNMISKGVEHALSVMYNEGFEDMNIVSLSGNYCTDKKAAAINVIDGRGKSVVAEAIIPADVVKNVLKTDVDTLVELNVNKNTIGSAMAGSMGGFNAHAANIVAAIFLATGQDPAQVVESANCITLMRNLRGNLQISVSMPSIEVGTLGGGTILEPQSAMLDMLGVRGPHPTNPGENARRLARIVAAAVLAGELSLCSALAAGHLVKAHMAHNRSAPPTRTSTPAPAAAAGLTMTSSNPNAAAVERSRR

SEQ ID NO: 180 - protein, 1045 aa, Saccharomyces cerevisiae
MSLPLKTIVHLVKPFACTARFSARYPIHVIVVAVLLSAAAYLSVTQSYLNEWKLDSNQYSTYLSIKPDELFEKCTHYYRSPVSDTWKLLSSKEAADIYTPFHYYLSTISFQSKDNSTTLPSLDDVIYSVDHTRYLLSEEPKIPTELVSENGTKWRLRNNSNFILDLHNIYRNMVKQFSNKTSEFDQFDLFIILAAYLTLFYTLCCLFNDMRKIGSKFWLSFSALSNSACALYLSLYTTHSLLKKPASLLSLVIGLPFTVVIIGEKHKVRLAAFSLQKFHRISIDKKITVSNIIYEAMFQEGAYLIRDYLFYISSFIGCAIYARHLPGLVNFCILSTFMLVFDLLLSATFYSAILSMKLFINIIHRSTVIRQTLEEDGVVPTTADIIYKDETASEPHFLRSNVAIILGKASVIGLLLLINLYVFTDKLNATILNTVYFDSTIYSLPNFINYKDIGNLSNQVIISVLPKQYYTPLKKYHQIEDSVLLIIDSVSNAIRDQFISKLLFFAFAVSISINVYLLNAAKIHTGYMNFQPQSNKIDDLVVQQKSATIEFSETRSMPLASGLETPVTAKDIIISEEIQNNECVYALSSQDEPIRPLSNLVELMEKEQLKNMNNTEVSNLVVNGKLPLYSLEKKLEDTLRAVLVRRKALSTLAESPILVSEKLPFRNYDYDRVFGACCENVIGYMPIPVGVIGPLITDGTSYHIPMATTEGCLVASAMPGCKAINAGGGATTVLTKDGMTRGPVVRFPTLIRSGACKIWLDSEEGQNSIKKAFNSTSRFARLQHTQTCLAGDLLFMRFRTTTGDAMGMNMISKGVEYSLKQMVEEYGWEDMEVVSVSGNYCTDKKPAAINWIEGRGKSVVAEATIPGDVVKSVLKSDVSALVELNISKNIVGSAMAGSVGGFNAHAANLVTALFLALGQDPAQNVESSNCITLMKEVDGDLRISVSMPSIEVGTIGGGTVLEPQGAMLDLLGVRGPHPTEPGANARQLARIIACAVLAGELSLCSALAAGHLVQSHMTHNRKTNKANELPQPSNKGPPCKTSALL

SEQ ID NO: 181 - protein, 999 aa, Yarrowia lipolytica
MLQAAIGKIVGFAVNRPIHTVVLTSIVASTAYLALLDIAIPGEEGTQPISYYHPAAKSYDNPADWTHIAEADIPSDAYRLAFAQIRVSDVQGGEAPTIPGAVAVSDLDHRIVMDYKQWAPWTASNEQIASENHIWKHSFKDHVAFSWIKWFRWAYLRLSTLIQGADNFDIAVVALGYLAMHYTFFSLFRSKRKVGSHFWLASMALVSSFFAFLLAVVASSSLGYRPSMITMSEGLPFLVVAIGFDRKVNLASEVLTSKSSQLAPMVQVITKIASKALFEYSLEVAALFAGAYTGVPRLSQFCFLSAWILIFDYMFLLTFYSAVIAIKFLINHIKFNRMIQDALKEDGVSAAVAEKVADSSPDAKLDRKSDVSLFGASGAIAVFKIFMVLGFLGLNLINLTAIPHLGKAAAAAQSVTPITLSPELLHAIPASVPVVVTFVPSVVYEHSQLILQLEDALTFFLAACSKTIGDPVISKYIFLCLMVSTALNVYLFGATREVVRTQSVKVVEKHVPIVIEKPSEKEEDTSSEDSIELTVGKQPKPVTETRSLDDLEATMKAGKTKLLEDHEVVKLSLEGKLPLYALFKQLGDNTRAVGIRRSIISQQSNTKTLETSKLPYLHYDYDRVFGACCENVIGYMPLPVGVAGPMNTDGKNYHIPMATTEGCLVASTMRGCKAINAGGGVTTVLTQDGMTRGPCVSFPSLKRAGAAKIWLDESEGLKSMRKAFNSTSRFARLQSLHSTLAGNLLFIRFRTTTGDAMGMNMISKGVEHSLAVMVKEYGFPLMDIVSVSGNYCTDKKPAAINWIEGRGKSVVAEATIPAHIVKSVLKSEVDALVELNISKNLIGSAMAGSVGGFNAHAANLVTAIYLATGQDPAQNVESSNCITLMSNVDGNLLISVSMPSIEVGTIGGGTILEPQGAMLEMLGVRGPHIETPGANAQQLARIIASGVLAAELSLCSALAAGHLVQSHMTHNRSQAPTPAKQSQADLQRLQNGSNICIRS

SEQ ID NO: 182 - protein, 1199 aa, Artificial Sequence
  synthetic consensus
MASXLLXXREXXEXXXXXXAXPSWXXKXLTXPIGXISRFAAXHPIHTIVLVALLASTAYLGLLQXSLFXWXLXSNXXXXXXDXTSLXXGSRXLRXGXXTXWRWXXIDXXXIXXXXXXXXXXXAADAXHLALXTLVFPDTQSXEXASTIPXAXXVPVPXNXSIXLLPXTXXIFTXYSQDSSLXFSLPYSXXXXXXXXXXXXXXXXXXXXXXXXXXXXXXXXXXXXXXXXXXXDIVXWXXNAWXXFSDLIKNADTFDIIIMNLGYLAMHYTFNSLFNSMRKLGSKFWLATSXLFSSIFAFLLGLLVTTKLGXVPISMLLLSEGLPFLVVTIGFEKKIVLTRAVLSXAIDXRRXXXXXXXXXXXXXXSXXXSIGXAIQXAIKEXGFEIIRDYAIEISILIAGAASGVQGGXLXQFCFLAAWILFFDXILLFTFYSAILAIKLEINRIKRHVIIRXALEEDGVSXSVAEKVAKSEXDWXXXKGSDSXFXXXXXXXXXXKSXAIXIFKVLMILGFVLINLVNLTAIPFRNXAXSXXXXXXXXXXLXFVXSPXXVDPFKVAXNLLDAXAAAKSXXRETLVTVVTPIKYELEYPSIHYXEXXXSXXXXXXXXLXXXXXXXXGXGGXMLGSVSKSIEDPVISKWIVIALALSIALNVYLFNAARWXIKDPNVXXXXXEVXELXXXQXXNXXXSAXLXXXXXIXXTXXXXXXXXXTPAXTDDEXXSXXSXXXXVXKIXXXXXXIRSLEELEALLAAKKTKXLXDEEVVXLSLXGKLPLYALEKTLGXXXXXXXXXDFTRAVKIRRSIISRXXATSALTXSLESSKLPYKNYNYDRVFGACCENVIGYMPLPVGVAGPLVIDGQSYHIPMATTEGVLVASASRGCKAINAGGGAVTVLTADGMTRGPCNXFPTLXRAGAAKIWLDSEEGQXSMKKAFNSTSRFARLQHIKTALAGTLLFIRFKTTTGDAMGMNMISKGVEHALSVMVXEYGFEDMEIVSVSGNYCTDKKPAAINWIDGRGKSVVAEATIPGDVVKSVLKSDVDALVELNISKNLIGSAMAGSVGGFNAHAANIVTAIFLATGQDPAQNVESSNCITLMKNVDGNLQISVSMPSIEVGTIGGGTILEPQGAMLDLLGVRGPHPTNPGDNARQLARIIAAAVLAGELSLCSALAAGHLVQAHMTHNRSAAPTRSNTPXXAXXXXXXTXIXS

SEQ ID NO: 183 - DNA, 34 bp, Artificial Sequence
  synthetic oligonucleotide primer MO4890
ctctagacacaaaaatgtcgcaaccccagaacgt

SEQ ID NO: 184 - DNA, 27 bp, Artificial Sequence
  synthetic oligonucleotide primer MO4891
cacgcgtctactgcttgatctcgtact

SEQ ID NO: 185 - DNA, 36 bp, Artificial Sequence
  synthetic oligonucleotide primer MO4982
cgctagccacaaaaatggactacatcatttcggcgc

SEQ ID NO: 186 - DNA, 27 bp, Artificial Sequence
  synthetic oligonucleotide primer MO4983
cacgcgtctaatgggtccagggaccga

SEQ ID NO: 187 - DNA, 35 bp, Artificial Sequence
  synthetic oligonucleotide primer MO4984
ctctagacacaaaaatgaccacctattcggctccg

SEQ ID NO: 188 - DNA, 29 bp, Artificial Sequence
  synthetic oligonucleotide primer MO4985
cggcgcgccctacttgaaccccttctcga

SEQ ID NO: 189 - DNA, 36 bp, Artificial Sequence
  synthetic oligonucleotide primer MO4986
ctctagacacaaaaatgatccaccaggcctccacca

SEQ ID NO: 190 - DNA, 27 bp, Artificial Sequence
  synthetic oligonucleotide primer MO4987
cacgcgtctacttgctgttcttcagag

SEQ ID NO: 191 - DNA, 34 bp, Artificial Sequence
  synthetic oligonucleotide primer MO4988
ctctagacacaaaaatgacgacgtcttacagcga

SEQ ID NO: 192 - DNA, 27 bp, Artificial Sequence
  synthetic oligonucleotide primer MO4989
cacgcgtctacttgatccaccgccgaa

SEQ ID NO: 193 - DNA, 35 bp, Artificial Sequence
  synthetic oligonucleotide primer MO4990
ctctagacacaaaaatgtccaaggcgaaattcgaa

SEQ ID NO: 194 - DNA, 27 bp, Artificial Sequence
  sythetic oligonucleotide primer MO4991
cacgcgtctacttctgtcgcttgtaaa

SEQ ID NO: 195 - DNA, 34 bp, Artificial Sequence
  synthetic oligonucleotide primer MO4992
ctctagacacaaaaatgttacgactacgaaccat

SEQ ID NO: 196 - DNA, 27 bp, Artificial Sequence
  synthetic oligonucleotide primer MO4993
cacgcgtctagtcgtaatcccgcacat

SEQ ID NO: 197 - DNA, 34 bp, Artificial Sequence
  synthetic oligonucleotide primer MO4996
cgctagccacaaaaatgccgcagcaagcaatgga

SEQ ID NO: 198 - DNA, 27 bp, Artificial Sequence
  synthetic oligonucleotide primer MO4997
cacgcgtttaaccatgcagccgctcaa

SEQ ID NO: 199 - DNA, 34 bp, Artificial Sequence
  synthetic oligonucleotide primer MO5000
ctctagacacaaaaatgttccgaacccgagttac

SEQ ID NO: 200 - DNA, 27 bp, Artificial Sequence
  synthetic oligonucleotide primer MO5001
cacgcgtttaagggttctgcttgacaa

SEQ ID NO: 201 - DNA, 34 bp, Artificial Sequence
  synthetic oligonucleotide primer MO5004
ctctagacacaaaaatgacacaaacgcacaatct

SEQ ID NO: 202 - DNA, 27 bp, Artificial Sequence
  synthetic oligonucleotide primer MO5005
cacgcgtttacatcttgtacgcagggt

SEQ ID NO: 203 - DNA, 34 bp, Artificial Sequence
  synthetic oligonucleotide primer MO5008
ctctagacacaaaaatggaagccaaccccgaagt

SEQ ID NO: 204 - DNA, 27 bp, Artificial Sequence
  synthetic oligonucleotide primer MO5009
cacgcgttcatttcagaaggtacttct

SEQ ID NO: 205 - DNA, 32 bp, Artificial Sequence
  synthetic oligonucleotide primer MO4852
ctctagacacaaaaatgcgactcactctgccc

SEQ ID NO: 206 - DNA, 29 bp, Artificial Sequence
  synthetic oligonucleotide primer MO4853
cacgcgtctactcgacagaagagaccttc

SEQ ID NO: 207 - DNA, 37 bp, Artificial Sequence
  synthetic oligonucleotide primer MO4741
ttctagacccaaaaatgtctgccaacgagaacatctc

SEQ ID NO: 208 - DNA, 29 bp, Artificial Sequence
  synthetic oligonucleotide primer MO4743
aacgcgtctatgatcgagtcttggccttg

SEQ ID NO: 209 - DNA, 37 bp, Artificial Sequence
  synthetic oligonucleotide primer MO4760
ttctagacacaaaaatgtcagcgaaatccattcacga

SEQ ID NO: 210 - DNA, 26 bp, Artificial Sequence
  synthetic oligonucleotide primer MO4861
cacgcgttaaactccgagaggagtgg

SEQ ID NO: 211 - DNA, 34 bp, Artificial Sequence
  synthetic oligonucleotide primer MO4994
ctctagacacaaaagtggttaaagctgtcgttgc

SEQ ID NO: 212 - DNA, 27 bp, Artificial Sequence
  synthetic oligonucleotide primer MO4995
cacgcgtttacttggcaggaggagggt

SEQ ID NO: 213 - DNA, 34 bp, Artificial Sequence
  synthetic oligonucleotide primer MO4998
ctctagacacaaaaatgactggcaccttacccaa

SEQ ID NO: 214 - DNA, 27 bp, Artificial Sequence
  synthetic oligonucleotide primer MO4999
cacgcgttcacgaggagcccttggtga

SEQ ID NO: 215 - DNA, 34 bp, Artificial Sequence
  synthetic oligonucleotide primer MO5002
ctctagacacaaaaatgactgacacttcaaacat

SEQ ID NO: 216 - DNA, 27 bp, Artificial Sequence
  synthetic oligonucleotide primer MO5003
cacgcgtttaagcatcgtaagtggaag

SEQ ID NO: 217 - DNA, 34 bp, Artificial Sequence
  synthetic oligonucleotide primer MO5006
ctctagacacaaaaatgctcaaccttagaaccgc

SEQ ID NO: 218 - DNA, 27 bp, Artificial Sequence
  synthetic oligonucleotide primer MO5007
cacgcgtctacttgagtcgcttgataa